(19) United States
(12) Patent Application Publication
(10) Pub. No.: US 2011/0104052 A1
(43) Pub. Date: May 5, 2011

(54) METHODS OF SYNTHESIS AND USE OF CHEMOSPHERES

(75) Inventors: Bradley Powers Barnett, Baltimore, MD (US); Jeffrey Geschwind, Baltimore, MD (US)

(73) Assignee: THE JOHNS HOPKINS UNIVERSITY, Baltimore, MD (US)

(21) Appl. No.: 12/746,030

(22) PCT Filed: Dec. 3, 2008
(86) PCT No.: PCT/US08/13336

§ 371 Date: Jan. 20, 2011

Related U.S. Application Data
(60) Provisional application No. 61/005,228, filed on Dec. 3, 2007.
(60) Provisional application No. 61/093,619, filed on Sep. 2, 2008.

(51) Int. Cl. A61K 51/12; A61K 9/14; A61K 49/18; A61K 35/12; A61K 39/395; A61K 9/127; A61P 7/04; A61P 35/00; A61K 31/704; B82Y 5/00

(52) U.S. Cl. 424/1.21; 424/484; 424/1.25; 424/9.1; 424/93.7; 424/130.1; 424/1.37; 424/450; 514/34; 977/773; 977/906; 977/927

(57) ABSTRACT

The present invention provides, in general, compositions comprising a hydrogel and an agent, for example a therapeutic agent or an imaging agent, for locoregional delivery. In certain preferred embodiments of the invention, the hydrogel compositions are detectable by Magnetic Responance and CT Scan and are used for locoregional delivery of therapeutic agents, for example chemotherapeutic agents. The invention also features polymer matrix compositions comprising nanoparticles that can be loaded after polymerization with bioactive agents, for example a diagnostic agent or therapeutic agent.

### PRIORITY

This application claims priority to U.S. Provisional Application No. 61/005,228, as filed on Dec. 3, 2007, and to U.S. Provisional Application No. 61/093,619, as filed on Sep. 2, 2008. The disclosures of both Applications are incorporated herein by reference in their entireties.

### INCORPORATION BY REFERENCE

Each of the applications and patents cited in this text, as well as each document or reference cited in each of the applications and patents (including during the prosecution of each issued patent; “application cited documents”), and each of the PCT and foreign applications or patents corresponding to and/or paragraphing priority from any of these applications and patents, and each of the documents cited or referenced in each of the application cited documents, are hereby expressly incorporated herein by reference. More generally, documents or references are cited in this text, either in a Reference List, or in the text itself; and, each of these documents or references (“herein-cited references”), as well as each document or reference cited in each of the herein-cited references (including any manufacturer's specifications, instructions, etc.), is hereby expressly incorporated herein by reference.

### BACKGROUND OF THE INVENTION

The controlled release of low-molecular-weight species from polymer-based devices via a diffusion process is well understood. Accordingly, hydrogels have been investigated extensively for potential applications in the biomedical field since their water content is similar to soft tissues, and because of their good record of biocompatibility to tissues. Biodegradable hydrogels have two main advantages. First, there is no need to remove residual biomaterials from the implant site. Secondly, biodegradable hydrogels allow a wider range of drug release profiles. Biodegradable hydrogels permit the entrapped therapeutic factor to be released in a controlled manner through both drug diffusion and hydrogel degradation.

Poly(ethylene glycol) (PEG) is widely used as a water soluble carrier for polymer-drug conjugates. Photopolymerization is a technique that employs light to generate radicals from photoinitiators, which will further react with the active end group on polymers to form a covalent crosslink. Compared with other approaches, photopolymerized hydrogel systems provide better temporal and spatial control over the gelation process; however such a system has not been successfully applied to in vivo therapeutic uses.

Many current regimens to treat disease, while efficacious in treating or killing the disease (e.g. cancer cells) result in deleterious side effects to the subjects. For instance, previous reports using a rabbit model for liver cancer have shown that 3-bromopyruvate in a single bolus intraarterial injection kills up to 90% of the tumor cells within tumors growing within the liver; however though bolus injection provides good tumor kill it has also damages surrounding liver tissue. A delivery strategy to minimize damage to non-cancerous tissue would provide a significant advantage to current techniques. Such a delivery strategy may minimize damage to healthy tissue while enabling enhanced tumor kill.

Accordingly, a need remains in the art for delivery strategies that minimize damage to healthy tissue while treating and eliminating diseased or target tissue.

Moreover, as therapeutic agent-eluting microspheres and stents show promise in the burgeoning field of interventional radiology, many therapeutics in the current clinical armaterium are unstable at the high temperatures required for traditional microsphere synthesis, and so current commercially available drug eluting beads are loaded after synthesis. Commonly, loading is based upon ionic interaction between a charged drug and an oppositely charged microsphere. Such a technique limits loading of spheres with charged compounds. Further, preliminary studies have shown much of the drug that is loaded on the sphere by ionic interaction is not eluted under physiological conditions. Alternate strategies of loading spheres in order to provide more precise dosing and higher percentage of drug elution are needed.

An alternate strategy for enabling slow release of agent from a polymer is through incorporation of nanoparticles in the polymer that interact with the therapeutic agent. In such a system a synergistic effect is achieved between the polymer and nanoparticle components thereby enabling a slower release profile of therapeutic agents that either polymer or nanoparticle alone.

A post loading-strategy, for example of pre-formed microspheres, can be preferable for numerous reasons. For example, such a design strategy could enable loading of preformed microspheres containing liposomes by the pharmacy or clinician just prior to administration to patient thereby largely circumventing the current issues of shelf-life with preloaded formulations. Additionally, such a design strategy gives the pharmacy and clinician the ability to load a variety of therapeutic agents including numerous therapeutic agents and provides the ability to control the relative concentration of each therapeutic agent in the liposome.

Several methods have been developed for liposome loading but to date no methods have been described for loading a hybrid polymer liposome matrix. Current commercially available loadable drug eluting beads rely on direct chemical or ionic interaction between the drug and the polymer. Commonly, the transmembrane potential is created by a concentration gradient which is formed by having differing concentrations of a particular species on either side of the liposomal membrane. Neutralization of the concentration gradient is coupled to flow of the substance being loaded into the liposome. pH gradients (U.S. Pat. Nos. 4,946,683; 5,192,549; 5,204,112; 5,262,168; 5,380,531, incorporated by reference in their entireties herein), Na+/K+ gradients (U.S. Pat. Nos. 5,171,578; 5,077,056) and NH4+ gradients (U.S. Pat. No. 5,316,771) have been used to load a variety of drugs into liposomes. One limitation of using ion gradients is that the substance being loaded must be an ionizable or protonatable substance. Therefore, the substances loaded by these methods are typically ionizable compounds, often weakly acidic or basic or amphipathic molecules. Other chemical potential driven methods for liposome loading after liposome formation have used a concentration gradient of the solute itself to drive the loading process by employing precursor liposomes with low ionic strength interiors and raising the temperature above the crystal/liquid transition temperature Tc or temporarily disrupting the liposome membrane with shear stresses. Despite the availability of these methods for liposome loading, it is desirable to describe novel formulations and methods that enable loading of polymer and liposome matrices. This invention fulfills this and other needs.

### SUMMARY OF THE INVENTION

The present invention provides, in general, compositions comprising a hydrogel and an agent, for example a therapeutic agent or an imaging agent, for locoregional delivery. In certain preferred embodiments of the invention, the hydrogel compositions are detectable by Magnetic Responance and CT Scan and are used for locoregional delivery of therapeutic agents, for example chemotherapeutic agents 3-Bromopyruvate and Doxorubicin.

The experiments described herein feature new techniques for enabling loading of liposome polymer matrices by a creating a transmembrane potential. The experiments demonstrate that the combination of polymer and liposome has an unexpected effect of synerginicity in which compound is released slower from a polymer/liposome matrix at a far reduced rate as compared to a polymer or liposome.

The experiments described herein demonstrate that the combination of polymer and liposome has an unexpected effect of synerginicity in which compound is released slower from a polymer/liposome matrix at a far reduced rate as compared to a polymer or liposome.

In one embodiment, the invention described herein relies upon polymers that are relatively inert once polymerized and thus do not crosslink with the drug to be loaded. Alone, these polymers when loaded with small mw therapeutic agents result in a burst release of therapeutic agents. When liposomes are incorporated into these polymers on the other hand they act as local slow release depots within the polymer thereby drastically reducing the rate of drug release. Further, when liposomes are incorporated into polymer a synergistic effect is achieved in that the liposomes are largely immunoisolated thereby preventing the normal means of immunoclearance that occurs with free liposomes. Though pegylating liposomes has served to increase circulating time of liposomes, the liposome polymer matrices can not be readily phagocytized and thus can persist for extended periods in the body thereby maximizing the time of drug release.

A particularly unexpected feature of the present invention is the synergicitiy of extended drug release from liposome loaded hydrogels. This effect is due to stabilization of liposomes with a hydrogel scaffold. Another novel feature as described herein is that this hydrogel scaffold prevents immunoclearance of liposomes by reticuloendothelial system.

In another aspect, the invention features a polymer matrix comprising nanoparticles, wherein the nanoparticles are loaded after polymerization with one or more bioactive agents.

The term “matrix” is meant to refer to any polymer that can entrap nanoparticles. In preferred embodiments, the polymer is formed by mixing nanoparticles with unpolymerized monomeric units and inducing polymerization. In exemplary embodiments, the polymer is a hydrogel. In certain preferred examples, nanoparticles are premixed with unpolymerized monomeric units, polymerization is induced and post polymerization entrapped nanoparticles are loaded with therapeutic agent. In one embodiment, the one or more bioactive agents is selected from the group consisting of a diagnostic agent, an imaging agent, a contrast agent, a radioactive isotope, and a therapeutic agent.

In one embodiment, the radioactive isotope is selected from iodine-131, cobalt-60, iridium-192, yttrium-90, strontium-89, samarium-153, rhenium-186, technetium-99 m, and any combination thereof.

In another embodiment, the nanoparticles irreversibly interact with the bioactive agent. In a related embodiment, the nanoparticles reversibly interact with the bioactive agent.

In one embodiment, the nanoparticles are undecorated metallic nanoparticles. In another embodiment, the nanoparticles are decorated metallic nanoparticles. In a further related embodiment, the decorated metallic nanoparticles are decorated with one or more elements selected from the group consisting of dextran, PEG, streptavidin, biotin, antibodies, antibody fragments, ligands and aptamers.

In another embodiment, the therapeutic agent is operably linked to the nanoparticles through ionic interaction. In still another embodiment, the therapeutic agent is operably linked to said nanoparticles through covalent interaction.

In still another embodiment, the nanoparticle is also a contrast agent.

In another embodiment, the bioactive agent is conjugated to biotin.

In a related embodiment, the biotin conjugated bioactive agent is selected from the group consisting of yttrium 90-y biotin, biotin conjugated P-32, P-33, Sc-47, Cu-64, Cu-67, As-77, Ph-105, Pd-109, Ag-111, 1-125, Pr-143, Sm-153, Tb-161, Ho-166, Lu-177, Re-186, Re-188, Re-189, Ir-194, Au-199, Pb-212, and Bi-213.

In one embodiment, the nanoparticles are loaded after entrapment in polymer. In a further related embodiment, the polymer matrix composition comprises streptavidin coated nanoparticles that are loaded after entrapment in polymer with a biotin conjugated therapeutic agent.

In still another embodiment, the matrix forms a microsphere. In related embodiments, the polymer matrix forms radioactive microspheres.

In another aspect, the invention features a radioactive stent for preventing restenosis comprising a polymer matrix composition comprising nanoparticles that can be loaded after polymerization with a diagnostic agent or therapeutic agent.

In another aspect, the invention features a tubular device comprising a polymer matrix composition coating comprising nanoparticles that can be loaded after polymerization with a diagnostic agent or therapeutic agent.

In one embodiment, the polymer matrix further comprises a radioisotope, potential radioactive isotope, a chemotherapeutic agent, or any combination thereof.

In one embodiment, the potentially radioactive isotope is an isotope possessing a high absorption cross-section to neutrons, protons, electrons, or high energy photons. In a related embodiment, the potentially radioactive isotope has a high absorption cross-section to neutrons, and is selected from the group consisting of boron-10, -samarium-149, gadolinium-157, and gadolinium-155 or any combination thereof.

In another embodiment, the invention features a method of administering the polymeric matrix of the aspects described herein to a subject thereby reducing the rate of clearance of nanoparticle compared to a non-polymer entrapped nanoparticle as the nanoparticle is immunoisolated from antibody and direct phagocytosis by teh polymer membrane.

The invention features in another aspect a method of producing a therapeutic polymer matrix comprising nanoparticles loaded with one or more bioactive agents comprising polymerizing a matrix comprising polymers and nanoparticles; and incubating the nanoparticles and the polymer matrix after polymerization with one or more bioactive agents for a time sufficient to load the nanoparticle and polymer matrix with the agent, thereby producing a polymer matrix comprising nanoparticles loaded with one or more bioactive agents.

In one embodiment, the method further comprises the steps of administering the nanoparticles to a subject prior to loading the nanoparticles with bioactive agent.

In another embodiment, the nanoparticles are administered systemically.

In another aspect, the invention features a method of producing a therapeutic polymer matrix in a subject tissue comprising administering a nanoparticle and polymer matrix to a subject; and loading the nanoparticles with a bioactive agent, thereby producing a therapeutic polymer matrix in a subject tissue.

In another aspect, the invention features a method of producing a therapeutic polymer matrix composition in a patient tissue comprising streptavidin coated gold nanoparticles operably linked to a yttrium 90-y biotin conjugated diagnostic or therapeutic agent comprising the steps of first administering the nanoparticle/polymer matrix to a patient in need thereof, and then loading a therapeutic agent to the nanoparticles within the nanoparticle/polymer matrix by site directed administration.

In one embodiment, the method further comprises the step of emobilizing polymer microspheres containing streptavidin coated nanoparticles in a patient tumor and systemically infusing yttrium 90-y biotin into the patient so that yttrium 90-y biotin accumulates at the site of embolization and interacts with the streptavidin coated nanoparticles.

In one embodiment, loading of the nanoparticles within the nanoparticle and polymer matrix is by site directed administration.

In another embodiment of the above aspects, the bioactive agent is selected from one or more of the group consisting of: a diagnostic agent, an imaging agent, a contrast agent, a radioactive isotope, and a therapeutic agent.

In another embodiment, the polymer matrix comprises streptavidin coated gold nanoparticles linked to a yttrium 90-y biotin conjugated bioactive agent.

In another aspect, the invention features a method of emobilizing polymer microspheres containing streptavidin coated nanoparticles in a patient tumor comprising systemically infusing yttrium 90-y biotin into the patient so that yttrium 90-y biotin accumulates at the site of embolization and interacts with the streptavidin coated nanoparticles.

In one embodiment, the polymer is alginate.

In another embodiment, the method further comprises the step of dissolving non-target embolization by alginate lyase and EDTA prior to delivery of therapeutic agent.

In another embodiment, the diagnostic or therapeutic agent is delivered to the patient in need thereof at multiple time points to allow repeated accumulation of drug on the polymer matrix in order to produce an effective amount of therapeutic drug on the polymer matrix. In another embodiment of any one of the aspects described herein the nanoparticle is a liposome with an aqueous core. In a related embodiment, the polymer matrix contains a plurality of liposomes and a polymer core.

In another embodiment, the liposome comprises elements from the group consisting of fatty acids, fatty acids, derivatives, mono-, di and triglycerides, phospholipids, sphingolipids, cholesterol and steroid derivatives, oils, vitamins and terpenes including but not limited to egg yolk L-phosphatidylcholine (EPC), 1,2-dimyristoyl-sn-glycero-3-phosphatidylcholine (DMPC), 1,2-dipalmitoyl-sn-glycero-3-phosphatidylcholine (DPPC), 1,2-dioleoyl-sn-glycero-3-phosphatidylcholine (DOPC), 1,2-distearoyl-sn-glycero-3-phosphatidylcholine (DSPC), 1,2-dilauroyl-sn-glycero-3-phosphatidylcholine (DLPC), 1,2-dioleoyl-sn-glycero-3-phosphaethanolamine (DOPE), 1-palmitoyl-2-oleoyl-sn-glycero-3-phosphaethanolamine (POPE), 1,2-dimyristoyl-sn-glycero-3-phosphaethanolamine (DMPE), 1,2-dipalmitoyl-sn-glycero-3-phosphaethanolamine (DPPE), and 1,2-distearoyl-sn-glycero-3-phospharthanolamine (DSPE), phosphatidic acids, phosphatidyl cholines with both saturated and unsaturated lipids, phosphatidyl ethanolamines, phosphatidylglycerols, phosphatidylserines, phosphatidylinositols, lysophosphatidyl derivatives, cardiolipin, 13-acyl-y-alkyl phospholipids, dioleoylphosphatidylcholine, dimyristoylphosphatidylcholine, dipentadecanoylphosphatidylcholine, dilauroylphosphatidylcholine, dipalmitoylphosphatidylcholine; distearoylphosphatidylcholine, diarachidoylphosphatidylcholine, dibehenoylphosphatidylcholine, ditricosanoylphosphatidylcholine, dilignoceroylphatidylcholine; and phosphatidylethanolamines.

In another embodiment, the matrix is formed of a polymer selected from the group consisting of poly(acrylamide), poly(N-isopropylacrylamide), poly(N-isopropylacrylamide-co-1-vinylimidazole), poly(N,N-dimethylacrylamide), poly(N,N-diethylacrylamide), poly(1-vinylimidazole), poly(sodium acrylate), poly(sodium methacrylate), poly(2-hydroxyethylmethacrylate) (HEMA), poly(N,N-dimethylaminoethyl methacrylate) (DMAEMA), poly(N tris(hydroxymethyl)methylacrylamide), poly(1-(3-methacryloxy)propylsulfonic acid) (sodium salt), poly(allylamine), poly(N-acryloxysuccinimide), poly(N-vinylcaprolactam), poly(1-vinyl-2-pyrrolidone), poly(2-acrylamido-2-methyl-1-propanesulfonic acid) (sodium salt), poly((3-acrylamidopropyl) trimethylammonium chloride), and poly(diallyldimethylammonium chloride), poly(hydroxy acids), polyanhydrides, polyorthoesters, polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polysiloxanes, poly(vinyl alcohols), poly(vinyl acetate), polystyrene, polyurethanes and co-polymers thereof, synthetic celluloses, polyacrylic acids, poly(butyric acid), poly(valeric acid), and poly(lactide-co-caprolactone), ethylene vinyl acetate, copolymers and blends thereof.

In another aspect, the invention features a method of producing a polymer matrix composition comprising nanoparticles comprising initiating polymerization—with a cross-linker selected from a group consisting of —N,N-methylenebisacrylamide, N,N-(1,2-dihydroxyethylene)bisacrylamide, ethylene glycol diacrylate, di(ethylene glycol) diacrylate, tri(ethylene glycol) diacrylate, tetra(ethylene glycol) diacrylate, ethylene glycol dimethacrylate, di(ethylene glycol) dimethacrylate, tri(ethylene glycol) dimethacrylate, tetra(ethylene glycol) dimethacrylate, and pentaerythritol triacrylate.

In another aspect, the invention features a method of producing a polymer matrix composition comprising nanoparticles comprising adding a photoinitiator selected from the group consisting of 2,2-diethoxyacetophenone, 2,2-dimethoxy-2-phenylacetophenone (IRGACURE 651), 1-4-(2-hydroxyethoxy)phenyl-2-hydroxy-2-methyl-1-prop an-1-one (IRGACURE 2959), 2-hydroxy-2-methylpropiophenone, and 2-hydroxy-4-(2-hydroxyethoxy)-2-methylpropiophenone, initiating polymerization of the matrix through exposure to a UV or visible light source f, wherein exposure of the matrix and photoinitator causes polymerization of the matrix.

In one embodiment, polymerization is achieved through by a redox initiator, and wherein the redox initiator is selected from a group consisting of ammonium persulfate, potassium persulfate, 2,2-azobis2-methyl-N-(2-hydroxyethyl)propionamide (VA-086), 2,2-azobis(2-amidinopropane)dihydrochloride (V-50), 4,4-azobis(4-cyanovaleric acid). A method of producing a polymer matrix composition comprising nanoparticles comprising the step of: adding a redox initiator, and wherein the redox initiator is selected from a group consisting of ammonium persulfate, potassium persulfate, 2,2-azobis2-methyl-N-(2-hydroxyethyl)propionamide (VA-086), 2,2-azobis(2-amidinopropane)dihydrochloride (V-50), 4,4-azobis(4-cyanovaleric acid), wherein exposure of the matrix to the redox initiator causes polymerization of the matrix.

In another aspect, the invention features a method of producing a polymer matrix comprising nanoparticles wherein the nanoparticles are liposomes with an aqueous core comprising producing a liposome suspension; and forcing the liposome suspension through a calibrated porous membrane before introduction of the liposome into the polymer, thereby producing a polymer matrix comprising nanoparticles wherein the nanoparticles are liposomes with an aqueous core.

In one embodiment, the method further comprises the step of forming liposome vesicles under agitation, wherein the average size of the liposome vesicles obtained by agitation is inversely proportional to the degree of agitation.

In another embodiment, the matrix consists of a single liposome with a polymer core.

In another embodiment, the method, further comprises the step of preloading an agent into liposomes prior to liposome incorporation into polymer.

In still another embodiment, the method further comprises the step of loading an agent into liposomes after lipid and polymer are combined.

In one embodiment, the matrix consists of a single liposome with a polymer core.

In another embodiment, each liposome surrounds polymeric subunits that are polymerized after liposome synthesis.

In another related embodiment, the liposome contains activated groups in the membrane allowing conjugation of proteins or other receptor affinic molecules to these activated groups.

In still another embodiment, the liposomes comprise activated groups in the membrane conjugated to receptor binding proteins.

In one embodiment, liposomes are conjugated with an IgM or IgG class antibody, a fragment thereof or a construct from the IgM or IgG classes of antibodies.

In another embodiment, the receptor binding proteins a monoclonal or a polyclonal antibody. In a related embodiment, the antibody, antibody fragment or construct conjugated to the liposomes is selected from the group consisting of a murine antibody, chimeric antibody, human monoclonal or polyclonal antibody.

In another embodiment, the aqueous core is water.

In another aspect, the invention features a method of producing a polymer matrix composition comprising nanoparticles wherein the nanoparticles are liposomes with an aqueous core, comprising the steps of: preloading an agent into liposomes prior to liposome incorporation into polymer.

In another aspect, the invention features a method of producing a polymer matrix composition comprising nanoparticles wherein the nanoparticles are liposomes with an aqueous core, comprising the steps of: loadingan agentinto liposomes after lipid and polymer are combined.

In another aspect, the invention features a method of producing a polymer matrix composition comprising nanoparticles wherein the nanoparticles are liposomes with an aqueous core, comprising the steps of: Loading a substance into liposomes by incubating polymer lipid matrices at a temperature above the membrane lipid transition temperature Tc, whereby said substance to be encapsulated penetrates into said vesicles by trans-membrane permeation.

In one embodiment, the incubation is allowed to proceed by heating the liposome containing polymer to a temperature between Tc and about 150° C. for a period of time until the concentrations of a substance dissolved—in the aqueous liquid carrier phase outside—micropshere liposomes and inside the core thereof are substantially balanced.

In another embodiment, the heating temperature is above 100° C. and the heating time is sufficient to ensure sterilization of the polymer containing liposomes.

In another embodiment, sterilization is achieved by a method selected from the group consisting of irradiation, liquid chemical sterilization, gas sterilization, and ethanol sterilization.

In another aspect, the invention features a method of producing a polymer matrix composition comprising nanoparticles wherein the nanoparticles are liposomes with an aqueous core, comprising the steps of: increasing the volume encapsulated the internal core of the liposomes entrapped in the polymer—by repeated freeze-and-thaw steps or dehydration/rehydration steps.

In another aspect, the invention features a method of producing a polymer matrix composition comprising nanoparticles wherein the nanoparticles are liposomes with an aqueous core, comprising the steps of: loading an agent into the composition by utilization of a transmembrane potential.

In another aspect, the invention features a method of producing an alginate polymer matrix composition in which the nanoparticles are strepatvidien gold coated and operably linked to a yttrium 90-y biotin therapeutic agent, comprising the steps of loading an agent into the composition by utilization of a transmembrane potential wherein the transmembrane potential has been produced by creating a concentration gradient of one or more charged species across the liposome membranes.

In one embodiment, the concentration gradient is a Na+/K+ concentration gradient. In another embodiment, the concentration gradient is a pH gradient.

In one embodiment, the substance to be encapsulated may be ionic or non-ionic and is selected from the group consisting of therapeutic and diagnostic agents.

In one embodiment, the method further comprises the steps of incubating polymer liposome combinations in an external solution comprising a weakly basic agent and an ionophore to form drug-loaded liposomes, wherein liposomes entrapped in the polymer encapsulate a medium comprising a divalent metal ion salt.

In another embodiment, the external solution further comprises a chelating agent.

In one embodiment, the method further comprises terminating the incubation by removing unloaded drug and isolating—liposome polymer matrix loaded with an agent.

In another embodiment, the divalent metal ion is a member selected from the group consisting of Mn++, Mg++, Ca++, Fe++ and Ba++.

In another embodiment, the ionophore is a member selected from the group consisting of A23187, X-537A, ionomycin and 4Br-A23187. In a related embodiment, the ionophore is present in an amount of from about 10 ng to about 2000 ng per μmol of lipid. In another embodiment, the ionophore is present in an amount of from about 100 ng to about 500 ng per μmol of lipid.

In one embodiment, the chelating agent is present in said external solution in a concentration of from about 1 mM to about 50 mM. In a further embodiment, the chelating agent is selected from the group consisting of ethylene diamine tetraacetic acid, ethylene glycol-bis((3-aminoethyl ether) N,N,N′,N′-tetraacetic acid and 2-(2-bis carboxymethyl!amino-5-methylphenoxy)methyl!-6-methoxy-8-bis-carb oxymethyl aminoquinoline.

In another embodiment, the weakly basic agent is a member of the group consisting of mitoxantrone, epirubicin, daunorubicin, doxorubicin, vincristine, vinblastine, lidocaine, chlorpromazine, ciprofloxacin, dibucaine, propranolol, timolol, quinidine, pilocarpine, physostigmine, dopamine, serotonin, imipramine, diphenhydramine, quinine, chloroquine, quinacrine and codeine.

In another embodiment, the external solution has a pH of about 5.0-6.5, said drug is doxorubicin, said divalent metal ion is Mn++, said ionophore is A23187, said liposomes comprise a lipid bilayer consisting essentially of sphingomyelin and cholesterol encapsulated in a hydrogel, and said chelator is present and is ethylene diamine tetraacetic acid. In still another embodiment, the external solution has a pH of about 6.0-7.5, said drug is vincristine, said divalent metal ion is Mn++, said ionophore is A23187, said liposomes comprise a lipid bilayer consisting essentially of sphingomyelin and cholesterol encapsulated in a hydrogel, and said chelator is present and is ethylene diamine tetraacetic acid.

In a further embodiment, the isolating further comprises removing said ionophore from said drug-loaded lipid and polymer matrix.

In another aspect, the invention features a method of loading a weakly basic drug into a lipid polymer matrix comprising the steps of; incubating said lipid polymer matrix having an encapsulated medium comprising a salt of a monovalent metal ion with an external solution, wherein the external solution comprises said weakly basic drug and an ionophore to form drug loaded liposomes, wherein said ionophore facilitates the electroneutral transport of a metal ion out of the liposome in exchange for inward movement of hydrogen ions and is present in said external medium in an amount of from about 0.1 ng to about 2000 ng per μmol of lipid.

In one embodiment, the ionophore is initially present in said external medium in an amount of from about 0.1 ng to about 100 ng per μmol of lipid. In another embodiment, the ionophore is initially present is said external medium in an amount of from about 0.5 ng to about 5.0 ng per μmol of lipid.

In another embodiment, the step of terminating said incubation by removing unloaded drug and ionophore, and isolating said drug-loaded liposomes to produce a lipid polymer matrix loaded with a weakly basic drug.

In one embodiment, the monovalent metal ion is selected from the group consisting of K± and Nat

In another embodiment, the ionophore is a selected from the group consisting of nigericin, monensin and dianemycin.

In another embodiment, the weakly basic drug is a selected from the group consisting of mitoxantrone, epirubicin, daunorubicin, doxorubicin, vincristine, vinblastine, lidocaine, chlorpromazine, ciprofloxacin, dibucaine, propranolol, timolol, quinidine, pilocarpine, physostigmine, dopamine, serotonin, imipramine, diphenhydramine, quinine, chloroquine, quinacrine and codeine.

In another embodiment, the external solution has a pH of about 5.0-6.5, said drug is doxorubicin, said monovalent metal ion is K±, said ionophore is nigericin, and said liposomes comprise a lipid bilayer consisting essentially of sphingomyelin and cholesterol. In a related embodiment, the external solution has a pH of about 5.0-6.5, said drug is irinotecan, said monovalent metal ion is K±, said ionophore is nigericin, and said liposomes comprise a lipid bilayer consisting essentially of sphingomyelin and cholesterol. In another embodiment, the solution has a pH of about 5.0-6.5, said drug is vincristine, said monovalent metal ion is K±, said ionophore is nigericin, and said liposomes comprise a lipid bilayer consisting essentially of sphingomyelin and cholesterol.

In another aspect, the invention features a method of producing a polymer matrix comprising nanoparticles wherein the nanoparticle is a liposome with an aqueous core comprising the steps of: loading a therapeutic or diagnostic agent on to a liposome polymer matrix by combining a suspending matrix in a mixture of an aqueous and organic solvent which increases the membrane permeability of the liposomes to the solute, wherein the concentration of the organic solvent is at least about 10% v/v, whereby the solute enters the liposome by transmembrane permeation using a solute concentration gradient, and diluting the concentration of the organic solvent to an extent that decreases the membrane permeability of the liposome to the solute and trapping the solute in the liposome to provide a liposome loaded with solute.

In one embodiment, the organic solvent is added to a mixture of liposomes and solute in a solution. In another embodiment, the organic solvent is an alcohol.

In another embodiment, the alcohol is selected from the group consisting of methyl alcohol, ethyl alcohol, n-propyl alcohol, isopropyl alcohol and n-butyl alcohol, sec-butyl alcohol and tert-butyl alcohol.

In a further embodiment, the method combines heat and the use of an organic solvent to load agent into a polymer liposome matrix.

In another embodiment, the matrix is in a pharmaceutically acceptable carrier for topical application or application to a mucosal surface.

In another embodiment, the matrix is in a pharmaceutically acceptable carrier for injection. In another embodiment, the matrix is formulated for administration rectally or vaginally. In another embodiment, the microparticles are formulated for pulmonary administration.

In another aspect, the invention features a method for modulating the rate of release of an ionizable, biologically-active agent from a polymer lipid matrix comprising the steps of: generating a transmembrane potential across the liposome membranes which has an orientation such that if the agent is positively charged, the internal potential of the liposomes is negative relative to the potential of the external medium, and if the agent is negatively charged, the internal potential of the liposomes is positive relative to the potential of the external medium.

In one embodiment, the transmembrane potential is generated by creating a concentration gradient of one or more charged species across the liposome membranes. In another embodiment, the transmembrane potential is generated by a—Na+/K+ concentration gradient. In another embodiment, the transmembrane potential is generated by a—a pH gradient.

In another aspect, the invention features a method for reducing the rate of release of an agent from a polymer and lipid containing matrix by reducing the temperature of the matrix to a temperature between 0 and 30° C., wherein the reduction of temperature results in the reduction of the rate of release of an agent from a polymer and lipid containing matrix.

In another aspect, the invention features a method for reducing the rate of release of an agent from a polymer and lipid matrix comprising the step of lyophilization of the agent containing liposome entrapped in a polymer.

In another aspect, the invention features a method for producing a polymer matrix composition comprising the steps of: preloading a therapeutic agent A in liquid polymer subunits along with liposomes so that agent A and liposomes are entrapped throughout polymer matrix and loading therapeutic agent B into liposomes entrapped in polymer.

In one embodiment, the therapeutic agent is a radioisotope. In a related embodiment, the radioisotope is an alpha-emitting radionuclide. In one embodiment, the radisotope is a gamma emitter selected from the group consisting of Tc-99 m, In and 67Ga.

In another embodiment, a plurality of radioisotopes, drugs or contrast agents is loaded into a single liposome polymer matrix.

In one embodiment, a contrast agent that can act as a raman spectroscopy reporter, thereby allowing detection of said polymer liposome matrix with raman spectroscopy.

In one embodiment, the polymer matrix comprises a photoacoustic contrast agent, thereby allowing detection of said polymer liposome matrix acoustically.

In one embodiment, liposomes have extended half-lives in the body as they are encapsulated in a microporous polymer matrix that prevents immunoclearance

In one embodiment, the polymer matrix is in a frozen state whereby prolonged storage of a loaded liposome polymer matrix is achieved. In a further embodiment, the polymer matrix comprises a cryoprotectant. In a related embodiment, the cryoprotectant is a disaccharide selected from the group consisting of sucrose, maltose, trehalose, and lactose. In another embodiment, the cryoprotectant is has a concentration selected from the group consisting of 5%, 10%, 12%, 15%, 20%, and 25%.

In one embodiment, the invention features a method of using the polymer matrix composition of the aspects described that comprises the step of transporting the frozen liposome formulation and thawing said formulation at another location.

In another aspect, the invention features a method of freezing a polymer matrix comprising nanoparticles, which comprises the step of lyophilizing the matrix as part of a lyophilization process. In one embodiment, the method further comprises transporting the dehydrated liposome polymer matrix and dehydrating said formulation at another location.

In another embodiment, the liposome polymer contains at least one nanoparticle that changes drug release properties from the liposome polymer matrix based upon an external stimulus.

In one embodiment, the nanoparticle is selected from the group consisting of Cobalt Nanoparticles, Iron Oxide Nanopowder, Niobium Oxide Nanopowder, Thulium Nanoparticles, Cobalt Oxide Nanopowder, Lanthanum Nanoparticles, Palladium Nanoparticles, Tin Nanoparticles, Aluminum Oxide Nanopowder, Copper Nanoparticles, Lanthanum Oxide Nanopowder, Platinum Nanoparticles, Tin Oxide Nanopowder, Antimony Nanoparticles, Copper Oxide Nanopowder, Praseodymium Nanoparticles, Titanium Carbide Nanoparticles, Antimony Oxide Nanopowder, Dysprosium Nanoparticles, Lithium Manganese Oxide Nanoparticles, Praseodymium Oxide Nanopowder, Titanium Nanoparticles, Antimony Tin Oxide (ATO) Nanoparticles, Dysprosium Oxide Nanopowder, Lithium Nanoparticles, Rhenium Nanoparticles, Titanium Nitride Nanoparticles, Barium Titanate Nanoparticles, Erbium Nanoparticles, Lithium Titanate Nanoparticles, Ruthenium Nanoparticles, Titanium Oxide Nanopowder, Beryllium Nanoparticles, Erbium Oxide Nanopowder, Lithium Vanadate Nanoparticles, Samarium Nanoparticles, Tungsten Carbide Nanoparticles, Bismuth Oxide Nanopowder, Europium Nanoparticles, Lutetium Nanoparticles, Samarium Oxide Nanopowder, Tungsten Nanoparticles, Boron Carbide Nanoparticles, Europium Oxide Nanopowder, Magnesium Nanoparticles, Silicon Carbide Nanoparticles, Tungsten Oxide Nanopowder, Boron Nitride Nanoparticles, Gadolinium Nanoparticles, Magnesium Oxide Nanopowder, Silicon Nanoparticles, Vanadium Oxide Nanopowder, Calcium Carbonate Nanoparticles, Gadolinium Oxide Nanopowder, Manganese Nanoparticles, Silicon Nanotubes, Ytterbium Nanoparticles, Calcium Chloride Nanoparticles, Gold Nanoparticles, Manganese Oxide Nanopowder, Silicon Nitride Nanoparticles, Yttria stabilized Zirconia, Calcium Oxide Nanopowder, Hafnium Oxide Nanopowder, Molybdenum Nanoparticles, Silicon Oxide Nanopowder, Yttrium Nanoparticles, Calcium Phosphate Nanoparticles, Holmium Nanoparticles, Molybdenum Oxide Nanopowder, Silver Nanoparticles, Zinc Oxide Nanopowder, Carbon Nanohorns, Indium Nanoparticles, Neodymium Nanoparticles, Strontium Carbonate Nanoparticles, Zirconium Nanoparticles, Carbon Nanoparticles Indium Oxide Nanopowder, Neodymium Oxide Nanopowder, Strontium Titanate Nanoparticles, Zirconium Oxide Nanopowder, Carbon Nanotubes, Iridium Nanoparticles, Nickel Nanoparticles, Tantalum Nanoparticles, Cerium Nanoparticles, Iron Cobalt Nanopowder, Nickel Oxide Nanopowder, Tantalum Oxide Nanopowder, Cerium Oxide Nanopowder, Iron Nanoparticles, Nickel Titanium Nanopowder, Terbium Nanoparticles, Chromium Oxide Nanopowder, Iron Nickel Nanopowder, Niobium Nanoparticles, Terbium Oxide Nanopowder, Carbon 60 fullerenes, Carbon 70 fullerens and Carbon 85 fullerenes, single wall carbon nanotubes, multi-wall carbon nanotubes, and carbon nanofibers.

In one embodiment, the external stimulus is an alternating magnetic field. In one embodiment, the external stimulus is UV light. In one embodiment, the external stimulus is ultrasound, including lower high field ultrasound. In one embodiment, the external stimulus is heat from a thermal ablation probe). In one embodiment, the external stimulus is cold from a cryoablation probe). In one embodiment, the external stimulus is radiofrequency—from a radiofrequency probe).

In another embodiment, the nanoparticles are coated with dextran, peg or another agent that increases biocompatibility. In a further embodiment, the particle is an iron oxide. In another embodiment, the particle is feridex.

In another aspect, the invention features a liposome polymer matrix comprising a radioactive isotope, wherein the radioactive isotope is selected from the group consisting of iodine-131, cobalt-60, indium-192, yttrium-90, strontium-89, samarium-153, rhenium-186, technetium-99 m, or any combination thereof.

In another aspect, the invention features a liposome polymer matrix comprising a potentially radioactive isotope, wherein the potentially radioactive isotope is an isotope possessing a high absorption cross-section to neutrons, protons, electrons, or high energy photons.

In one embodiment, the potentially radioactive isotope is an isotope possessing has a high absorption cross-section to neutrons, and is one of boron-10, a lanthanide such as samarium-149, gadolinium-157. and gadolinium-155 or any combination thereof.

In another aspect, the invention features a treatment method comprising the step of administering an alginate liposome polymer matrix comprising a potentially radioactive isotope, wherein the desired treatment area is exposed to neutrons, protons, electrons, or high energy photons to activate the potentially radioactive isotope in combination with AMF thermotherapy.

In another aspect, the invention features a method of treating a subject comprising administering an alginate liposome polymer matrix to a treatment area of the subject, wherein the polymer matrix comprises a potentially radioactive isotope; exposing the treatment area of the subject to neutrons, protons, electrons, or high energy photons to activate the potentially radioactive isotope; and providing AMF thermotherapy, thereby treating the subject.

In one embodiment, the alginate liposome polymer matrix further comprises one or more of a radioisotope, potential radioactive isotope or a chemotherapeutic agent.

In another embodiment of any one of the aspects, the nanoparticle is a silica nanoparticle.

In another embodiment of any one of the aspects, the therapeutic agent is phosphorylated.

In a further embodiment, the phosphorylated therapeutic agent binds directly to the polymer. In a related embodiment, the phosphorylated therapeutic agent binds to the nanoparticle.

In another aspect, the invention features a kit comprising the polymer matrix comprising nanoparticles wherein the nanoparticles are loaded after polymerization with one or more bioactive agents, and instructions for use.

In another aspect, the invention features a kit comprising the polymer matrix comprising nanoparticles wherein the nanoparticles are loaded after polymerization with one or more bioactive agents, and instructions for use in embolization.

In still another aspect, the invention features a kit comprising the polymer matrix comprising nanoparticles wherein the nanoparticles are loaded after polymerization with one or more bioactive agents, and instructions for therapeutic use.

In another aspect, the invention features compositions comprising a hydrogel and a bioactive agent that forms one or more microspheres.

In a first embodiment, the hydrogel is selected from polyethyleneglycoldiacrylate (PEGDA) or polyethyleneglycoldimethacrylate (PEGDMA).

In another embodiment, the bioactive agent is selected from the group consisting of: anti-cancer agents, antibiotics, antihistamines, hormones, steroids, therapeutic proteins, biocompatible materials, imaging agents and contrast agents. In a further embodiment, the bioactive agent is contained in liposomes.

In a related embodiment, the contrast agent is selected from the group consisting of magnetic resonance contrast agents, radiopaque contrast agents, ultrasound contrast agents, and nuclear medicine imaging contrast agents.

In another related embodiment, the biocompatible material is selected from the group consisting of polyvinyl alcohol, sodium polyacrylate, acrylate polymers, Hyaluronase Polymers, collagen membrane, Porous HA/TCP ceramic composite, Hydroxyapatite bone cement, PVP/PMMA, tricalcium phosphate, Hydroxyapatite coated collagen fibres, calcium sulphate, Hydroxyapatite (HAp), Phosphorylcholine (PC), silicone, ultrahigh molecular weight polyethylene, polyethylene, acrylic, nylon, Polyurethane, Polypropylene, poly(methyl methacrylate), Teflon, Dacron, acetal, polyester, silicone-collagen composite, polyaledehyde, poly(vinyl chloride), silicone-acrylate, poly(tetrafluoroethylene), hydroxyethyl methacrylate (HEMA), poly(methyl methacrylate) (PMMA), poly(glycolide lactide), poly(glycolic acid), tetrafluoroethylene, hexafluoropropylene, poly(glycolic acid), poly(lactic acid), desaminotyrosyltyrosine ethyl ester, polydioxanone, fibrin, gelatin, hyaluronan, tricalcium phosphate, polyglycolide (PGA), polycaprolactone, poly (lactide-co-glycolide), polyhydroxybutyrate, polyhydroxyvalerate, trimethylene carbonate, polyanhydrides, polyorthoesters, poly(vinyl alcohol), poly(N-vinyl 2-pyrrolidone), poly(ethylene glycol), poly(hydroxyethylmethacrylate), n-vinyl-2-pyrrolidone, methacrylic acid, methyl methacrylate, and maleic anhydride, polycaprolactone, poly(amino acids) ie poly(L-lysine), poly(1-ornithine), poly(glutamic acid), polycyanoacrylates, polyphosphazenes, poly(lactic acid), poly(glycolic acid), crown ethers, cyclodextrins, cyclophanes, ethylene glycol, Methylacrylate, Para-xylylene, Biodegradable Copolymers, Copolymer Surface Coatings, Starch Polymers, Polylactic Acid, Cellophane, Tyrosine Polycarbonates Lactide and Glycolide Polymers, Collagen, PTFE, silicone, Keratin-Based Materials, Fibrous Composites—Carbon Fiber and Particles, Polymer Composites, Artificial/Natural Material Composites, Glass-Ceramic/Metal Composites, Glass-Ceramic/Nonmetal Composites, Dental Composites, Ormocer, hydrogels, timed-release foams, and polymeric carriers.

In a further embodiment, the anti-cancer agent is selected from the group consisting of: the anti-cancer agent is a 3 halopyruvate selected from the group consisting of: 3-fluoropyruvate, 3-chloropyruvate, 3-bromopyruvate and 3-iodopyruvate. In a related embodiment, the 3-bromopyruvate is modified. In still a further embodiment, the modification is with a pHLIP peptide.

In another related embodiment, the anti-cancer agent is a chemotherapeutic.

In another aspect, the invention features a method of forming one or more microspheres comprising bioactive agent, the method comprising adding bioactive agent to a hydrogel solution, adding the agent and hydrogel solution to a mineral oil bath, and initiating polymerization, thereby forming one or more microspheres comprising bioactive agent.

In one embodiment, the hydrogel is selected from polyethyleneglycoldiacrylate (PEGDA) or polyethyleneglycoldimethacrylate (PEGDMA).

In another particular embodiment, the bioactive agent is selected from the group consisting of: anti-cancer agents, antibiotics, antihistamines, hormones, steroids, therapeutic proteins, biocompatible materials, imaging agents and contrast agents.

In one embodiment, the bioactive agent is contained in liposomes.

In another embodiment, UV irradiation is used to initiate polymerization.

In another embodiment, the method further comprises a step of adding a contrast agent.

In another aspect the invention features methods for treating a subject having a vascular or non-vascular condition, the method comprising the step of administering to the subject a composition comprising a hydrogel and a bioactive agent that forms one or more microspheres.

In one embodiment, the vascular or non-vascular condition is selected from the group consisting of: arteriovenous malformation, neurovascular lesions, telangiectasias, varicoceles, varicose veins, inflammatory lesions, hemorrhage, occlusion, embolism, neoplastic growth, venous disease, and phlebitis.

In another aspect the invention features methods for treating a subject having a vascular or non-vascular hemorrhage, the method comprising the step of administering to the subject a composition comprising a hydrogel and a bioactive agent that forms one or more microspheres.

In a particular embodiment, the hemorrhage is an intracranial hemorrhage.

In another aspect the invention features methods for treating a subject having a neoplastic growth, the method comprising the step of administering to the subject a composition comprising a hydrogel and a bioactive agent that forms one or more microspheres, thereby treating the subject.

In one embodiment, the hydrogel comprises one or more anti-cancer agents.

In another embodiment, the anti-cancer agent is selected from the group consisting of: chemotherapeutics, an antibodies, and biological agents.

In another related embodiment, the anti-cancer agent is selected from the group consisting of abiraterone acetate, altretamine, anhydrovinblastine, auristatin, bexarotene, bicalutamide, BMS184476, 2,3,4,5,6-pentafluoro-N-(3-fluoro-4-methoxyphenyl) benzene sulfonamide, bleomycin, N,N-dimethyl-L-valyl-L-valyl-N-methyl-L-valyl-L-proly-1-Lproline-t-butylamide, cachectin, cemadotin, chlorambucil, cyclophosphamide, 3′,4′-didehydro-4′-deoxy-8′-norvin-caleukoblastine, docetaxol, doxetaxel, cyclophosphamide, carboplatin, carmustine, cisplatin, cryptophycin, cyclophosphamide, cytarabine, dacarbazine, dactinomycin, daunorubicin, dolastatin, doxorubicin, etoposide, 5-fluorouracil, finasteride, flutamide, hydroxyurea and hydroxyureataxanes, ifosfamide, liarozole, lonidamine, lomustine, mechlorethamine (nitrogen mustard), melphalan, mivobulin isethionate, rhizoxin, sertenef, streptozocin, mitomycin, methotrexate, 5-fluorouracil, nilutamide, onapristone, paclitaxel, prednimustine, procarbazine, RPR109881, stramustine phosphate, tamoxifen, tasonermin, taxol, tretinoin, vinblastine, vincristine, vindesine sulfate, and vinflunine.

In a further related embodiment, the hydrogel contains an iron oxide or alternate metal that heats in the presence of an alternating magnetic field thereby creating local hyperthermia.

In another aspect the invention features methods for the selective delivery of a therapeutic agent to a targeted non-occluded vessel by administering to the subject a hydrogel and a bioactive agent that forms one or more microspheres based biomaterial.

In another aspect the invention features methods for the controlled release of an agent in a subject, the method comprising the steps of: administering to the subject a hydrogel and a bioactive agent that forms one or more microspheres.

In one embodiment, the agent is a therapeutic agent.

In another embodiment, the therapeutic agent is any water-soluble agent.

In a related embodiment, the subject has a vascular or non-vascular condition.

In still a further embodiment, the bioactive agent is a nanomaterial. In a related embodiment, the bioactive agent is contained within a nanomaterial. In a further related embodiment, the bioactive agent is bound to a nanomaterial.

In one embodiment of the above-mentioned aspects, the nanomaterial is selected from the group consisting of: microboxes, microchips, microfluidic pumps, magnetic resonance microcoil, quantum dots, antibody targeted nanomaterials, nanocontainers, and nanoboxes.

In another embodiment, the bioactive agent is contained within therapeutic liposomes. In a related embodiment, the therapeutic liposomes are coated with protein. In another related embodiment, the protein is selected from the group consisting of: antibodies, receptors, and cell surface markers.

In still a further embodiment of the aspect, the therapeutic agent is selected from the group consisting of: chemotherapeutic agents, anti-inflammatory agents, antimicrobial agents, hormonal therapy agents, metalloproteinase inhibitors, sclerosing agents, angio-active agents, plasmids for gene therapy, adenoviral vectors for gene therapy, RNAi, antisense, lentivirus, microbubbles, toxins, antibiotics, vaccines, photodynamic agents, and analgesics.

In a further embodiment, the therapeutic agent is further combined with a second agent selected from the group consisting of: contrast agents, quantum dots, antibodies, liposomes, and nanoboxes.

In still another embodiment, the bioactive agent is a cell secreting a therapeutic factor.

In another aspect, the invention features methods for the controlled release of a label in a subject, the method comprising the steps of administering to the subject a hydrogel and a bioactive agent that forms one or more microspheres.

In one embodiment, the controlled release of the label is used for diagnostic purposes. In another embodiment, the diagnostic purpose is the selected angiography of a labeled vessel.

In another related embodiment, the label is selected from the group consisting of a: radiolabel, fluorescent label, and tissue dye. In still a further embodiment, the label is contained within a micelle.

In another embodiment, the radiolabel is selected from the group consisting of: carbon 14, carbon 14 intermediates, tritium-labeled radioisotopes, iodine 125 labeled radioisotopes, and antibody targeted radioisotopes.

In another embodiment, the fluorescent label is selected from the group consisting of: cadmium selenide, quantum dots, fluorophores and their amine-reactive derivatives, thiol-reactive probes, reagents for modifying groups other than thiols or amines, biotin derivatives, haptens, crosslinking reagents, and photoactivatable reagents.

In another related embodiment, the label is contained within a liposome.

In another aspect, the invention features methods for the controlled release of a label to mark lesions for radiosurgery, the method comprising the steps of: administering to the subject a hydrogel and a bioactive agent that forms one or more microspheres.

In one embodiment, the label is selected from the group consisting of a: radiolabel, fluorescent label, and tissue dye. In another embodiment, the label is contained within a micelle.

In another related embodiment, the radiolabel is selected from the group consisting of: carbon 14, carbon 14 intermediates, tritium-labeled radioisotopes, iodine 125 labeled radioisotopes, and antibody targeted radioisotopes.

In another further embodiment, the fluorescent label is selected from the group consisting of: cadmium selenide, quantum dots, fluorophores and their amine-reactive derivatives, thiol-reactive probes, reagents for modifying groups other than thiols or amines, biotin derivatives, haptens, crosslinking reagents, and photoactivatable reagents. In another embodiment, the tissue dye is methylene blue.

In another embodiment, the label is contained within a liposome. In a related embodiment, the liposome is selected from the group consisting of: heat sensitive liposomes, ultraviolet sensitive liposomes and ph sensitive liposomes.

In a further aspect, the invention provides methods for the controlled release of a contrast agent in a subject, the method comprising the steps of administering to the subject a hydrogel and a bioactive agent that forms one or more microspheres.

In one embodiment, the contrast agent is selected from the group consisting of: magnetic resonance contrast agents, radiopaque contrast agents, ultrasound contrast agents, and nuclear medicine imaging contrast agents.

In another embodiment, the magnetic resonance contrast agent is selected from the group consisting of: Manganese Oxide, perfluorocarbons, Feridex, Gadolinium, Combidex, Bang Magnetic Particles, Gd-DTPA, Gadolinium And Manganese Derivatives, Superparamagnetic Iron Oxide Particles, gadopentetate dimeglumine, Gd-DOTA, Gd-DTPA-BMA, Gd-HP-DO3A, Gd-DTPA-BMEA, Gd-DO3A-butrol, Gd-BOPTA, Mn-DPDP, Gd-EOB-DTPA, Gd-BOPTA, AMI-25, SH U 555A, gadoflourine-M, AMI-227, EP-2104R, P947, Gd-DTPA mesophorphryn, SH U 555 C, NC-100150, MS-325, gadoflourine-M, gadomelitolm manganese chloride, ferric amonium citrate, and barium sulfate suspensions.

In another aspect, the invention features methods of administering a cell or therapeutic compound in a hydrogel that forms one or more microspheres to a target area in a patient.

In one embodiment, the target area is selected from the group consisting of: liver, pancreas, thyroid, heart, peripheral nerve scaffold, breast, bladder, cartilage, bone, tendon, ligament, blood vessel, and spinal cord.

In another aspect the invention features methods of administering a biocompatible material with a PEGDA hydrogel or unpolymerized PEGDA and a bioactive agent that forms one or more microsphere in vivo.

In still another aspect the invention features methods for the selective control of bulking or remodeling in a subject, the method comprising the steps of administering to the subject a hydrogel based biomaterial to a targeted area; and polymerizing the hydrogel, thereby controlling bulking or remodeling in a subject.

In one embodiment, the subject is undergoing plastic or reconstructive procedures.

In one embodiment of any one of the above aspects, the hydrogel is selected from polyethyleneglycoldiacrylate (PEGDA) or polyethyleneglycoldimethacrylate (PEGDMA).

In another embodiment of any one of the above aspects, the hydrogel is self-polymerizing.

In another further embodiment of any one of the above aspects, the hydrogel polymerizes by exposure to UV light.

In another aspect, the invention features kits for performing any one of the methods of the invention as described herein, and instructions for use.

In one embodiment, the kits comprise a hydrogel is selected from polyethyleneglycoldiacrylate (PEGDA) or polyethyleneglycoldimethacrylate (PEGDMA) and a bioactive agent.

### BRIEF DESCRIPTION OF THE DRAWINGS

FIG. 1 (A and B) is two panels. Panel A shows chemical structure of PEGDA. Panel B shows a preferred setup for encapsulation of cellular therapeutics in which a PEGDA/photoinitator/cell solution is fed at a constant flow rate through a micropipette into a flowing inert liquid causing PEGDA sphere formation. External UV light is then applied to cause polymerization of the resulting ChemoSpheres.

FIG. 2 is a graph showing optimization of Ultragel photoinitiator concentration. Time to gelation is shown with varying photoinitiator concentration. Error bars are +/−SD of 15 replicates.

FIG. 3 (A-C) is three panels showing delivery of ChemoGel in glass phantom. A) Glass saccular aneurysm phantom; B) Polymerized ChemoGel in glass aneurysm phantom; C) Angiography after administration of isohexyl in a saline phantom flow model demonstrating stable exclusion of aneurismal sac.

FIG. 4 is a Table showing the viability of HepG2 Cells encapsulated in PEGDA chemospheres over a 28 day period containing non contrast, Feridex, Barium, Iohexyl, Feridex+Iohexyl, PFOB and PFCE.

FIG. 5 (A-D) is four panels of images. A) Image Feridex and HepG2 containing chemospheres. B) Fluorescent microscopy of rhodamine containing liposomes encapsulated in ChemoSpheres. C) Percent viability of HEPG2 cells in ChemoSpheres without contrast and with Feridex demonstrates no statistically significant difference. D) Release profile of rhodamine from liposomes in chemospheres over a 25 day period as measured by the fluorescene intensity units of the solution in which Chemosphere were incubated.

FIG. 6 is a graph showing viability of HepG2 cells incubated with the solution eluted from Feridex labeled bland ChemoSpheres (closed circles) and Feridex labeled ChemoSpheres loaded with 3 Bromopyruvate (open circles). Gradual increase in viability of HepG2 cells demonstrates gradual release over an 8 day period.

FIG. 7 is a Table showing viability of HepG2 cells incubated with the solution eluted from control ChemoSpheres containing no contrast and no 3 bromopyruvate, no contrast and 3 bromopyruvate, Feridex and 3 BrP, Barium sulfate and 3 BrP, Ioxehol and 3 BrP, Feridex, Iohexyl and 3 BrP, PFOB and 3 BrP and PFCE and 3 BrP.

FIG. 8 is a Table showing viability of HepG2 cells incubated with the solution eluted from control ChemoSpheres containing no contrast and no doxorubicin, no contrast and doxorubicin loaded liposomes, Feridex and doxorubicin loaded liposomes, Barium sulfate and doxorubicin loaded liposomes, Ioxehol and doxorubicin loaded liposomes, Feridex, Iohexyl and doxorubicin loaded liposomes, PFOB and doxorubicin loaded liposomes, PFCE and doxorubicin loaded liposomes, Gold-dextran and doxorubicin loaded liposomes.

FIG. 9 is a Table showing viability of HepG2 cells incubated with the solution eluted from a 2 cubic centimeter piece of ChemoGel containing no contrast and no doxorubicin, no contrast and doxorubicin loaded liposomes, Feridex and doxorubicin loaded liposomes, Barium sulfate and doxorubicin loaded liposomes, Ioxehol and doxorubicin loaded liposomes, Feridex, Iohexyl and doxorubicin loaded liposomes, Gold-dextran and doxorubicin loaded liposomes, PFOB and doxorubicin loaded liposomes, PFCE and doxorubicin loaded liposomes.

FIG. 10 is an Ultrasound (US) image of needle in ex vivo kidney from New Zealand white rabbit (upper white box) and delivery of a single PFOB containing chemosphere demonstrating single capsule resolution with standard clinical grade portable US unit.

FIG. 11 is a confocal image of chemosphere at greatest diameter reveals sphere size of approximately 100 micrometers.

FIG. 12 (A and B) is two panels that shows in vitro imaging of chemospheres. 50 ml conical tubes with chemospheres were suspended at a concentration of 10, 5 and 1 from top to bottom of the tube. In panel A) Gold and Iron containing chemospheres and in panel B) Iron containing chemospheres were detectable at the single sphere level and created greater hypointensity with increasing sphere number.

FIG. 13 shows imaging of chemosphere embolization in VX-2 rabbit tumor model. Targeted injection of ChemoSpheres demonstrated good tumor coverage with hypointensity present at the center of the tumor (white arrow). Small areas of non-targeted injection could also be seen on MR (white arrowheads).

FIG. 14 shows unilateral embolization of kidneys in New Zealand white rabbit demonstrated the ability to detect ChemoSpheres post-embolization as hypointense areas (white arrows).

FIG. 15 is an image showing live (green) and dead (red) stain of HepG2 cells encapsulated in chemospheres.

FIG. 16 is an image showing ChemoSpheres loaded with gold-dextran loaded in a 10 mL syringe and overlayed on the abdomen of a rabbit reveals ability to detect individual chemospheres.

FIG. 17 is two panels showing spectranetics fiberoptic catheter and sheaths (image on left). The image on the right shows a connection box to hook spectranetics catheter to standard 365 nm UV wand.

FIG. 18 shows gold-dextran containing chemospheres suspended in gelatin in a 50 mL conical tube as point sources of 1 and 5 microcapsules.

FIG. 19 shows a slice from a 64 slice CT scan of a New Zealand white rabbit after unilateral kidney embolization with 1 mL packed volume of gold-dextran chemospheres.

FIG. 20 shows ChemoSpheres loaded with manganese oxide and citric acid containing liposomes prior to drug loading.

FIG. 21 shows H&E stained histological section of embolized bland manganese oxide loaded chemosphere. The chemospere demonstrates high biocompatibility with little sign of inflammation.

FIG. 22 shows H&E stained histological section of embolized bland manganese oxide loaded chemosphere. The chemosphere demonstrates high biocompatibility with little sign of inflammation.

FIG. 23 shows H&E stained histological section of iron oxide loaded chemospheres in vessel. The chemospheres demonstrate compressibility and serial embolization of adjacent spheres

FIG. 24 shows H&E stained histological section of iron oxide loaded chemosphere. The chemosphere demonstrates mild inflammation surrounding the site of embolization.

FIG. 25 shows results of MR spectroscopy of manganese oxide or feridex 3 Bromo Pyruvate loaded chemospheres 8-channel Siemens Knee Coil Used. Samples were provided in 50 cc centrifuge tubes. Sample #1. Manganese oxide+200 mg/ml polymer+liposome+200 mg 3-bromopyruvate+gelatin, 40 mL total volume, MW 166.95->3-BP concentration=(200/166.95)/0.04=29.95 mM Scan Parameters: 13×25×13 mm=4.225 cm3, TR/TE 2000/30 ms, 128 averages scan time 4 min 26 sec. Water suppression 75 Hz BW. Remove oversampling checked. TE 140 13×25×13 mmTE 30 10×10×10 mm (=1 cc) No water suppression (8 averages). Sample #2. Same as #1, except feridex instead of MnO. 20% by volume 11.2 mg Fe+5.6−9.1 mg dextran/mL TE=30 ms, 13×25×13 mm 128 averages TE=30 no water suppression (8 averages) TE=30 ms, 10×10×10 mm 128 averages TE=30 no water suppression (8 averages) Sample #3. 50 mM doxorubicin (adriamycin) TE=30 ms, 13×25×13 mm 128 averages, TE=30 no water suppression (8 averages).

FIG. 26 shows the MR spectra of solution eluted from 3 Bomopyruvate loaded spheres after 24 hours demonstrates 3br-pyruvate peak (3.65 peak.) The other peaks are breakdown products. The soft hump around 4.8 is water. The peak at 0 is chemical shift standard.

FIG. 27 shows the NMR spectra of solution eluted from 3 Bomopyruvate loaded spheres after eight days demonstrates 3br-pyruvate peak (3.65 peak.) The breakdown products are increased as compared to the 24 hour elution but active 3 bromopyruvate is still eluted.

FIG. 28 is a blow up of main peak of FIG. 27 that shows increased breakdown products as minor component and active 3-Bromopyruvate as major component of eluted solution at 8 days.

FIG. 29 shows the MR Imaging of alginate microspheres at 3T. (A) GdO containing EmboCaps in gelatin phantom. One (B) and five (C) MnO EmboCaps. Kidney (D) and Vx-2 tumor embolization in New Zealand white rabbit.

FIG. 30 is an image showing that after all doxorubicin was eluted from the spheres, spheres were dissolved with alginate lyase and EDTA. EDTA (Sigma, St. Louis, Mo.) was added at a concentration of 5 mg/mL to normal saline. The pH of this preparation was then adjusted to pH 7.0. Alginate lyase isolated from Flavobacterium sp. (Sigma, St. Louis, Mo.) was then added at a concentration of 2 mg/ml to the EDTA solution. This solution was then filter sterilized through a syringe tip filter (Millipore). As seen in this image, liposomes remained largely intact even after all drug was released.

FIG. 31 shows Rabbit Vx-2 PET/CT scan 24 hrs after embolization with 3 BromoPyruvate loaded iron oxide PEGDA chemospheres.

FIG. 32 shows PEGDA microspheres containing streptavidin coated gold nanoparticles incubated in biotin-4-fluorescein indicates proof of principle labeling of spheres with biotin conjugated therapeutic agent (ie yttrium-90 biotin).

FIG. 33 shows a stent coated with a macroporous PTFE membrane. By polymerizing hydrogel containing streptavidin coated nanoparticles on the membrane or alternatively directly on struts such a stent is loaded with therapeutic agent conjugated to biotin.

FIG. 34 (A-C) shows (A) macroscopic image of pegda containing streptavidin coated gold nanoparticles polymerized on porous stent-graft pictured in FIG. 33. (B)—Auto-fluorescence of membrane prior to incubation with biotin-4-fluorescein. (C) Fluorescence of stent membrane after incubation with biotin-4-fluorescein shows high labeling efficiency. Proof of principle that such a strategy could be employed to make a loadable stent or other device decorated with streptavidin.

FIG. 35 shows an exemplary microfluidic device utilized for formation and polymerization of microspheres containing fluorescene loaded liposomes. The use of the microfluidic device minimizes loss of liposomes to the organic/hydrophobic liquid utilized in emulsion based polymerization techniques.

FIG. 36 shows a diagram of microfluidic droplet generator chip for creating microspheres of approximately 50 micrometers.

FIG. 37 shows gadolinium oxide loaded pegda chemospheres loaded in gelatin loaded 50 mL tube. Concentration of gadolinium oxide per sphere must be optimized to ensure solely hyperintense signal on standard T1 weighted scan. Note in the second and third gelatin phantoms from the left a hypointense center surrounded by a hyperintense rim whereas in image at far right only a hyperintense image is seen.

FIG. 38 shows fluoroscopic angiography of kidney with catheter in renal artery pre-embolization (left frame) and post-embolization (right frame) with iron-oxide containing chemospheres demonstrates effective embolization resulting in stagnant blood flow.

FIG. 39 shows fluoroscopic angiography of rabbit VX-2 liver tumor pre transarterial embolization (left frame) and post trans-arterial embolization (right frame) with iron-oxide containing chemospheres demonstrates effective embolization resulting in stagnant blood flow.

FIG. 40 is a list of photoinitiators that may be employed to induce polymerization of photocrosslinkable polymers such as PEGDA. Listed is optimal absorbances and chemical properties of each of the photoinitiators.

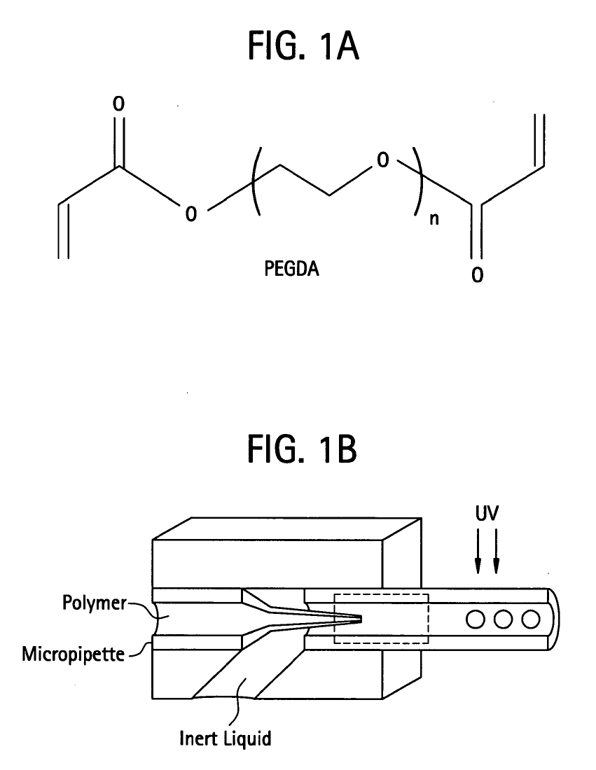

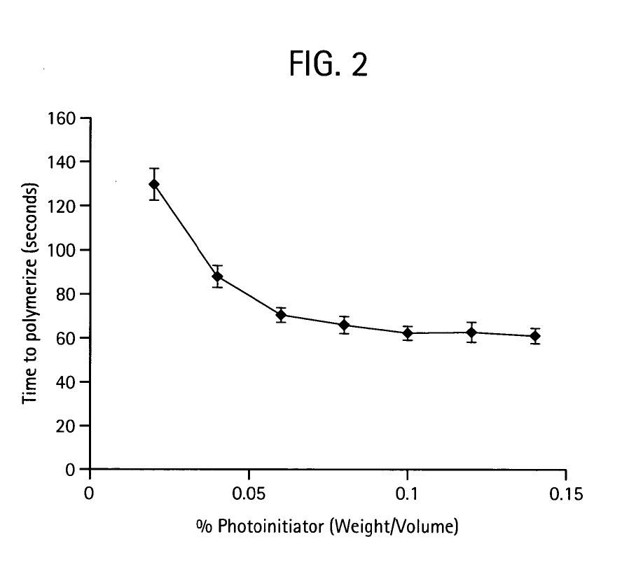

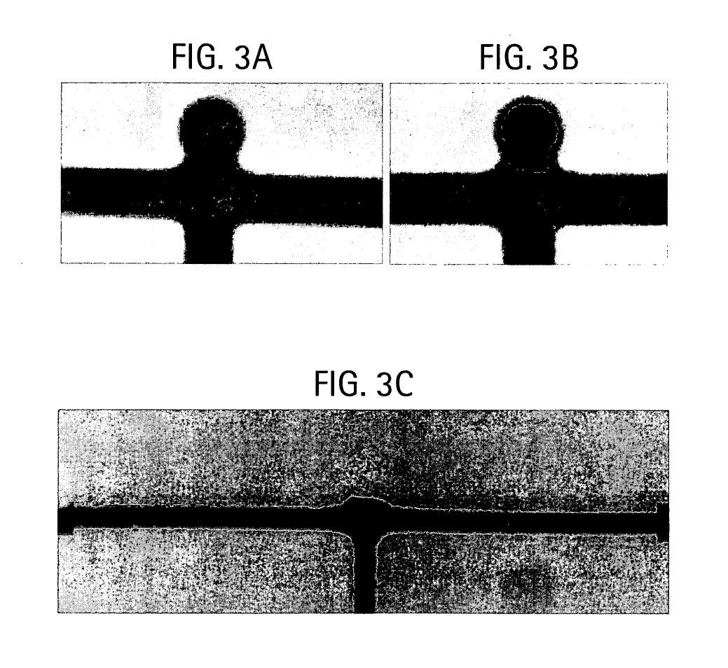

### DETAILED DESCRIPTION

The present invention provides, in general, compositions comprising a hydrogel and an agent. In one aspect, the invention features a hydrogel and a bioactive agent that forms one or more microspheres. The bioactive agent may be a therapeutic agent, a diagnostic agent, or an imaging agent, that is used, for example, for locoregional delivery. In certain embodiments, the hydrogel comprises poly (ethylene glycol)-diacrylate (PEGDA). In other certain preferred embodiments of the invention, the hydrogel compositions are detectable by Magnetic Responance and CT Scan and are used for locoregional delivery of therapeutic agents, for example chemotherapeutic agents 3-Bromopyruvate and Doxorubicin.

The invention also features compositions comprising nanoparticles, e.g. microspheres, that can be loaded after polymerization with a bioactive agent. The composition may be a polymer matrix composition. The bioactive agent may be a therapeutic agent, a diagnostic agent, or an imaging agent.

### DEFINITIONS

In this disclosure, “comprises,” “comprising,” “containing” and “having” and the like can have the meaning ascribed to them in U.S. patent law and can mean “includes,” “including,” and the like; “consisting essentially of or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

The term “biocompatible material” is meant to refer to any synthetic or natural material that can be used to replace part of a living system, or any synthetic or natural material that can function in intimate contact with living tissue.

The term “contrast agent” is meant to refer to agents that are useful in imaging techniques or methods, such as, but not limited to, magnetic resonance imaging, CT scan, ultrasound, nuclear magnetic imaging. Contrast agents can be, but are not limited to, magnetic resonance contrast agents, radiopaque contrast agents, ultrasound contrast agents, and Nuclear Medicine Imaging contrast agents.

The term “diagnosis” refers to a process of determining if an individual is afflicted with a disease or ailment, for example a vascular or non-vascular condition. A vascular condition can include arteriovenous malformation, neurovascular lesions, telangiectasias, varicoceles, varicose veins, inflammatory lesions, hemorrhage, occlusion, embolism, neoplastic growth, venous disease, and phlebitis.

The term “hydrogel” is meant to refer to any material forming, to various degrees, a microsphere. In certain preferred embodiments, the hydrogel is a PEGDA based biomaterial.

The term “loaded” is meant to refer to a process of impregnating or saturating or filling another material or container. In certain embodiments, the material or container is biocompatible.

The term “matrix” is meant to refer to any polymer that can entrap nanoparticles. In preferred embodiments, the polymer is formed by mixing nanoparticles with unpolymerized monomeric units and inducing polymerization. In exemplary embodiments, the polymer is a hydrogel. In certain preferred examples, nanoparticles are premixed with unpolymerized monomeric units, polymerization is induced and post polymerization entrapped nanoparticles are loaded with therapeutic agent. The term “nanomaterial” is meant to refer to a particle having one or more dimensions of the order of 100 nm or less. Examples of nanomaterials according to the invention include, but are not limited to, microboxes, microchips, microfluidic pumps, magnetic resonance microcoil, quantum dots, antibody targeted nanomaterials, nanocontainers, and nanoboxes.

The term “neoplastic growth” or “neoplasia” is meant to refer to any disease that is caused by or results in inappropriately high levels of cell division, inappropriately low levels of apoptosis, or both. For example, cancer is an example of a neoplasia. Examples of cancers include, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). Lymphoproliferative disorders are also considered to be proliferative diseases.

The term “subject” is intended to include vertebrates, preferably a mammal. Mammals include, but are not limited to, humans.

The term “treatment” or “treating” can mean: treating or ameliorating disease and or symptoms.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

The phrase “therapeutically-effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The term “vascular condition” is meant to refer to a condition that affects the blood vessels. Vascular conditions can include vascular disease, which affects the body's network of blood vessels (arteries and veins) that distribute oxygen and nutrient-rich blood to the body, and bring back deoxygenated blood to the heart and lungs from the rest of the body. Vascular disease can include, but is not limited to, arterial vascular disease and venous vascular disease. A vascular condition can be a vascular lesion. A vascular condition can be, but is not limited to, an occlusion, an embolism, or a hemorrhage.

### Microspheres

Therapeutic agent-eluting microspheres show promise in various clinical areaas, for example in the field of interventional radiology. Many therapeutics in the current clinical armamentarium are unstable at the high temperatures required for traditional microsphere synthesis, therefore current commercially available drug eluting beads are loaded after synthesis. Commonly, loading is based upon ionic interaction between a charged drug and an oppositely charged microsphere. Such a technique limits loading of spheres with charged compounds. Further, preliminary studies have shown much of the drug that is loaded on the sphere by ionic interaction is not eluted under physiological conditions.

Alternatively as described in Hubbell et al, therapeutic agents can be chemically cross-linked to the monomeric components of a polymer prior to polymerization. Such a strategy as employed in U.S. Pat. No. 7,413,739, incorporated by reference in its entirety herein, was used to overcome the burst release of therapeutic factors that occurred when therapeutic agents were simply entrapped in the hydrogel matrix. However, there are limitations of such an approach related to the difficulty of creating chemical cross-links that are broken under physiological conditions to release therapeutic agents.

The primary strength of hydrogels such as PEGDA used in microspheres, namely the high biocompatibility due to water content similar to human tissue, can also be problematic when using such a polymer for drug delivery applications.

Described herein are microspheres with a number of novel aspects.

As many nanoparticles are rapidly cleared from the body by the reticuloendothelial system or are marked for clearance by immune components, incorporation of nanoparticles in highly biocompatible hydrogels prevents the rapid clearance of such nanoparticles. Encapsulation in hydrogel matrices largely isolates through size exclusion interaction with antibody, complement and other arms of the host immune system thus drastically increasing half-life of the nanoparticle in the body of the host. With nanoparticles largely immunoisolated, the microspheres described herein achieve drug release on the order of weeks to months after administration, for example preferably parenteral administration, instead of a maximum half-life of two days as obtained from traditional lipid-PEG conjugates, nanoparticle drug formulations, or the widely employed pegylated liposomal formulation found in products such as Doxil. The considerably increased half life of liposomes in hydrogel matrices is unexpected especially in light of the documentation of liposome half-lives on the order of days even when pegylated.

The instant invention described post loading strategies. In certain embodiments, a post loading-strategy is preferable. In certain cases, such a design strategy could enable loading of preformed microspheres containing liposomes by the pharmacy or clinician just prior to administration to patient thereby largely circumventing the current issues of shelf-life with preloaded formulations. Several methods have been developed for liposome loading but to date no methods have been described for loading a hybrid polymer liposome matrix. Described herein are new techniques to load liposome polymer matrices, for example, by creating a transmembrane potential. Further described herein are experiments wherein the combination of polymer and liposome has an unexpected effect of synerginicity in which compound is released slower from a polymer/liposome matrix at a far reduced rate as compared to a polymer or liposome below. Neutralization of the concentration gradient is coupled to flow of the substance being loaded into the liposome. pH gradients, Na+/K+ gradients and NH4+ gradients have been used to load a variety of drugs into liposomes.

Described herein are microsphere formulations that contain liposomes that have a polymer instead of aqueous core. The use of liposome encapsulated hydrogels has been explored. In Karzakov et al. (US 2003/0035842, incorporated by reference in its entirety herein), the individual liposomes with a polymer core spontaneously aggregate to form “giant lipobeads.” Such unstable formulations that spontaneous aggregate can be problematic for standardized drug delivery. Instability of liposomes largely result from a hydrogel core and an aqueous surrounding thereby creating an unstable environment for maintenance of liposome. A preferable embodiment is to stabilize liposomes with both a polymeric core and polymeric external environment. For such formulations, it is preferable to leave the unpolymerized units ie PEGDA and photoinitiator in the liposome and then incorporate the liposomes in unpolymerized PEGDA and then use UV light to polymerize both simultaneously. This prevents aggregation of the individual liposomes as they are spatially entrapped throughout the matrix and gives them long term stability and sustained drug release capability.

The invention features hydrogel compositions comprising, in certain embodiments, agents, in particular, therapeutic agents. The hydrogel compositions in preferred embodiments, form microspheres.

In a particular embodiment, said microspheres are comprised of the hydrogel polyethyleneglycoldiacrylate (PEGDA). In another embodiment, microspheres are composed of polyethyleneglycoldimethacrylate (PEGDMA).

In certain preferred aspects, the invention features compositions comprising a hydrogel formed into a microsphere comprising an active agent. The microspheres of the instant invention, referred to herein as chemospheres, can be loaded with a variety of hydrophilic compounds and can also be used to encapsulate drug-loaded liposomes.

Any active agent that can be compounded into liposomes, microspheres, nanospheres, or other suitable encapsulation vehicle can be confined within the hydrogel matrices of the present invention to create the therapeutic hydrogels of the present invention.

For example, in certain embodiments, the microsphere composition comprises one or more of the inhibitors in hydrogel microsphere.

The hydrogels of the present invention in preferred embodiments serve as support material for a variety of liposomal therapeutics. Any therapeutic agent suitable for encapsulation in a liposome, microsphere, nanosphere or the like can be utilized in the present invention. For example, active agents useful in the present invention include anticancer agents, antibiotics, antihistamines, hormones, steroids, therapeutic proteins, imaging agents, contrast agents, and the like.

It will be appreciated by those of ordinary skill in the art that the desired concentration of active agent within a hydrogel loaded on a substrate will vary depending upon the characteristics of the chosen active agent. For example, as between an antibiotic and a therapeutic protein, the required concentration of antibiotic, which are generally active in the microgram range, will likely be higher than the concentration of a therapeutic protein, many of which are active in the nanogram range. Other standard dosing criteria will also be considered in selecting the concentration ranges of active agent loaded onto the substrate in accordance with standard practice in the art.

In a particular embodiment of the method, the PEGDA based chemosphere comprises one or more anti-cancer agents. In a particular embodiment the anti-cancer agent is a 3 halopyruvate selected from the group consisting of: 3-fluoropyruvate, 3-chloropyruvate, 3-bromopyruvate and 3-iodopyruvate. The concentration of 3 halopyruvate may be added at a concentration ranging from 1 mg/ml to 500 mg/ml of PEGDA solution as described above.

For example, in certain embodiments, the microsphere composition comprises one or more anti-cancer agents. Anti-cancer agents can include one or more chemotherapeutics typically used in the treatment of a neoplasm, such as abiraterone acetate, altretamine, anhydrovinblastine, auristatin, bexarotene, bicalutamide, BMS184476, 2,3,4,5,6-pentafluoro-N-(3-fluoro-4-methoxyphenyl)benzene sulfonamide, bleomycin, N,N-dimethyl-L-valyl-L-valyl-N-methyl-L-valyl-L-proly-1-Lproline-t-butylamide, cachectin, cemadotin, chlorambucil, cyclophosphamide, 3′,4′-didehydro-4′-deoxy-8′-norvin-caleukoblastine, docetaxol, doxetaxel, cyclophosphamide, carboplatin, carmustine (BCNU), cisplatin, cryptophycin, cyclophosphamide, cytarabine, dacarbazine (DTIC), dactinomycin, daunorubicin, dolastatin, doxorubicin (adriamycin), etoposide, 5-fluorouracil, finasteride, flutamide, hydroxyurea and hydroxyureataxanes, ifosfamide, liarozole, lonidamine, lomustine (CCNU), mechlorethamine (nitrogen mustard), melphalan, mivobulin isethionate, rhizoxin, sertenef, streptozocin, mitomycin, methotrexate, 5-fluorouracil, nilutamide, onapristone, paclitaxel, prednimustine, procarbazine; RPR109881, stramustine phosphate, tamoxifen, tasonermin, taxol, tretinoin, vinblastine, vincristine, vindesine sulfate, and vinflunine. Other examples of chemotherapeutic agents can be found in Cancer Principles and Practice of Oncology by V. T. Devita and S. Hellman (editors), 6th edition (Feb. 15, 2001), Lippincott Williams & Wilkins Publishers.

3-Bromopyruvate Enhanced with pHlip Peptide

In certain embodiments, those inhibitors are 3-halopyruvates. In still other embodiments, the pharmaceutical composition comprises one or more of the inhibitors and a second chemotherapeutic agent. In another embodiment, 3-halopyruvates are entrapped in hydrogel microspheres.

In a particular embodiment of the method, the PEGDA based chemosphere comprises one or more anti-cancer agents. In a particular embodiment the anti-cancer agent is a 3 halopyruvate selected from the group consisting of: 3-fluoropyruvate, 3-chloropyruvate, 3-bromopyruvate and 3-iodopyruvate. The concentration of 3 halopyruvate may be added at a concentration ranging from 1 mg/ml to 500 mg/ml of PEGDA solution as described above.

The 3 bromopyruvate contained in chemospheres or chemogel may be modified prior to incorporation into the hydrogel to enable it to preferably accumulate in tumor. One example of such a modification involves the use of a pH (Low) Insertion Peptide (phlip). Phlip peptides have been shown to selectively target a wide spectrum of cancer cell lines and tumors and act as nanosyringes capable of injecting them with a variety of cargo molecules at concentrations 5 times higher than in normal tissues. The selectivity of these peptides towards cancer cells is pH-dependent as they have three states: soluble in water, bound to the surface of a membrane, and inserted across the membrane as an alpha-helix. At physiological pH, the equilibrium is towards water, which explains its low affinity for cells in healthy tissue. However, at the acidic extracellular pH that characterizes cancer cells, several Asp residues of the pHLIP are titrated and this peptide becomes rigid (as a syringe needle) and shifts the equilibrium towards membrane insertion. Once inserted into the cancer cell the peptide is capable of injecting a cargo molecule by means of a reversible disulfide bond with a cysteine group located at the extreme that penetrates cancer cells. In preliminary tests in a mouse breast adenocarcinoma model, the fluorescently labeled peptide was reported to find solid acidic tumors with high accuracy and accumulate in them even at a very early stage of tumor development.

A potential example of a phlip residue that can be bound to 3-bromopyruvate include the phlip peptide with a single cysteine residue at its C terminus:

(AAEQNPIYWARYADWLFTTPLLLLDLALLVDADEGTCG)

The peptide (pHLIP) predominantly inserts across a cell membrane at low pH (<7.0) but not at normal physiological pH. By translocating a molecule into a cell and releasing it in the cytoplasm, pHLIP functions, in effect, as a nanosyringe. The peptide does not exhibit any elements of helical secondary structure in solution or on the cell membrane at neutral pH; however, it becomes rigid (as a syringe needle) when it inserts into a lipid bilayer, and it forms a transmembrane helix and injects molecules into cells.

### Methods of Delivery and Release

In addition to intraarterial delivery to hepatic tumors, 3-bromopyruvate microspheres may be delivered intrayscularly or percutaneously to kill numerous other tumors. Further 3-Bromopyruvate microspheres may be created in a range of sizes from nanometer to millimeter scale to enable different release strategies for various applications. ChemoSpheres can be made at a base concentration of 1 mg-1 g of 3-Bromopyruvate per mL of PEGDA solution. Chemospheres can be formed in a solution of FDA approved iohexyl, barium sulfate, bismuth sulfate, perfluorooctylbromide (PFOB) in micelles, tantalum silver or gold nanoparticles such as dextran covered 50 nm gold nanoparticles (Nanocs) and other radiopaque agents to provide a significant radiopacity for detection with clinical x-ray modalities. To enable visibility with MRI, iron oxides such as Feridex or Resovist, perfluoropolyether in micelles can be added to PEGDA prior to polymerization. For detection with ultrasound, PFOB within micelles or other US agents may be added to the PEGDA polymer prior to polymerization. As apparent to one skilled in the art, numerous contrast agents of sizes larger than the pore size in PEGDA chemospheres can be entrapped within the matrix whereas smaller agents will slowly elute from the particles. Depending upon the particular application, chemospheres may be designed to retain x-ray, mr or us visibility or lose contrast over time to for example minimize beam-hardening artifacts on CT.

### Liposome-Polymer Matrices

### Methods of Loading

According to certain preferred embodiments of the invention, the simplest method of loading a polymer liposome matrix is a passive entrapment of a water soluble material in a dehydrated polymer liposome matrix by hydration of lipid components. The loading efficiency of this method is generally low. Loading efficiency can be increased by the dehydration-rehydration method in which a preformed liposome polymer matrix is dehydrated in the presence of solute and subsequently reconstituted.

An alternate method for loading a polymer-liposome matrix involves the heating the polymer liposome matrix while bathed in a solution of the agent to be loaded. For heat based loading of the time of incubation may vary in function to the rates of permeation into lipids typical of the substances to be encapsulated, the nature and concentrations of the liposomes in the carrier phase, and the temperature of incubation. The factor that will generally determine the end of the incubation time is the condition where the concentrations of the encapsulated substances are the same inside and outside the liposomes. At this moment, equilibrium has been reached and prolonging incubation has no further purpose. Of course, the higher the temperature, the faster equilibrium is established; however too high temperatures may be detrimental to the liposome properties, namely to the specific encapsulation capacity, i.e., the ratio of core volume to weight of lipids; hence the incubation temperatures may range from about T c to about 150°-200° C., the preferred range being from about 40° to 130° C. It should be noted in this connection that if the incubation temperature is in the high portion of the given range, say, 100° to 150° C., substantial sterilization of the liposomes will occur simultaneously with incubation. Alternatively, one may effect sterilization and incubation independently and subsequently.

In addition, transmembrane permeation can be induced by use of ethanol to increase the permeability polymer matrices. Solutes that can be loaded by this ethanol mediated process include both small nonpolar molecules and larger species, such as proteins and carbohydrates. Specifically the method involves combining an aqueous solution having liposomes dispersed therein with the solute and an organic solvent which increases the membrane permeability of the liposomes to the solute, whereby the solute enters the liposome by transmembrane permeation, and diluting the concentration of the organic solvent thereby decreasing the membrane permeability of the liposome to the solute and trapping the solute in the liposome to provide a liposome loaded with solute. The method comprises: combining an aqueous solution having liposome polymer matrices dispersed therein with the solute and an organic solvent which increases the membrane permeability of the liposomes to the solute, whereby the solute enters the liposome by transmembrane permeation, and diluting the concentration of the organic solvent, thereby decreasing the membrane permeability of the liposome to the solute and trapping the solute in the liposome, to provide a liposome loaded with solute. Preferably, the organic solvent is an alcohol and is added to a mixture of the solute and liposomal dispersion. Other acceptable polar solvents include alcohols, glycols, ethers, dimethoxyethane, acetone, chloroform, dimethyl sulfoxide and the like which is capable of increasing the membrane permeability of the liposome in the liposome polymer matrix. In certain instances the polar solvent may also act to sterilize the polymer [symbol] liposome matrix.

In one aspect of the invention, a method is provided for loading a cationic substance into liposomes. The method comprises forming liposomes in an aqueous medium including a metal-free exchange cation of high liposomal membrane permeability containing carbon and nitrogen, and an anion of low liposomal membrane permeability, to produce liposomes which include a portion of the medium containing the cation and anion in the interior aqueous space and which are dispersed in the aqueous medium which is external to the liposomes. Preferably, the exchange cation is an amine, and most preferably a primary, secondary or tertiary amine. The concentration of the cation and anion in the external aqueous medium is then lowered, such as by ultrafiltration or other buffer exchange means, and the cationic substance to be loaded is added to the external phase of the dispersion.

In another aspect of the invention, a method is provided for loading an anionic substance into a liposome polymer matrix. The method comprises forming liposomes, in an aqueous medium having a given pH and containing an exchange anion of high liposomal membrane permeability and a cation of low liposomal membrane permeability, to produce liposomes which include a portion of the medium containing the anion and cation in the interior aqueous space and which are dispersed in the aqueous medium which is external to the liposomes. The concentration of the anion and cation in the external aqueous medium is lowered while maintaining the pH of the medium equal to that of the interior aqueous space, and the anionic substance to be loaded is added to the external phase of the dispersion.

An additional substance, such as a chelator or chelatable material that is capable of binding to the exchange ion with high membrane permeability noted above while not binding to the compound of interest, can also be included in or added to the external aqueous phase in order to trap the permeable ion thus assisting in driving equilibrium towards complete (100%) entrapment.

pH Gradients

An alternate approach to the use of ionic gradients has been the application of pH (H+) gradients to load drugs or other agents of interest into preformed liposome polymer matrices. In order to most efficiently load and take advantage of the maximum ionization potential of a counterion, pH changes on the order of 1.5-2 units or more are typically required. The general rule is that for every unit of pH difference a tenfold accumulation of the drug occurs. For drugs containing several titrable groups the accumulation behavior is altered. Thus a drug which has two amino groups, having pKa's that are greater than the pH of the final solution, can be accumulated a hundred—fold with a pH gradient of one unit. A drug with three such units can be accumulate a thousand-fold in the presence of a one-unit pH gradient, etc. Conversely for a multi-acid drug, its pKa must be less than the pH of the final solution, for such substantial accumulation to occur.

The vesicles can be prepared by the entrapment of a buffer which will not permeate the membrane in the preparation of the vesicle. Specifically they are prepared in a buffer that is either more acidic or more alkaline that the physiological pH that they will encounter in the animal. The resulting vesicles will then have a pH gradient between their interior and exterior.

The practical limits of the pH gradient are set by the tolerance of the lipid-like material and particular polymer that is used in preparing the vesicles. For simple biological lipids like soybean phosphatides pH extremes of 4 and about 10.5 are readily tolerated for extended periods of time. Vesicles to be loaded with amines are prepared in the presence of an acidic and membrane-impermeable buffer such as citrate that has one or more pKa's in the range of interest (usually about 5) and a pH of 4. In cases where the liposome polymer matrices are to be loaded with acidic molecules (carboxyl groups), the liposome polymer matrices are prepared by sonication in the presence of impermeable alkaline buffer that has a pKa of about 10.

Examples of appropriate acidic buffers other than citrate are tartrate or succinate. Appropriate alkaline buffers include, in addition to carbonate, lysine, lysine/phosphate and TAPS (Sigma). The buffer may not be permeable to the membrane and should be chloride free since chloride can promote gradient decay at non-physiological pH.

The compounds loading rate will depend on the pKa and will be complete within less than a minute for low molecular weight (MW less than 500) amine chemicals with a pKa less than 10 and having no charge or strongly polar groups other than the amino group. Analogously, weak mono-acids having a pKa greater than 4 will accumulate in the liposome polymer matrix in about one minute, unless they bear strongly polar groups other than their carboxyl's. Generally polar groups will slow the transmembrane diffusion of drugs.

Compounds which do not contain amino groups or equivalent basic groups or carboxyl or equivalent acid groups are first converted to a derivative containing either an acid or a base moiety that will not adversely affect a drug's therapeutic effect. In some instances it is desirable to prepare pro-drug moieties which will be converted into their desired active species by intracellular enzymes.

Phosphorylated hydrophobic compounds are driven into liposome polymer matrices by a pH gradient, alkaline inside, and are subsequently released sufficiently slowly to ensure that appreciable drug concentrations will remain within the liposome polymer matrices. The phosphorylated drugs can be loaded into liposome polymer matrices that have been prepared in alkaline buffers and then suspended in solutions of the phosphorylated drug in buffers of lower pH. Accumulation of the phosphorylated drugs into the liposome polymer matrices should occur within ten minutes, possibly using slightly elevated temperature to accelerate loading. Retention of the phosphorylated drugs within the liposome polymer matrix can be enhanced with increasing pH gradients. After completion of the loading procedure the pH of the liposome polymer matrix suspension is adjusted to physiological pH (pH=7.4). It is possible that some phosphorylated drugs will have physiological activity, but generally physiological activity will only be manifested after enzyme-catalyzed hydrolysis has released the phosphate group.

One approach for avoiding pH extremes is by creating differences in transmembrane ammonium (NH 4) ion concentrations between the liposome and polymer matrix. An advantage of this technique is that the loading process can be conducted at or near neutral pHs without the necessity of exposing the preparation to severe pH ranges. Neutral ammonia exits the internal aqueous space through the membrane bilayer to create a pH gradient. This is followed by the influx of a deprotonated amphiphilic drug to replace the departed ammonium.

### Ionophoric Loading

Another method for loading liposome polymer matrices involves loading liposomes that have an encapsulated medium comprising a salt of a divalent metal ion. These liposomes are then loaded into a polymer matrix and the uptake of a weakly basic drug is accomplished by incubating these liposome polymer matrices with an external solution comprising the drug and an ionophore which is capable of the electroneutral exchange across the liposome polymer matrix of one divalent metal ion for two protons. Preferably, the external medium containing the weakly basic drug will further comprise a chelating agent which coordinates any metal ion released to the external medium. In a similar strategy a monovalent metal ion can be used. Specifically a liposome polymer matrix comprising a salt of a monovalent metal ion are incubated with an external solution comprising the weakly basic drug and an ionophore to form drug-loaded polymer [symbol] liposome matrices.

Irrespective of the ionically charged component, the lipids or mixture of lipids to be used in the present invention substantially include all compounds commonly used in the field of liposomes, i.e. glycerophospholipids, non-phosphorylated glycerides, glycolipids, sterols and other additives intended to impart modified properties to liposomic membranes. Preferably, they comprise at least a polarizable component (even in minor quantity), namely a cationic or anionic function carrying lipid or an ionizable tenside such as a fatty alcohol diphosphate ester, e.g. dicetyl phosphate (DCP) or a higher alkyl amine like stearylamine (SA). Charged phospholipids, i.e. fatty acid glycerides phosphatides like phosphatidic acid (PA), phosphatidylglycerol (PG), phosphatidyl—inositol (PI), phosphatidyl-serine (PS) from natural sources or synthetic (such as dipalmitoyl-phosphatidic acid (DPPA), dipalmitoyl-phosphatidyl glycerol (DPPG), etc.) are convenient polarizable lipid components. The glycerophospholipids may include for instance the following synthetic compounds: Dipalmitoyl-phosphatidyl-choline (DPPC), dipalmitoyl-phosphatidyl-ethanolamine (DPPE) and the corresponding distearoyl- and dimyristyl-phosphatidyl-choline and -ethanolamine (DSPC; DSPE; DMPC and DMPE). Phospholipids may also include natural phospholipids which have been subjected to more or less extensive hydrogenation, for instance egg and soy phosphatidylcholine.

The glycolipids may include cerebrosides, galactocerebrosides, glucocerebrosides, sphingomyelins, sulfatides and sphingolipids derivatized with mono-, di- and trihexosides. The sterols, which should be used with parsimony, as too much may impede membrane permeation, encompass cholesterol, ergosterol, coprostanol, cholesterol esters such as hemisuccinate (CHS), tocopherol esters and the like.

### UV Initiated Photopolymers

Although numerous polymers can be used for the generation of liposome-polymer matrices, it is important that the polymerization conditions to not substantially compromise the integrity of the liposomes or the agents they may contain. For this reason UV initiated photopolymers are an optimal choice. Potential UV polymers that can be utilized in this invention include but are not limited to polyethylene glycol diacrylate mol. weight 3400 (PEGDA)—from Nektar Therapeutics (Huntsville, Ala), polyethylene oxide diacrylate (PEODA), other synthetic polymers such as partially or fully hydrolyzed poly (vinyl alcohol), poly (vinylpyrrolidone), poly (ethyloxazoline), poly (ethylene oxide)-co-poly (propylene oxide) block copolymers (poloxamers and meroxapols), poloxamines, carboxymethyl cellulose, and hydroxyalkylated celluloses such as hydroxyethyl cellulose and methylhydroxypropyl cellulose, and natural polymers such as polypeptides, polysaccharides or carbohydrates such as Ficoll) polysucrose, hyaluronic acid, dextran, heparan sulfate, chondroitin sulfate, heparin, or alginate, and proteins such as gelatin, collagen, albumin, or ovalbumin (note: for photopolymerization of natural polymers they must first be modified—ie Methacrylated Hylauronic Acid)

Potential photoinitiators that can be used in this invention include but are not limited to Igracure 2959 (commercially available from Ciba Specialty Chemicals Corp., Tarrytown, N.Y.), HIM (Polysciences), or 2-hydroxyethyl mathacrylate (HEMA). With both initiators the active hydroxy group can be reacted with suitable functionalized unsaturated polymers. In addition, various dyes and an amine catalyst are known to form an active species when exposed to external radiation. Specifically, light absorption by the dye causes the dye to assume a triplet state, which subsequently reacts with the amine to form the active species that initiates polymerization. Numerous dyes can be used for photopolymerization, and these include erythrosin, phloxime, rose bengal, thonine, camphorquinone, ethyl eosin, eosin, methylene blue, riboflavin, 2,2-dimethyl-2-phenylacetophenone, 2-methoxy-2-phenylacetophenone, 2,2-dimethoxy-2-phenyl acetophenone, other acetophenone derivatives, and camphorquinone. Suitable cocatalyst include amines such as N-methyl diethanolamine, N,N-dimethyl benzylamine, triethanol amine, triethylamine, dibenzyl amine, N-benzylethanolamine, N-isopropyl benzylamine. Triethanolamine is a preferred cocatalyst with one of these dyes.

When UV polymers are utilized, polymerization can be initiated by irradiation with light at a wavelength of between about 200-700 nm, most preferably in the long wavelength ultraviolet range or visible range, 320 nm or higher, and most preferably between about 365 and 514 nm. Photoinitiators (in a concentration not toxic to the cells, less than 0.1% by weight, more preferably between 0.05 and 0.01% by weight percent initiator) will crosslink upon exposure to light equivalent to between one and 3 mWatts/cm2.

While photopolymers are preferred for making the hydrogels, because it is convenient to control polymerization using external radiation supplied through a surgical scope, the present invention can be practiced using other polymer materials and polymerization initiators. Examples of other materials which can be used to form a hydrogel include (a) modified alginates, (b) polysaccharides (e.g. gellan cum and carrageenans) which gel by exposure to monovalent cations, (c) polysaccharides (e.g., hyaluronic acid) that are very viscous liquids or are thiotropic and form a gel over time by the slow evolution of structure, and (d) polymeric hydrogel precursors (e.g., polyethylene oxide-polypropylene glycol block copolymers and proteins). U.S. Pat. No. 6,224,893 B1, incorporated by reference in its entirety herein, provides a detailed description of the various polymers, and the chemical properties of such polymers, that are suitable for making hydrogels in accordance with the present invention, and this patent is incorporated herein by reference in its entirety.

The list of hydrogels described in U.S. Pat. No. 6,224,893 B1 are reproduced below. The polymerizable agent of the present invention may comprise monomers, macromers, oligomers, polymers, or a mixture thereof. The polymer compositions can consist solely of covalently crosslinkable polymers, or blends of covalently and ionically crosslinkable or hydrophilic polymers.

Suitable hydrophilic polymers include, but are not limited to, synthetic polymers such as poly(ethylene glycol), poly(ethylene oxide), partially or fully hydrolyzed poly(vinyl alcohol), poly(vinylpyrrolidone), poly(ethyloxazoline), poly(ethylene oxide)-co-poly(propylene oxide) block copolymers (poloxamers and meroxapols), poloxamines, carboxymethyl cellulose, and hydroxyalkylated celluloses such as hydroxyethyl cellulose and methylhydroxypropyl cellulose, and natural polymers such as polypeptides, polysaccharides or carbohydrates such as FICOLL™, polysucrose, hyaluronic acid, dextran, heparan sulfate, chondroitin sulfate, heparin, or alginate, and proteins such as gelatin, collagen, albumin, or ovalbumin or copolymers or blends thereof. As used herein, “celluloses” includes cellulose and derivatives of the types described above; “dextran” includes dextran and similar derivatives thereof.

Examples of materials that can be used to form a hydrogel include modified alginates. Alginate is a carbohydrate polymer isolated from seaweed, which can be crosslinked to form a hydrogel by exposure to a divalent cation such as calcium, as described, for example in WO 94/25080, the disclosure of which is incorporated herein by reference in its entirety. Alginate is ionically crosslinked in the presence of divalent cations, in water, at room temperature, to form a hydrogel matrix. Modified alginate derivatives may be synthesized which have an improved ability to form hydrogels. The use of alginate as the starting material is advantageous because it is available from more than one source, and is available in good purity and characterization. As used herein, the term “modified alginates” refers to chemically modified alginates with modified hydrogel properties. Naturally occurring alginate may be chemically modified to produce alginate polymer derivatives that degrade more quickly. For example, alginate may be chemically cleaved to produce smaller blocks of gellable oligosaccharide blocks and a linear copolymer may be formed with another preselected moiety, e.g. lactic acid or epsilon-caprolactone. The resulting polymer includes alginate blocks which permit ionically catalyzed gelling, and oligoester blocks which produce more rapid degradation depending on the synthetic design. Alternatively, alginate polymers may be used wherein the ratio of mannuronic acid to guluronic acid does not produce a film gel, which are derivatized with hydrophobic, water-labile chains, e.g., oligomers of epsilon-caprolactone. The hydrophobic interactions induce gelation, until they degrade in the body.

Additionally, polysaccharides which gel by exposure to monovalent cations, including bacterial polysaccharides, such as gellan gum, and plant polysaccharides, such as carrageenans, may be crosslinked to form a hydrogel using methods analogous to those available for the crosslinking of alginates described above. Polysaccharides which gel in the presence of monovalent cations form hydrogels upon exposure, for example, to a solution comprising physiological levels of sodium. Hydrogel precursor solutions also may be osmotically adjusted with a nonion, such as mannitol, and then injected to form a gel.

Polysaccharides that are very viscous liquids or are thixotropic, and form a gel over time by the slow evolution of structure, are also useful. For example, hyaluronic acid, which forms an injectable gel with a consistency like a hair gel, may be utilized. Modified hyaluronic acid derivatives are particularly useful. As used herein, the term “hyaluronic acids” refers to natural and chemically modified hyaluronic acids. Modified hyaluronic acids may be designed and synthesized with preselected chemical modifications to adjust the rate and degree of crosslinking and biodegradation. For example, modified hyaluronic acids may be designed and synthesized which are esterified with a relatively hydrophobic group such as propionic acid or benzylic acid to render the polymer more hydrophobic and gel-forming, or which are grafted with amines to promote electrostatic self-assembly. Modified hyaluronic acids thus may be synthesized which are injectable, in that they flow under stress, but maintain a gel-like structure when not under stress. Hyaluronic acid and hyaluronic derivatives are available from Genzyme, Cambridge, Mass. and Fidia, Italy.

Other polymeric hydrogel precursors include, but are not limited only to, polyethylene oxide-polypropylene glycol block copolymers such as PLURONICS™ or TETRONICS™, which are crosslinked by hydrogen bonding and/or by a temperature change, as described in Steinleitner et al., Obstetrics & Gynecology, 77:48-52 (1991); and Steinleitner et al., Fertility and Sterility, 57:305-308 (1992). Other materials which may be utilized include proteins such as fibrin, collagen and gelatin. Polymer mixtures also may be utilized. For example, a mixture of polyethylene oxide and polyacrylic acid which gels by hydrogen bonding upon mixing may be utilized. In one embodiment, a mixture of a 5% w/w solution of polyacrylic acid with a 5% w/w polyethylene oxide (polyethylene glycol, polyoxyethylene) 100,000 can be combined to form a gel over the course of time, e.g., as quickly as within a few seconds.

### Ionic Cross Linkable Polymers

Water soluble polymers with charged side groups may be crosslinked by reacting the polymer with an aqueous solution containing ions of the opposite charge, either cations if the polymer has acidic side groups or anions if the polymer has basic side groups. Examples of cations for cross-linking of the polymers with acidic side groups to form a hydrogel are monovalent cations such as sodium, divalent cations such as calcium, and multivalent cations such as copper, calcium, aluminum, magnesium, strontium, barium, and tin, and di-, tri- or tetra-functional organic cations such as alkylammonium salts. Aqueous solutions of the salts of these cations are added to the polymers to form soft, highly swollen hydrogels and membranes. The higher the concentration of cation, or the higher the valence, the greater the degree of cross-linking of the polymer. Additionally, the polymers may be crosslinked enzymatically, e.g., fibrin with thrombin.

Suitable ionically crosslinkable groups include, but are not limited to, phenols, amines, imines, amides, carboxylic acids, sulfonic acids and phosphate groups. Aliphatic hydroxy groups are not considered to be reactive groups for the chemistry disclosed herein. Negatively charged groups, such as carboxylate, sulfonate and phosphate ions, can be crosslinked with cations such as calcium ions. The crosslinking of alginate with calcium ions is an example of this type of ionic crosslinking. Positively charged groups, such as ammonium ions, can be crosslinked with negatively charged ions such as carboxylate, sulfonate and phosphate ions. Preferably, the negatively charged ions contain more than one carboxylate, sulfonate or phosphate group.

The preferred anions for cross-linking of the polymers to form a hydrogel are monovalent, divalent or trivalent anions such as low molecular weight dicarboxylic acids, for example, terephthalic acid, sulfate ions and carbonate ions. Aqueous solutions of the salts of these anions are added to the polymers to form soft, highly swollen hydrogels and membranes, as described with respect to cations.

A variety of polycations can be used to complex and thereby stabilize the polymer hydrogel into a semi-permeable surface membrane. Examples of materials that can be used include polymers having basic reactive groups such as amine or imine groups, having a preferred molecular weight between 3,000 and 100,000, such as polyethylenimine and polylysine. These are commercially available. One polycation is poly(L-lysine); examples of synthetic polyamines are: polyethyleneimine, poly(vinylamine), and poly(allyl amine). There are also natural polycations such as the polysaccharide, chitosan.

Polyanions that can be used to form a semi-permeable membrane by reaction with basic surface groups on the polymer hydrogel include polymers and copolymers of acrylic acid, methacrylic acid, and other derivatives of acrylic acid, polymers with pendant SO 3 H groups such as sulfonated polystyrene, and polystyrene with carboxylic acid groups. These polymers can be modified to contain active species polymerizable groups and/or ionically crosslinkable groups. Methods for modifying hydrophilic polymers to include these groups are well known to those of skill in the art.

The polymers may be intrinsically biodegradable, but are preferably of low biodegradability (for predictability of dissolution) but of sufficiently low molecular weight to allow excretion. The maximum molecular weight to allow excretion in human beings (or other species in which use is intended) will vary with polymer type, but will often be about 20,000 daltons or below. Usable, but less preferable for general use because of intrinsic biodegradability, are water-soluble natural polymers and synthetic equivalents or derivatives, including polypeptides, polynucleotides, and degradable polysaccharides.

The polymers can be a single block with a molecular weight of at least 600, preferably 2000 or more, and more preferably at least 3000. Alternatively, the polymers can include can be two or more water-soluble blocks which are joined by other groups. Such joining groups can include biodegradable linkages, polymerizable linkages, or both. For example, an unsaturated dicarboxylic acid, such as maleic, fumaric, or aconitic acid, can be esterified with hydrophilic polymers containing hydroxy groups, such as polyethylene glycols, or amidated with hydrophilic polymers containing amine groups, such as poloxamines.

Covalently crosslinkable hydrogel precursors also are useful. For example, a water soluble polyamine, such as chitosan, can be cross-linked with a water soluble diisothiocyanate, such as polyethylene glycol diisothiocyanate. The isothiocyanates will react with the amines to form a chemically crosslinked gel. Aldehyde reactions with amines, e.g., with polyethylene glycol dialdehyde also may be utilized. A hydroxylated water soluble polymer also may be utilized.

Alternatively, polymers may be utilized which include substituents which are crosslinked by a radical reaction upon contact with a radical initiator. For example, polymers including ethylenically unsaturated groups which can be photochemically crosslinked may be utilized, as disclosed in WO 93/17669, the disclosure of which is incorporated herein by reference in its entirety. In this embodiment, water soluble macromers that include at least one water soluble region, a biodegradable region, and at least two free radical [symbol] polymerizable regions, are provided. The macromers are polymerized by exposure of the polymerizable regions to free radicals generated, for example, by photosensitive chemicals and or light. Examples of these macromers are PEG-oligolactyl-acrylates, wherein the acrylate groups are polymerized using radical initiating systems, such as an eosin dye, or by brief exposure to ultraviolet or visible light. Additionally, water soluble polymers which include cinnamoyl groups which may be photochemically crosslinked may be utilized, as disclosed in Matsuda et al., ASAID Trans., 38:154-157 (1992).

The term “active species polymerizable group” is defined as a reactive functional group that has the capacity to form additional covalent bonds resulting in polymer interlinking upon exposure to active species. Active species include free radicals, cations, and anions. Suitable free radical polymerizable groups include ethylenically unsaturated groups (i.e., vinyl groups) such as vinyl ethers, allyl groups, unsaturated monocarboxylic acids, unsaturated dicarboxylic acids, and unsaturated tricarboxylic acids. Unsaturated monocarboxylic acids include acrylic acid, methacrylic acid and crotonic acid. Unsaturated dicarboxylic acids include maleic, flimaric, itaconic, mesaconic or citraconic acid. In one embodiment, the active species polymerizable groups are preferably located at one or more ends of the hydrophilic polymer. In another embodiment, the active species polymerizable groups are located within a block copolymer with one or more hydrophilic polymers forming the individual blocks. The preferred polymerizable groups are acrylates, diacrylates, oligoacrylates, dimethacrylates, oligomethacrylates, and other biologically acceptable photopolymerizable groups. In certain preferred embodiments, acrylates are the most preferred active species polymerizable group.

In general, the polymers are at least partially soluble in aqueous solutions, such as water, buffered salt solutions, or aqueous alcohol solutions. Methods for the synthesis of the other polymers described above are known to those skilled in the art. See, for example Concise Encyclopedia of Polymer Science and Polymeric Amines and Ammonium Salts, E. Goethals, editor (Pergamen Press, Elmsford, N.Y. 1980). Many polymers, such as poly(acrylic acid), are commercially available. Naturally occurring and synthetic polymers may be modified using chemical reactions available in the art and described, for example, in March, “Advanced Organic Chemistry,” 4th Edition, 1992, Wiley [symbol] Interscience Publication, New York.

Preferably, the hydrophilic polymers that include active species or crosslinkable groups include at least 1.02 polymerizable or crosslinkable groups on average, and, more preferably, each includes two or more polymerizable or crosslinkable groups on average. Because each polymerizable group will polymerize into a chain, crosslinked hydrogels can be produced using only slightly more than one reactive group per polymer (i.e., about 1.02 polymerizable groups on average). However, higher percentages are preferable, and excellent gels can be obtained in polymer mixtures in which most or all of the molecules have two or more reactive double bonds. Poloxamines, an example of a hydrophilic polymer, have four arms and thus may readily be modified to include four polymerizable groups.

### Ex Vivo Labelling Method of In Vivo Imaging

In one aspect, the instant invention provides methods of ex-vivo labeling of a liposome polymer matrix for in vivo imaging by contacting a liposome polymer matrix ex vivo with a labeling agent such that liposome polymer matrix becomes labeled, thereby labeling a liposome polymer matrix for in vivo imaging. In one embodiment, the liposome polymer matrix is transplanted into a subject. In another embodiment, the labeling agent is detectable by a modality selected from the group consisting of X-ray, CT, ultrasound, Raman, and magnetic resonance. In another embodiment, the labeling agent is a multimode-detectable labeling agent, e.g., it is detectable by at least two modalities, e.g., such as X-ray, CT, ultrasound, Raman, and magnetic resonance. In one embodiment, the labeling agent is a perfluorocarbon (PFC), e.g., perfluoro-15-crown-5-ether (PFCE), perfluorooctylbromide (PFOB). In another embodiment, the labeling agent is a colloidal metal particle, e.g., a colloidal gold or silver particle. In another embodiment, the particle is a core-shell particle. In some embodiments, the shell of the core-shell particle is derivatized with functional groups for the conjugation of a bioactive molecule, e.g., a peptide or polypeptide such as an antibody of fragment thereof.

In another embodiment, the labeling agent is detectable by a modality selected from the group consisting of X-ray, CT, ultrasound, Raman, and magnetic resonance.

In another embodiment, the labeling agent is a multimode-detectable labeling agent, e.g., it is detectable by at least two modalities, e.g., such as X-ray, CT, ultrasound, Raman, and magnetic resonance. In another embodiment, the particle is a core-shell particle. In some embodiments, the shell of the core-shell particle is derivatized with functional groups for the conjugation of a bioactive molecule, e.g., a peptide or polypeptide such as an antibody of fragment thereof.

In one aspect, the invention provides methods of locating a liposome polymer matrix comprising a multimode-detectable labeling agent in a subject comprising, obtaining two or more images of the subject or a portion thereof, overlaying the images, and analyzing the images to determine the location of the liposome polymer matrix in the subject. In one embodiment, the images are selected from X-ray, CT, ultrasound, Raman, and magnetic resonance images. In another embodiment, the analysis step is preformed using a computer program.

In another aspect, the instant invention provides methods of measuring the presence of a liposome polymer matrix labeled with a fluorescent agent by labeling a liposome polymer matrix with a fluorescent agent, irradiating a tissue comprising the liposome polymer matrix with radiation, detecting a fluorescence emission spectrum of the fluorescent agent, thereby measuring the presence of a liposome polymer matrix labeled with a fluorescent contrast agent.

In another aspect, the instant invention provides methods for determining if a liposome polymer matrix contains a single or multiple contrast agents that produce a Raman spectra by a) labeling a liposome polymer matrix with a raman reporting contrast agent by the method of any one disclosed herein by administering a contrast agent with antibody bound to the contrast agent so after systemic administration it binds to the antibody target; b) irradiating the tissue with a beam of infrared monochromatic light; c) obtaining the infrared Raman spectrum from the labeled liposome polymer matrix d) comparing said infrared Raman spectrum so obtained from the labeled liposome polymer matrixs with the infrared Raman spectra correspondingly obtained from known samples of liposome polymer matrixs non containing contrast agent.

### Monitoring

In another aspect the instant invention provides systems for monitoring the presence of a raman detectable agent in or on a liposome polymer matrix using low-resolution Raman spectroscopy using a catheter having a first end and a second end with an excitation fiber extending therebetween, the excitation fiber suitable to transmit multi-mode radiation from the first end to the second end to irradiate a target region; a multi-mode laser coupled to the first end of the excitation fiber, the laser generates multi-mode radiation for irradiating the target region to produce a Raman spectrum consisting of scattered electromagnetic radiation; a low-resolution dispersion element positioned to receive and separate the scattered radiation into different wavelength components; a detection array, optically aligned with the dispersion element for detecting at least some of the wavelength components of the scattered light; and a processor for processing the data from the detector array to monitor a Raman detectable agent.

Contrast agents that can be incorporated into either the aqueous portion of the liposome or directly into the polymer matrix include, for example, perfluorocarbons (PFCs). Representative perfluorocarbons include bis(F-alkyl) ethanes such as F-44E, i-F-i36E, and F-66E; cyclic fluorocarbons, such as F-decalin, perfluorodecalin or “FDC), F-adamantane (“FA”), F [symbol] methyladamantane (“FMA”), F-1,3-dimethyladamantane (“FDMA”), F-di- or F [symbol] trimethylbicyclo[3,3,1]nonane (“nonane”); perfluorinated amines, such as F [symbol] tripropylamine (“FTPA”) and F-tri-butylamine (“FTBA”), F-4-methyloctahydroquinolizine (“FMOQ”), F-n-methyl-decahydroisoquinoline (“FMIQ”), F [symbol] n-methyldecahydroquinolin (“FHQ”), F-n-cyclohexylpurrolidine (“FCHP”) and F-2-butyltetrahydrofuran (“FC-75” or “RM101”). Brominated perfluorocarbons include 1-bromo-heptadecafluoro-octane (sometimes designated perfluorooctylbromide or “PFOB”), 1-bromopenta-decafluoroheptane, and I-bromotridecafluorohexane (sometimes known as perfluorohexylbromide or “PFHB”). PFOB is a preferred labeling agent for use in the methods of the invention. Other brominated fluorocarbons are disclosed in U.S. Pat. No. 3,975,512. Other suitable perfluorocarbons are mentioned in EP 908 178 A1.

Representative nanoparticles that can be incorporated into the polymer matrix include Cobalt Nanoparticles, Iron Oxide Nanopowder, Niobium Oxide Nanopowder, Thulium Nanoparticles, Cobalt Oxide Nanopowder, Lanthanum Nanoparticles, Palladium Nanoparticles, Tin Nanoparticles, Aluminum Oxide Nanopowder, Copper Nanoparticles, Lanthanum Oxide Nanopowder, Platinum Nanoparticles, Tin Oxide Nanopowder, Antimony Nanoparticles, Copper Oxide Nanopowder, Praseodymium Nanoparticles, Titanium Carbide Nanoparticles, Antimony Oxide Nanopowder, Dysprosium Nanoparticles, Lithium Manganese Oxide Nanoparticles, Praseodymium Oxide Nanopowder, Titanium Nanoparticles, Antimony Tin Oxide (ATO) Nanoparticles, Dysprosium Oxide Nanopowder, Lithium Nanoparticles, Rhenium Nanoparticles, Titanium Nitride Nanoparticles, Barium Titanate Nanoparticles, Erbium Nanoparticles, Lithium Titanate Nanoparticles, Ruthenium Nanoparticles, Titanium Oxide Nanopowder, Beryllium Nanoparticles, Erbium Oxide Nanopowder, Lithium Vanadate Nanoparticles, Samarium Nanoparticles, Tungsten Carbide Nanoparticles, Bismuth Oxide Nanopowder, Europium Nanoparticles, Lutetium Nanoparticles, Samarium Oxide Nanopowder, Tungsten Nanoparticles, Boron Carbide Nanoparticles, Europium Oxide Nanopowder, Magnesium Nanoparticles, Silicon Carbide Nanoparticles, Tungsten Oxide Nanopowder, Boron Nitride Nanoparticles, Gadolinium Nanoparticles, Magnesium Oxide Nanopowder, Silicon Nanoparticles, Vanadium Oxide Nanopowder, Calcium Carbonate Nanoparticles, Gadolinium Oxide Nanopowder, Manganese Nanoparticles, Silicon Nanotubes, Ytterbium Nanoparticles, Calcium Chloride Nanoparticles, Gold Nanoparticles, Manganese Oxide Nanopowder, Silicon Nitride Nanoparticles, Yttria stabilized Zirconia, Calcium Oxide Nanopowder, Hafnium Oxide Nanopowder, Molybdenum Nanoparticles, Silicon Oxide Nanopowder, Yttrium Nanoparticles, Calcium Phosphate Nanoparticles, Holmium Nanoparticles, Molybdenum Oxide Nanopowder, Silver Nanoparticles, Zinc Oxide Nanopowder, Carbon Nanohorns, Indium Nanoparticles, Neodymium Nanoparticles, Strontium Carbonate Nanoparticles, Zirconium Nanoparticles, Carbon Nanoparticles, Indium Oxide Nanopowder, Neodymium Oxide Nanopowder, Strontium Titanate Nanoparticles, Zirconium Oxide Nanopowder, Carbon Nanotubes, Iridium Nanoparticles, Nickel Nanoparticles, Tantalum Nanoparticles, Cerium Nanoparticles, Iron Cobalt Nanopowder, Nickel Oxide Nanopowder, Tantalum Oxide Nanopowder, Cerium Oxide Nanopowder, Iron Nanoparticles, Nickel Titanium Nanopowder, Terbium Nanoparticles, Chromium Oxide Nanopowder, Iron Nickel Nanopowder, Niobium Nanoparticles, Terbium Oxide Nanopowder, Carbon 60 fullerenes, Carbon 70 fullerens and Carbon 85 fullerenes, single wall carbon nanotubes, multi-wall carbon nanotubes, carbon nanofibers.

In one embodiment, the labeling agent is a radiopaque agent. As disclosed therein, monobromo, and dibromo perfluorocarbons, including both aliphatic and cyclic compounds, exhibit radiopaque properties which make such brominated perfluorocarbons useful.

The methods of the instant invention can use fluorescent labeling agents. Numerous fluorescent agents are available for use in the methods of the invention. Exemplary fluorescent labeling agents include, Rhodamine 101, Nile Red, Nileblue A, Fluorescein, Sulforhodamine B, Sulforhodamine G, PdTFPP, DiA, 5(6)-Carboxyfluorescein, 2,7 DDichlorofluorescein, 1,1\-Diethyl-4,4\-carbocyanine iodide, 3,3-Diethylthiadicarbocyanine iodide, Lucifer Yellow CH Dilitium salt 5(6)-Carboxytetramethylrhodamine B, N,N-Bis(2,4,6-trimethylphenyl)-3, 4:9, 10-perylenebis(dicarboximide, Rhodamine B, 2-Di-1-ASP, Dichlorotris(1,10-phenanthroline)ruthenium(II), Tris(4,7-diphenyl-1,10 phenanthroline)ruthenium(II) TMS, Tris(4,4-diphenyl-2,2-bipyridine)ruthenium(II), chloride, Resorufin, Ethyl Eosin, Ethyl Eosin, Coumarin 6, Rhodamine 6G, 8-Benzyloxy-5,7-diphenylquinoline, 8-Benzyloxy-5,7-diphenylquinoline (protonated), DY-500XL, DY-554, DY-633, DY-615, DY-590, DY-650, DY-490XL, DY-520XL, DY-485XL, DY-480XL, DY-555, DY-590, DY-630, DY-631, DY-635, DY-636, DY-647, DY-651, DY-656, DY-673, DY-675, DY-676, DY-680, DY-681, DY-700, DY-701, DY-730, DY-731, DY-750, DY-751, DY-776, DY-782, EVOblue-30, Adams Apple Red 680, Adirondack Green 520, Birch Yellow 580, Catskill Green 540 Fort Orange 600, Hemo Red 720, Hops Yellow 560, Lake Placid 490, Maple Red-Orange 620, Snake-Eye Red 900, QD525, QD565, QD585, QD605, QD655, QD705, QD800, ATTO 465, ATTO 425, ATTO 488, ATTO 495, ATTO 520, ATTO 550, ATTO 565, ATTO 590, ATTO 610, ATTO 620, ATTO 635, ATTO 647, ATTO 655, ATTO 680, ATTO 700, Alexa Fluor 350, Alexa Fluor 430, Alexa Fluor 480, Alexa Fluor 633, 5-FAM, DyLight 549 5-TAMRA, 6-HEX, 6-carboxyrhodamine 6G, 6-JOE, 6-TET, BOBO [symbol] 1, BOBO-3, POPO-1, POPO-3, TOTO-1, TOTO-3, YOYO-1, YOYO-3, aminomethylcoumarin, APC, BCECF, Amplex Gold (product), dichlorofluorescein, TO [symbol] PRO-1, TO-PRO-3, SYTO 11, SYTO 13, SYTO 17, SYTO 45, PO-PRO-1, PO-PRO-3, propidium iodide, Pro-Q Diamond, Pro-Q Emerald, quinine, resorufin, rhod-2, rhodamine 110, rhodamine 123, Rhodamine Green, YO-PRO-1, YO-PRO-3, SYTOX Blue, SYTOX Green, SYTOX Orange, Rhodamine Red-X rhodamine, Rhodol Green, R-phycoerythrin, SBFI, Sodium Green sulforhodamine 101, SYBR Green I, SYPRO Ruby, tetramethylrhodamine, Texas Red-X, X-rhod-1, Alexa Fluor 500, Alexa Fluor 514, Alexa Fluor 610, Alexa Fluor 635 Calcein red-orange, Carboxynaphthofluorescein, DiIC18(3, ELF 97, Ethidium bromide, Alexa Fluor 532, Alexa Fluor 546, Alexa Fluor 555, Alexa Fluor 568, Alexa Fluor 594, Alexa Fluor 610-R-PE, Alexa Fluor 647, Alexa Fluor 647-R-PE Alexa Fluor 660, Alexa Fluor 680, Alexa Fluor 680-APC, Alexa Fluor 680-R-PE Alexa Fluor 700, Alexa Fluor 750, FITC, Fluo-3, Fluo-4, fluoro-emerald, FM 1-43, FM 4-64, Hoechst 33258, JC-1, JOJO-1, LOLO-1, lucifer yellow CH, LysoSensor Blue, DND-192, LysoSensor Green DND-153, YoYo-1 ssDNA, YoYo-1 dsDNA, YoYo-1, Yakima Yellow, tdTomato, Tb (Soini), SYTO RNASelect, SYTO RNASelect, Calcofluor white 2MR, DAPI, DDAO, Deep Purple, Diversa Cyan-FP, Diversa Green-FP, Dragon Green Envy Green, Ethidium bromide, Ethyl Nile Blue A, Eu (Soini), Eu203 nanoparticles, EvaGreen, mBanana, mCherry, Methylene Blue, Methylene Blue, Flash Red EX, mHoneyDew, mOrange, mPlum, mRaspberry, mRFP1.2 (Wang), mStrawberry (Shaner), tangerine (Shaner), Pacific Orange, Plum Purple, Pontamine fast scarlet 4B, Surf Green EX, Suncoast Yellow, Cresyl Violet Perchlorate, DyLight 488Allophycocyanin, Coumarin 6, C-Phycocyanin, CryptoLight CF1, CryptoLight CF2, CryptoLight CF3, CryptoLight CF4, CryptoLight CF5, CryptoLight CF6, R [symbol] phycoerythrin, SensiLight PBXL-1, SensiLight PBXL-3, Spectrum Aqua, Spectrum Blue, Spectrum Fred, Spectrum Gold, Spectrum Green, Spectrum Orange, Spectrum Red, 1,4-Diphenylbutadiene, 1,2-Diphenylacetylene, 1,4-Diphenylbutadiyne, 1,6-Diphenylhexatriene, Ir(Cn)2(acac), 7-Methoxycoumarin-4-Acetic Acid 9,10-Bis(Phenylethynyl)Anthracene, 9,10-Diphenylanthracene, Acridine OrangeAcridine Yellow, Anthracene, Auramine 0, Benzene, Cy3B Biphenyl, C3-Indocyanine, C3-Indocyanine, C3-Oxacyanine, C3-Thiacyanine Dye (EtOH), C3-Thiacyanine Dye (PrOH), C5-Indocyanine, C5-Oxacyanine, C5-Thiacyanine, C7-Indocyanine, C7-Oxacyanine, Coumarin 1, Dye-33, Dye-28, Dye-45, Cy3, DRAQ5, Ethyl-p [symbol] Dimethylaminobenzoate, Cy3.5, Cy2, CBQCA, Oregon Green 514, Oregon Green 488, nile red, nile blue, NeuroTrace 500525, NBD-X, monobromobimane, MitoTracker Red CMXRos, MitoTracker Orange CMTMRos, MitoTracker Green FM, Marina Blue, Magnesium Orange, Magnesium Green, LysoTracker Red DND-99, LysoTracker Green DND-26, LysoTracker Blue DND-22, LysoSensor YellowBlue DND-160, LysoSensor Green DND-153, LysoSensor Blue DND-192, lucifer yellow CH, JC-1, indo-1, fura-2, Fura Red, Coumarin 343, Cy3Cy5 ET, Cy5.5, Cy5, Cy7, CypHer5, Coumarin 30, Coumarin 314, ECF, ECL Plus, PA-GFP (post-activation), PA-GFP (pre-activation), WEGFP (post-activation), CHOxAsH-CCXXCC, F1AsH-CCXXCC, ReAsH-CCXXCC, NIR1, NIR2, NIR3, NIR4, NIR820, SNIR1, SNIR2, SNIR4, AmCyanl, AsRed2, Azami Green monomeric, Azami Green, CFP (Campbell Tsien 2003), Citrine (Campbell Tsien 2003), DsRed, DsRed, DsRed Dimer2 (Campbell Tsien 2003), DsRed-Express T1, EBFP (Patterson 2001), ECFP (Patterson 2001), EGFP (Campbell Tsien 2003), EGFP (Patterson 2001), Eosin Y, Fluorescein, Fluorescein-Dibase, Hoechst-33258, Hoechst-33258, Kaede Green, Magnesium Octaethylporphyrin, DyLight 680, AAA, DyLight 649, DyLight 633, Magnesium Phthalocyanine, Magnesium Phthalocyanine, Magnesium Tetraphenylporphyrin Merocyanine 540, Naphthalene, Nile Blue (EtOH), Nile Blue, Nile Red, Octaethylporphyrin, Oxazine 1, Oxazine 170, Perylene, Phenol, Phenylalanine, Phthalocyanine, Pinacyanol-Iodide, Piroxicam, POPOP, Porphin, Lucifer Yellow CH, P [symbol] Quaterphenyl, Proflavin, P-Terphenyl, Pyrene, Quinine Sulfate, Rhodamine 123, Ethyl-p [symbol] Dimethylaminobenzoate, 1,6-Diphenylhexatriene, 2-Methylbenzoxazole, Rhodamine 6G, Rhodamine B, Riboflavin, Rose Bengal, Squarylium dye III, Stains All, Stilbene, Sulforhodamine 101, Tetrakis(o-Aminophenyl)Porphyrin, Tetramesitylporphyrin, Tetraphenylporphyrin, Tetraphenylporphyrin, Tetra-t-Butylazaporphine, Tetra-t [symbol] Butylnaphthalocyanine, Toluene, Tris(2,2-Bipyridyl)Ruthenium(II) chloride, Tryptophan.

Methods of Making—Liposome polymer matrix

### Bioactive Agents

The substances to be entrapped in the liposome polymer matrix according to the invention include any imaginable therapeutically or diagnostically active compounds. As such, one may recite drugs like analgesics, narcotics, antibiotics, sulfamides, steroids, X-ray opacifiers, NMR contrast agents and the like. X-ray opacifiers include for instance organic iodinated compounds like N,N′-bis[2-hydroxy-1-(hydroxymethyl)-ethyl]-5-[(2-hydroxy-1-oxopropyl)-amino]-2,4,6-triiodo-1,3-benzene-dicarboxyamide (iopamidol); metrizamide; diatrizoic acid; sodium diatrizoate; meglumine diatrizoate; acetrizoic acid and its soluble salts; diprotrizoic acid; iodamide; sodium iodipamide; meglumine diopamide; iodohippuric acid and the soluble salts thereof; iodomethamic acid; iodopyracetiodo-2-pyridone-N-acetic acid; 3,5-diiodo-4-pyridone-N-acetic acid (Iodopyracet) and its diethyl ammonium salt; iothalmic acid; metrizoic acid and its salts; the ipanoic, iocetamic and iophenoxic acids and their salts; sodium tyropanoate; sodium opidate and other like iodized compounds.

### Organic Solvents for Liposome Construction

Hydrophilic, low molecular weight water miscible alcohols with less than 10 carbon atoms, preferably less than 6 carbon atoms are preferred. Typical alcohols used in this invention are methyl alcohol, ethyl alcohol, n-propyl alcohol, isopropyl alcohol and n-butyl alcohol, sec-butyl alcohol and tert-butyl alcohol, pentanol and ethylene glycol and propylene glycol. Generally, the organic solvent is added to the liposomes up to about 10% (v/v), preferably up to about 20%, more preferably up to about 30%. At these concentrations mean vesicle diameter remains substantially unchanged (i.e., changes by less than about 30%, preferably less than about 20%, more preferably less than about 10%), but membrane permeability is increased by 106 to 107 fold, or at least 104 fold over the permeability in the absence of solvent for small solutes (molecular weights less than about 1000). Consequently, solutes equilibrate across the vesicle membrane in seconds rather than months to years. High molecular weight solutes, for example macromolecular solutes such as proteins, enzymes, polysaccharides and the like also exhibit increased membrane permeation rates in the presence of an organic solvent, though typically the increase is not as dramatic as with low molecular weight solutes. With high molecular weight solutes (>1000 daltons, typically greater than 10,000 daltons and as high as 1 million daltons) increases in permeation rates of as much as 1000 fold to 10,000 fold can be obtained. At organic solvent concentrations substantially in excess of 30%, vesicle size increases dramatically and vesicle fusion and membrane solubilization occur. Vesicle fusion is not required for solute equilibration, since EPC vesicles maintained in 30% ethanol show no signs of fusion but have completely lost their ability to retain entrapped sucrose. Consequently this invention provides liposomes which are structurally stable and maintain their size over a range of ethanol concentrations that are sufficient to substantially increase membrane permeability to nonpolar species such as for example but not limited to, sucrose, carbohydrates, oligosaccharides, polysaccharides, peptides and the like. Nonpolar species are generally those that are substantially uncharged under the loading conditions. Preferably, the net charge on the molecule will be about 0-2, more preferably approximately zero. However, higher net charges can be tolerated if the charge is sufficiently diffuse. One skilled in the art will recognize that the organic solvent may be present admixed with the liposomes before the addition of the solute to be loaded. Without being bound by any particular theory, it is believed that the organic solvent perturbs the liposomal membrane to an extent sufficient to increase the permeability of the membrane to the solute thus allowing traversal of the solute into, across and through the membrane. This diffusion of solute through the membrane will continue until the concentration gradient of solute between the interior and exterior of the liposome is removed. Therefore, it is apparent that the order in which the liposome, solute and organic solvent mixture is generated from its constituent parts is not critical to the practice of the invention. Since the method rests on the presence of a concentration gradient of solute between the liposome and its exterior, it will also be apparent that the transmembrane permeation of solute will continue as long as this gradient exists. Therefore, any mechanism which “removes” the solute from the “low concentration” side of the membrane, by chemical reaction, adsorption and the like, will act to preserve the gradient and drive continued permeation of the solute. For example, if the solute is an enzyme substrate and is turned over by enzyme which is present only in the “low concentration” side of the membrane, transmembrane permeation of the solute can continue until the enzyme no longer turns over solute and the free solute concentrations on both sides are equal. Another example occurs when the solute is trapped by a binding substance, such as an antibody, which thereby reduces the effective concentration of the free solute, thus preserving the concentration gradient of free solute between the two sides of the membrane.

As should be apparent, the quantity of organic solvent used is such that the liposomal membrane is rendered permeable to the solute without permanently or irreparably disrupting or destroying the liposome. This is generally evidenced by the fact that liposome size remains substantially constant during the process and that liposomal fusion is not observed until much higher concentrations of organic solvent are used. The liposome can be resealed and rendered impermeable to the solute either when the solute concentrations in and outside the liposomes are equalized or at some earlier point in the process. The organic solvent-induced transmembrane permeation disclosed by the present invention does not require elevated temperatures. In particular, there is no necessity, unlike some of the methods of the prior art, that the loading process be practiced at temperatures at or in excess of the Tc of the liposomal membrane. Therefore, it is possible to employ in this invention heat-labile solutes which might otherwise be degraded. Loading or release of solutes can therefore be accomplished at room temperature or lower, such as at 4 degrees C. The ability to temporarily eliminate, or drastically reduce, the permeability barrier of vesicles without altering their size is of interest because it means that solutes added to a suspension of vesicles in ethanol will rapidly cross the membrane and equilibrate with the internal aqueous space. As described above, the permeability coefficient for sucrose is 106 to 107 fold greater for EPC vesicles in 30% ethanol compared to buffer alone. Consequently, in ethanol, sucrose equilibrates across the vesicle membrane in seconds rather than months to years. However, this process is not of practical use with respect to loading solutes (such as drugs) into preformed vesicles unless the permeability barrier can be restored rapidly enough to effectively trap solute. In order for this method to effectively trap solutes after membrane permeation, the permeability barrier must be restored rapidly enough to prevent solute re-equilibration. Generally this is accomplished by diluting the organic solvent by at least a factor of at least about two, preferably by a factor of ten, more preferably by a factor of at least twenty. For example, when sucrose is loaded into liposomes at a 30% ethanol concentration, diluting the ethanol concentration to about 10 [symbol] 15% was sufficient to reduce the permeability coefficient by several orders of magnitude, thus effectively trapping the contents of the liposome in the liposome. Dilution can be accomplished by any available means ranging from the direct addition of additional aqueous media, flash evaporation and gel filtration, the latter being particularly effective at removing substantially all of the organic solvent. Any other method of rapidly removing or reducing the concentration of the organic solvent, such as, for example, selective absorption may also be used. The type of buffer in which the liposomes are initially dispersed is not critical to the invention. Thus, unlike some of the methods disclosed in the prior art, there is generally no requirement that the aqueous medium in which the liposomes are suspended be of low ionic strength or osmolality. Typical buffers include NaCl, KCl, sodium or potassium phosphate, carbonate, bicarbonate, borate, tris, HEPES (N-2-hydroxyethyl-piperazine-N′-2-ethanesulfonic acid), IVIES (2-N-morpholinoethanesulfonic acid), PIPES (piperazine-N,N′-bis(2-ethanesulfonic acid)) and the like. The pH is whatever is required to maintain liposomal integrity and can range from 2 to 11 and is typically about 6 to 8. A variety of solutes can be induced to cross the liposomal membrane by transmembrane permeation and loaded into liposomes using the solvent loading method disclosed herein. Unlike the ion gradient methods disclosed in the prior art, the solvent loading method is not limited to ionizable solutes. Thus, uncharged or neutral species or substances that are not capable of being induced to carry a charge by protonation, cation or anion binding and the like, can be loaded into the liposomes. The transmembrane permeation of the solute can be simply accomplished by adding an organic solvent to an aqueous dispersion of liposome and solute, waiting for the appropriate length of time, i.e., for equilibration to occur or until the solute concentration in the liposome reaches the desired level, and diluting out or removing the organic solvent. As was explained previously, the order of addition is unimportant and the solute can be added to a mixture of liposome and organic solvent. Alternatively, a mixture, suspension or solution and the like of the solute in the organic solvent can be added to the liposomes. Drugs, diagnostics, hormones, carbohydrates, oligo- and polysaccharides, vitamins, steroids, pesticides, plant nutrients or growth factors, proteins, antibodies, enzymes, chromophores, fluorophores, enzyme inhibitors and activators, cosmetics and the like may be loaded into the liposomes. Generally, highly negatively charged species such as polynucleotides do not cross liposomal membranes permeabilized by the solvent technique disclosed herein and are loaded with low levels of efficiency. This allows selective release or entrapment of neutral species in the presence of highly charged species by increasing membrane permeability by the solvent loading methods disclosed herein. It will be recognized that the charge on a molecule can be adjusted by a variety of methods, including but not limited to, varying the pH of the medium, providing counter ions which diffuse the charge, covalent modification. For example, charged oligonucleotides can be converted to less highly charged analogs which continue to display biological activity by methylation or conversion to the corresponding phosphorothioates, methylphosphonates and the like. Diagnostic reagents that may be used in this invention include radioactive materials, enzymes, chemiluminescent substances, spin labels, chromogens including fluorescent dyes and visible dyes, pH indicators (e.g., pyranine) and the like. Representative enzymes that may be loaded into liposomes or induced to cross the liposomal membrane by the organic solvent mediated increase in membrane permeability include, horseradish peroxidase, lactase, alkaline phosphatase, diaphorase, beta-galactosidase, ribonuclease, trypsin, chymotrypsin, amylase, esterase, phospholipase and the like. Representative drugs that may be used likewise in the present invention include anticancer agents such as doxorubicin and amphotericin, anti-inflammatory agents such prednisone, cortisone and the like, antihistamines such as chlorpromazine, antidepressants, anticonvulsants, anti-emetics, alkaloids such as vincristine and vinblastine, analgesics, tranquilizers etc. Other representative solutes that may be used in the present invention are disclosed in U.S. Pat. No. 4,389,330, col. 5 and 6 and U.S. Pat. No. 5,171,578, col. 6, the full disclosures of which are incorporated by reference in their entireties herein. Antibiotics such as gentamicin and the like, and other aminoglycosides, penicillins, cephalosporins, fluoroquinolones such as ciprofloxacin may also be loaded by the methods disclosed herein. Generally, the loading of all drugs that can cross the vesicle bilayer in the presence of up to 30% ethanol are contemplated by this invention. Such drugs can be readily identified by encapsulating the drug of interest and then adding ethanol to see if the drug is released from the vesicle. One will recognize that this method of loading and/or release is therefore independent of any particular molecular characteristic of the drug (e.g., charge, molecular weight, etc.).

Any cationic, lipophilic or amphiphilic drug which can partition into a lipid bilayer is suitable for use in practicing this invention, e.g., dibucaine, pilocarpine, quinine, prodipine, timolol, pentamidine, benadryl, dopamine, epinephrine, codeine, morphine, atropine, imipramine, quinidine, chloropromazine, and others.

Cationic compounds having antineoplastic activity against cancerous tissues or liposome polymer matrixes, including daunorubicin, doxorubicin, aclacinomycin A, vinblastine, vincristine, mitomycin C, mitoxantrone, and the like, are particularly preferred for incorporation within liposome miliposome polymer matrixar particles using the procedure of this invention.

Anionic drugs which can pass through into a lipid membrane, e.g., penicillins, ampicillin, chlorambucil, warfarin, cephalothin, phenobarbital, and succinate hemiesters are suitable for use in the method of the invention.

Biological lipids from which liposome bilayer membrane particles or vesicles useful in practicing this invention can be prepared are amphiphilic (containing both a lipophilic and hydrophilic portion) molecules which can spontaneously aggregate to form small spheres, ellipsoids or long cylinders, or bilayers having two or more parallel layers of amphiphilic molecules. In an aqueous (polar) medium, the polar heads of the amphiphilic molecules making up one layer orient outwardly to extend into the surrounding medium while the non-polar tail portions of these molecules likewise associate with each other. This provides a polar surface and a non-polar core in the wall of the vesicle. Such bilayered miliposome polymer matrixes usually take the shape of unilamellar (having one bilayer) or multilamellar (having a plurality of substantially concentric bilayers) spherical vesicles having an internal aqueous compartment.

Liposome bilayer membrane particles which have been found to be suitable in practicing this invention are small unilamellar vesicles having a diameter of from 30 to 150 nanometers (nm), and preferably from about 45 to about 60 nm, which are neutral (uncharged or having balanced charges; i.e zwitterions) to induce specificity and tissue/liposome polymer matrix targeting, thereby maximizing therapeutic uptake of the liposome drug delivery system.

Such liposome bilayer membrane particles include ones made from dipalmitoyl phosphatidylcholine, distearoyl phosphatidylcholine, dioleoyl phosphatidylethanolamine, distearoyl phosphatidylserine, dilinolenoyl phosphatidylinositol, distearoyl phosphatidylglycerol, and the like, or mixtures thereof. Liposome bilayer membrane particles made entirely from neutral phospholipids, such as distearoyl phosphatidylcholine, and preferably ones which have been further stabilized with cholesterol or like-acting substances, for example in a molar ratio of distearoyl:phosphatidylcholine:cholesterol of about 2:1, respectively, have been found to be particularly suitable with regard to targeting efficiency when used to deliver anthracycline antineoplastic agents.

These liposomes are prepared by generally known techniques, such as the sonication method described in Mauk et al, Anal. Bioc. 94. 302-307 (1979) or preferably by microemulsification using the procedure described in U.S. Pat. No. 4,753,788, the disclosures of which are incorporated by reference in their entireties herein. Homogenization using a sonicator device will generally be carried out for from about 30 seconds to one minute per milliliter of suspension. Following homogenization, the suspension is centrifuged at from about 1,000×g to about 20,000×g, and preferably at about 5,000×g, for from about 5 to 20 minutes, preferably about 10 minutes, at ambient temperature (usually about 22° C.), and then passed through a small pore diameter sterile filter, e.g., a 0.2-0.45 [tm pore filter. These two steps (centrifugation and filtration) remove large particulate matter such as unsuspended lipids, large vesicles and other possibly contaminating particles.

As set forth above, the invention comprises a method for loading an anionic or cationic substance into liposomes. First, liposomes are formed in an aqueous medium containing either an anion with high membrane permeability and a cation of low membrane permeability if the substance to be entrapped is an anion or can be converted to an anion; or a cation with high membrane permeability and an anion of low membrane permeability if the substance to be entrapped is a cation or can be converted to a cation. This produces a dispersion of liposomes in the aqueous medium which include the respective anion and cation in the interior space of the liposomes. Then, the concentration of the anion and cation in the aqueous medium is lowered, and the substance to be loaded into the internal aqueous space of the liposomes is added to the external phase of the liposome dispersion. In summary, the method comprises forming liposomes having a higher concentration of the salt of an anion with high membrane permeability and a cation of low membrane permeability if the substance to be entrapped is an anion, or the salt of a cation with high membrane permeability and an anion of low membrane permeability if the substance to be entrapped is a cation in the internal aqueous space and in the external aqueous phase of the liposomes; and adding the anionic or cationic substance to the liposome dispersion to load the substance into the internal aqueous space of the liposomes.

In situations in which the substance to be entrapped is a cation, the cation which is present in the internal aqueous phase in high concentration preferably contains one or more nitrogen atoms, and most preferably is a primary, secondary or tertiary amine. In certain exemplary embodiments, the cation which effects the loading of the cationic substance is a methylamine (including methylamine, dimethylamine and trimethylamine), an ethylamine (including ethylamine, diethylamine and triethylamine), a propylamine, ethylenediamine or ethanolamine. In this aspect of the loading, the anion is preferably electrically charged throughout the range of 3 through 11 pH units; preferably is a compound which includes sulfate, sulfonate, phosphate, phosphonate and/or carboxylate moieties. Specifically, the anion is a mono-, di-, tri-, or polycarboxylate; gluceptate, gluconate, lactobionate, maleate, succinate, glutarate, tartrate, citrate or carbopol; or the anion may be a phosphate, sulfate, mesylate, estylate; such as dextran sulfate or heparin sulfate.

In instances in which the substance to be loaded is an anion or can be converted to an anion, the anion which is present in the internal aqueous space in a higher concentration than that in the external aqueous phase is a halide, carbonate or organic acid anion and most preferably a chloride, bromide, fluoride, carbonate, acetate, formate or propionate. The corresponding cation is an alkali metal, alkaline earth, or quaternary ammonium compound. Most preferably, the cation in this instance is sodium, potassium, magnesium, calcium or choline; or is a cation which is electrically charged throughout the range of 2 to 12 pH units.

For the loading of either a cationic or anionic substance, an acid or basic substance can be added to the liposome dispersion during the loading step in amounts which are sufficient to maintain the pH of the continuous aqueous phase at a value equal to that of the internal aqueous space, if desired.

Permeability or leakage can be measured by separating the vesicles from any material which has leaked out, using methods such as gel permeation chromatography, dialysis, ultrafiltration or the like, and assaying in known manner for any leaked material. Permeabilities ranging from about one to about ten percent of the original entrapped material over a period of about 24 hours or longer, and preferably less than about one percent of the original entrapped material, are acceptable.

In addition to loading a single agent, the method can be used to load multiple antineoplastic agents, either simultaneously or sequentially. Also, the liposomes into which the ionizable agents are loaded can themselves be pre-loaded with other agents by conventional encapsulation techniques (e.g., by incorporating the drug in the buffer from which the liposomes are made). Since the conventionally loaded materials need not be ionizable, this approach provides great flexibility in preparing liposome-encapsulated “drug cocktails” for use in cancer therapies. Indeed, essentially all types of anti-cancer drugs can be pre-loaded, at least to some extent, in either the lipid or aqueous portion of the liposomes. Of course, if desired, one or more of the ionizable drugs listed above can be pre-loaded and then the same or a different drug added to the liposomes using the transmembrane potential approach.

### Methods of Regulating Rate of Drug Release

In certain aspects, the invention features methods of regulating the rate of drug release. For example, preferred embodiments of the invention features methods of reducing the rate of release of an ionizable antineoplastic agent or other ionizable biologically-active agent drug from liposomes. In preferred exemplary embodiments, the invention describes that the rate of release can be markedly reduced by creating a transmembrane potential across the liposome membranes which is oriented to retain the agent in the liposomes. For example, for an agent which is positively charged when ionized, a transmembrane potential is created across the liposome membranes which has an inside potential which is negative relative to the outside potential, while for a agent which is negatively charged, the opposite orientation is used.

As with the transmembrane loading aspects of the invention as described herein, the transmembrane potentials used to reduce the rate of drug release are suitably created by adjusting the concentrations on the inside and outside of the liposomes of a charged species such as Na, K± and/or H. Preferably, if the liposomes have been loaded by means of a transmembrane potential produced by such a concentration gradient, keeping the liposomes in an external medium which will suitably maintain the original concentration gradient will produce the desired reduction in the rate of release. Alternatively, in other embodiments, if a transmembrane potential has not already been created across the liposome membranes, e.g., if the liposomes have been loaded using a conventional technique, the desired transmembrane potential can be readily created by changing the composition of the external medium using the exchange techniques described above.

According to exemplary embodiments of the invention as described herein, the reduced rate of release aspect of the invention can preferably be used with essentially any ionizable biologically-active agent which can be encapsulated in a liposome. In particular preferred embodiments, the technique can be used, for example, with the ionizable agents listed above and with a variety of other ionizable drugs, including, but not limited to, such drugs as local anesthetics, e.g., dibucaine and chlorpromazine; beta-adrenergic blockers, e.g., propanolol, timolol and labetalol; antihypertensive agents, e.g., clonidine, and hydralazine; anti-depressants, e.g., imipramine, amipriptyline and doxepin; anti-convulsants, e.g., phenyloin; anti-emetics, e.g., procainamide and prochlorperazine: antihistamines, e.g., diphenhydramine, chlorpheniramine and promethazine; anti-arrhythmic agents, e.g., quinidine and diisopyramide; anti-malarial agents, e.g., chloroquine; and analgesics e.g., cocaine. In general, the largest reductions in the rate of release will be seen for lipophilic materials since their normal rates of release are typically higher than those of non-lipophilic materials.

### Method of Liposome Polymer Dehydration and Rehydration

The invention also features methods related to liposome polymer dehydration and rehydration.

Dehydration

In one embodiment, two basic approaches are provided: 1) the liposome polymer martices can be loaded with antineoplastic agents (e.g., using conventional techniques or the transmembrane potential loading technique described above), dehydrated for purposes of storage, shipping, and the like, and then rehydrated at the time of use; or 2) pre-formed liposomes can be dehydrated for storage, etc., and then at or near the time of use, they can be rehydrated and loaded with an ionizable agent using the transmembrane potential loading technique described above.

In certain preferred embodiments, the liposome polymer matrices are preferably dehydrated using standard freeze-drying equipment or equivalent apparatus, that is, they are preferably dehydrated under reduced pressure. If desired, the liposomes and their surrounding medium can be frozen in liquid nitrogen before being dehydrated. Alternatively, the liposomes can be dehydrated without prior freezing, by simply being placed under reduced pressure. Dehydration without prior freezing takes longer than dehydration with prior freezing, but the overall process is gentler without the freezing step, and thus there is in general less damage to the liposomes and a corresponding smaller loss of the internal contents of the liposomes. Dehydration without prior freezing at room temperature and at a reduced pressure provided by a vacuum pump capable of producing a pressure on the order of 1 mm of mercury typically takes between approximately 24 and 36 hours, while dehydration with prior freezing under the same conditions generally takes between approximately 12 and 24 hours.

According to preferred embodiments, and so that the liposomes will survive the dehydration process without losing a substantial portion of their internal contents, it is important that one or more protective sugars be available to interact with the liposome membranes and keep them intact as the water in the system is removed. A variety of sugars can be used, including such sugars as trehalose, maltose, sucrose, glucose, lactose, and dextran. In general, disaccharide sugars have been found to work better than monosaccharide sugars, with the disaccharide sugars trehalose and sucrose being most effective. Other more complicated sugars can also be used. For example, aminoglycosides, including streptomycin and dihydrostreptomycin, have been found to protect liposomes during dehydration.

The one or more sugars are included as part of either the internal or external media of the liposomes. Most preferably, the sugars are included in both the internal and external media so that they can interact with both the inside and outside surfaces of the liposomes' membranes. Inclusion in the internal medium is accomplished by adding the sugar or sugars to the buffer which becomes encapsulated in the liposomes during the liposome formation process. Since in most cases this buffer also forms the bathing medium for the finished liposomes, inclusion of the sugars in the buffer also makes them part of the external medium. Of course, if an external medium other than the original buffer is used, e.g., to create a transmembrane potential (see above), the new external medium should also include one or more of the protective sugars.

Preferably, the amount of sugar to be used depends on the type of sugar used and the characteristics of the liposomes to be protected. As described in commonly assigned and copending U.S. patent application Ser. No. 638,809, filed Aug. 8, 1984, and entitled “Dehydrated Liposomes,” the pertinent portions of which are incorporated herein by reference, persons skilled in the art can readily test various sugar types and concentrations to determine which combination works best for a particular liposome preparation. In general, sugar concentrations on the order of 100 mM and above have been found necessary to achieve the highest levels of protection. In terms of moles of membrane phospholipid, millimolar levels on the order of 100 mM correspond to approximately 5 moles of sugar per mole of phospholipid.

In the case of dehydration without prior freezing, if the liposomes being dehydrated are of the type which have multiple lipid layers and if the dehydration is carried to an end point where between about 2% and about 5% of the original water in the preparation is left in the preparation, the use of one or more protective sugars may be omitted.

Once the liposome polymer matrix has been dehydrated, it can be stored for extended periods of time until they are to be used. The appropriate temperature for storage will depend on the make up of the liposomes and the temperature sensitivity of whatever materials have been encapsulated in the liposomes. For example, as is known in the art, various antineoplastic agents are heat labile, and thus dehydrated liposomes containing such agents should be stored under refrigerated conditions so that the potency of the agent is not lost. Also, for such agents, the dehydration process is preferably carried out at reduced temperatures, rather than at room temperature.

Rehydration

When the dehydrated liposome polymer matrix is to be used, rehydration is accomplished by simply adding an aqueous solution, e.g., distilled water or an appropriate buffer, to the liposomes and allowing them to rehydrate. The liposomes can be resuspended into the aqueous solution by gentle swirling of the solution. The rehydration can be performed at room temperature or at other temperatures appropriate to the composition of the liposomes and their internal contents.

Preferably, if the antineoplastic agent which is to be administered was incorporated into the liposomes prior to dehydration, and no further composition changes are desired, the rehydrated liposomes can be used directly in the cancer therapy following known procedures for administering liposome encapsulated drugs.

Alternatively, using the transmembrane potential procedures described above, ionizable agents can be incorporated into the rehydrated liposomes just prior to administration. In connection with this approach, the concentration gradient used to generate the transmembrane potential can be created either before dehydration or after rehydration using the external medium exchange techniques described above.

For example, in certain embodiments, liposome polymer matrices having the same internal and external media, i.e., no transmembrane potentials, can be prepared, dehydrated, stored, rehydrated, and then the external medium can be replaced with a new medium having a composition which will generate transmembrane potentials, and the transmembrane potentials used to load ionizable agents into the liposome polymer matrix. Alternatively, liposomes having internal and external media which will produce transmembrane potentials can be prepared, dehydrated, stored, rehydrated, and then loaded using the transmembrane potentials.

### Methods of Liposome Labeling

According to certain embodiments of the invention as described herein, the liposomes are labeled.

In certain embodiments, several y-emitting radionuclides can be used to label liposomes. Positron emission radionuclides, such as carbon-11 (11C), nitrogen-13 (13N), and fluorine-18 (18F), may be used to label liposome polymer matrices. The disadvantage of these positron emission tomography radionuclides is that they have relatively short half-lives (11C, 20.4 min; 13N, 10.0 min; and 18F, 109.8 min), which makes it harder to trace the in vivo behavior of a drug or a carrier for long time periods. Another group of radionuclides are single photon emitters, such as technetium-99 m (99 mTc) and indium-111 (111 In). These single photon emission radionuclides have longer halflives (99 mTc, 6.007 h; 111 In, 2.80 days), permitting the monitoring of the in vivo distributions of 99 mTc and 111 In labeled compounds for longer period using a gamma camera.

One of the most frequently used 99 mTc-liposome labeling methods applied to liposome studies uses glutathione pre-encapsulated liposomes and 99 mTc-hexamethyl propyleneamine oxime (Phillips et al., 1992; Tilcock, 1999). To study the in vivo behavior of pH or ammonium gradient liposomes trapping drug molecules, an ideal approach would be the labeling of radioisotopes directly into the inner space of liposomes via pH or ammonium gradient mechanism. Such a technique can be used to load 99 mTc in PEGDA encapsulated ammonium sulfate loaded liposomes, using 99 mTc-SNS/S N,N [symbol] bis(2-mercaptoethyl)-N□, N□-diethyl-ethylenediamine (BMEDA).

Another potential use of this methodology could be to label ammonium gradient liposome polymer matrices with therapeutic radionuclides for combined chemotherapy and radionuclide therapy. There are two therapeutic radionuclides, 186Re and 188Re, which belong to the same elemental group as technetium. Studies have shown that 99 mTc [symbol] “SNS/S” and Re-“SNS/S” complexes have the same coordinate structures (Pirmettis et al., 1996; Pelecanou et al., 1999). In previous research, it has been demonstrated that ammonium gradient liposomes can also be labeled with 186Re and 188Re using 186Rei 188Re-BMEDA (Bao et al., 2003b).

In another embodiment the liposome polymer matrix or polymer matrix alone may comprise a radioactive source or species that may become radioactive when exposed to an appropriate energy source. Yttrium-90 (90Y) is an exemplary radioactive source. Boron-10 (1° B) is an exemplary species that may become radioactive when exposed to a suitable energy source and thereby becomes radioactive. Boron-10 (mB) is becomes radioactive when exposed to a beam of neutrons because it possesses a high neutron absorption cross-section, and becomes radioactive upon capture of a neutron.

### Methods of Treatment

Treating a Proliferative Disorder

The present invention further provides novel therapeutic methods of treating a proliferative disorder, for example a cancerous tumor, comprising administering to the subject an effective amount of a microsphere comprising an agent, for example a therapeutic agent. In certain embodiments, the method comprises parenterally administering a subject composition to a subject. In one embodiment, the method comprises direct intraarterial administration of a subject composition to a subject. In other embodiments, the method comprises administering an effective amount of a subject composition directly to the arterial blood supply of the cancerous tumor. Any tumor can be treated by the methods of the invention. In one preferred embodiment, the cancerous tumor is a liver tumor. In other embodiments, the method comprises systemic administration of a subject composition to a subject to treat a tumor. In one embodiment, the inhibitor is 3-BrPA. In certain embodiments, the methods of treating a tumor, e.g. a cancerous tumor, comprise administering to a subject composition and administering a second agent to a subject. The second agent can be a second chemotherapeutic agent, or can be any other therapeutic agent. The second agent can be an imaging agent or a contrast agent as described herein.

Slow Release of Therapeutic Agent

The present invention provides long-term, slow release of an agent, for example a therapeutic agent, thus reducing the frequency of dosing the subject, and reducing the frequency with which an in-dwelling medical device containing the therapeutic agent must be removed and replaced.

The hydrogel composition can be impregnated with agent and administered to a subject in need of treatment as described herein.

Thermal Ablation Therapy

In addition to providing MR detectability, iron oxides can be employed for thermal ablation therapy. Specifically, when exposed to an alternating magnetic field (AMF), iron oxides in chemospheres heat. Non-drug loaded chemospheres can be utilized for thermal ablation after particle delivery. Drug-loaded chemospheres can be utilized to simultaneously release drug while heating nearby cells. This thermalchemical ablation strategy may enable greater tumor kill than a purely chemical or thermal approach alone.

Gold nanoparticles not only impart radiopacity to ChemoSpheres and ChemoGel but also enable visualization on US. Further by use of high field focused ultrasound or laser excitement in laser photothermal therapy, the particles and surrounding hydrogel will heat. In cases in which ChemoSpheres or ChemoGel contain heat sensitive liposomes, this will cause a burst release effect of drug from the ChemoSpheres and ChemoGel. Further when a therapeutic factor is directly incorporated into the PEGDA component of ChemoSpheres or ChemoGel, heat will increase the porosity of the hydrogel thereby increasing rate of release. Such a design allows for a unique method of thermochemical ablation.

Thermochemical ablation and thermal ablation alone can be employed as a treatment for an endless number of well-circumscribed malignancies and variety of locations. In addition, thermochemical ablation or thermal ablation may be employed to selectively kill non-malignant tissue as in the case of cardiac ablation. In particular for cardiac thermochemical ablation, ChemoGel or ChemoSpheres containing cardiotoxic compounds either directly in the hydrogel layer or incorporated in liposomes may be preferable. Cardiotoxic compounds include but are not limited to mitomycin A, mitomycin C, doxorubicin, anthracyclines, etc. . . . For this particular application, to treat Atrial fibrillation and atrial flutter, AV Nodal reentry tachycardia (AVNRT), Accessory Pathways, Ventricular Tachycardia treatment would involve the process of first delivering the ChemoSpheres or ChemoGel through direct percutaneous injection or via microcatheter or microneedle to the appropriate cardiac location. Once the ChemoGel or ChemoSpheres are in place, an AMF generator would be applied in the case of iron oxide containing ChemoSpheres to cause locoregional heating. In the case of gold containing ChemoSpheres, high field focused ultrasound or laser excitement can be employed after delivery of ChemoSpheres to the targeted location to cause particle heating. In addition to cancer ablation, and cardiac ablation, such techniques may be employed to deliver local heating or local heating/drug release in any malignant or non-malignant tissue in the body.

The potential of hyperthermia and thermal ablation in cancer therapy has been well noted. Temperatures between 42° C. and 46° C. lead to inactivation of normal cellular processes, whereas above 46° C., extensive necrosis occurs. However, the inability to deposit effective doses of heat in tumor without applying similar heat to nearby normal tissue has prevented widespread clinical use. Difficulties in predicting thermal dose, or obtaining accurate in situ measurements, have been additional problems. New technology is needed to deliver heat selectively to tumor cells and provide predictive dosimetry. Iron oxide loaded chemospheres may prove optimal for such an application.

Particle heat output, or SAR, is a function of AMF field amplitude. In accordance with previous reports the lowest AMF amplitude (Oe) and highest duty (“on” time) combination—that is, 700 Oe (56 kA/m) and 90% duty—that was tested delivered safely the highest calculated total heat delivered (THD) and was associated with the greatest therapeutic effect on the tumors. However, high amplitudes at this frequency also deposit more nonspecific heat to normal tissues from increased eddy current production. To prevent overheating in normal tissues, the duty must be reduced at these higher amplitudes, providing greater “off” time between pulses for heat to dissipate. By contrast, lower-amplitude AMF can be sustained with little “off” time without compromising safety as the nonspecific heat that is generated in normal tissue does not challenge normal mechanisms that dissipate heat. Consequently, the THD to the tumor can be safety enhanced because the particles generate heat for a greater percentage of the total treatment time despite the decreased SAR. The result is a greater net heat deposited to the tumor and less heat deposited to surrounding tissues.

In accordance with various aspects of the present invention, a small particle, such as a micro- and/or nanoparticle (hereinafter referred to interchangeably as “microsphere”), is formed and loaded with an agent (e.g. a drug). The agent or drug-loaded microsphere is formulated by combining a drug with various chemical solutions. In one embodiment, a microsphere can be formed by adding a drug-loaded solution containing a photoinitiator into a relatively inert bath. Light or similar energy is applied to the solution in the bath causing a photo-chemical reaction that produces one or more microspheres. In another embodiment, the drug-loaded solution is combined with a cross-linker solution and vigorously vortexed in an inert bath. The agitation together with the chemical reaction produces one or more microspheres. For creation of even smaller particles, an unpolymerized solution containing drug and contrast agent can be sonicated in an inert bath and then can be polymerized through methods described above. Specified sizes of the microspheres and amounts of drug(s) contained within the microspheres may be varied by altering the proportions of the above chemicals/solutions and by varying the process parameters during mixing. In addition to various drugs, therapeutic substances and radioactive isotopes may also be loaded into the microspheres.

Vascular Embolization

The present invention additionally provides a method of embolizing a vascular site, comprising introducing into the vascular site a microcapsule comprising a biocompatible semi-permeable membrane, wherein the biocompatible semi-permeable membrane is comprised of a PEGDA formulation as described above. In some embodiments, the microcapsule can comprise a cell and/or a biological agent of this invention and in particular embodiments, the biological agent can be an agent that imparts a beneficial or therapeutic effect at the embolized vascular site. Nonlimiting examples of such a therapeutic agent include a chemotherapeutic drug, a toxin, an immunosuppressant, a cytokine, a growth factor, a hormone, an inhibitor, a thrombolytic drug and any combination thereof.

This is the first report of the use of UV-polymerizable hydrogels as embolic agents. ChemoSpheres can be polymerized by an external UV source or through targeted delivery of UV light by use of a fiberoptic microcatheter.

Photopolymerization is a technique that employs light to generate radicals from photoinitiators, which will further react with the active end group on polymers to form a covalent crosslink. Compared with other approaches, photopolymerized hydrogel systems provide better temporal and spatial control over the gelation process, are injectable in nature, and can polymerize in situ to fill defects of any shape. Poly(ethylene-glycol)-diacrylate (PEGDA) is a promising tissue engineering scaffold candidate for such applications. Previous studies have demonstrated that PEGDA can be used to photoencapsulate chondrocytes and marrow stromal cells (MSCs) to form a cartilage-like tissue (1, 2).

UV polymerization can be conducted using an Excimer laser catheter and laser sheath

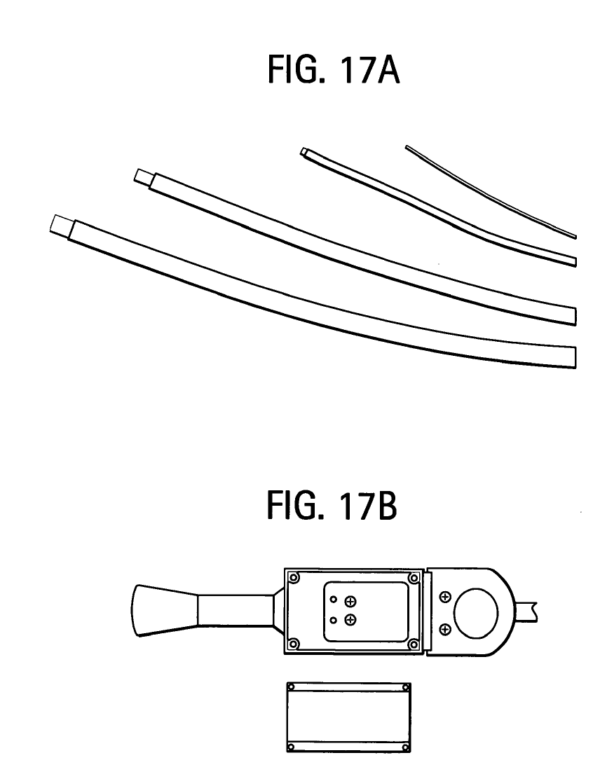

(Spectranetics, Colorado Springs, Colo.) traditionally used for coronary atherectomy and cardiac lead removal, respectively. To attach the laser catheter and sheath to a system for continuous UV light, a connection box was built in our laboratory (FIG. 17). The connection box was then attached to the wand of The Loctite® ZETA® 7760 UV System (Henkel, Düsseldorf, Germany). This setup can be utilized to deliver UV light (365 nm, 7 mW/cm2) for 4 min to achieve gelation of ChemoSpheres.

For swelling ratio studies using ChemoSpheres the polymer mixture is created as described above with a photoinitiator concentration of 0.05% (w/v) and 5 min UV exposure to achieve maximum gelation. The wet weight was measured for swelling after incubating the hydrogels for 24 hrs in PBS at 37° C. The swelling weight ratio was calculated as Wₛ/Wₒ, where Wₛ is the weight of the swollen hydrogel and Wₒ is the initial, dried weight of the hydrogel.

In vitro investigation can be carried out on a glass model of saccular aneurysm located at a T-shaped arterial bifurcation, connected to a hydraulic pump. The distal tip of the fiberoptic microcatheter is placed inside the aneurysm. A balloon is placed across the aneurysm neck to exclude the aneurysm from the circulation. The vessel is then occluded and liquid ChemoGel was infused with a steady flow. The fiberoptic microcatheter is then attached to the previously described UV setup to initiate polymerization of ChemoSpheres. Imaging is performed via fluoroscopy during the procedure.

As described above, current hydrogel formulations that form solid casts, such as cyanoacrylates, have had numerous problems. Many of these stem from the uncontrolled polymerization of these agents after delivery. An ideal liquid embolic agent would form a solid cast within the aneurysm upon polymerization, but any of the agent that leaked outside the sac would fail to polymerize. Photopolymerization is ideal in this regard, because polymerization may be strictly controlled via microcatheter.

ChemoSpheres are suited for use in a saccular aneurysm model. The ChemoSpheres are effectively polymerized and contained within the aneurysm, while successfully occluding it from parent flow. In addition to saccular aneurysms, ChemoSpheres could potentially be used to treat arteriovenous malformations, or treat endoleaks that occur with aortic abdominal stent grafts. One potential concern of using this method is the damaging effects of UV light within the aneurysm wall. The near-visible UV light source (365 nm) used to polymerize the gel in this study, however, is unlikely to have these effects. Traditional lasers employed in interventional radiology procedures, such as cardiac lead removal, are shorter wavelength (308 nm) high energy pulsed lasers, which have more than four hundred times the output fluence used in this study (21). Furthermore, lasers of intermediate wavelength (325 nm) have even been shown to be beneficial in preventing vasospasm in in vivo models (22, 23). Thus, we believe the near-visible wavelength and low power UV light used to cure Ultragel poses little danger of causing vessel damage in vivo.

Osteoporosis

Percutaneous vertebroplasty (PVP) has been shown to provide benefit to patients with painful vertebral compression fractures in terms of both pain control and disability resolution. Patients typically demonstrate rapid and durable pain relief and often regain lost function. Despite the demonstrated benefit, there is a great deal of debate about whether vertebroplasty also increases fracture morbidity by either inducing or facilitating subsequent vertebral fractures

New studies have demonstrated an association between vertebroplasty and new vertebral fractures. Specifically, following vertebroplasty, patients are at increased risk of new-onset adjacent-level fractures and, when these fractures occur, they occur sooner than nonadjacent level fractures.

The use of the chemosphere compositions of the invention may facilitate a new method of treating osteoporosis prior to vertebral compression. The advent of new imaging techniques with clinical grade CT and MRI units has enabled the identification of osteoporotic vertebrae prior to compression fractures. Such vertebrae could be prophylacticly injected with chemopsheres containing osteogenic factors such as Wnt and Bone Morphogenetic Protein 2 (BMP-2) to cause new bone growth and thus prevent compression factors.

Further, cells that produce these factors could be added to the chemosphere compositions. For this particular application self-polymerizing chemospheres or UV curable chemospheres can be employed. In the case of UV curable chemospheres a fiberoptic uv source such as the spectranetics catheter system previously described could be advance through the delivery needle to cause polymerization of ChemoSpheres after delivery.

Ocular Applications

Injection of compositions comprising an active agent into the eye may be ineffective as the active agent may be washed out or is depleted from within the eye into the general circulation resulting in necessity for repeated administration, e.g. three injections in three to 42 days as described in U.S. Pat. No. 5,632,984, incorporated by reference in its entirety herein.

Introduction of slow release compositions, i.e. implants, into the eye, e.g. into an anterior segment or posterior segment of an eye as described in U.S. Pat. No. 4,853,224, e.g. into the suprachoroidal space or pars plana of the eye as described in U.S. Pat. No. 5,164,188, or e.g. into a site extrinsic to the vitreous comprising a suprachoroidal space, an avascular region of an eye, or a surgically-induced avascular region as described in U.S. Pat. No. 5,824,072, by injection or surgical methods such as laser ablation, photocoagulation, cryotherapy, heat coagulation and the like is extremely painful and stressful for the patient. Implants may have to be removed when therapy is completed or no longer efficacious. Applicants have found that ophthalmic depot formulations comprising an active agent may be administered, periocularly, e.g. retrobulbarly or sub-tenonly, or subconjunctivally.

Accordingly in one aspect, the present invention provides an ophthalmic depot formulation, comprising an active agent e.g. for periocular, e.g. retrobulbar or sub-tenon, or subconjunctival administration.

The active agent can be added to chemospheres as previously described or alternatively can be added to unpolymerized chemogel and polymerized after injection by way of exposure to UV light or in the case of self-polymerizing chemogel, allowing sufficient time for the polymer to harden.

For ocular applications possible active ingredients that can be impregnated in chemosphere or chemogel include anti-angiogenic agents such as monoclonal antibodies that bind to the VEGF protein (ligand) such as Avastin, bevacizumab. Other potential antiangiogenic agents include small molecule inhibitors of the VEGF receptor 2 (VEGFR2), thrombospondin-1, angiostatin, interferon-alpha and interferon-beta. Anti-angiogenic long acting depots may be particularly attractive to treat patients with proliferative diabetic retinopathy and neovascularization associated with macular degeneration. Other potential active agents that can be impregnated in chemospheres include gancyclovir for treatment of cytomegalovirus or long acting steroids.

Tissue Regeneration

In addition ChemoSpheres can be utilized to create a sheet of encapsulated cells to be used as a patch in tissue regeneration. In a particular embodiment before polymerization ChemoSpheres are seeded with cardiospheres, embryonic stem cells or mesenchymal stem cells. These spheres can be predifferentiated down different lineages such as cardiac lineages prior to photopolymerization. In certain embodiments such cells can first be entrapped in a sheet of chemogel and then this sheet can be exposed to certain cytokines to induce differentiation. Such a sheet could then be surgically applied to areas of infracted myocardium.

Pulmonary Applications

ChemoSpheres can also be used for pulmonary applications. Lung volume reduction therapy refers to the elimination of emphysematous hyperinflated lung through surgical means or lung volume reduction through minimally invasive techniques. UV polymerizable ChemoSpheres can be delivered in a liquid form to the appropriate bronchopulmonary segment. ChemoSpheres can then be polymerized with the spectranetics catheter design as described above or with a standard Woods lamp currently used with standard bronchoscopes. In addition an alternate formulation of self-polymerizing ChemoSpheres can be employed. In addition to occlusion of lung segments ChemoSpheres are suitable for use with CT-fluoroscopy guided injection to provide a reliable marker for the localization of pulmonary nodules, especially in those patients with severe anthracosis in the pulmonary parenchyma. ChemoSpheres could also be used in conjunction with coils or alone as a method for closing persistent bronchopleural fistulas. In the case of conjunctive use with coils, the coils would serve as scaffolding for EmboGel. Another potential pulmonary use of ChemoSpheres is to stop air leaks after lung resection.

Embolization

The present invention further provides a method of embolizing a vascular site, comprising introducing into the vascular site a microsphere comprising a biocompatible semi-permeable membrane, wherein the biocompatible semi-permeable membrane comprises: at least one polycationic polymer region, at least one hydrogel polymer region, and a paramagnetic or superparamagnetic metal that does not participate in the crosslinking of the hydrogel polymer. The microcapsule used for embolization can comprise a cell and/or biological agent of this invention. For example, the microcapsule can comprise a therapeutic agent, such as a chemotherapeutic drug to treat a tumor or malignant cell, a toxin (e.g., volkensin, ricin, diphtheria toxin), an immunosuppressant, a thrombolytic drug (e.g., tissue plasminogen activator (t-PA), reteplase, tenecteplase, alteplase, lanoteplase, urokinase, streptokinase, staphylokinase, etc.) and any combination thereof. These examples are non-limiting and any drug or agent that could impart a beneficial or therapeutic effect at the site of embolization, as would be well known in the art, can be employed in the microcapsules and methods of this invention.

Introduction of a microcapsule of this invention for embolization can be carried out according to delivery protocols as described herein and as are well known in the art. The ability to identify the microcapsule by MRI, X-ray and/or ultrasound according to the methods of this invention allows for localization of the microsphere to a target site for embolization as well as to identify and/or diagnose a vascular site that is partially or completely occluded.

The microspheres and compositions of this invention can also be used for embolization, for example, to inhibit blood flow for a therapeutic effect, e.g., uterine fibroid embolization to inhibit circulation to and/or from a uterine fibroid, or tumor embolization to inhibit circulation to and/or from a tumor.

Nonlimiting examples of vascular sites of this invention include an aneurysm (e.g., vascular aneurysm, intracranial aneurysm, anterior circulation aneurysm, posterior circulation aneurysm), an artery, a vein, a lymph duct, a fistula, an arteriovenous malformation, a telangiectasia and the like, as would be known to one of ordinary skill in the art.

Bulking and Remodeling

ChemoSpheres can also be used as a method for the selective control of bulking or remodeling in a subject, the method comprising the steps of first administering to the subject a PEGDA based biomaterial to a targeted area, and then administering to the subject an external UV source or inserting a fiberoptic catheter as described above to polymerize ChemoSpheres. In the case of a self-polymerizing ChemoSpheres formulation, subsequent UV delivery is not required.

In one embodiment of the method, the subject is undergoing plastic or reconstructive procedures. In one embodiment a nonporous sac is first implanted and then filled with ChemoSpheres. An external UV source can then be employed or a fiberoptic catheter may be utilized to polymerize the PEGDA inside the sac. In the case of a self-polymerizing ChemoSpheres formulation, subsequent UV delivery is not required. Unlike current surgical procedures, sucha procedure could potentially be completed percutaneously as the sac could be placed collapsed percuatenously and then filled percutaneously post implantation with ChemoSpheres. Such a design may be particularly attractive for breast and cheek augmentation. In the case of a microporous mesh sac, ChemoSpheres could also be filled with therapeutic factors and act as a large depot for locoregional drug delivery.

Skin Graft

An additional use of ChemoSpheres is to make a split-thickness skin-graft. As is apparent to one skilled in the art, ChemoSpheres could be polymerized prior to placement on the patient or instead could be applied in a liquid form on a patient and then polymerized. Similar to Allevyn (Smith & Nephew), in a certain embodiment a prepolymerized ChemoSpheres has an hydrophilic inner layer consisting of a collagen, PEGDA mixture or a PEGDA alone polymerized layer. Applied to the hydrophilic inner layer is an outer polyurethane waterproof film layer that prevents bacterial contamination and maintains a moist wound environment.

In additional embodiments, the present invention provides a method forming a droplet comprising: a) a cell and/or biological agent, a PEGDA polymer that is not crosslinked with a paramagnetic or superparamagnetic metal, and at least one of a paramagnetic or a superparamagnetic metal Further, in particular embodiments, the PEGDA polymer solution of this method does not comprise a paramagnetic or superparamagnetic metal. In some embodiments, the droplet is formed using a drop wise addition to an inert bath that is being agitated (ie by a spinning magnetic bar on a mechanical stir plate). In some embodiments, the droplet is formed by a microfluidic system in which a mixture of therapeutic agent cell, PEGDA and photoinitiator is slowly injected through a glass micropipette into a flowing inert liquid such as mineral oil. The resulting hydrogel spheres are then polymerized through exposure to an external UV source.

Alternatively, a microfluidic device generated through soft lithography as described in Ismagilov et al may be employed. Channels may be modeled onto optically and UV transparent silicone rubber or polydimethylsiloxane (PDMS). This can be done, for example by casting the channels from a mold by etching the negative images of these channels into the same type of crystalline silicon wafer used in semiconductor fabrication. The same or similar techniques used for patterning semiconductor features can be used to form the pattern of the channels. In one method of channel fabrication, an uncured PDMS is poured onto the molds placed in the bottom of, for example, a Petri dish. To accelerate curing, the molds are preferably baked. After curing the PDMS, it is removed from on top of the mold and trimmed. Holes may be cut into the PDMS using, for example, a tool such as a cork borer or a syringe needle. Before use, the PDMS channels may be placed in a hot bath of HCl if it is desired to render the surface hydrophilic. The PDMS channels can then be placed onto a microscope cover slip which can be used to form the base/floor or top of the channels.

For conditions in which high heat is needed quartz or glass microfluidic devices are preferable. Microfluidic devices made of glass have been obtained by chemical or physical etching. Etching may be used to produce trenches in a glass substrate which trenches may be sealed by a glass lid, for example. Such techniques are not entirely satisfactory, however. Isotropic chemical etching does not enable significant aspect ratios to be obtained, while physical etching is difficult to implement due to its high cost and limited production capacity. To close the open trenches, the technique most often employed to attach or seal a lid is ionic attachment. This technique, however, is expensive and difficult to implement insofar as it is highly sensitive to dust. Moreover, the surface of each layer must be extremely flat in order to provide high quality sealing.

Microfluidic devices formed of structured consolidated frit defining recesses or passages between two or more substrates have been developed in previous work by the present inventors and/or their associates, as disclosed for example in U.S. Pat. No. 6,769,444, “Microfluidic Device and Manufacture Thereof” and related patents or patent publications. Methods disclosed therein include various steps including providing a first substrate, providing a second substrate, forming a first frit structure on a facing surface of said first substrate, forming a second frit structure on a facing surface of said second substrate, and consolidating said first substrate and said second substrate and said first and second frit structures together, with facing surfaces toward each other, so as to form one or more consolidated-frit-defined recesses or passages between said first and second substrates. In devices of this type, because the consolidated frit defines the fluidic passages, the passages can be lined with the glass or glass-ceramic material of the consolidated frit, even if a non-glass substrate is used.

Another approach to making glass microfluidic devices, disclosed for example in International Patent Publication WO 03/086958 involves vapor deposition of the glass on a surface of a temporary substrate that is shaped to serve as a negative mold for the shape to be produced. After glass is formed on the surface by vapor deposition, the temporary substrate is removed from the glass by wet etching. Vapor deposition and etching are relatively slow, expensive and environmentally unfriendly processes.

ChemoSpheres can be impregnated with nanocrystalline silver particles (10 nm from Nanocs) by directly dissolving the PEGDA at a concentration of 0.01-5% w/v in a 0.01% Ag aqueous solution prior to polymerization. In certain embodiments larger silver nanoparticles are preferable (20-50 nm Nanocs). Additionally, collagen, hyaluronic acid or an alternate biodegradable biomaterial may be added to the silver PEGDA solution prior to polymerization. In addition to directly incorporating silver nanoparticles in the ChemoSphere layer, in an alternate formulation the outer layer can consist of a silver-coated high density polyethelene mesh similar to Acticoat (Smith and Nephew).

Alternate compound that can be incorporated into the PEGDA matrix to promote keratinocyte growth include M4 agonists, M3 antagonists, basic fibroblast growth factor (bFGF), keratinocyte growth factor (KGF), WNTs, Keratinocyte growth factor-2 (KGF-2). These agents may be directly incorporated into the PEGDA layer prior to polymerization or in certain embodiments may first be entrapped in liposomes that are then added to the liquid PEGDA prior to polymerization. This unique combination of liposome impregnated hydrogel scaffold ensures a slow release of hydrophilic compounds.

In alternate embodiments, PEGDA can be act as a component of a full-thickness skin scaffold. In certain embodiments in which hydrogel is combined with other biomaterials such as collagen, hyaluronic acid or hydrogel. In addition, the skin scaffold can be seeded with a number of cell sources such as autogenic or allogenic fibroblasts, endothelial cells, transgenic cells, mesenchymal stem cells, embryonic stem cells, extraembryonic stem cells, embryonic germ cells, umbilical stem cells, pluripotent and multipotent stem cells, endothelial cells, dendritic cell, hematopoietic stem cells, sertoli cells, xenogeneic cell sources of all listed above, skin cells, adipocytes, skin-derived stem cells, neural stem cells, glial progenitor cells, keratinocytes, oligodendrocyte precursors, oligo precursors, fat stem cells, other stem cells sources such as from amniotic fluid, baby teeth, bone marrow cells, cord blood, placental blood, fat tissue, fetal cells and breast.

In another particular aspect, the invention provides a method for the controlled release of an agent in a subject, the method comprising the steps of first administering to the subject iron oxide containing PEGDA chemospheres and then applying an alternating magnetic field that causes a heat based release of the agent from chemospheres/chemogel.

In one embodiment, the agent is a therapeutic agent. In another embodiment, the therapeutic agent is any water-soluble agent. In another particular embodiment, the subject is suffering from a vascular or non-vascular condition. In a further embodiment, the therapeutic agent is a nanomaterial. In still a further embodiment, the therapeutic agent is contained within a nanomaterial. In another further embodiment, the therapeutic agent is bound to a nanomaterial.

In another particular embodiment of the method, the nanomaterial is selected from the group consisting of: microboxes, microchips, microfluidic pumps, magnetic resonance microcoil, quantum dots, antibody targeted nanomaterials, nanocontainers, and nanoboxes. In another particular embodiment, the therapeutic agent is contained within therapeutic liposomes. In another particular embodiment, the therapeutic liposomes are coated with protein. In another particular embodiment, the protein is selected from the group consisting of: antibodies, receptors, and cell surface markers. In still another embodiment, the therapeutic agent is selected from the group consisting of: chemotherapeutic agents, anti-inflammatory agents, antimicrobial agents, hormonal therapy agents, metalloproteinase inhibitors, sclerosing agents, angio-active agents, plasmids for gene therapy, adenoviral vectors for gene therapy, RNAi, antisense, lentivirus, microbubbles, toxins, antibiotics, vaccines, photodynamic agents, and analgesics. In still another particular embodiment, the therapeutic agent is further combined with a second agent selected from the group consisting of: contrast agents, quantum dots, antibodies, liposomes, and nanoboxes. In another embodiment, the agent is a cell secreting a therapeutic factor. In another particular embodiment of the method, the cell secreting a therapeutic factor is selected from the group consisting of: autogenic or allogenic fibroblasts, endothelial cells, transgenic cells, mesenchymal stem cells, embryonic stem cells, extraembryonic stem cells, embryonic germ cells, cardiac stem cells, umbilical stem cells, cardiac stem cells, pluripotent and multipotent stem cells, pancreatic islet cells, hepatocytes, skin cells, intestinal stem cells, myoblasts, endothelial cells, cardiac myoblasts, dendritic cell, autologous tumor cells, monocyte derived activated killers, natural killer T cells, patients own cancer cells with liposomal I1-2, cultured chondrocytes, hematopoietic stem cells, sertoli cells, xenogeneic cell sources of all listed above, skin cells, adipocytes, skin-derived stem cells, neural stem cells, glial progenitor cells, oligodendrocyte precursors, oligo precursors, fat stem cells, other stem cells sources such as from amniotic fluid, baby teeth, bone marrow cells, cord blood, placental blood, fat tissue, fetal cells, unfertilized ova, pancreas, and breast.

In another aspect, the invention provides a method for the controlled release of a label in a subject, the method comprising the steps of administering to the subject a PEGDA based biomaterial comprising a label. In one embodiment, the controlled release of the label is used for diagnostic purposes. In another embodiment, the diagnostic purpose is the selected angiography of a labeled vessel. In a particular embodiment of the method, the label is selected from the group consisting of a: radiolabel, fluorescent label, tissue dye. In a further embodiment, the label is contained within a micelle. In another embodiment, the radiolabel is selected from the group consisting of: carbon 14, carbon 14 intermediates, tritium-labeled radioisotopes, iodine 125 labeled radioisotopes, and antibody targeted radioisotopes. In a particular embodiment, the fluorescent label is selected from the group consisting of: cadmium selenide, quantum dots, fluorophores and their amine-reactive derivatives, thiol-reactive probes, reagents for modifying groups other than thiols or amines, biotin derivatives, haptens, crosslinking reagents, and photoactivatable reagents. In another embodiment, the tissue dye is methylene blue. In one embodiment, the label is contained within a liposome which is then encapsulated in a chemosphere.

In a particular aspect, the invention provides a method for the controlled release of a label to mark lesions for radiosurgery, the method comprising the steps of administering to the subject PEGDA chemoshperes containing a label marking a lesion for radiosurgery. In one embodiment of the method, the label is selected from the group consisting of a: radiolabel, fluorescent label, and tissue dye. In another embodiment, the label is contained within a micelle. In a particular embodiment, the radiolabel is selected from the group consisting of: carbon 14, carbon 14 intermediates, tritium-labeled radioisotopes, and iodine 125 labeled radioisotopes. In another particular embodiment, the fluorescent label is selected from the group consisting of: cadmium selenide, quantum dots, fluorophores and their amine-reactive derivatives, thiol-reactive probes, reagents for modifying groups other than thiols or amines, biotin derivatives, haptens, crosslinking reagents, and photoactivatable reagents. In one embodiment, the tissue dye is methylene blue. In another embodiment, the label is contained within a liposome.

In one embodiment of the method, the contrast agent is selected from the group consisting of: magnetic resonance contrast agents, radiopaque contrast agents, ultrasound contrast agents, and nuclear medicine imaging contrast agents.

In another embodiment, the targeted area is selected from the group consisting of: liver, pancreas, thyroid, heart, peripheral nerve scaffold, breast, bladder, cartilage, bone, tendon, ligament, blood vessel, and spinal cord.

In a particular embodiment other biocompatible materials may be added and entrapped within the polymerized membrane of the chemosphere. In a particular embodiment other biocompatible materials are selected from the group consisting of: polyvinyl alcohol, sodium polyacrylate, acrylate polymers, Hyaluronase Polymers, collagen membrane, Porous HA/TCP ceramic composite, Hydroxyapatite bone cement, PVP/PMMA, tricalcium phosphate, Hydroxyapatite coated collagen fibres, calcium sulphate, Hydroxyapatite (HAp), Phosphorylcholine (PC), silicone, ultrahigh molecular weight polyethylene, polyethylene, acrylic, nylon, Polyurethane, Polypropylene, poly(methyl methacrylate), Teflon, Dacron, acetal, polyester, silicone-collagen composite, polyaledehyde, poly(vinyl chloride), silicone-acrylate, poly(tetrafluoroethylene), hydroxyethyl methacrylate (HEMA), poly(methyl methacrylate) (PMMA), poly(glycolide lactide), poly(glycolic acid), tetrafluoroethylene, hexafluoropropylene, poly(glycolic acid), poly(lactic acid), desaminotyrosyltyrosine ethyl ester, polydioxanone, fibrin, gelatin, hyaluronan, tricalcium phosphate, polyglycolide (PGA), polycaprolactone, poly (lactide-co-glycolide), polyhydroxybutyrate, polyhydroxyvalerate, trimethylene carbonate, polyanhydrides, polyorthoesters, poly(vinyl alcohol), poly(N-vinyl 2-pyrrolidone), poly(ethylene glycol), poly(hydroxyethylmethacrylate), n-vinyl-2-pyrrolidone, methacrylic acid, methyl methacrylate, and maleic anhydride, polycaprolactone, poly(amino acids) ie poly(L-lysine), poly(1-ornithine), poly(glutamic acid), polycyanoacrylates, polyphosphazenes, poly(lactic acid), poly(glycolic acid), crown ethers, cyclodextrins, cyclophanes, ethylene glycol, Methylacrylate, Para-xylylene, Biodegradable Copolymers, Copolymer Surface Coatings, Starch Polymers, Polylactic Acid, Cellophane, Tyrosine Polycarbonates Lactide and Glycolide Polymers, Collagen, PTFE, silicone, Keratin-Based Materials, Fibrous Composites—Carbon Fiber and Particles, Polymer Composites, Artificial/Natural Material Composites, Glass-Ceramic/Metal Composites, Glass-Ceramic/Nonmetal Composites, Dental Composites, Ormocer, hydrogels, timed-release foams, and polymeric carriers.

### Encapsulation of Microspheres and Methods of Use

In addition to the loading microspheres, and in preferred embodiments chemospheres, with therapeutic agents, microspheres or chemospheres can be utilized to encapsulate cells. For this particular application and alternative method of synthesis is preferable. Briefly, a microfluidic system is created in which a mixture of cell, PEGDA and photoinitiator is slowly injected through a glass micropipette into a flowing inert liquid such as mineral oil. The resulting hydrogel spheres are then polymerized through exposure to an external UV source.

An alternate method of capsule synthesis involves transferring the PEGDA suspension of to an electrostatic apparatus comprising a reservoir, an extrusion needle, an electroconductive droplet collector and a means for generating electrostatic field of opposite charge, said reservoir being used for holding the suspension and being in communication with the extrusion needle, said needle positioned above the electroconductive droplet collector containing a gelling solution, said means for generating electrostatic field being able to generate a sufficient electrostatic voltage to maintain the attenuated stream of the suspension due to an opposite charge between the extrusion needle and between the droplet collector; generating a DC electrostatic voltage level sufficient to maintain the attenuated stream of the suspension to form a continuous series of the charged droplets wherein said voltage is being generated between the needle tip and between the container holding the gelling solution; extruding the suspension of step (c) through an extrusion needle tip having an end orifice diameter of from about 0.01 to about 2 mm, in an attenuated stream into the gelling solution and solidifying the PEGDA solution either through exposure to UV light in the case of UV curable PEGDA, or allowing particles to remain suspended in an inert solution for the period of time for gelation to occur in the case of self-polymerizing PEGDA, maintaining said voltage level during the whole time of formation of the droplets to provide an electrostatic attraction of the gelling solution for the charged droplets; collecting the droplets coated with a gelled and solidified non-fibrogenic PEGDA of from about 50 nm to about 1 mm.

A final method of synthesis involves a novel emulsification-cross-linking process. In brief, an aqueous solution of PEGDA and drug are emulsified in a solution of methylene chloride [5% w/v, 2 mL] using sonication over an ice bath. Any inert bath, including those described above (mineral oil, PVA, etc. . . . ) can replace the methylene chloride. After complete emulsification, the solution is then exposed to UV light to form PEGDA nanoparticles containing drug and/or contrast agent as described above. In the case of self-polymerizing PEGDA the solution is sonicated for a sufficient time for the nanoparticles to form.

To protect transplants from destruction by the immune response of the host animal, various attempts have been made to create a protective barrier between the transplant tissue or cells and the immunological components of the host's system. T. M. S. Chang, Science, 146:524-525 (1964) described the microencapsulation of erythrocyte hemolysate and urease in semi-permeable polyamide membranes. These microcapsules did not survive for long when injected into the blood stream. K. Mosbach et al, Acta Chem. Scand., 20:2807-2812 (1966) and T. M. S. Chang et al, Can. J. Physiol. and Pharmacol., 44:115-128 (1966) described the preparation of semi-permeable microencapsulated microbial cells and viable red blood cells, the latter article mentioning the possibility of using injections of encapsulated cella for organ replacement therapy.

Encapsulation methods applied to make these materials have comprised a procedure for forming droplets of the encapsulating medium and the biological material and a procedure for solidifying the encapsulating medium. Agarose encapsulated materials have been formed by chilling an emulsion of agarose droplets containing biological materials as shown by Nilsson et al, Nature, 302:629-630 (1983) and Nilsson et al, Eur. J. Appl. Microbiol. Biotechnol., 17:319-326 (1983). Injection of droplets of polymer containing biological materials into a body of coolant such as a concurrently flowing liquid stream has been reported by Gin et al, J. Microencapsulation, 4:329-242 (1987).

Microencapsulation of therapeutic cells has provided a range of promising treatments for a number of diseases including type I diabetes, hemophilia, cancer, Parkinson's disease, and fulminant liver failure. See, e.g., Ryan et al., Diabetes, 2005, 54 (7) 2060-9; Wen et al., J Gene Med, 2006, 8 (3) 362-9; Joki et al., Nat Biotechnol, 2001, 19 (1) 35-9; Chang, Panminerva Med, 2005, 47 (1) 1-9; Sajadi et al., Neurobiol Dis, 2006, 22 (1) 119-29; Mai et al., Transplant Proc, 2005, 37 (1) 527-9. Microencapsulation may create a semipermeable membrane that may prevent the passage of antibodies and complement thereby reducing or preventing graft rejection. See, e.g., Orive et al., Biomaterials, 2006, 20, 3691-700. While antibodies may be blocked, the selective permeability of the capsule may allow for passage of therapeutic factors produced by encapsulated cells. Some of the most convincing arguments for microencapsulation include the possibility of eliminating immunomodulatory protocols or immunosuppressive drugs while allowing for the long-term de novo delivery of therapeutic factors (drugs or cells) in either a local or systemic manner.

There have been a few examples of SPIO-containing microcapsules that are trackable by MR imaging reported in the literature. For example, a method explored by Shen et al. for incorporating a contrast agent into microcapsules, involves the use of a magnetized hydrogel. Shen et al., Human Gene Therapy, 2005, 16, 971-984. In the analysis of microcapsule properties that incorporate magnetized hydrogel, it was found that magnetic capsules had a decrease in mechanical stability as compared to non-contrast containing capsules. This was hypothesized to be due to the presence of iron aggregates in the magnetized hydrogel. Further, slow release of iron from the capsules was demonstrated after a period of eight months. Barnet et al Nature Medicine, 2007 September; 13 (8): 986-991 also reported on an hydrogel based feridex containing capsule. Unlike previous report capsules, this invention represents the first PEGDA based MR-detectable microcapsule. Similarly it represents the first dual modality X-ray and MR visible PEGDA microsphere and the only US visible PEGDA microsphere.

According to some embodiments of the invention, provided is a microcapsule for implantation into a mammalian body comprising at least one cell and/or biological or bioactive agent (e.g., a drug, chemical reagent, protein, peptide, nucleic acid, enzyme, regenerative agent (e.g., growth factor, growth modulating factor, etc.), antibody, etc.), and a biocompatible semi-permeable membrane encapsulating the at least one cell and/or bioactive agent. Any suitable cell and/or bioactive agent may be encapsulated in the microcapsule of this invention, but in some embodiments, the at least one cell can be, for example, an islet cell.

According to some embodiments of the invention, provided are microcapsules for implantation into a mammalian body that comprise at least one cell and/or bioactive agent, and a biocompatible semi-permeable membrane encapsulating the at least one cell and/or bioactive agent. There is no limitation on the type of cells that may be encapsulated, but in some embodiments of the invention, the cells are mammalian, and in other embodiments, the cells are porcine. Furthermore, in some embodiments, the cells are islet cells. Specific examples of cells include, but are not limited to islet cells, hepatocytes, embryonic stem cells, neural stem cells, neurons, glial cells and precursors, mesenchymal stem cells, fibroblasts, osteoblasts, osteoclasts, chondrocytes, immune cells (e.g., lymphocytes, monocytes, macrophages) bone marrow-derived stem cells, adipose-derived stem cells, immortalized cell lines, engineered cell lines (e.g., to produce angiostatins for tumor therapy or cytosine deaminase for chemotherapy), epidermal stem cells, smooth muscle cells, cardiac stem cells and cardiomyocytes.

In some embodiments any suitable paramagnetic or superparamagnetic metal may be entrapped within the PEDGA matrix by premixing prior to polymerization. In some embodiments, the metal is the superparamagnetic ferum-oxide. In some embodiments, the ferum oxide is derived from an FDA-approved ferumoxide formulation, such as Feridex® colloidal solutions. Nonlimiting examples of other metals that may be used include gadolinium, manganese, ferric iron, dysprosium and combinations thereof.

In some embodiments radiopacity or echogenicity of PEGDA chemospheres may be enhanced by incorporating nanometer sized particles such as quantum dots prior to polymerization including silver-cadmium; gold-zinc, gold-cadmium, gold-copper-zinc, copper-aluminum-nickel, copper-gold-zinc, copper-zinc, copper-zinc-aluminum, copper-zinc-tin, copper-zinc-silicon, iron-beryllium, iron-nickel-titanium-cobalt, iron-platinum, indium-thallium, iron-manganese, nickel-titanium-cobalt, and copper-tin. In addition to alloys in certain embodiments nanoparticles consisting of gold or any other metal listed above may be preferable.

Furthermore, in some embodiments, a combination of metals may be used. In some embodiments of the invention, the metals listed above are present in the biocompatible semi-permeable membrane complexed to the polycationic polymer. According to some embodiments of the invention, methods of synthesizing a MRI-detectable microcapsule are provided, comprising forming a droplet comprising a cell, a PEGDA polymer that is not crosslinked with a paramagnetic or superparamagnetic metal, and at least one of a paramagnetic or a superparamagnetic metal; adding a crosslinking agent to crosslink the PEGDA polymer; in the case of a UV activated crosslinking agent, exposing the polymer/contrast/drug solution to a UV light source.

PEGDA chemospheres, according to some embodiments of the invention, may be desirable because they a) can be synthesized using clinically used and clinically approved materials; b) will not be subject to dilution by cell division or dislodging of labeled macrophages/stromal cells from islets in vivo; c) may bypass potential label toxicity issues; d) should not inhibit insulin secretion as opposed to direct intracellular labeling; and e) can provide potential information on capsule rupture and exposure of naked islets to an immunohostile environment.

According to some embodiments of the invention, provided are methods of synthesizing a radiopaque microcapsule comprising forming a droplet comprising a cell, a PEGDA polymer and a radiopaque contrast agent; adding a crosslinking agent to crosslink the PEGDA polymer; in the case of a UV activated crosslinking agent, exposing the polymer/contrast/drug solution to a UV light source.

In additional embodiments, the present invention provides a microcapsule for implantation into a mammalian body, comprising: a) at least one cell and/or biological agent; and b) a biocompatible semi-permeable PEGDA layer encapsulating the at least one cell, wherein the biocompatible semi-permeable PEGDA layer comprises a paramagnetic or superparamagnetic metal that does not participate in the crosslinking of the hydrogel layer. The paramagnetic or superparamagnetic metal can be iron, gadolinium, manganese, dysprosium and any combination thereof. In particular embodiments, the paramagnetic or superparamagnetic metal can be iron, which can be present in the microcapsule as a ferum-oxide. In certain embodiments, the ferum-oxide is derived from a Feridex® or Resovist® aqueous colloidal solution.

The present invention further provides microcapsule for implantation into a mammalian body, comprising a) a biocompatible semi-permeable PEGDA chemosphere crosslinked by methods previously described b) encapsulating at least one cell and/or biological agent, wherein the biocompatible semi-permeable PEGDA layer comprises a paramagnetic or superparamagnetic metal that does not participate in the crosslinking of the PEGDA layer. In some embodiments, the paramagnetic or superparamagnetic metal can be iron, gadolinium, manganese, dysprosium and any combination thereof. In particular embodiments, the paramagnetic or superparamagnetic metal is iron, which is present in the microspheres as a ferum-oxide. The ferum-oxide can be derived from a Feridex® or Resovist® aqueous colloidal solution.

Furthermore, methods of delivering a cell and/or biological agent to a mammal (e.g., a human) comprising introducing the microcapsule according to any embodiment of the invention into the mammal are provided herein. In some embodiments, the microcapsule is introduced by injecting the microcapsule into the mammal via an intravascular catheter. In addition, in some embodiments, the microcapsule is injected into the mammal, e.g., into the portal vein, the heart, the muscle, the brain, the arterial supply, etc., of the mammal, in a pharmaceutically acceptable carrier.

In addition, provided herein are methods of synthesizing microcapsules. In some embodiments, a method of synthesizing an MRI-detectable microcapsule comprises forming a droplet comprising a cell and/or biological agent, a PEGDA polymer and a crosslinking agent (creating a self-polymerizing or UV crosslinked gel) and a US, MR or X-Ray visible contrast agent that is not crosslinked and is added dropwise to an inert bath. Also provided is a method of synthesizing an MRI-detectable microcapsule, comprising forming a droplet comprising a cell and/or biological agent, and a PEGDA polymer as described above

### Hydrogel Compositions

The invention features hydrogel compositions that form microspheres.

In one aspect, the invention features a composition comprising a PEGDA based biomaterial and a contrast agent. In one embodiment, the contrast agent is selected from the group consisting of: magnetic resonance contrast agents, radiopaque contrast agents, ultrasound contrast agents, and nuclear medicine imaging contrast agents.

In some embodiments of the invention, the biocompatible semi-permeable membrane PEGDA and a paramagnetic or superparamagnetic metal that does not participate in the crosslinking of the PEGDA polymer. In some embodiments, the paramagnetic or superparamagnetic metal can be iron, gadolinium, manganese, dysprosium and any combination thereof. For example, a superparamagnetic iron compound, ferum-oxide, may be used. In some embodiments, the iron compound is provided to the microcapsule via a clinical grade ferum-oxide composition, such as via a FERIDEX or RESOVIST colloidal solution.

In some embodiments of the invention, the biocompatible PEGDA semi-permeable membrane comprises a radiopaque contrast agent. In some embodiments, the radiopaque contrast agent includes bismuth, and in some embodiments, the radiopaque contrast agent includes barium. In other embodiments, the radiopaque contrast agent can include iodinated compounds, or metal nanoparticles or quantum dots comprised of a radiopaque or ultrasound visible metal such gold or tantalum that are made biocompatible by coating with dextran, silica or peg and/or tantalum.

In some embodiments of the invention, the PEGDA biocompatible semi-permeable cell membrane comprises a fluorocarbon (or perfluorocarbon). In some embodiments, the fluorocarbon is detectable by MRI and ultrasonography, and in some embodiments, the fluorocarbon is also radiopaque. Exemplary fluorocarbons include perfluorooctylbromides and perfluoro-crown ethers.

In a particular embodiment, the magnetic resonance contrast agent is selected from the group consisting of: Manganese Oxide, perfluorocarbons, Feridex, Gadolinium, Combidex, Bang Magnetic Particles, Gd-DTPA, Gadolinium And Manganese Derivatives, Superparamagnetic Iron Oxide Particles, gadopentetate dimeglumine, Gd-DOTA, Gd-DTPA-BMA, Gd-HP-DO3A, Gd-DTPA-BMEA, Gd-DO3A-butrol, Gd-BOPTA, Mn-DPDP, Gd-EOB-DTPA, Gd-BOPTA, AMI-25, SH U 555A, gadoflourine-M, AMI-227, EP-2104R, P947, Gd-DTPA mesophorphryn, SH U 555 C, NC-100150, MS-325, gadoflourine-M, gadomelitolm manganese chloride, ferric amonium citrate, and barium sulfate suspensions.

In another particular aspect, the invention features a composition comprising a PEGDA based biomaterial and a biocompatible material. In another embodiment, the biocompatible material is selected from the group consisting of: polyvinyl alcohol, sodium polyacrylate, acrylate polymers, Hyaluronase Polymers, collagen membrane, Porous HA/TCP ceramic composite, Hydroxyapatite bone cement, PVP/PMMA, tricalcium phosphate, Hydroxyapatite coated collagen fibres, calcium sulphate, Hydroxyapatite (HAp), Phosphorylcholine (PC), silicone, ultrahigh molecular weight polyethylene, polyethylene, acrylic, nylon, Polyurethane, Polypropylene, poly(methyl methacrylate), Teflon, Dacron, acetal, polyester, silicone-collagen composite, polyaledehyde, poly(vinyl chloride), silicone-acrylate, poly(tetrafluoroethylene), hydroxyethyl methacrylate (HEMA), poly(methyl methacrylate) (PMMA), poly(glycolide lactide), poly(glycolic acid), tetrafluoroethylene, hexafluoropropylene, poly(glycolic acid), poly(lactic acid), desaminotyrosyltyrosine ethyl ester, polydioxanone, fibrin, gelatin, hyaluronan, tricalcium phosphate, polyglycolide (PGA), polycaprolactone, poly (lactide-co-glycolide), polyhydroxybutyrate, polyhydroxyvalerate, trimethylene carbonate, polyanhydrides, polyorthoesters, poly(vinyl alcohol), poly(N-vinyl 2-pyrrolidone), poly(ethylene glycol), poly(hydroxyethylmethacrylate), n-vinyl-2-pyrrolidone, methacrylic acid, methyl methacrylate, and maleic anhydride, polycaprolactone, poly(amino acids) ie poly(L-lysine), poly(1-ornithine), poly(glutamic acid), polycyanoacrylates, polyphosphazenes, poly(lactic acid), poly(glycolic acid), crown ethers, cyclodextrins, cyclophanes, ethylene glycol, Methylacrylate, Para-xylylene, Biodegradable Copolymers, Copolymer Surface Coatings, Starch Polymers, Polylactic Acid, Cellophane, Tyrosine Polycarbonates Lactide and Glycolide Polymers, Collagen, PTFE, silicone, Keratin-Based Materials, Fibrous Composites-Carbon Fiber and Particles, Polymer Composites, Artificial/Natural Material Composites, Glass-Ceramic/Metal Composites, Glass-Ceramic/Nonmetal Composites, Dental Composites, Ormocer, hydrogels, timed-release foams, and polymeric carriers.

In certain preferred embodiments, the invention features a nucleophilic component and a component containing a conjugated unsaturated bond, whereby the composition crosslinks within a target structure (e.g. a blood vessel, an intraocular space). In situ gelling biomaterials are attractive for use in the methods of the invention because of increased ease of use and reduced invasiveness associated with their application as implanted materials.

In situ Gelling Biomaterials

United States Patent Application 20060148897, incorporated by reference in its entirety herein, describes a conjugated unsaturated compound for use as an in situ gelling biomaterial. In certain preferred aspects, the instant invention is based on a chemical reaction in which two or more precursor components, namely a nucleophilic component and a component containing a conjugated unsaturated bond, are polyermized or crosslinked in situ in a self-selective manner. These two precursor components are self-selective in their reaction rates. In other words, the nucleophilic component reacts faster with the component containing a conjugated unsaturated bond than with other components present during the reaction, and the component containing a conjugated unsaturated bond reacts faster with the nucleophilic compound than with other components present during the reaction.

The functionalities of the precursor components will affect the resulting polymerization product. The word “functionality” as used herein refers to the number of reactive sites, as generally used in polymer science. Mixing two components each having a functionality of two results in a linear polymeric biomaterial. If one of the components has a functionality of more than two, mixing of the components will result in a cross-linked polymeric biomaterial. In cross-linked biomaterials, the components can be very hydrophilic, and the overall material can yet remain as an intact solid, not dispersing throughout the body. If such a non-dispersing system is desired for a linear polymeric biomaterial, it is useful if at least one precursor component be hydrophobic, such that the resulting biomaterial also be insoluble in water or body fluids.

The present invention makes use of a Michael-type addition reaction between the nucleophilic component and the component containing a conjugated unsaturated bond. The reaction can be exemplified as follows:

Such Michael-type addition reactions can be performed on a wide variety of conjugated unsaturated compounds in accordance with the invention. Exemplary conjugated unsaturated compounds include those having structures 1 to 20 set forth below. In these structures, P indicates an oligomeric or polymeric structure, examples of which are discussed further below. In structures 1 to 20, P is intended as terminated with a CH.sub.2, CH or C group. Reactive double bonds can be conjugated to one or more carbonyl groups in a linear ketone, ester or amide structure (1, 2) or to two in a ring system, as in a maleic or paraquinoid derivative (3, 4, 5, 6, 7, 8, 9, 10). In the latter case, the ring can be fused to give a naphthoquinone (6, 7, 10) or a 4,7-benzimidazoledione (8), and the carbonyl groups can be converted to an oxime (9, 10). The double bond can be conjugated to a heteroatom-heteroatom double bond, such as a sulfone (11), a sulfoxide (12), a sulfonate or a sulfonamide (13), or a phosphonate or phosphoamide (14). Alternatively, the double bond can be conjugated to an electron-poor aromatic system, such as a 4-vinylpirydinium ion (15). Triple bonds can be used in conjugation with carbonyl or heteroatom-based multiple bonds (16, 17, 18, 19, 20).

Structures such as 1 and 2 are based on the conjugation of a carbon-carbon double bond with one or two electron-withdrawing groups. One of them is always a carbonyl, increasing the reactivity passing from an amide, to an ester, and then to a phenone structure. The nucleophilic addition is easier upon decreasing the steric hindrance, or increasing the electron-withdrawing power in the alpha-position: CH.sub.3<H<COOW<CN.

The higher reactivity obtained by using the last two structures can be modulated by varying the bulkiness of the substituents in the beta-position. where the nucleophilic attack takes place. The reactivity decreases in the order P<W<Ph<H. So the position of P too can be used to tune the reactivity towards nucleophiles. This family includes some 10 compounds for which a great deal is known about their toxicology and use in medicine. For example, water-soluble polymers with acrylates and methacrylates on their termini are polymerized (by free radical mechanisms) in vivo, in hydrogel sealants and bone cements, respectively. Thus, acrylate and methacrylate-containing polymers have been seen in the body before in clinical products, but for use with a dramatically different chemical reaction scheme.

The structures 3 to 10 exhibit very high reactivity towards nucleophiles, due both to the cis configuration of the double bond and the presence of two electron-withdrawing groups. Unsaturated ketones react faster than amides or imides, due to the stronger electronegativity of these carbonyl groups. So, cyclopentendione derivatives react faster than maleimidic ones (3), and para-quinones react faster than maleic hydrazides (4) and also faster than cyclohexanones, due to more extended conjugation. The highest reactivity is shown by naphthoquinones (7).

P can be placed in positions where it does not reduce the reactivity of the unsaturated group, that is in the opposite part of the ring, on another ring or O-linked through a para-quinone mono-oxime. P can be also linked to the reactive double bond, particularly if the nucleophilic addition rate is to be decreased.

The activation of double bonds to nucleophilic addition can be obtained also by using hetheroatom-based electron-withdrawing groups. In fact, heteroatom-containing analogs of ketones, esters and amides provide a similar electronic behavior. Structures 13 and 14 can also be used as easily hydrolyzable groups that can promote a quick gel degradation. The reactivity towards nucleophilic addition increases with electronegativity of the group, that is in the order 11>12>13>14, and is enhanced by the linkage with an aromatic ring. A strong activation of double bonds can also be obtained, using electron-withdrawing groups based on aromatic rings. Any aromatic structure containing a pyridinium-like cation (e.g., derivatives of quinoline, imidazole, pyrazine, pyrimidine, pyridazine, and similar sp containing compounds) strongly polarizes the double bond and makes possible quick Michael-type additions.

Carbon-carbon triple bonds, conjugated with carbon- or heteroatom-based electron-withdrawing groups, can easily react with sulphur nucleophiles, to give products from simple and double addition. The reactivity is influenced by the substituents, as for the double bond-containing analogous compounds.

Particularly preferred conjugated unsaturated compounds for use in the invention include acrylates, vinylsulfones, acrylamides, quinones and vinylpyridiniums, with acrylates being particularly preferred.

The nucleophiles that are useful are those that are reactive towards conjugated unsaturated groups by way of Michael-type addition reactions. The reactivity of the nucleophile depends on the identity of the unsaturated group, but the identity of the unsaturated group is first limited by its reaction with water at physiologic pH. Thus, useful nucleophiles will generally be more nucleophilic than water at physiologic pH. Preferred nucleophiles are those that are commonly found in biological systems for reasons of toxicology, but ones that are not commonly found free in biological systems outside of cells. Thus, while there may be examples in which amines can be employed as effective nucleophiles, the most preferred nucleophile is the thiol. Thiols are present in biological systems outside of cells in paired form, as disulfide linkages. When the highest degree of self-selectivity is desired (e.g., when a therapeutic protein is incorporated, when the gelation reaction is conducted in the presence of tissue and chemical modification of that tissue is not desirable), then a thiol will represent the strong nucleophile of choice.

There are other situations, however, when the highest level of self-selectivity may not be necessary. This would include situations when no therapeutic protein is incorporated and when the gelation reaction is conducted in situ, but when chemical bonding to the tissue is either desirable or is not undesirable. In these cases, an amine may serve as an adequate nucleophile. Here, particular attention is paid to the pH, in that the deprotonated amine is a much stronger nucleophile than the protonated amine. Thus, for example, the alpha amine on a typical amino acid (pK as low as 8.8 for asparagine, average of 9.0 for all 20 common amino acids except proline) has a much lower pK than the side chain epsilon amine of lysine (pK 10.80). As such, if particular attention is paid to the pK of an amine used as the strong nucleophile, substantial self-selectivity can be obtained. Proteins have only one alpha amine (on the N-terminus). By selection of an amine with a low pK, and then formulation of the final precursor solution such that the pH were near that pK, one could favor reaction of the unsaturation provided with the amine provided, rather than other amines present in the system. In cases where no self selectivity is desired, one need pay less attention to the pK of the amine used as the nucleophile. However to obtain reaction rates that are acceptably fast one must adjust the pH of the final precursor solution such that an adequate number of these amines are deprotonated.

The term “nucleophilic group” as used herein includes not only the functional groups themselves (e.g., thiol or amine), but also molecules that contain the functional group (e.g., cysteine or cystyl residue, or lysine or lysyl residue). The nucleophilic groups may be contained in molecules with great flexibility in overall structure. For example, a difunctional nucleophile could be presented in the form of Nuc-P-Nuc, where P has the meaning discussed above, and Nuc refers to the nucleophile. Likewise, a branched polymer P could be derivatized with a number of nucleophiles to create P-(Nuc)i, where i=2 would be useful. Nuc needs not be displayed at the chain termini of P. For example, a repeating structure could be envisioned: (P Nuc).sub.i where i=2 would be useful. Clearly, not all of the P or Nuc groups in such a structure need to be identical. It is only necessary that one nucleophilic precursor contain greater than or equal to two such Nuc groups.

Likewise, similar structures of P and the conjugated unsaturated groups described above may be formed. It is only necessary that one conjugated unsaturated precursor contain greater than or equal to two such conjugated unsaturated groups.

It should be noted and understood, that it is not necessary that both precursor components, for example, both the nucleophilic precursor component and the conjugated unsaturated precursor component, actually be polymeric in the usual sense of the word. It is only the functionality that matters. In practice, it is convenient if at least one component is polymeric in the usual sense of the word, but this is not absolutely necessary. For example, useful materials result from the reaction of a PEG triacrylate with dicysteine, and likewise, useful materials result from the reaction of a PEG trithiol and a low molecular weight diacrylate. Further, useful materials for some applications also result from reaction of a dicysteine and a low molecular diacrylate.

In practice, it is convenient and useful when one or more precursor component is polymeric in the usual sense of the word. In these cases, P can be a synthetic hydrophilic polymer, a synthetic hydrophobic polymeric liquid, a synthetic hydrophobic polymer that is soluble in solvents of acceptable toxicity or biological influence for the envisioned application, a biosynthetic protein or peptide, a naturally occurring protein or processed naturally occurring protein, or a polysaccharide.

As noted above, thiols are of particular interest as the nucleophilic component. Although proteins contain the amino acid cysteine, the side chain of which terminates in a thiol, there are very few free thiols within proteins. Most proteins contain an even number of cysteine residues, and these are then paired and form disulfide-cross-links between various regions of the protein. Some proteins contain an odd number of cysteine residues, and most of these are present as disulfide linked dimers, again resulting in no free thiol residues being present in the native protein. Thus, there are very few free thiols in proteins. Some important electron transferring molecules, such as glutathione, contain a free thiol, but these molecules are generally restricted in their spatial location to the inside of a cell. Conjugated unsaturated structures presented outside the cell will be substantially unreactive with most proteins at near-physiological conditions. Accordingly, using a thiol with the component containing a conjugated unsaturated bond in the mixture of the invention will react in a very self-selective manner.

In the above structures, the group P can be a polymer such as poly(ethylene glycol), poly(ethylene oxide), poly(vinyl alcohol), poly(ethylene-co-vinyl alcohol), poly(acrylic acid), poly(ethylene-co-acrylic acid), poly(ethyloxazoline), poly(vinyl pyrrolidone), poly(ethylene-co-vinyl pyrrolidone), poly(maleic acid), poly(ethylene-co-maleic acid), poly(acrylamide), or a poly(ethylene oxide)-co-poly(propylene oxide) block copolymers. This is not an exhaustive list, as other hydrophilic polymers could also be used. P can also be a copolymer, a block copolymer, a graft copolymer, or a random copolymer. Blocks, which are polymerized on the ends of the hydrophilic polymers, can be composed of, for example, lactic acid, glycolic acid, epsilon-caprolactone, lactic-co-glycolic acid oligomers, trimethylene carbonate, anhydrides, and/or amino acids, for example, to confer degradability by hydrolytic or enzymatic means.

P can also be selected to create a hydrophobic system, for example, by using a water-dispersible liquid such as polypropylene glycol. Even if P is not a hydrophobic polymer, the component containing P can be made hydrophobic, such as with pentaerythritol-tetrakis (3-mercaptopropionate) and pentaerythritol triacrylate (where the P group is pentaerythritol).

Random copolymers can be based on vinyl alcohol, such as poly(N vinylpyrroliclone-co-vinyl alcohol) or poly(ethylene-co-vinyl alcohol), with different compositions, can be derivatized with conjugated unsaturated groups, such as acrylates, 5 benzoquinones, naphthoquinones and others. The vinyl alcohol copolymers can be functionalized with (CH.sub.2).sub.nCOOH groups by reaction with .omega.-bromo-carboxylic acids. The resulting polymers or acrylic or methacrylic acid copolymers can be used for the attachment of quinone groups. Comonomer composition and extent of functionalization do not dramatically influence the reaction rates, unless they determine solubility or phase transition. On the other hand, they determine the final mechanical properties.

Alternatively, P may be a protein or peptide. Examples of suitable proteins and peptides for use in the invention are disclosed in International Patent Publication No. WO 00/44808, the entire disclosure of which is incorporated herein by reference.

Utilizing terminology from polymer science, polymers can be made by reaction of monomers with a functionality of 2. Cross-linked networks of polymers can be made if some or all of the monomers have a functionality greater than 2. Molecules are described herein having a functionality greater than or equal to 2 (monomers or macromers), which can be reacted together to form a cross-linked network, where functionality is defined in terms of addition reactions. As used herein, polymerization refers to the reaction of monomers or macromers with functionality of 2, and cross-linking refers to the reaction of monomers or macromers some or all of which have a functionality greater than 2. The term monomers here is not limited to small molecules, but can also refer to polymers and biopolymers.

The monomers described are of two classes, which when reacted together form a linear or cross-linked biomaterial. Both classes of monomers are required to be mixed together for cross-linking to occur. One class of monomer contains 2 or more conjugated unsaturated groups (thus, a functionality of 2 or more), preferably conjugated. The other class of monomer contains 2 or more nucleophiles (thus, a functionality of 2 or more), preferably nucleophiles that are stronger nucleophiles than others present as other components of the system.

When water-soluble or water-dispersible precursor monomers are mixed together (referred to as the final precursor solution), linear or cross-linked gels or networks are formed, and such reactions can proceed in water at physiologic or nearly-physiologic salt concentrations and pH. It is not necessary that the monomers be entirely soluble in water, and indeed it is sometimes beneficial that they not be soluble in water. In such cases, gels may not be obtained as the final material, but rather more hydrophobic, less water-swelling materials. These can be particularly useful in the delivery of hydrophobic drugs and in the formation of materials with substantial structural strength. It is only necessary that the two components be either soluble in each other or at least finely dispersible in each other, perhaps in the presence of an emulsifying agent. In this manner, the two components can come close enough to each other to react to form the linear or cross-linked material.

It is also possible to work with solutions of monomers formed in a solution other than water. For example, the use of N-methylpyrrolidone (NMP) as a solvent in injectable biomaterial systems is known, and as such it is possible, when one wishes to work with the precursor components in solution, but with precursor components that are not freely soluble in water, to employ certain organic solvents that are acceptable for use with the sensitive biological material under consideration.

When the biomaterial is being formed in the body, as in the present invention, the list of acceptable solvents is dominated by toxicity concerns. For this application, NMP is a particularly favorable organic solvent. The toxicity of the solvent system can also be modulated by employing a mixed solvent system, comprising the organic solvent and water, to lower the overall concentration of organic solvent but to still provide good solubility or dispersability in the mixed solvent system.

Mixing to form the final precursor solution can occur in a variety of ways. Most straightforwardly, one solution contains the nucleophilic precursor component and one solution contains the conjugated unsaturated precursor component. These two components are formulated in solvent and buffer systems such that the pH and concentrations obtained after mixing are appropriate for the chemical reaction to proceed. Such mixing could occur in a static mixer at the function of two syringes, for example. Other mixing approaches can be imagined. For example, mixing can occur between fine particles of each of the two precursor solutions in an air spray.

Alternatively, one solution can be prepared from both precursor components, but at a pH, for example, such that the reaction does not proceed or proceeds only slowly. After or just immediately preceding placement of the pre-mixed precursor solution, the pH can be adjusted (e.g., by change of temperature, or mixing with acid or base, or by a chemical reaction to create an acid or base, or diffusion of an acid or base), to result in a final condition in the final precursor solution that is appropriate for the chemical reaction to proceed.

Another approach is to prepare the final precursor solution at a temperature such that the reaction does not proceed or proceeds only very slowly, either related to the activation energy of the reaction or to a buffer with temperature-sensitive characteristics or both. Upon warming or cooling (most usefully warming) to the final application temperature (e.g., to body temperature after injection), the conditions in the final precursor solution are appropriate for the chemical reaction to proceed.

Other considerations are important for the reaction occurring it situ. For example, the reactants are desirably stable in water when the precursor solutions are prepared in water. Stable is defined as reacting slowly, with slowly defined as sufficiently slow to allow the reaction between the two components to proceed and still result in the formation of the desired biomaterial. Additionally, the addition reaction in the final precursor solution is preferably not exothermic to the point of causing tissue damage, drug breakdown or other detrimental results to the biological material under consideration. The temperature of the gelling solution generally should not be raised above 60.degree. C during gelation, and preferably even cooler maximum reaction temperatures are desirable. Further, the components of the precursor solution must not be toxic at concentrations that diffuse out of the final precursor solution as it is applied, with the word toxic being defined as inducing a medically unacceptable tissue reaction in a medically relevant context.

In a preferred embodiment, the invention employs a thiol as the nucleophilic component and an acrylate as the component containing a conjugated unsaturated bond. Particularly preferred thiols include pentaerythritol-tetrakis (3-mercaptopropionate) (QT) and poly(ethylene glycol) hexathiol. (PEGHT). Particularly preferred acrylates include poly(ethyleneglycol)diacrylate 570 (PEGDA), poly(propylene glycol) diacrylate 900 (PPODA), pentaerythritol triacrylate (TA), and poly(ethylene glycol) tetraacrylate (QA). Acrylates react orders of magnitude faster with thiols than with amines and other nucleophiles present in biological samples, where free thiols are present in negligible leachable content. Such a system is also waterborned and, before gelations, possesses low viscosity, allowing delivery through a microcatheter. The Michael-type addition reaction, being pH-activated, allows, for certain combinations of reagents, premixing of the reagents without reaction, while the reaction can be started at a desired time by addition of a base. The above-noted monomeric multifunctional materials are dispersed in water at high solid content, typically 75-wt %. Further, these materials can be made radiopaque by including in the reaction mixture a suitable radiopaque agent, such as barium sulfate, tantalum, iohexyl (commercially available under the name Omnipaque from Amersham Health, Princeton, N.J.), iothalamate meglumine (commercially available under the name Contray from Mallinkckrodt, St. Louis, Mo.), ioxaglate meglumine and ioxaglate sodium (commercially available as a mixture under the name Hexabrix from Mallinckrodt, St. Louis, Mo.).

The liquid mixture containing the nucleophilic component and the component containing a conjugated unsaturated bond is introduced into a target vessel or structure.

The liquid mixture preferably also comprises a buffer, such as phosphate buffered saline (PBS). Other components can also be included within the liquid mixture, such as a base for adjusting the pH of the mixture and/or a surfactant. If a base and/or a surfactant is included in the liquid mixture, they are preferably included within a buffer solution. Exemplary bases for use in the present invention include sodium hydroxide, triethanolamine, and choline. Exemplary surfactants for use in the present invention include sorbitan monooleate, polyethylene glycol-co-polypropylene glycol, Tween 20 and Tween 80.

The mixture gels within the body to form a gelled composition. In accordance with this step of the inventive method, the Michael-type addition reaction between the nucleophilic component and the component containing a conjugated unsaturated bond is occurring predominantly, if not entirely, within the body. Accordingly, the rate of the Michael-type reaction desirably occurs over a clinically relevant period of time at a clinically relevant temperature and pH. Preferably gelation occurs over a period of less than about 60 minutes, more preferably less than about 30 minutes, still more preferably less than about 15 minutes, even more preferably less than about 5 minutes.

The speed at which the reaction occurs is largely a function of the pH of the reaction mixture, as well as the strength of the buffer solution employed. For example, a liquid mixture containing pentaerythritol-tetrakis (3-mercaptopropionate) (QT) and poly(ethyleneglycol)-diacrylate 570 (PEGDA) in 100 mM PBS solution at pH 7.4 reacts to form a gel in about 5 minutes. In contrast, a liquid mixture containing QT and PEGDA in 10 mM PBS solution will react in about 10 minutes if adjusted to a pH of about 9. The strength of the buffer solution is preferably sufficient to deprotonate the thiols in the liquid mixture. Preferably the liquid mixture contains a PBS solution having a strength ranging from about 1 mM to about 300 mM, more preferably from about 10 mM to about 150 mM, still more preferably from about 75 mM to about 125 mM. Preferably the pH of the liquid mixture, when it is being introduced into the body, is at least 7, more preferably from about 7 to about 12. If the liquid mixture has a pH outside of this range, the pH can be adjusted immediately before introduction into the patient by addition of a suitable base, as noted above.

In certain embodiments the present compositions may contain a basic functional group, such as amino or alkylamino, and are, thus, capable of forming pharmaceutically-acceptable salts with pharmaceutically-acceptable acids. The term “pharmaceutically-acceptable salts” in this respect, refers to the relatively non-toxic, inorganic and organic acid addition salts of compounds of the present invention. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, for example, Berge et al. (1977) “Pharmaceutical Salts”, J. Pharm. Sci. 66:1-19)

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra)

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

As used herein, a radiopaque contrast agent is one that renders the microcapsule detectable using X-ray radiological methods, including fluoroscopy and computed tomography. Examples of radiopaque contrast agents include radiopaque bismuth or barium compounds, such as barium sulfate and bismuth sulfate, and stabilized complexes containing Bi or Ba. Iodine containing compounds, such as 2,3,5 Triiodobenzoic acid, 3,5-Diacetamido-2,4,6-triiodobenzoic acid (Hypaque), 5-(acetyl-(2,3 dihydroxypropyl)amino)-N,N-bis(2,3-dihydroxypropyl)-2,4,6-triiodo-benzene-1,3-dicarboxamide (iohexyl), etc., can also be added to the microspheres. Tantalum and tungsten compounds may also be used. Combinations of radiopaque contrast agents may also be used. Furthermore, radiopaque contrast agents may be used in microcapsules in combination with the paramagnetic and/or superparamagnetic metals described above. Thus, in some embodiments of the invention, the microcapsules can be both radiopaque and detectable by MRI.

In some embodiments, the radiopaque contrast agent is also detectable by magnetic resonance imaging and/or by ultrasonography. For example, in some embodiments, the radiopaque contrast agent may be a perfluorocarbon (PFC). A perfluorocarbon refers to a hydrocarbon compound wherein most or all of the hydrogen atoms have been substituted with fluorine atoms. Exemplary PFCs include brominated PFC such as perfluorooctylbromide (PFOB) and perfluoropolyethers (PFPE) such as perfluoro (crown ethers). PFOB (C₈F₁₇Br) is a linear molecule containing a residual bromine atom that has significant radiopacity to be detected under CT. PFPE is crown ether that is particularly attractive as a MR imaging agent as all fluorine atoms are spectroscopically equivalent. While both are suitable MRI contrast agents, PFOB microcapsules display trimodal imaging capabilities and are detectable under ¹⁹F MRI, CT, and US.

In some embodiments, the perfluorocarbon is also detectable by magnetic resonance and by ultrasonography.

The incorporation of PFCs into microspheres is attractive for a number of reasons. By exploiting the various features of PFCs, fluorinated biomaterials can be used to create smart scaffolds capable of providing information on perfusion of the encapsulated graft by monitoring O₂ tension noninvasively with MRI. In addition to providing a means of assessing pO₂, PFCs can also increase local oxygen tension. The ability to increase oxygen availability is paramount for the advancement of encapsulation therapy as many studies have suggested that graft failure occurs due to the lack of direct vascularization of the enclosed cells. This results in gradual tissue necrosis and death of encapsulated cells. In some embodiments, in vivo applications of these capsules with perfluorocarbon reservoirs could “reload” themselves by picking up O₂ from plasma that perfuses through the matrix. By acting as oxygen sinks, PFC containing microspheres may have broad implications for increasing the viability of many encapsulated cell types.

Emulsions suitable for use in the microsphere preparations of this invention may be prepared, for example, by adding two parts by volume of a brominated perfluorocarbon to 1 part by volume of lactated Ringer's solution containing a small amount (e.g., 6%) of an emulsifing agent, e.g., Pluronic F-68, and agitating on a vortex or sonicator until a stable emulsion is formed. More concentrated emulsions are formed by adding neat perfluorocarbon, up to a ratio of 12:1 by volume, and mixing until a stable emulsion is formed. Concentrated emulsions of this type, particularly those having perfluorocarbon/aqueous phase ratios of 6:1 to 10:1, will most likely be most useful for this microcapsule approach.

In addition to increasing local oxygen concentrations, there is some evidence that PFCs may have the additional advantage of enhancing the immunoisolatory properties of PEGDA microcapsules by acting in an immunomodulatory manner. Thus, PFC loaded PEGDA microspheres could further reduce rejection of cellular therapeutics in immunocompetent hosts.

A final potential advantage of incorporating PFCs in microspheres is that it provides a means of tracking cells using X-ray imaging modalities, MRI or ultrasound. X-ray and ultrasound guided procedures are the preferred method for minimally invasive interventions at present. For this reason, PFC microspheres could prove an ideal vehicle for targeted delivery of cellular agents. Further, as PFC containing microspheres are detectable with MRI, follow-up examinations with MRI may be performed while avoiding radiation exposure.

Like radionuclide tracers, there is essentially no endogenous fluorine signal in vivo. Thus, ¹⁹F “hotspot” MRI can be performed for tracking of the microcapsules. In phantom studies using a high field scanner (e.g., 9.4 T), fluorocapsules may be detected. Since the first clinical 7T MR scanners are currently being installed, it can be expected that ¹⁹F MRI will be possible in humans. However, the advantage of being able to deliver and visualize fluorocapsules alone is of great benefit, and the potential for MRI/MRS spectroscopy enhances enthusiasm for the PFC microcapsules.

As certain PFCs can be imaged with ultrasound (US), MRI and x-ray modalities, a final potential advantage of incorporating PFCs into PEGDA microspheres is the ability to non-invasively monitor capsule location. Such information could prove invaluable in determining fundamental questions such as ideal transplantation site and best means of delivery of such grafts. Although detectable under ¹H MRI, superparamagnetic iron oxides are not detected directly but instead are detected from a misalignment of the orientation of water protons, caused by microscopic disturbances of the magnetic field. PFC contrast agents take a different approach to molecular labeling than traditional contrast agents. Fluorinated contrast agents are detected directly by ¹⁹F MRI, assuring a lack of background signal as the body lacks any endogenous fluorine. As a result, when imaging fluorinated contrast agents, there is no uncertainty about the signal source. Furthermore, the fluorine signal offers a hotspot interpretation when superimposed on anatomical ¹H scans, which can be taken during the same session. Additionally, certain PFCs have significant radiopacity for visualization under X-ray imaging.

A means of assessing adequate perfusion by determining local oxygen saturation could prove invaluable for a better understanding of the long-term viability of encapsulated grafts after transplantation. In the case of PFCs, apolar oxygen imparts paramagnetic relaxation effects on ¹⁹F nuclei associated with spin-lattice relaxation rates (R₁) and chemical shifts. This effect is proportional to the partial pressure of oxygen (pO₂). If incorporated into grafts containing encapsulated cells, PFCs in combination with ¹⁹F MRI could provide a non-invasive means of determining graft perfusion. Furthermore, as superimposition of CT and MRI scans, using hybrid X-Ray/MR imaging systems, becomes more frequent, capsules with multimodal contrast agents, such as PFOB, will allow researchers and clinicians to accurately monitor encapsulated cells in vivo.

Also provided are compositions comprising a microcapsule described herein, in a pharmaceutically acceptable carrier. The term “pharmaceutically acceptable carrier” is used herein and in the claims to refer to a carrier medium that does not significantly alter the biological activity of the active ingredient (e.g., the antiviral activity of a compound according to the present invention) to which it is added. The one or more substances of which the pharmaceutically acceptable carrier is comprised, typically depend on factors (or desired features for its intended use) of the pharmaceutical composition such as the intended mode of administration, desired physical state (e.g., solid, liquid, gel, suspension, etc.), desired consistency, desired appearance, desired taste (if any), desired pharmacokinetic properties once administered (e.g., solubility, stability, biological half life), desired release characteristics (e.g., (a) immediate release (e.g., fast-dissolving, fast-disintegrating), or (b) modified release (e.g., delayed release, sustained release, controlled release)), and the like. As known to those skilled in the art, a suitable pharmaceutically acceptable carrier is typically sterile and may comprise one or more substances, including but not limited to, a diluent, water, buffered water, saline, 0.3% glycine, aqueous alcohol, isotonic aqueous buffer; a water-soluble polymer, glycerol, polyethylene glycol, glycerin, oil, salt (e.g., such as sodium, potassium, magnesium and ammonium), phosphonate, carbonate ester, fatty acid, saccharide, polysaccharide, stabilizing agent (e.g., glycoprotein, and the like for imparting enhanced stability, as necessary and suitable for manufacture and/or distribution of the pharmaceutical composition), excipient, preservative (e.g., to increase shelf-life, as necessary and suitable for manufacture and distribution of the pharmaceutical composition), bulking agent (e.g., microcrystalline cellulose, and the like), suspending agent (e.g., alginic acid, sodium hydrogel, and the like), viscosity enhancer (e.g., methylcellulose), taste enhancer (e.g., sweetener, flavoring agent, taste-masking agent), binder (generally, to impart cohesive quality to a tablet or solid formulation; e.g., gelatin, natural and/or synthetic gums, polyvinylpyrrolidone, polyethylene glycol, and the like), extender, disintegrant (e.g., sodium starch glycolate, sodium carboxymethyl cellulose, starch, and the like), dispersant, coating (generally to impart a surface active agent to a tablet or solid formulation; e.g., polysorbate, talc, silicon dioxide, and the like), lubricant (e.g., magnesium stearate, calcium stearate, sodium lauryl sulphate, and the like), or colorant.

Furthermore, methods of delivering a cell to a mammal (e.g., a human) comprising introducing the microsphere according to an embodiment of the invention into the mammal, are provided herein. In some embodiments, the microcapsule is introduced by injecting the microcapsule into the mammal via a magnetic resonance-detectable needle. In addition, in some embodiments, the microcapsule is injected into the mammal, e.g., into the portal vein of the mammal, in a pharmaceutically acceptable carrier. Various methods of delivering cells to animal are well known in the art. In further embodiments as described herein, the microspheres of this invention can be used as embolic agents and their detection by MRI, X-ray and/or ultrasound enables verification of successful embolization.

### Compositions

The present invention provides compositions, e.g. polymer matrices, comprising nanoparticles that can be loaded after polymerization.

The present invention further provides microspheres that comprise various biological or bioactive agents, such as drugs, factors, and/or other cytokines that may be included within the capsules either with or without cells of this invention. Thus, in some embodiments, the biological or bioactive agent can be present in the microsphere in the absence of any cells in the microsphere In further embodiments, the microspheres of this invention can comprise cells that are genetically engineered to produce various bioactive agents, such as, for example, cytosine deaminase, angiostatin, inhibiting factors for tumors etc, as well as enhancing/stimulating factors such as cytokines that stimulate immune cells to fight cancer (e.g., interferon beta, interferon gamma, interleukins etc). These bioactive agents and/or cells and/or genetically engineered cells can be present in any combination in the microspheres of this invention.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

The phrase “therapeutically-effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

As set out above, certain embodiments of the present compounds may contain a basic functional group, such as amino or alkylamino, and are, thus, capable of forming pharmaceutically-acceptable salts with pharmaceutically-acceptable acids. The term “pharmaceutically-acceptable salts” in this respect, refers to the relatively non-toxic, inorganic and organic acid addition salts of compounds of the present invention. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, for example, Berge et al. (1977) “Pharmaceutical Salts”, J. Pharm. Sci. 66:1-19).

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra)

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

In certain embodiments, the present invention provides pharmaceutical compositions comprising a bioactive agents. In certain embodiments, the pharmaceutical composition preferably comprises one or more of the agents in hydrogel microsphere. The present invention further provides novel therapeutic methods of treating a cancerous tumor comprising administering to the subject an effective amount of an agent (for example a chemotherapeutic) loaded microsphere. In certain embodiments, the method comprises parenterally administering a subject composition to a subject. In one embodiment, the method comprises direct intraarterial administration of a subject composition to a subject. In other embodiments, the method comprises administering an effective amount of a subject composition directly to the arterial blood supply of the cancerous tumor. In one embodiment, the cancerous tumor is a liver tumor. In other embodiments, the method comprises systemic administration of a subject composition to a subject. In certain embodiments, the methods of treating a cancerous tumor comprise administering a subject composition and administering a second agent to a subject.

In certain embodiments, PEGDA can be combined with magnetic resonance imaging and/or ultrasound contrast agents, in order to provide visibility during procedures performed with these imaging modalities. Hydrogel compositions according to the invention can use magnetic resonance (MR) contrast agents such as iron-based agents, gadolinium-based agents, and fluorinated contrast agents. Specific contrast agents include bang magnetic particles, manganese oxide, gadopenteltatedimeglumine, gadoteratemeglumine (Gd-DOTA), gadodiamide injection (Gd-DTPA-BMA), gadoleridol injection (Gd-HP-DO3A), gadoversetamide (Gd-DTPA-BMEA), gadobutrol (Gd-DO3A-butrol), gadobenate dimeglumine (Gd-BOPTA), megafodipir trisodium (Mn-DPDP), gadoxetic acid (Gd-EOB-DTPA), feuromoxides (AMI-25), ferucarbotran (SH U 555A), gadofluorine-M, ferumoxtran (AMI-227), EP-2104R, P947, Gd-DTPA mesoporphyrin, PEG-feron (NC-100150), ferucarbotran (SH 555 C), gadofosveset (MS-325), ferumoxytol (Code 7228), gadomer-17, gadomelitol (p792), MnHa/PEG, ferric ammonium citrate, manganese chloride, manganese-loaded zeolite, ferristene (OMP), ferumoxsil (AMI-121), perfluoro-octylbromide, barium sulfate, bismuth sulfate, miscellaneous perfluorocarbons, hexafluorobenze, perfluoropolyether, Gd-DTPA, gadolinium and manganese derivatives, miscellaneous superparamagnetic iron oxide particles. In particular, bromofluorocarbons provide Hotspot imaging on 19F magnetic resonance imaging (MRI), and have sufficient radio-opacity to be conspicuous on CT, and thus are attractive agents to use.

Potential ultrasound agents that can be incorporated with PEGDA include AI-700, Albunex, BG1135, BiSphere™, BR14, BY 963, CARDIOSPHERE, DEFINIEY, ECHOGEN, ECHOVIST-200, IMAGENT, IMAVIST, LEVOVIST, M1091, M1134, MP1950, MRX 115, MRX 408, MYOMAP, OPTISON, PESDA, Quantison, QW7437, SONAZOID, SONOGEN, SONORX, SONOVIST, SONOVUE, VISIPAQUE, ultra-small air bubbles, silica nanoparticles, perfluorocarbons, lipospheres, or any combination of shell composed of albumin, lipid, or polymer confining a gas such as nitrogen, or a perfluorocarbon.

Radiopaque contrast agents are useful in particular embodiments of the invention. Potential radiopaque contrast agents that are useful for dissolving or combining with PEGDA include ethiodized oil, tantalum powder, barium sulfate, bismuth sulfate, Acetrizoic Acid Derivatives, Diatrizoic Acid Derivatives, lothalamic Acid Derivatives, Ioxithalamic Acid Derivatives, Metrizoic Acid Derivatives, Iodamide, Lypophylic Agents, Aliphatic Acid Salts, Iodipamide, Ioglycamic Acid, Ioxaglic Acid Derivatives, Metrizamide Iopamidol, Iohexyl, Iopromide, Iobitridol, lomeprol, Iopentol, Ioversol, Ioxilan, Iodixanol, Iotrolan, and Perfluorocarbons (PFOB).

In exemplary embodiments of the methods, the PEGDA based biomaterial comprises one or more anti-cancer agents. The anti-cancer agent can be a therapeutic. Further, the anti-cancer agent can be selected from, but not limited to, any of the following: abiraterone acetate, altretamine, anhydrovinblastine, auristatin, bexarotene, bicalutamide, BMS184476, 2,3,4,5,6-pentafluoro-N-(3-fluoro-4-methoxyphenyl) benzene sulfonamide, bleomycin, N,N-dimethyl-L-valyl-L-valyl-N-methyl-L-valyl-L-proly-1-Lproline-t-butylamide, cachectin, cemadotin, chlorambucil, cyclophosphamide, 3′,4′-didehydro-4′-deoxy-8′-norvin-caleukoblastine, docetaxol, doxetaxel, cyclophosphamide, carboplatin, carmustine (BCNU), cisplatin, cryptophycin, cyclophosphamide, cytarabine, dacarbazine (DTIC), dactinomycin, daunorubicin, dolastatin, doxorubicin (adriamycin), etoposide, 5-fluorouracil, finasteride, flutamide, hydroxyurea and hydroxyureataxanes, ifosfamide, liarozole, lonidamine, lomustine (CCNU), mechlorethamine (nitrogen mustard), melphalan, mivobulin isethionate, rhizoxin, sertenef, streptozocin, mitomycin, methotrexate, 5-fluorouracil, nilutamide, onapristone, paclitaxel, prednimustine, procarbazine, RPR109881, stramustine phosphate, tamoxifen, tasonermin, taxol, tretinoin, vinblastine, vincristine, vindesine sulfate, and vinflunine.

A neoplastic growth can be any disease that is caused by or results in inappropriately high levels of cell division, inappropriately low levels of apoptosis, or both. For example, cancer is an example of a neoplasia. Examples of cancers include, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma).

In particular embodiments, it is preferable to use bland particles, those that do not contain any toxic agent, so non-targeted particles can be dissolved without the non-targeted release of agent. Alternatively if a toxic agent is used it is safer if the agent becomes activated in some way by, for example, ultraviolet (UV) light, or ultrasound (US) rupture. In the event of non-targeted delivery, the PEGDA capsules can be lysed, and the non-activated agent can clear the system before activation of the targeted agent.

The present invention relates in other embodiments to a method of selectively delivering a therapeutic agent to a targeted vessel. Delivery of the therapeutic agent is achieved in a highly selective manner through the use of a PEGDA based biomaterial to occlude the vessels in the area where the agent is not desirably delivered, and leaving non-occluded vessels free for agent delivery in the area of treatment.

Water-soluble drugs can easily be dissolved in aPEGDA and become trapped in the resulting matrix, once the sample is gelled, allowing for drug-enhanced embolization, a characteristic further enhanced by the fact that PEGDA gels have a porous structure that allows for controlled drug diffusion. Exemplary agents include, but are not limited to, chemotherapeutic agents, anti-inflammatory agents, antimicrobial agents, hormonal therapy agents, metalloproteinase inhibitors, sclerosing agents, angio-active agents, plasmids for gene therapy, adenoviral vectors for gene therapy, RNAi, antisense, lentivirus, microbubbles, toxins (ricin toxin, conotoxin, botulin toxin a-g, diphtheria toxin, cholera toxin, tetanus toxin, shiga-like toxin antibiotics, vaccines, photodynamic agents, alpha emitters, beta emitters, antibodies, hormones, recombinant glycoproteins and analgesics.

The present invention relates in exemplary embodiments to a method for the controlled release of an agent in a subject. Any agent is suitable for use in this method. Exemplary agents include, but are not limited to, chemotherapeutic agents, anti-inflammatory agents, antimicrobial agents, hormonal therapy agents, metalloproteinase inhibitors, sclerosing agents, angio-active agents, plasmids for gene therapy, adenoviral vectors for gene therapy, RNAi, antisense, lentivirus, microbubbles, toxins, antibiotics, vaccines, photodynamic agents, and analgesics.

In particular embodiments, the therapeutic agent is a nanomaterial. In other particular embodiments, the therapeutic agent is contained within the nanomaterial. In other particular embodiments, the therapeutic agent is bound to the nanomaterial. A nanomaterial can be, but is not limited to nanocotainers, biological nanomotors, peptide-based self-assembling materials, nanorobots, smart nanodevices as anticancer therapeutics, nanocomposite devices, nanoparticles comprised of carbohydrates, virus particles, lipids, DNA dendrimers, microchips, drug-loaded microchips, micropumps, hyperbranched polymers, polymer brushes, nanofibers, polymeric nanotubes, nanocapsules, Biosensors, nanotubes, nanowires, chemical sensors, nanohorns, nanorods, MEMS Micro-Electro-Mechanical systems, fluorescent nanoparticles, magnetic nanoparticles, colloidal gold nanoparticles, colloidal gold biofunctionalized nanomodules, magnetic nanoparticles for magnetic guided ‘tag and drag delivery’, nanoparticles conjugated with biological ligands, metal nanoclusters, dendrimer nanocomposites, DNA-linked nanoparticles, nanocolloids (organosols and hydrosols), metal nanopowders (Ag, Au, Pt, Pd), metal nanoparticles and magnetic fluids, palladium nanoparticles, nanomaterials comprised of silicon, aluminum nitride, zinc oxide, platinum, titatium dioxide, silicon dioxide, silicon carbide, cobalt, carbon (graphite), aluminum oxide, cerium oxide, aluminum, gold, silver, copper, nickel. Nano-glasses, nano-ceramics, Cu alloys, Ni alloys, Zn alloys, Co alloys, Zr alloys, nobel metals, light metals, Ti, Ti—Al, Ti transition metals alloy (Fe or Ni or Cu), Mg—Ni, Fe—Cu—Nb—Si—B alloy, Fe-transition metal alloy (Co, Ni, Cr, Cu, Zr), Al-transition metal alloy (Fe, Ni, Ti, Zr), Mg, Al—Mg alloy.

In exemplary embodiments, the nanomaterial is selected from, but not limited to, microboxes, microchips, microfluidic pumps, magnetic resonance microcoil, quantum dots, antibody targeted nanomaterials, nanocontainers, and nanoboxes.

Nanomaterials can be colloidal metals. A colloidal metal includes any water-insoluble metal particle or metallic compound dispersed in liquid water. Typically, a colloid metal is a suspension of metal particles in aqueous solution. Any metal that can be made in colloidal form can be used, including gold, silver, copper, nickel, aluminum, zinc, calcium, platinum, palladium, and iron. In some cases, gold nanoparticles are used, e.g., prepared from HAuCl.sub.4. Nanoparticles can be any shape and can range in size from about 1 nm to about 10 nm in size, e.g., about 2 nm to about 8 nm, about 4 to about 6 nm, or about 5 nm in size. Methods for making colloidal metal nanoparticles, including gold colloidal nanoparticles from HAuCl.sub.4, are known to those having ordinary skill in the art. For example, the methods described herein as well as those described elsewhere (e.g., US 2001/005581; 2003/0118657; and 2003/0053983) can be used to make nanoparticles.

A nanoparticle can have at least one agent linked to its surface. Any of the agents described herein can be linked covalently, non-covalently, or coordinately to the surface of the microsphere. For example, all the bonds from an agent to a nanoparticle can be covalent bonds to the surface of the nanoparticle. In some cases, some of the bonds are covalent to the surface of the nanoparticle, and some are noncovalent to the surface of the nanoparticle. In some cases, some of the bonds are covalent to the surface of the nanoparticle, and some are coordinate to the surface of nanoparticle.

In certain cases, a nanoparticle can have two, three, four, five, six, or more agents linked to its surface. Typically, many molecules of an agent are linked to the surface of the nanoparticle at many locations. Accordingly, when a microsphere is described as having, for example, two agents linked to it, the nanoparticle has two distinct agents, each having its own unique molecular structure, linked to its surface. In some cases, one molecule of an agent can be linked to the nanoparticle via a single attachment site or via multiple attachment sites.

An agent can be linked directly or indirectly to a nanoparticle surface. For example, an agent can be linked directly to the surface of a nanoparticle or indirectly through an intervening linker. Any type of molecule can be used as a linker. For example, a linker can be an aliphatic chain including at least two carbon atoms (e.g., 3, 4, 5, 6, 7, 8, 9, 10 or more carbon atoms), and can be substituted with one or more functional groups including ketone, ether, ester, amide, alcohol, amine, urea, thiourea, sulfoxide, sulfone, sulfonamide, and disulfide functionalities. In cases where the nanoparticle includes gold, a linker can be any thiol-containing molecule. Reaction of a thiol group with the gold results in a covalent sulfide (—S—) bond. Linker design and synthesis are well known in the art. Any type of agent can be linked to a nanoparticle. For example, an agent can be a therapeutic agent that has a therapeutic effect in the body. Examples of therapeutic agents include, without limitation, anti-angiogenic agents, chemotherapeutic agents, anti-inflammatory agents, anti-bacterial agents, anti-fungal agents, growth factors, immunostimulatory agents, anti-cholinergic agents, insulin, and insulin analogs.

As discussed herein, a therapeutic agent can be in any physical or chemical form, including an antibody, an antibody fragment, a receptor, a receptor fragment, a small-molecule, a peptide, a nucleic acid, and a peptide-nucleic acid. A therapeutic agent can function as a targeting agent in addition to functioning as a therapeutic agent. A targeting functionality can allow nanoparticles to accumulate at the target at higher concentrations than in other tissues. In general, a targeting molecule can be one member of a binding pair that exhibits affinity and specificity for a second member of a binding pair. For example, an antibody or antibody fragment therapeutic agent can target a nanoparticle to a particular region or molecule of the body (e.g., the region or molecule for which the antibody is specific) while also performing a therapeutic function.

A microsphere can have a diagnostic agent linked thereto. In some cases, a diagnostic agent and a therapeutic agent can both be linked to a nanoparticle. A diagnostic agent can allow the imaging of a nanoparticle in vivo. For example, a patient administered a microsphere having a diagnostic agent and a therapeutic agent linked thereto can be imaged once, e.g., to locate and/or stage a tumor, or at multiple time points, e.g., to monitor the efficacy of the therapeutic agent.

Any type of diagnostic agent can be linked to a nanoparticle, including, for example, an MR imaging agent, a radio-imaging agent, an X-ray imaging agent, and a near-IR imaging agent. Two or more diagnostic agents can also be linked to a nanoparticle, such as an MR imaging agent and an X-ray imaging agent, or a near-IR imaging agent and an MR imaging agent. An MR imaging agent can be a metal chelate, e.g., can include a chelating ligand and a paramagnetic metal ion coordinated thereto. Any type of chelating ligand can be used, including cyclic and acyclic chelating ligands such as DTPA, DOTA, DOTMA, DTPA-BMA, DOTAGA, and HP-DO3A. Examples of paramagnetic metal ions include, without limitation, Gd(III), Fe(III), Mn(II), Cr(III), Cu(II), Dy(III), Ho(III), Er(III), Eu(III), Tb(II), Tb(III), and Tb(IV).

In particular exemplary embodiments, the agent is contained within therapeutic liposomes. Liposomes are formed when phospholipids and their derivatives are dispersed in water. Upon dispersion in water the phospholipids form closed vesicles called “liposomes”, which are characterized by lipid bilayers encapsulating an aqueous core. Various liposomes have been used as carriers for entrapped therapeutic agents, such as drugs, enzymes and genetic sequences for use in medical science, in pharmaceutical science and in biochemistry.

Examples of liposome compositions include U.S. Pat. Nos. 4,983,397; 6,476,068; 5,834,012; 5,756,069; 6,387,397; 5,534,241; 4,789,633; 4,925,661; 6,153,596; 6,057,299; 5,648,478; 6,723,338; 6,627218; U.S. Pat. App. Publication Nos: 2003/0224037; 2004/0022842; 2001/0033860; 2003/0072794; 2003/0082228; 2003/0212031; 2003/0203865; 2004/0142025; 2004/0071768; International Patent Applications WO 00/74646; WO 96/13250; WO 98/33481; Papahadjopolulos D, Allen T M, Gbizon A, et al. “Sterically stabilized liposomes. Improvements in pharmacokinetics and antitumor therapeutic efficacy” Proc Natl Acad Sci U.S.A. (1991) 88: 11460-11464; Allen T M, Martin F J. “Advantages of liposomal delivery systems for anthracyclines” Semin Oncol (2004) 31: 5-15 (suppl 13). Weissig et al. Pharm. Res. (1998) 15: 1552-1556.

Examples of lipid compositions including targeting factors include U.S. Pat. Nos. 5,049,390; 5,780,052; 5,786,214; 6,316,024; 6,056,973; 6,245,427; 6,524,613; 6,749,863; 6,177,059; 6,530,944; U.S. Pat. App. Publication. Nos. 2004/0022842; 2003/0224037; 2003/143742; 2003/0228285; 2002/0198164; 2003/0220284; 2003/0165934; 2003/0027779; International Patent Application Nos. WO 95/33841; WO 95/19434; WO 2001037807; WO 96/33698; WO 2001/49266; WO 9940789; WO 9925320; WO 9104014; WO 92/07959; EP 1369132; JP 2001002592; Iinuma H, Maruyama K, et al., “Intracellular targeting therapy of cisplatin-encapsulated transferrin-polyethylene glycol liposome on peritoneal dissemination of gastric cancer” Int J Cancer (2002) 99 130-137; Ishida 0, Maruyama K, Tanahashi H, Iwatsuru M, Sasaki K, et al., “Liposomes bearing polyethylene glycol-coupled transferrin with intracellular targeting property to the solid tumors in vivo.” Pharmaceutical Research (2001) 18: 1042-1048; Holmberg et al., Biochem. Biophys. Res. Comm. (1989) 165(3): 1272-1278; Nam et al., J. Biochem. Mol. Biol. (1998) 31(1): 95-100; Nag et al., J. Drug Target. (1999) 6(6): 427-438.

A variety of drugs or agents may be included in the lipid-containing compositions of the present invention, for example, a compound or a gene. In certain embodiments, the drug may be an anticancer agent, for example, an anticancer agent suitable for encapsulation in a liposome. The amount of drug to be included in the lipid-containing compositions, and formulations thereof, as described herein can be readily determined by the skilled artisan in view of the teaching herein provided and depending on the drug selected and the use intended for the composition or formulation, taking into account factors specific to both the drug and the individual to be treated, as described further herein. In certain embodiments, the drug may be a nucleic acid, for example, but not limited to, antisense oligonucleotides, ribozymes, etc.

The lipid-containing compositions described herein can be modified with targeting factors and directed to a particular target cell. The term “targeting factor” refers to a moiety that can bind to a receptor or a surface antigen present on the surface of a target cell. In certain embodiments, the targeting factors are directed to cell surface receptors on a particular target cell. The targeting factor is often a protein or a peptide that can be attached to a lipid component of the lipid-containing composition. Most effectively, targeting factors are selected such that the targeted receptor or antigen is present only on cells that are targeted for the delivery of the drug or labeled compound (e.g., pathogenic cells) and not present on healthy cells. Alternatively, a greater number of receptors or antigens are expressed on the target cells (e.g., pathogenic or diseased cells) compared to non-targeted (e.g., healthy) cells. Preferably, the receptor or antigen that binds the targeting factor is either not present or present in low numbers on healthy cells such that binding with the targeting factor does not occur with frequency. In other words, targeting factors need to selectively deliver the liposomes as described herein (including encapsulated drug) to the targeted cells (e.g., pathogenic, unhealthy, etc.). Selective delivery of the encapsulated drug to the targeted cells thus reduces the occurrence of adverse effects due to the effect of encapsulated drug or labeled compound on non-targeted (e.g., healthy) cells, thereby also reducing the adverse effects experienced by the individual to whom the composition, or formulation thereof, is administered. Exemplary targeting factors include, but are not limited to, transferrin, folic acid, folate, hyaluronic acid, sugar chains (e.g., galactose, mannose, etc.), fragments of monoclonal antibodies, asialoglycoprotein, etc., as well as other targeting factors known to the skilled artisan. In particular embodiments, the targeting factor is a protein or peptide directed to a cell surface receptor (e.g., transferrin, folate, folic acid, asialoglycoprotein, etc.). In other embodiments, the targeting factor is directed to an antigen (e.g., fragments of monoclonal antibodies (e.g., Fab, Fab′, F(ab′).sub.2, Fc, etc.)). It is not intended that targeting factors include intact or whole monoclonal antibodies. The term-“whole antibody” or “intact antibody,” and cognates thereof, as used herein generally refer to antibody IgG of immune globulin. A fragment of a monoclonal antibody generally refers to a decomposition product of the monoclonal antibody, for example, a fragment obtained by using protease digestion, such as pepsin, etc. In certain embodiments, the targeting factor is not directed to an antigen (e.g., is not a fragment of a monoclonal antibody, e.g., Fab, Fab′, F (ab′).sub.2, Fc, etc).

In exemplary embodiments, the therapeutic liposomes are coated with protein. The protein can be, but is not limited to, antibodies, receptors, and cell surface markers.

It is desirable, according to the invention, to further combine the agent with a second agent selected from, but not limited to, contrast agents, quantum dots, antibodies, liposomes, and nanoboxes. The agent, in exemplary embodiments, is a cell secreting a therapeutic factor. The cell secreting factor can be, but is not limited to, any of the following: autogenic or allogenic fibroblasts, endothelial cells, transgenic cells, mesenchymal stem cells, embryonic stem cells, extraembryonic stem cells, embryonic germ cells, cardiac stem cells, umbilical stem cells, cardiac stem cells, all pluripotent and multipotent stem cell sources, pancreatic islet cells, hepatocytes, skin cells, intestinal stem cells, myoblasts, endothelial cells, cardiac myoblasts, dendritic cell, autologous tumor cells (method of sensitization and potential vaccine delivery), Monocyte derived activated killers, Natural Killer T Cells, patients own cancer cells with liposomal I1-2, cultured chondrocytes, hematopoietic stem cells, sertoli cells, xenogeneic cell sources of all listed above, skin cells, adipocytes, skin-derived stem cells, neural stem cells, glial progenitor cells, oligodendrocyte and oligo precursors, fat stem cells, other stem cells sources such as from amniotic fluid, baby teeth, bone marrow cells, cord and placental blood, fat tissue, fetal cells, unfertilized ova, pancreas, breast.

Autogenic or allogenic fibroblasts, endothelial cells or transgenic cells secreting therapeutic factors may be added to the hydrogel prior to delivery in order to create a bioactive tissue scaffold that may provide tissue regrowth from the inside out.

An exemplary use of the method of the invention is for diagnostic purposes. In one example, the method is used for selected angiography of a labeled vessel.

The label used in the method of the invention can be any label that is suitable for incorporation in to an PEGDA based biomaterial, and for use in, for example, diagnostic purposes. The label can be selected from the group that consists of, but is not limited to, radiolabel, fluorescent label, tissue dye. The label can be contained within a micelle. The radiolabel can be, but is not limited to any one of carbon 14, carbon 14 intermediates, tritium-labeled, iodine 125, and antibody targeted radioisotopes. The fluorescent label can be, but is not limited to, cadmium selenide, quantum dots, fluorophores and their amine-reactive derivatives, thiol-reactive probes, reagents for modifying groups other than thiols or amines, biotin derivatives, haptens, crosslinking reagents, and photoactivatable reagents. The tissue dye can be, but is not limited to, methylene blue.

In exemplary embodiments, the label is contained within a liposome. A variety of labeled compounds may be included in the lipid-containing compositions of the present invention. The labeled compound may be an agent useful in carrying out in vivo diagnostic procedures. As with the incorporation of agents as described herein, the amount of labeled compound to be included in the lipid-containing compositions, and formulations thereof, as described herein can be readily determined by the skilled artisan in view of the teaching herein provided and depending on the labeled compound selected and the use intended for the composition or formulation, taking into account factors specific to both the labeled compound and the individual to be diagnosed, as described further herein. Exemplary labeled compounds include, for example, materials comprising radioisotopes (e.g., ³H, ⁴C, ⁶⁷Ga, ¹¹¹In, ¹²⁵I, ¹²⁵I,), material comprising fluorescent moieties (e.g., fluorescein, fluorescein isothiocyanate, etc.), material comprising enzyme (e.g., peroxidase, alkaline phosohatase, etc.), as well as additional labeled compounds known to those of skill in the art. As will be appreciated by the skilled artisan, the selection of the labeled compound and methods used in diagnosis will depend upon the organ (e.g., liver, pancreas, prostate, etc.), tissue (e.g., malignant or non-malignant or tissue type (e.g., brain, cardiovascular, etc.) to be investigated.

The present invention relates in other embodiments to a method for the controlled release of a label to mark lesions for radiosurgery, the method comprising the steps of: administering to the subject administering to the subject a PEGDA based chemosphere.

The label can be selected from the group that consists of, but is not limited to, radiolabel, fluorescent label, tissue dye. The label can be contained within a micelle. The radiolabel can be, but is not limited to any one of carbon 14, carbon 14 intermediates, tritium-labeled, iodine 125, and antibody targeted radioisotopes. The fluorescent label can be, but is not limited to, cadmium selenide, quantum dots, fluorophores and their amine-reactive derivatives, thiol-reactive probes, reagents for modifying groups other than thiols or amines, biotin derivatives, haptens, crosslinking reagents, and photoactivatable reagents. The tissue dye can be, but is not limited to, methylene blue. In exemplary embodiments, the label is contained within a liposome.

The present invention relates in certain embodiments to a method for the controlled release of a contrast agent in a subject. In preferred embodiments, the contrast agent can be, but is not limited to, any of a magnetic resonance contrast agents, radiopaque contrast agents, ultrasound contrast agents, and Nuclear Medicine Imaging contrast agents.

Contrast agents can be, but are not limited to, optical agents, PET probe, ultrasound contrast agent, Radioisotopes, magnetic resonance image contrast agent, radiopaque contrast agent for visualization on X-ray modalities, for example DSA, Fluoroscopy, CT, X-Ray.

The targeted area according to the method of the invention is any area that is in need of a biocompatible material. The targeted area could be, but is not limited to, a target organ in need of drug treatment, a Composite for Artificial Muscle, Artificial Hearts and Pacemakers, Tissue-Engineered Human Heart Tissue, Artificial Pancreas, Artificial Liver, Artificial Blood Vessel, Artificial Nerves, drug/gene delivery stent, nerve graft, The targeted area is selected from the group consisting of: liver, pancreas, thyroid, heart, peripheral nerve scaffold, breast, bladder, cartilage, bone, tendon, ligament, blood vessel, and spinal cord.

PEGDA chemospheres can also incorporate any material that is transplantable in to the human body. The biocompatible material can be, but is not limited to: polyvinyl alcohol, sodium polyacrylate, acrylate polymers, Hyaluronase Polymers, collagen membrane, Porous HA/TCP ceramic composite, Hydroxyapatite bone cement, PVP/PMMA, tricalcium phosphate, Hydroxyapatite coated collagen fibres, calcium sulphate, Hydroxyapatite (HAp), Phosphorylcholine (PC), silicone, ultrahigh molecular weight polyethylene, polyethylene, acrylic, nylon, Polyurethane, Polypropylene, poly(methyl methacrylate), Teflon, Dacron, acetal, polyester, silicone-collagen composite, polyaledehyde, poly(vinyl chloride), silicone-acrylate, poly(tetrafluoroethylene), hydroxyethyl methacrylate (HEMA), poly(methyl methacrylate) (PMMA), poly(glycolide lactide), poly(glycolic acid), tetrafluoroethylene, hexafluoropropylene, poly(glycolic acid), poly(lactic acid), desaminotyrosyltyrosine ethyl ester, polydioxanone, fibrin, gelatin, hyaluronan, tricalcium phosphate, polyglycolide (PGA), polycaprolactone, poly (lactide-co-glycolide), polyhydroxybutyrate, polyhydroxyvalerate, trimethylene carbonate, polyanhydrides, polyorthoesters, poly(vinyl alcohol), poly(N-vinyl 2-pyrrolidone), poly(ethylene glycol), poly(hydroxyethylmethacrylate), n-vinyl-2-pyrrolidone, methacrylic acid, methyl methacrylate, and maleic anhydride, polycaprolactone, poly(amino acids) ie poly(L-lysine), poly(1-ornithine), poly(glutamic acid), polycyanoacrylates, polyphosphazenes, poly(lactic acid), poly(glycolic acid), crown ethers, cyclodextrins, cyclophanes, ethylene glycol, Methylacrylate, Para-xylylene, Biodegradable Copolymers, Copolymer Surface Coatings, Starch Polymers, Polylactic Acid, Cellophane, Tyrosine Polycarbonates Lactide and Glycolide Polymers, Collagen, PTFE, silicone, Keratin-Based Materials, Fibrous Composites-Carbon Fiber and Particles, Polymer Composites, Artificial/Natural Material Composites, Glass-Ceramic/Metal Composites, Glass-Ceramic/Nonmetal Composites, Dental Composites, Ormocer, hydrogels, timed-release foams, and polymeric carriers.

Certain preferred embodiments of the invention encompass a PEGDA based biomaterial and a contrast agent. Accordingly, the contrast agent can be selected from, but not limited to, magnetic resonance contrast agents, radiopaque contrast agents, ultrasound contrast agents, and nuclear medicine imaging contrast agents. The magnetic resonance contrast agent is selected from, but not limited to, any of: Manganese Oxide, perfluorocarbons, Feridex, Gadolinium, Combidex, Bang Magnetic Particles, Gd-DTPA, Gadolinium And Manganese Derivatives, Superparamagnetic Iron Oxide Particles, gadopentetate dimeglumine, Gd-DOTA, Gd-DTPA-BMA, Gd-HP-DO3A, Gd-DTPA-BMEA, Gd-DO3A-butrol, Gd-BOPTA, Mn-DPDP, Gd-EOB-DTPA, Gd-BOPTA, AMI-25, SH U 555A, gadoflourine-M, AMI-227, EP-2104R, P947, Gd-DTPA mesophorphryn, SH U 555 C, NC-100150, MS-325, gadoflourine-M, gadomelitolm manganese chloride, ferric amonium citrate, and barium sulfate suspensions.

Other compositions of the invention encompass a PEGDA based biomaterial and a biocompatible material. The biocompatible material can be, but is not limited to, polyvinyl alcohol, sodium polyacrylate, acrylate polymers, Hyaluronase Polymers, collagen membrane, Porous HA/TCP ceramic composite, Hydroxyapatite bone cement, PVP/PMMA, tricalcium phosphate, Hydroxyapatite coated collagen fibres, calcium sulphate, Hydroxyapatite (HAp), Phosphorylcholine (PC), silicone, ultrahigh molecular weight polyethylene, polyethylene, acrylic, nylon, Polyurethane, Polypropylene, poly(methyl methacrylate), Teflon, Dacron, acetal, polyester, silicone-collagen composite, polyaledehyde, poly(vinyl chloride), silicone-acrylate, poly(tetrafluoroethylene), hydroxyethyl methacrylate (HEMA), poly(methyl methacrylate) (PMMA), poly(glycolide lactide), poly(glycolic acid), tetrafluoroethylene, hexafluoropropylene, poly(glycolic acid), poly(lactic acid), desaminotyrosyltyrosine ethyl ester, polydioxanone, fibrin, gelatin, hyaluronan, tricalcium phosphate, polyglycolide (PGA), polycaprolactone, poly (lactide-co-glycolide), polyhydroxybutyrate, polyhydroxyvalerate, trimethylene carbonate, polyanhydrides, polyorthoesters, poly(vinyl alcohol), poly(N-vinyl 2-pyrrolidone), poly(ethylene glycol), poly(hydroxyethylmethacrylate), n-vinyl-2-pyrrolidone, methacrylic acid, methyl methacrylate, and maleic anhydride, polycaprolactone, poly(amino acids) ie poly(L-lysine), poly(1-ornithine), poly(glutamic acid), polycyanoacrylates, polyphosphazenes, poly(lactic acid), poly(glycolic acid), crown ethers, cyclodextrins, cyclophanes, ethylene glycol, Methylacrylate, Para-xylylene, Biodegradable Copolymers, Copolymer Surface Coatings, Starch Polymers, Polylactic Acid, Cellophane, Tyrosine Polycarbonates Lactide and Glycolide Polymers, Collagen, PTFE, silicone, Keratin-Based Materials, Fibrous Composites-Carbon Fiber and Particles, Polymer Composites, Artificial/Natural Material Composites, Glass-Ceramic/Metal Composites, Glass-Ceramic/Nonmetal Composites, Dental Composites, Ormocer, hydrogels, timed-release foams, and polymeric carriers.

### Dosage and Administration

The compositions of the invention as described herein are suitable for local or systemic administration.

Localized Administration

Localized administration of a therapeutic composition according to the invention is preferably by injection directly in to blood vessels or by means of a microcatheter, drip device, drug pump or drug-saturated solid matrix from which the composition can diffuse implanted at the target site.

In certain embodiments, therapeutic composition according to the invention may be used to deliver radiolabeled particles. Such use is particularly suited for the delivery of radiolabeled particles for locoregional radiotherapy.

Systemic Administration

Systemic administration of a therapeutic composition according to the invention may be performed by methods of whole-body drug delivery are well known in the art. These include, but are not limited to, intravenous drip or injection, subcutaneous, intramuscular, intraperitoneal, intracranial and spinal injection, or by the use of an implantable, time-release drug delivery device.

Systemic administration is advantageous when a pharmaceutical composition must be delivered to a target tissue that is widely-dispersed, inaccessible to direct contact or, while accessible to topical or other localized application, is resident in an environment (such as the digestive tract) wherein the native activity of the nucleic acid or other agent might be compromised, e.g. by digestive enzymes or extremes of pH.

A therapeutic composition of use in the invention can be given in a single- or multiple dose. A multiple dose schedule is one in which a primary course of administration can include 1-10 separate doses, followed by other doses given at subsequent time intervals required to maintain and or reinforce the level of the therapeutic agent. Such intervals are dependent on the continued need of the recipient for the therapeutic agent, and/or the half-life of a therapeutic agent. The efficacy of administration may be assayed by monitoring the reduction in the levels of a symptom indicative or associated with brain edema which it is designed to inhibit. The assays can be performed as described herein or according to methods known to one skilled in the art.

A therapeutically effective regimen may be sufficient to arrest or otherwise ameliorate symptoms of a disease. An effective dosage regimen requires providing the regulatory drug over a period of time to achieve noticeable therapeutic effects wherein symptoms are reduced to a clinically acceptable standard or ameliorated. The symptoms are specific for the therapeutic use.

### Microfluidic Droplet Generator

In certain aspects, the invention describes a microfluidic droplet generator to make very small spheres, e.g. microfluidic droplets that are 20-250 μm, preferably 20-150 μm, and more preferably 25-50 μm in size.

In preferred embodiments, a microfluidic droplet generator can be used with PEGDA, or can incorporate liposomes or other nanoparticles to suitably produce the microfluidic droplets that are of a small size.

Exemplary microfluidic droplet generators have been described, for example in U.S. Pat. No. 7,129,091, incorporated by reference in its entirety herein.

### Kits

The present compositions may be assembled into kits or pharmaceutical systems. Kits or pharmaceutical systems according to this aspect of the invention comprise a carrier means, such as a box, carton, tube or the like, having in close confinement therein one or more container means, such as vials, tubes, ampules, bottles and the like. The kits or pharmaceutical systems of the invention may also comprise associated instructions for using the PEGDA microspheres.

In certain aspects, this invention contemplates a kit including compositions as described herein, and optionally instructions for their use. Uses for such kits include, for example, therapeutic applications. In certain embodiments, the subject compositions contained in any kit have been lyophilized and require rehydration before use.

In other certain aspects, the present compositions may be assembled into kits or pharmaceutical systems for use in treating a subject suffering from a vascular or non-vascular condition, a vascular or non-vascular occlusion, a vascular or non-vascular hemorrhage, or a neoplastic growth. The kits or pharmaceutical systems of the invention may also comprise associated instructions for using the compounds of the invention for treating a subject suffering from a vascular or non-vascular condition, a vascular or non-vascular occlusion, a vascular or non-vascular hemorrhage, or a neoplastic growth.

In other aspects, the invention contemplates kits for selective delivery of a therapeutic agent to a targeted vessel in a subject using the microspheres as described herein, or kits for the controlled release of a label to mark lesions for radiosurgery in a subject, or the controlled release of a contrast agent in a subject, or any of the methods as described herein. The kits can comprise preformed chemospheres or the materials previously described to enable formation of chemospheres. Kits or pharmaceutical systems according to this aspect of the invention comprise a carrier means, such as a box, carton, tube or the like, having in close confinement therein one or more container means, such as vials, tubes, ampules, bottles and the like. The kits or pharmaceutical systems of the invention may also comprise associated instructions for using the compounds of the invention for selective delivery of a therapeutic agent to a targeted vessel in a subject.

In other embodiments, the subject compositions are suspended in a relatively inert hydrophobic material such as mineral oil, glycerol, fish oil, olive oil, vegetable oil, olive oil, soybean oil, safflower oil or any other non-toxic hydrophobic liquid that is washed from the spheres with an aqueous liquid prior to delivery. In certain embodiments the chemosphere in inert liquid stock can be frozen to prevent desiccation of the subject composition. In other embodiments, chemospheres are suspended in a relatively hydrophobic perfluorocarbon such as perfluorooctylbromide (PFOB) or perfluorocrownether that can be infused with chemospheres to provide in the case of PFOB MRI, US and X-RAY visible contrast and in the case of perfluorocrownether, MRI contrast. It will be cleared to one skilled in the art that above techniques can enable higher retention of aqueous drug within the hydrogel chemospheres.

In still other embodiments, this invention contemplates a kit including subject pharmaceutical compositions, and optionally instructions for their use. Uses for such kits include, for example, therapeutic applications. In certain embodiments, the subject compositions contained in any kit have been lyophilized and require rehydration before use. In other embodiments, the subject compositions are suspended in a relatively inert hydrophobic material such as mineral oil, glycerol, fish oil, olive oil, vegetable oil, olive oil, soybean oil, safflower oil or any other non-toxic hydrophobic liquid that is washed from the spheres with an aqueous liquid prior to delivery. In certain embodiments the chemosphere in inert liquid stock can be frozen to prevent desiccation of the subject composition. In other embodiments, chemospheres are suspended in a relatively hydrophobic perfluorocarbon such as perfluorooctylbromide (PFOB) or perfluorocrownether that can be infused with chemospheres to provide in the case of PFOB M RI, US and X-RAY visible contrast and in the case of perfluorocrownether, M RI contrast. It will be cleared to one skilled in the art that above techniques can enable higher retention of aqueous drug within the hydrogel chemospheres.

### EXAMPLES

Having now generally described the invention, the same will be more readily understood through reference to the following Examples, which are provided by way of illustration, and are not intended to be limiting of the present invention, unless specified.

### Example 1

### Methods of Preparation of Embolization Particles

In a preferred embodiment a polymer is prepared by mixing poly(ethylene glycol)-diacrylate (PEGDA; Nektar Therapeutics, Huntsville, Ala.) in sterile phosphate-buffered saline (PBS; GIBCO Invitrogen, Carlsbad, Calif.) to make a 15% (w/v) hydrogel. Photoinitiator, Igracure D2959 (Ciba Specialty Chemicals, Tarrytown, N.Y.) is added to the polymer at a concentration of 0.05% (w/v). The mixture is then added drop-wise to a reactant medium made up of an inert bath. An inert bath includes but is not limited to a formulation of 160 grams Span 80 and 3,840 grams USP mineral oil. The inert bath is then stirred at a rate of 200 RPM in a reaction vat. The Vat was then exposed to UV irradiation to initiate polymerization. Once reacted, the resultant crosslinked PEGDA sphere were cleaned of mineral oil and Span 8. The cleaning process involved dumping the resultant spheres over a 45-micron screen such that the beads were trapped on the screen while some of the reactants passed through. Further, the beads were then washed with Triton-X 100 using a standard ultrasonic cleaning machine. The particles were then dried and separated into size ranges using ASTM standard sieves.

### Example 2

### Methods of Preparation of Embolization Particles

In an alternate example, embolization particles were made using a slightly different process. The same procedure as above was followed, but the rpm of the reaction medium was increased to 300 rpm. The resulting particles exhibited the same mechanical properties as the spheres as described in Example 1, but the average size of these particles was smaller than the average size of the particles made according to the methods described in Example 1. Without being limited by theory, it is believed based on these results that the rpm of the reaction medium can be manipulated to obtain desired particle sizes.

### Example 3

### Methods of Preparation of Embolization Particles

In this example, embolization particles were made using a slightly different process. The same procedure as in Example 1 was followed, but a van de graaf generator was attached via an insulated wire to the tip of the needle extruding the pegda solution. The inert bath was grounded by placing an insulated wire that was attached to an external ground in the bath. With the bath grounded the van de graaf generator was turned on thereby creating an electrostatic charge between the needle tip and the inert bath. Without being limited by theory, it is believed that the electrostatic potential between the needle tip and inert bath causes PEGDA droplets to fall into the bath at a smaller size.

### Example 4

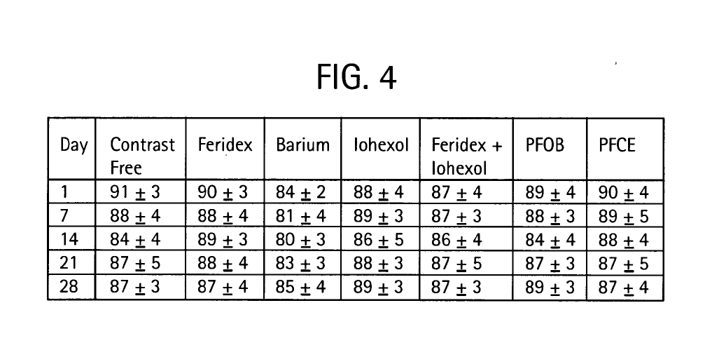

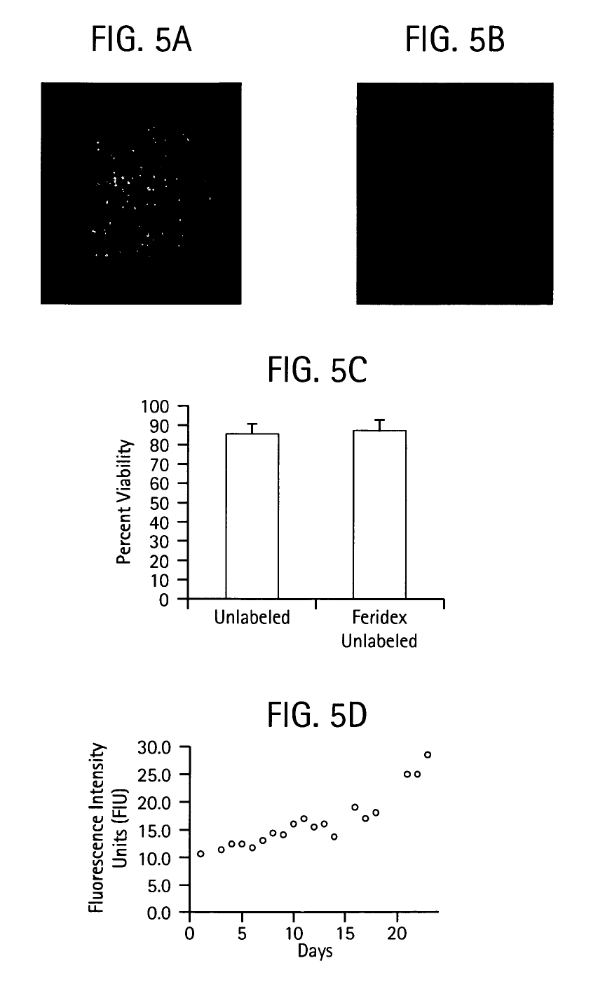

HepG2 cells were encapsulated in PEGDA chemospheres via a microfluidic system in which a mixture of therapeutic agent cell, PEGDA, photoinitiator and contrast at a concentration of 20% vol/vol is slowly injected through a glass micropipette into a flowing inert liquid, in this case mineral oil. The resulting hydrogel spheres are then polymerized through exposure to an external UV source for 10 minutes. In addition to Feridex the viability of HepG2 cells encapsulated in Barium, Iohexyl, Feridex+Iohexyl, PFOB and PFCE containing chemospheres was assessed (FIG. 4). The resulting percent viability of chemospheres for Hep G2 cells encapsulated in non-contrast containing chemospheres was 85.5+/−5.2 and 87.5+/−4.6 for Hep G2 cells encapsulated in Feridex containing chemospheres (FIG. 5c).

### Example 5

Rhodamine containing liposomes were encapsulated in ChemoSpheres with a variety of contrast. Rhodamine containing liposomes (FIG. 5c) demonstrated a 25 day release profile as demonstrated by the fluorescent intensity units measured in the solution eluted from the chemospheres (FIG. 5d). For this experiment a 1 mL packed volume of chemospheres were incubated in 10 mL of normal saline in a 15 mL falcon tube and a 250 microliter sample was taken daily and replaced with 250 microliters of fresh saline. liposomes were prepared using the extended hydration method. Phosphatidylcholine (Avanti Polar Lipids, Alabaster, Ala.) and cholesterol (Sigma, St. Louis, Mo.) in a 1:1 mole ratio in chloroform were dried in a rotary evaporator. The lipid film was solvated and annealed for 2 hours at 55 C with a solution of either rhodamine (Sigma, St. Louis, Mo.) or PBS. In order to form vesicles of a specific size, the lipid suspension was taken through twenty-one cycles of extrusion (LiposoFast, Avestin, Ottawa, Ontario, Canada) through two stacked polycarbonate filters (100 nm or 400 nm pore size). Unencapsulated drug was removed using size exclusion chromatography with sephadex G-50 resin (Sigma, St. Louis, Mo.). Dynamic light scattering (DLS) measurements of liposome suspensions was studied with a Malvern Instruments Nanosizer ZS90 (Southborough, Mass.), equipped with a 633 nm He—Ne laser light source. Scattering was detected at 90. All buffer solutions used were filtered with 0.22 μm filters just prior to vesicle preparation.

### Example 6

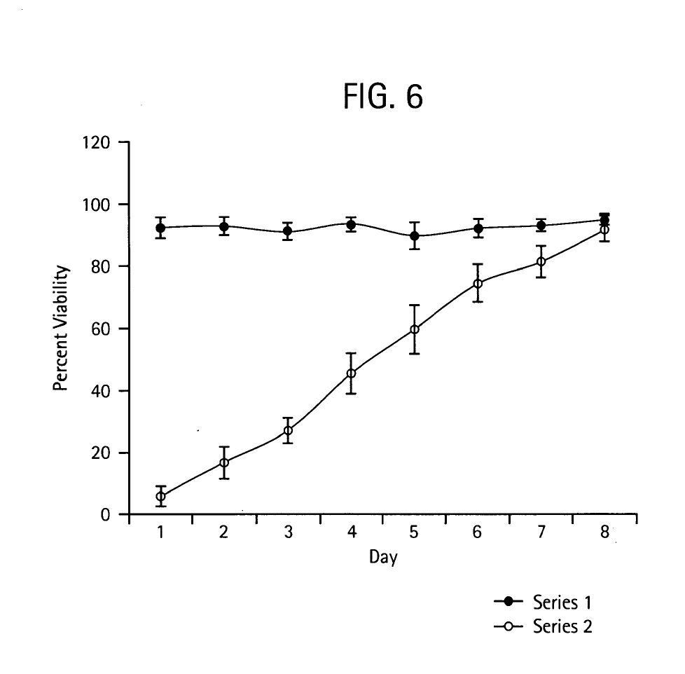

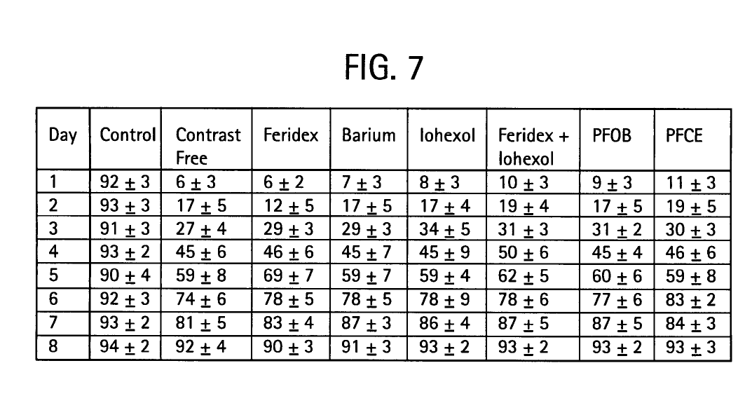

3BrPa (Sigma Chemical Co., St. Louis, Mo.) was first homogenously mixed in a solution of unpolymerized pegda as described in example 1 at a concentration of 200 mg in 1 mL of PEGDA/contrast solution. This solution was made to form chemospheres as described above. For control non 3BrPa chemospheres were utilized. HepG2 cells were cultured in EMEM substituted with 2 mM L-Glutamine, 1 mM Sodium pyruvate, 0.1 mM non-essential amino acids, 1.5 g/L sodium bicarbonate and 10% FBS in a humidified CO2 incubator at 37° C. and a 5% CO2 atmosphere. Cells were cultured in tissue culture plates and culture media was replaced every 3 days. To assess release of 3 Bromopyruvate from chemospheres, a packed volume of 1 mL of chemospheres was added to 10 mL of saline. Each day 1 mL of supernatant was removed and added to HepG2 cells in culture. Following removal of 1 mL of supernatant, the remaining supernatant was removed and replace with fresh saline. This was repeated daily and tumor kill was assessed through standard trypan exclusion assay (FIGS. 6 and 7).

### Example 7

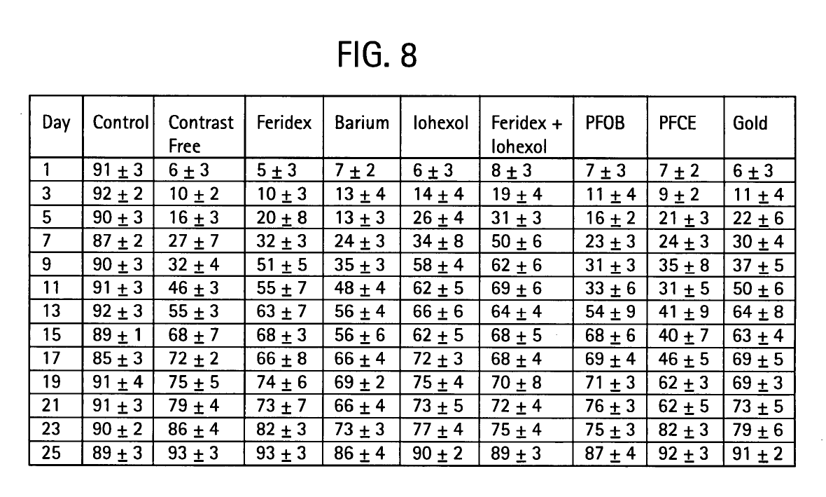

In addition to elution of rhodamine the release of doxorubicin from liposomes in pegda chemospheres was assessed by measuring the tumor kill of the solution eluted from chemospheres. Doxorubcin containing liposomes were first homogenously mixed in a solution of unpolymerized pegda as described in example 1 at a concentration of 200 mg of doxorubicin liposomes in 1 mL of PEGDA/contrast solution. This solution was made to form chemospheres as described above. HepG2 cells were cultured in EMEM substituted with 2 mM L-Glutamine, 1 mM Sodium pyruvate, 0.1 mM non-essential amino acids, 1.5 g/L sodium bicarbonate and 10% FBS in a humidified CO2 incubator at 37° C. and a 5% CO2 atmosphere. Cells were cultured in tissue culture plates and culture media was replaced every 3 days. To assess release of doxorubicin from chemospheres, a packed volume of 1 mL of chemospheres was added to 10 mL of saline. Each day 1 mL of supernatant was removed and added to HepG2 cells in culture. Following removal of 1 mL of supernatant, the remaining supernatant was removed and replace with fresh saline. This was repeated daily and tumor kill was assessed through standard trypan exclusion assays. In addition the effect of various contrast agents on release of doxorubicin was assessed (FIG. 8). For all experiments liposomes were prepared using the extended hydration method. Phosphatidylcholine (Avanti Polar Lipids, Alabaster, Ala.) and cholesterol (Sigma, St. Louis, Mo.) in a 1:1 mole ratio in chloroform were dried in a rotary evaporator. When encapsulating doxorubicin, it was dissolved in methanol and combined with the lipids prior to rotary evaporation. The lipid film was solvated and annealed for 2 hours at 55 C with a solution of either calcein (Sigma, St. Louis, Mo.) or PBS. In order to form vesicles of a specific size, the lipid suspension was taken through twenty-one cycles of extrusion (LiposoFast, Avestin, Ottawa, Ontario, Canada) through two stacked polycarbonate filters (100 nm or 400 nm pore size). Unencapsulated drug was removed using size exclusion chromatography with sephadex G-50 resin (Sigma, St. Louis, Mo.). Dynamic light scattering (DLS) measurements of liposome suspensions was studied with a Malvern Instruments Nanosizer ZS90 (Southborough, Mass.), equipped with a 633 nm He—Ne laser light source. Scattering was detected at 90. All buffer solutions used were filtered with 0.22 μm filters just prior to vesicle preparation.

### Example 8

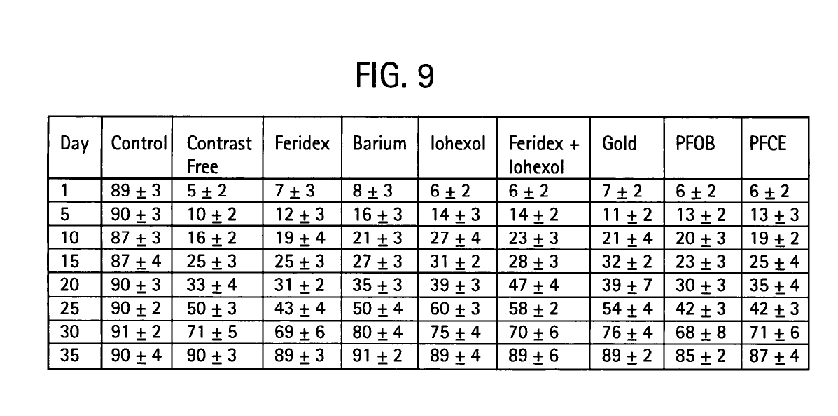

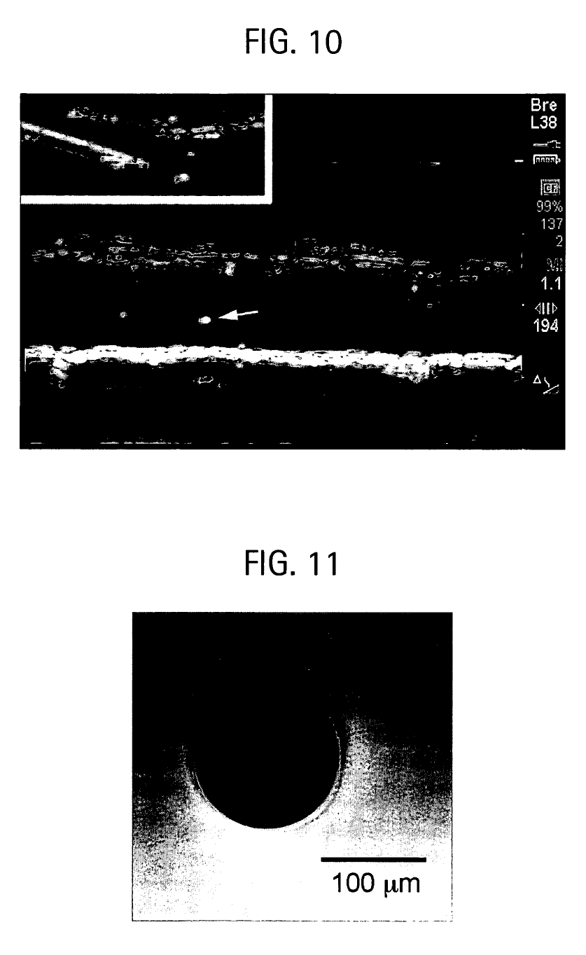

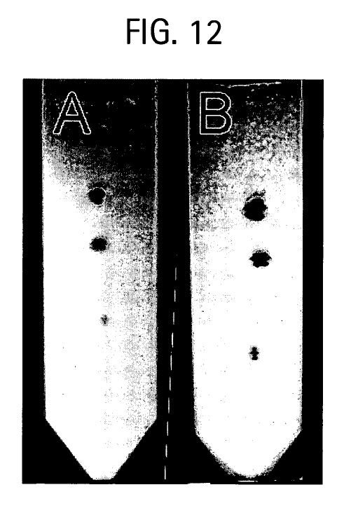

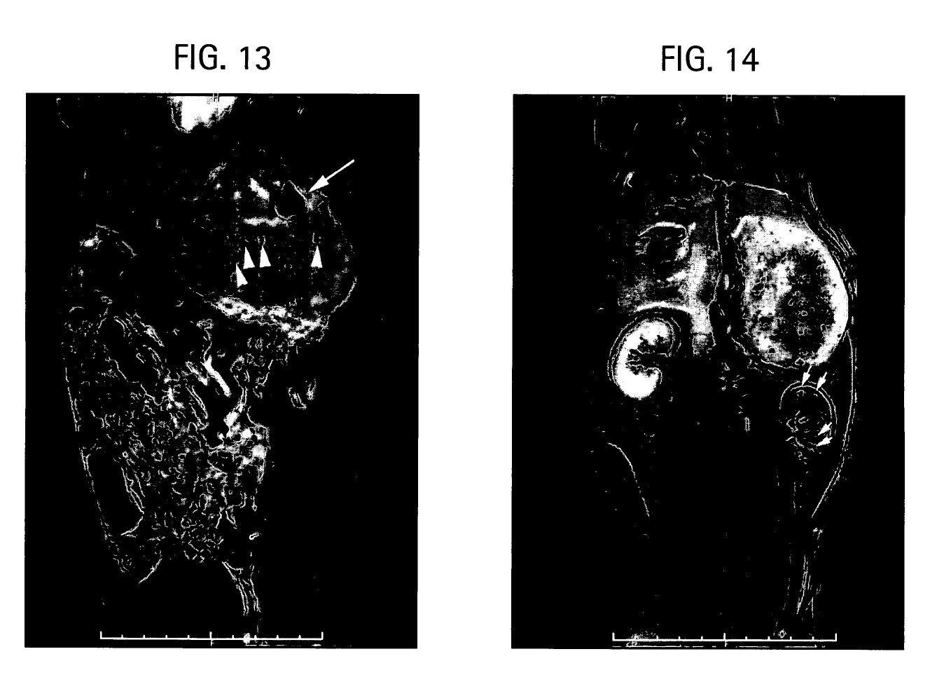

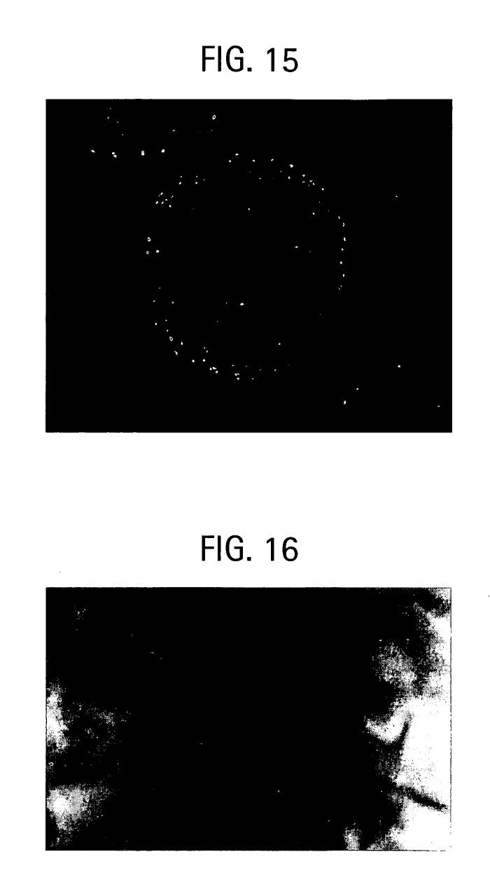

To assess the release of doxorubicin from liposomes entrapped in ChemoGel, a solution as described above for formation of doxorubicin containing chemospheres was injected into a 1 cubic centimeter mold and polymerized. This polymerized cube of chemogel was then added to 10 mL of saline. Each day 1 mL of supernatant was removed and added to HepG2 cells in culture. Following removal of 1 mL of supernatant, the remaining supernatant was removed and replace with fresh saline. This was repeated daily and tumor kill was assessed through standard trypan exclusion assays. In addition the effect of various contrast agents on release of doxorubicin was assessed (FIG. 9).

### Example 9

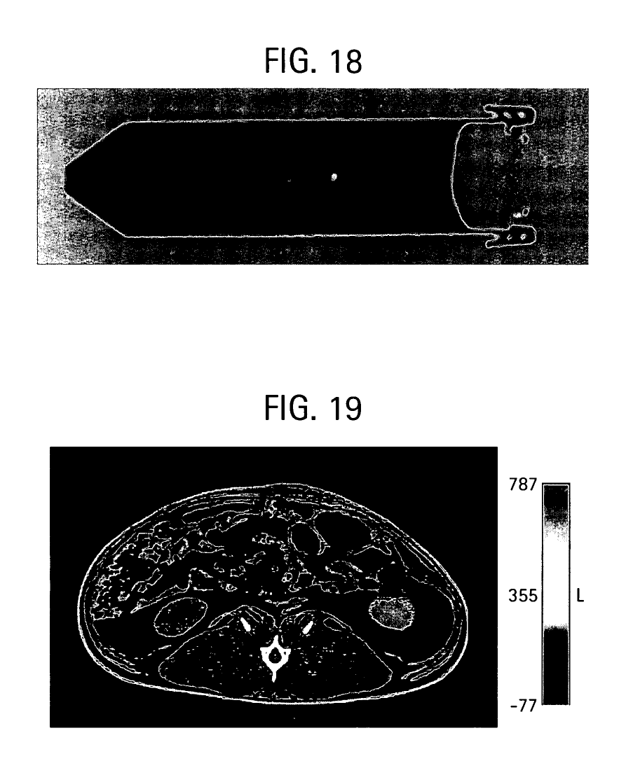

PFOB containing Chemospheres formed as described below were transplanted into the kidney of a new Zealand white rabbit and imaged with a standard portable US unit. Perfluorooctylbromide (Sigma Co) and perfluoro-15-crown-5 ether (PFPE, Exfluor) at a concentration of 1.97 g/mL and 1.88 g/mL respectively were emulsified (20% vol/vol) in a mixture of 5% lecithin, 2% safflower oil and water by sonication at 40% power. This solution was then added to PEGDA at a concentration of 20% vol/vol. Sonography was performed with a L25E 13-6 MhZ probe on a Micromaxx US system (Sonosite). Grayscale imaging was performed with a center probe frequency of 6.00 MHz, a dynamic range of 55 dB, and a persistence setting of two. Gray-scale gain was adjusted for baseline imaging. FIG. 18 shows gold-dextran containing chemospheres suspended in gelatin in a 50 mL conical tube as point sources of 1 and 5 microcapsules. FIG. 19 shows a slice from a 64 slice CT scan of a new zealand white rabbit after unilateral kidney embolization with 1 mL packed volume of gold-dextran chemospheres.

### Example 10

### Synthesis of Self-Polymerizing PEGDA Based Polymers

Pentaerythritol-tetrakis (3-mercaptopropionate) (QT) and pentaerythritol triacrylate (TA), Poly(ethylene glycol) diacrylate 570 MW (PEGDA), poly(propylene glycol) diacrylate 900 MW (PPODA 900), poly(propylene glycol-co-polyethylene glycol-co-polypropylene glycol) 3300 MW (PEP) Sorbitan monooleate (SM) were purchased from Sigma-Aldrich.

Synthesis of poly(ethylene glycol) hexathiol (PEGHT): QT (8.58 g, 17.5 mmol) was combined with 8 mL H.sub.2O and 2 mL 1N NaOH in 100 mL of tetrahydrofuran (THF). The PEGDA 570 (1 g, 1.75 mmol) was combined with 1 mg of 2,6-di-tertbutyl-p-chresol (radical inhibitor) in 10 mL di-chloromethane (DCM). The PEGDA solution was then diluted with 15 mL of THF. The diluted PEG solution was then dropwise added to the stirred QT solution. After 55 min the pH was adjusted to 7 using glacial acetic acid. To remove water, the solvent was evaporated and the product was redissolved in 100 mL of toluene. The toluene was evaporated and an additional 100 mL of toluene was added. About 13 g of sodium sulfate was added, and then the sodium sulfate was removed by filtering. Before precipitation in 10-fold excess diethyl ether, the solution was concentrated by evaporating some of the toluene. The diethyl ether was decanted and the liquid product was recovered. The product was dried under vacuum.

Synthesis of poly(ethylene glycol) tetraacrylate (PEGOA): The PEGDA 570 (20 g, 35 mmol) was combined with 20 mg of 2,6-di-tertbutyl-p-chresol in 10 mL DCM. This was then diluted with 90 mL THF. The NaOH (3 mL, 0.2N) and 1 mL of H.sub.20 were added. The QT (1 g, 2.0 mmol) was dissolved in 40 mL of THF. The QT solution was added dropwise to the stirred PEGDA solution. After 30 min, 7 .mu.L of glacial acetic acid was added to neutralize the reaction. The solvent was evaporated and 100 mL of toluene were added. After drying over sodium sulfate, the solution was filtered, concentrated and then precipitated using 10-fold excess diethyl ether. After precipitation the product was dried under vacuum.

Preparation of Crosslinked Biomaterials: Crosslinked Materials were Prepared as dispersions or reverse emulsions of precursors in modified phosphate buffered saline (PBS). The PBS, 10 mM solution, was obtained by mixing equal volumes of 10 mM PBS adjusted to pH 9 with the addition of triethanolamine or 1N NaOH, respectively.

Formation of self-polymerizing ChemoSpheres: As a typical procedure for 75-wt % dispersion materials, 424 mg QT and 997 mg of PEGDA 570 were combined and mixed well by vortexing. Air bubbles were removed by sonication. The PBS solution (473 mg) was added to the mixed precursors. The mixture was again vortexed for about 2 min to mix well and disperse the precursors in the aqueous solution. Following vortexing, the mixture was again sonicated to remove air bubbles. The materials were then loaded into a 1 mL tuberculin syringe and added dropwise to a mineral oil bath that was mechanically stirred with a magnetic stir bar/plate. The rate of stirring was adjusted until proper ChemoSphere size was achieved. The ChemoSpheres were left stirring in the inert bath until complete polymerization occurred. Polymerization of the spheres could be partially noted as once they polymerize they tend to settle to the bottom of the inert bath. To assess ability to polymerize in the presence of 3 bromopyruvate, 200 mg/mL of 3 bromopyruvate was first added to the PBS solution prior to addition of the precursors. The pH of this solution was adjusted to pH7 prior to addition of precursors. To ensure polymerization of polymer was still possible in the presence of contrast agent the above procedure was repeated with the addition of 20% vol/vol contrast of Feridex, gold dextran (Nanocs), PFOB emulsion, PFCE emulsions, barium sulfate solution, iohexyl and ioheoxl together with Feridex. All non-commercial emulsions were prepared as described above.

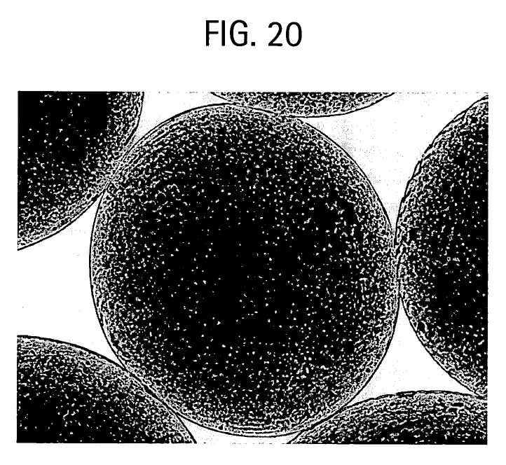

FIG. 20 shows ChemoSphere loaded with manganese oxide and citric acid containing liposomes prior to drug loading.

### Example 11

### Novel MR and CT Detectable ChemoSpheres For Locoregional Delivery of 3-Bromopyruvate and Doxorubicin

Current drug-eluting microspheres cannot be directly visualized with conventional imaging. In vivo visualization could permit a) real-time verification of delivery, b) detection of extrahepatic shunting, and c) more accurate assessment of particle resorption. The purpose of our study was to develop ChemoSpheres, novel iron-dextran or gold-dextran impregnated microspheres for detection on respectively MR and CT that elute 3-bromopyruvate or doxorubicin. To assess release profile of the compounds an in vitro tumor kill assay was undertaken. To assess sensitivity of detection on MR and CT, ChemoSpheres were imaged after point source injections in gelatin phantoms and after transplant in rabbits with VX2 liver tumors.

ChemoSpheres were formed by adding 3-bromopyruvate or liposomes loaded with doxorubicin at a concentration of 200 mg/mL to a solution containing poly(ethylene glycol)-diacrylate (15% w/v), the photoinitiator Igracure D2959 (0.05% w/v) and Feridex or Gold-Dextran nanoparticles (10% v/v). The mixture was then added drop-wise to a mechanically stirred mineral oil bath which was then exposed to UV irradiation to initiate polymerization. The resulting 100 μm-200 μm ChemoSpheres were then rinsed and injected through a 2.5 Fr (Renegade, Boston Sci.) transfemoral hepatic artery catheter into three rabbits with VX-2 tumors two weeks post implantation. Animals were imaged on a Siemens' Trio 3T MRI or Toshiba's CorE 64 Multislice CT. In vitro tumor kill of the solution eluted from ChemoSpheres containing either 3-bromopyruvate or doxorubicin was assessed on the HepG2 cell line.

In gelatin phantoms individual iron and iron/gold containing ChemoSpheres were detectable on MR. Pulse parameters for in vitro imaging were TR: 256, TE: 4.00, Image size: 384×512, view size: 1259×755, and slice thickness 3.00 mm.

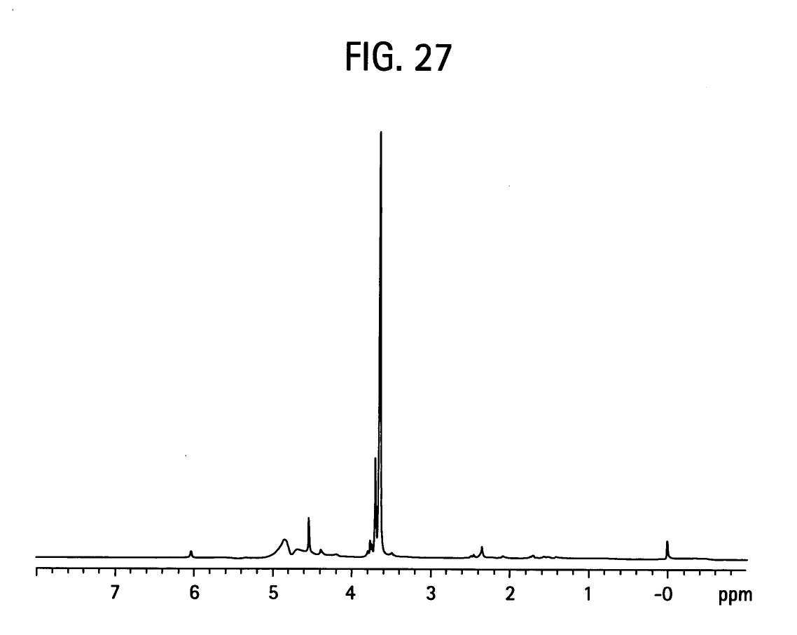

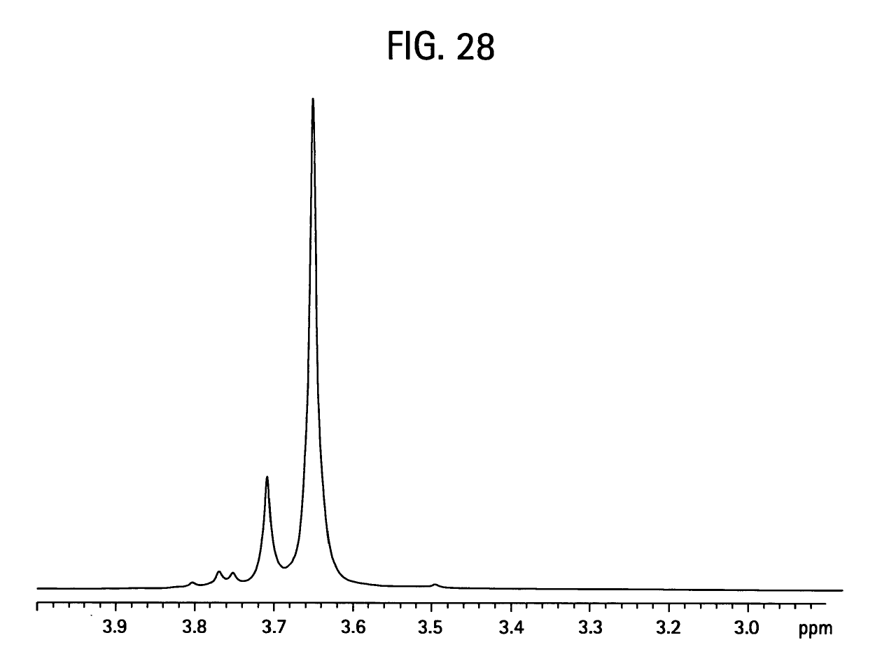

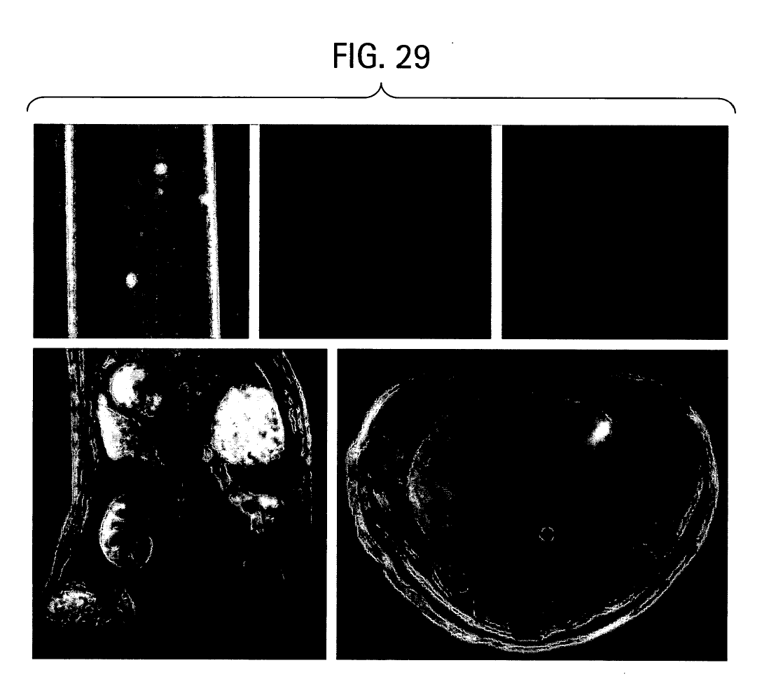

FIG. 27 shows the NMR spectra of solution eluted from 3 Bomopyruvate loaded spheres after eight days demonstrates 3br-pyruvate peak (3.65 peak.) The breakdown products are increased as compared to the 24 hour elution but active 3 bromopyruvate is still eluted. FIG. 28 is a blow up of main peak of FIG. 27 that shows increased breakdown products as minor component and active 3-Bromopyruvate as major component of eluted solution at 8 days.

Similar imaging results were achieved in vivo on rabbits imaged 24 hrs post embolization with a packed volume of 1 mL of ChemoSpheres with hypointense signal from iron-dextran containing spheres on MR. Pulse parameters for in vivo imaging were TR: 4.00, TEL 1.30, Image size: 160×125, view size 1262×755 and slice thickness 2.00 mm. ChemoSpheres could be clearly seen throughout the tumor bed. Further small areas of non-targeted injection could be seen. As proof of principle, ChemoSpheres were also used to embolize the kidney of a New Zealand white rabbit. Similar to liver embolization, chemospheres could be detected in the kidney as areas of hypointensity. For kidney in vivo imaging pulse parameters were TR: 100, TE: 4.00, images size: 240×320, view size 1260×755 and slice thickness 5.00 mm.

FIG. 38 shows fluoroscopic angiography of kidney with catheter in renal artery pre-embolization (left frame) and post-embolization (right frame) with iron-oxide containing chemospheres demonstrates effective embolization resulting in stagnant blood flow. FIG. 39 shows fluoroscopic angiography of rabbit VX-2 liver tumor pre transarterial embolization (left frame) and post trans-arterial embolization (right frame) with iron-oxide containing chemospheres demonstrates effective embolization resulting in stagnant blood flow.

Delivery of 3-bromopyruvate or doxorubicin loaded ChemoSpheres provide a significant advantage to current techniques as they provide a means of sustained release potentially minimizing toxicity while maximizing tumor kill. ChemoSpheres show promise for targeted locoregional delivery of 3-bromopyruvate and/or doxorubicin on MR and CT.

### Example 12

HepG2 cells were encapsulated as described above and viability was assessed with SYTO9 (Invitrogen) and PI (Invitrogen). Stock solutions of the dyes were prepared as follows: PI and SYTO9 were used from the LIVE/DEAD BacLight kit (Invitrogen) as proposed by the manufacturer. Samples stained with either a mixture of SYBR green (104-times diluted from the original stock) and PI (3 μM). Samples were incubated in the dark at room temperature for 20 min), before analysis (FIG. 16).

### Example 13

### Chemosphere and Chemogel for Ocular Applications

Injection of compositions comprising an active agent into the eye may be ineffective as the active agent may be washed out or is depleted from within the eye into the general circulation resulting in necessity for repeated administration, e.g. three injections in three to 42 days as described in U.S. Pat. No. 5,632,984.

Introduction of slow release compositions, i.e. implants, into the eye, e.g. into an anterior segment or posterior segment of an eye as described in U.S. Pat. No. 4,853,224, e.g. into the suprachoroidal space or pars plana of the eye as described in U.S. Pat. No. 5,164,188, or e.g. into a site extrinsic to the vitreous comprising a suprachoroidal space, an avascular region of an eye, or a surgically-induced avascular region as described in U.S. Pat. No. 5,824,072, by injection or surgical methods such as laser ablation, photocoagulation, cryotherapy, heat coagulation and the like is extremely painful and stressful for the patient. Implants may have to be removed when therapy is completed or no longer efficacious. Applicants have found that ophthalmic depot formulations comprising an active agent may be administered, periocularly, e.g. retrobulbarly or sub-tenonly, or subconjunctivally.

### Example 14

### Loading of Liposome Polymer Matrix Using a pH Gradient

This example describes the loading of a liposome polymer matrix utilizing a pH gradient. Liposomes were prepared using the extended hydration method. Phosphatidylcholine (Avanti Polar Lipids, Alabaster, Ala.) and cholesterol (Sigma, St. Louis, Mo.) in a 1:1 mole ratio in chloroform were dried in a rotary evaporator. The lipid film was solvated and annealed for 2 hours at 55 C with a solution of 300 mM Citrate, 450 mM ethylenediamine and 3M sucrose. In order to form vesicles of a specific size, the lipid suspension was taken through twenty-one cycles of extrusion (LiposoFast, Avestin, Ottawa, Ontario, Canada) through two stacked polycarbonate filters (100 nm or 400 nm pore size). Unencapsulated citrate was removed using size exclusion chromatography with sephadex G-50 resin (Sigma, St. Louis, Mo.). Dynamic light scattering (DLS) measurements of liposome suspensions was studied with a Malvern Instruments Nanosizer ZS90 (Southborough, Mass.), equipped with a 633 nm He—Ne laser light source. Scattering was detected at 90. All solutions used were filtered with 0.22 [tm filters just prior to vesicle preparation. Following liposome synthesis the liposome underwent a buffer exchange to remove the external, unentrapped, ethylenediamine citrate. This was accomplished by subjecting the microspheres to tangential flow ultrafiltration. This process removes water and small molecular weight solutes while retaining the liposomes. Using this process, the sucrose/ethylenediamine citrate buffer external to the vesicles was replaced with 3M sucrose and 750 mM Histidine HCl.

To this 100 mg/mL liposome solution was added a concentrated solution of poly(ethylene glycol)-diacrylate (PEGDA; Nektar Therapeutics, Huntsville, Ala.) in sterile phosphate-buffered saline (PBS; GIBCO Invitrogen, Carlsbad, Calif.) to make a 15% (w/v) hydrogel. Photoinitiator, Igracure D2959 (Ciba Specialty Chemicals, Tarrytown, N.Y.) was added to the polymer at a concentration of 0.05% (w/v). The mixture is then added drop-wise to a reactant medium made up of an inert bath. An inert bath includes but is not limited to a formulation of 160 grams Span 80 and 3,840 grams USP mineral oil. The inert bath is then stirred at a rate of 200 RPM in a reaction vat. The Vat was then exposed to UV irradiation for 5 minutes to initiate polymerization. Once reacted, the resultant crosslinked PEGDA sphere with the entrapped liposomes were cleaned of mineral oil and Span 8. The cleaning process involved dumping the resultant spheres over a 45-micron screen such that the beads were trapped on the screen while some of the reactants passed through. The spheres were then washed with a solution containing 3M sucrose, 750 mM Histidine HCl. Additionally to facilitate a method for large scale synthesis the spheres were loaded in a chromatography column and were washed with three exchanges of chilled 3M sucrose and 750 mM Histidine HCl solution equal to the packed volume of the spheres.

The spheres that were subjected to ultrafiltration or column washes were stored for a variable amount of time in a minimal amount of 3 M sucrose, 750 mM Histidine HCl to maintain hydration at 20° C. and 5° C. Following storage the loading efficiency of doxorubicin into the spheres was assessed (table 1). For drug loading a procedure that could easily be implement in the pharmacy was employed. Specifically a water bath or dry heat block was allowed to equilibrate at 58° C. (55°-60° C.). For each 1 mL packed volume of microspheres, 50 mg of lyophilized doxorubicin (for storage lactose and parahydroxybenzoate may be added) in a solution of 8 mL was prepared. This solution was then added to the equilibrated heating source for 10 min. Next a 2 mL concentrated solution of sodium carbonate was injected into the microsphere solution. The 2 mL of microspheres and 8 mL of doxorubicin were then combined creating a solution with pH of 7 (this step should be done within 2 minutes of adding sodium carbonate to the microspheres). The mixture should be shaken vigorously and allowed to sit for at least 10 minutes in the heat block or water bath and then brought to room temperature. After this period the spheres can be stored up to 10 hours at 25° C. and for 7 days at 2° C.-8° C.

Typically, if an appropriate polymer/lipid:drug ratio has been selected (approximately at least 50:1, mol/mol), greater than about 90% of the added doxorubicin will become vesicle entrapped as doxorubicin-citrate. If necessary, unentrapped, residual doxorubicin can be removed by removing the spheres from the drug containing solution using a 45 micron screen or other acceptable method. In certain embodiments a syringe like setup with a two way valve with a screen on one valve and an unobstructed opening on the other valve can be utilized to first remove the solution while maintaining the spheres and then

When Doxoroubicin HCl interacts with citrate it forms organized fiber bundles in the liposomes inside the polymer matrix. With a pH gradient established, doxorubicin accumulates in the vesicle interior and the ideal distribution of doxorubicin in solution inside (Dm) and outside (D out) the liposomes is expected to be related to the inner and outer E1+ concentrations by Equation 1 shown below:

$$\frac{{D}_{in}}{{D}_{out}} = \frac{{V}_{in}}{{V}_{out}} ( \frac{K + {[ {H}^{+} ]}_{in}}{K + {[ {H}^{+} ]}_{out}} ) \qquad ( Equation 1 )$$

where Vin and Vout are the internal and external aqueous volumes and K is the dissociation constant for doxorubicin (pK=8.22). However, we have found that D M in-out can exceed the value predicted by Eq. 1. One explanation for this enhanced accumulation is that internalized doxorubicin does not remain in solution but instead precipitates within the liposome with citrate thereby facilitating additional movement of doxorubicin from outside to satisfy the equilibrium relationship. To create MR visible chemospheres, the SPIO Feridex, dextran coated 50 nm manganese oxide nanoparticles, or dextran coated 50 nm gadolinium oxide particles were added to unpolymerized PEGDA at a concentration of 20% vol/vol to the ungelled PEGDA, liposome polymer mixture. To create x-ray visible chemospheres, dextran covered gold particles (Nanocs, 50 nm) were added at a concentration of respectively 20% w/volume or 20% volume/volume to the ungelled PEGDA, liposome polymer matrix. To create MR and X-ray visible chemosphers, gold-dextran nanoparticles and iron-oxide nanoparticles were added to chemospheres at respectively a concentration of 20% vol/vol and 20% volume/volume.

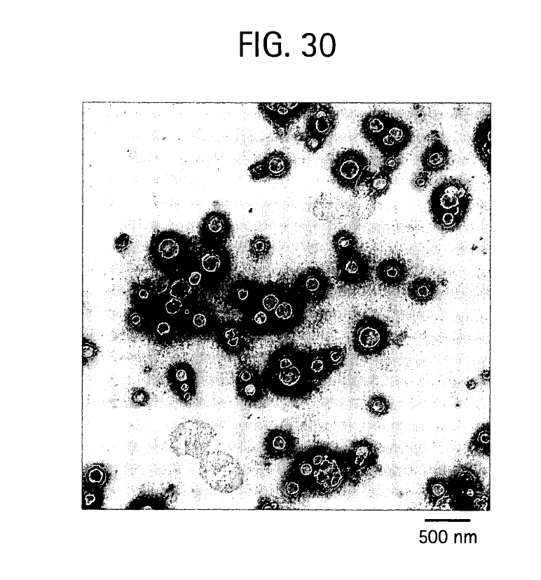

FIG. 30 shows that after all doxorubicin was eluted from the spheres described in example 2, spheres were dissolved with alginate lyase and EDTA. EDTA (Sigma, St. Louis, Mo.) was added at a concentration of 5 mg/mL to normal saline. The pH of this preparation was then adjusted to pH 7.0. Alginate lyase isolated from Flavobacterium sp. (Sigma, St. Louis, Mo.) was then added at a concentration of 2 mg/ml to the EDTA solution. This solution was then filter sterilized through a syringe tip filter (Millipore). As seen in this image, liposomes remained largely intact even after all drug was released.

The release profile from the preparation in micrograms/mL is listed in the top panel of the Tables below and the resulting viability of HepG2 cells incubated with the solution is listed in the bottom panels of the Tables below.

TABLE 1
Stored at 20° C. for 10 hours prior to drug loading.

| | 0 hrs | 6 hrs | 12 hrs | 1 day | 2 days | 3 days | 4 days | 5 days | 6 days | 7 days | 8 days |
|---|---|---|---|---|---|---|---|---|---|---|---|
| No Contrast | 0.438 | 7.698 | 22.132 | 49.875 | 110.395 | 158.453 | 185.569 | 232.598 | 275.895 | 298.458 | 301.394 |
| Iron Oxide | 0.128 | 8.321 | 19.557 | 52.145 | 100.440 | 166.329 | 203.456 | 249.489 | 265.984 | 309.985 | 312.496 |
| Gd Oxide | 0.613 | 6.984 | 17.659 | 47.498 | 95.460 | 102.489 | 194.382 | 215.489 | 283.490 | 295.698 | 302.499 |
| Mn Oxide | 0.439 | 7.893 | 24.343 | 43.981 | 89.498 | 121.458 | 213.460 | 245.988 | 274.398 | 310.498 | 316.499 |
| No Contrast | [symbol] | 0.629 | 0.512 | 0.114 | 0.094 | [symbol] | [symbol] | 0.039 | 0.064 | 0.017 | [symbol] |
| Iron Oxide | 0.962 | [symbol] | 0.422 | 0.125 | [symbol] | 0.039 | 0.049 | 0.043 | 0.047 | 0.022 | [symbol] |
| Gd Oxide | [symbol] | 0.611 | 0.521 | 0.118 | 0.042 | 0.015 | 0.098 | 0.040 | 0.024 | 0.011 | 0.022 |
| Mn Oxide | [symbol] | 0.753 | 0.455 | 0.253 | 0.025 | 0.031 | 0.070 | 0.086 | 0.017 | [symbol] | [symbol] |

[symbol]

indicates data missing or illegible when filed

TABLE 2
Stored at 5° C. for 7 days prior to drug loading.

| | 0 hrs | 6 hrs | 12 hrs | 1 day | 2 days | 3 days | 4 days | 5 days | 6 days | 7 days | 8 days |
|---|---|---|---|---|---|---|---|---|---|---|---|
| No Contrast | 0.517 | 4.985 | 22.456 | 56.454 | 85.695 | 123.460 | 224.355 | 240.321 | 251.456 | 301.584 | 304.577 |
| Iron Oxide | 0.388 | 7.984 | 20.867 | 39.695 | 92.456 | 148.395 | 210.569 | 256.496 | 259.696 | 293.549 | 295.486 |
| Gd Oxide | 0.814 | 6.954 | 17.596 | 42.859 | 104.598 | 158.392 | 189.543 | 262.460 | 268.458 | 285.985 | 289.858 |
| Mn Oxide | 0.269 | 9.459 | 28.586 | 40.398 | 102.586 | 122.985 | 187.596 | 228.596 | 234.589 | 320.494 | 324.549 |
| No Contrast | 0.949 | 0.635 | 0.596 | 0.438 | 0.068 | 0.020 | 0.038 | 0.048 | 0.049 | 0.030 | 0.085 |
| Iron Oxide | 0.986 | 0.755 | 0.635 | 0.240 | 0.058 | 0.038 | 0.028 | 0.035 | 0.049 | 0.058 | 0.058 |
| Gd Oxide | 0.868 | 0.658 | 0.387 | 0.385 | 0.029 | 0.089 | 0.039 | 0.029 | 0.024 | 0.038 | 0.038 |
| Mn Oxide | 0.937 | 0.726 | 0.585 | 0.294 | 0.035 | 0.028 | 0.069 | 0.087 | 0.043 | 0.048 | 0.019 |

### Example 15

The citrate loaded liposomes as described in the example above were suspended in 2% w/v ultrapurified sodium Protanal HF alginate (FMC Biopolymers) containing 300 mM citrate, 450 mM ethylenediamine and 3M sucrose. This solution was passed through a needle at a flow rate of about 200 ml/min using a nanoinjector pump. Droplets were collected in a Petri dish containing 100 mM CaCl2, 3M sucrose, 750 mM Histidine HCl and then washed three times with chilled 3M sucrose, 750 mM Histidine HCl. To create MR visible chemospheres, the SPIO Feridex was added at a concentration of 20% you vol to the ungelled alginate, liposome polymer mixture. To create x-ray visible EmboCaps, dextran covered gold particles (Nanocs, 50 nm) were added at a concentration of respectively 20% w/volume or 20% volume/volume to the ungelled alginate, liposome polymer matrix. To create MR and X-ray visible chemospheres, a colloidal solution of gold-dextran nanoparticles and iron-oxide nanoparticles were added to microspheres at respectively a concentration of 10% vol/vol and 10% volume/volume.

The same drug loading procedure in the example above (Example 14) was employed to load alginate-citrate loaded liposome spheres with doxorubicin.

The release profile from the preparation in micrograms/mL is listed in the top panel of the table below and the resulting viability of HepG2 cells incubated with the solution is listed in the bottom panel of the table.

TABLE 3

| | 0 hrs | 6 hrs | 12 hrs | 1 day | 2 days | 3 days | 4 days | 5 days | 6 days | 7 days | 8 days |
|---|---|---|---|---|---|---|---|---|---|---|---|
| No Contrast | 0.182 | 7.694 | 20.123 | 47.321 | 90.245 | 112.412 | 224.523 | 264.321 | 274.568 | 292.675 | 298.570 |
| Iron Oxide | 0.291 | 10.432 | 16.893 | 40.145 | 87.146 | 105.459 | 194.578 | 221.459 | 284.986 | 315.897 | 318.597 |
| Gd Oxide | 0.216 | 9.457 | 24.356 | 52.687 | 94.350 | 102.432 | 209.584 | 228.498 | 278.567 | 310.459 | 315.494 |
| Mn Oxide | 0.346 | 8.532 | 19.859 | 50.134 | 96.414 | 124.259 | 232.567 | 245.350 | 248.689 | 284.595 | 289.597 |
| No Contrast | 0.945 | 0.607 | 0.547 | 0.193 | 0.022 | 0.028 | 0.014 | 0.027 | 0.039 | 0.018 | 0.041 |
| Iron Oxide | 0.925 | 0.712 | 0.594 | 0.205 | 0.049 | 0.035 | 0.045 | 0.070 | 0.033 | 0.030 | 0.018 |
| Gd Oxide | 0.911 | 0.524 | 0.435 | 0.131 | 0.031 | 0.019 | 0.035 | 0.045 | 0.022 | 0.015 | 0.010 |
| Mn Oxide | 0.915 | 0.780 | 0.314 | 0.254 | 0.025 | 0.045 | 0.030 | 0.087 | 0.017 | 0.036 | 0.088 |

### Example 16

In this example the PEGDA and alginate spheres described in examples 14 and 15 were loaded by substituting 50 mg of lyophilized irinotecan for the same concentration of doxorubicin and following the identical loading protocol.

The release profile from the preparation in micrograms/mL is listed in the top panel of the table below and the resulting viability of HepG2 cells incubated with the solution is listed in the bottom panel of the table below.

TABLE 4

| | 0 hrs | 6 hrs | 12 hrs | 1 day | 2 days | 3 days | 4 days | 5 days | 6 days | 7 days | 8 days |
|---|---|---|---|---|---|---|---|---|---|---|---|
| No Contrast | 0.141 | 9.563 | 17.954 | 43.892 | 60.359 | 143.289 | 214.893 | 234.231 | 268.456 | 298.489 | 302.478 |
| Iron Oxide | 0.326 | 8.549 | 16.345 | 54.982 | 91.395 | 154.893 | 198.765 | 227.378 | 273.658 | 312.490 | 315.387 |
| Gd Oxide | 0.431 | 8.678 | 23.425 | 50.432 | 112.432 | 163.893 | 221.453 | 242.460 | 259.746 | 286.593 | 294.595 |
| Mn Oxide | 0.198 | 8.453 | 19.875 | 46.341 | 98.418 | 123.410 | 202.456 | 254.496 | 260.589 | 325.895 | 314.569 |
| No Contrast | 0.945 | 0.668 | 0.530 | 0.231 | 0.073 | 0.035 | 0.038 | 0.079 | 0.050 | 0.027 | 0.011 |
| Iron Oxide | 0.914 | 0.792 | 0.453 | 0.260 | 0.094 | 0.045 | 0.029 | 0.089 | 0.064 | 0.036 | 0.011 |
| Gd Oxide | 0.934 | 0.607 | 0.462 | 0.197 | 0.087 | 0.086 | 0.013 | 0.067 | 0.058 | 0.067 | 0.037 |
| Mn Oxide | 0.854 | 0.538 | 0.588 | 0.319 | 0.019 | 0.069 | 0.039 | 0.011 | 0.013 | 0.053 | 0.011 |

### Example 17

An alternate formulation of liposomes was created for incorporation into PEGDA and alginate spheres as described in the example above. Specifically, a homogenous dispersion of cholesterol and distearoylphosphatidylcholine at a 1:2 molar ratio was prepared by evaporation from a 1:1 chloroform/methanol solution This dispersion was hydrated with an aqueous solution containing 3M sucrose and a 300 mM salt of ethylenediamine citrate. The lipid concentration in the resulting suspension was about 20 to 25 mg/ml. This mixture was then heated to 65° C. Next, while maintaining that temperature, the mixture was homogenized using a sonicator to produce a suspension of small unilamellar vesicles. The vesicles thus obtained had diameters principally in the range of about 40 to 80 nm with little or no particles in excess of 100 nm. Following liposome synthesis the liposome underwent a buffer exchange to remove the external, unentrapped, ethylenediamine citrate. This was accomplished by subjecting the microspheres to tangential flow ultrafiltration. This process removes water and small molecular weight solutes while retaining the liposomes. Using this process, the sucrose/ethylenediamine citrate buffer external to the vesicles was replaced with 3M sucrose and 300 mM Histidine HCl.

These liposomes were then incorporated into PEGDA microspheres as described in example 1 and doxorubicin was loaded as previously described.

The release profile from the preparation in micrograms/mL is listed in the top panel of the table below and the resulting viability of HepG2 cells incubated with the solution is listed in the bottom panel of the table below.

TABLE 5

| | 0 hrs | 6 hrs | 12 hrs | 1 day | 2 days | 3 days | 4 days | 5 days | 6 days | 7 days | 8 days |
|---|---|---|---|---|---|---|---|---|---|---|---|
| No Contrast | [symbol] | [symbol] | [symbol] | [symbol] | [symbol] | [symbol] | [symbol] | [symbol] | [symbol] | [symbol] | 0.016 |
| Iron Oxide | 0.952 | [symbol] | [symbol] | 0.125 | 0.069 | [symbol] | [symbol] | 0.041 | [symbol] | 0.022 | [symbol] |
| Gd Oxide | [symbol] | [symbol] | [symbol] | [symbol] | 0.042 | 0.015 | [symbol] | [symbol] | 0.024 | 0.011 | 0.022 |
| Mn Oxide | [symbol] | [symbol] | [symbol] | [symbol] | 0.025 | 0.031 | [symbol] | [symbol] | 0.017 | 0.017 | 0.028 |
| No Contrast | 0.438 | 7.698 | 22.132 | 49.875 | 110.395 | 158.453 | 185.569 | 232.598 | 275.895 | 298.458 | 301.394 |
| Iron Oxide | 0.129 | 8.321 | 19.557 | 52.145 | 100.448 | 164.329 | 203.456 | 249.489 | 265.984 | 309.985 | 312.496 |
| Gd Oxide | 0.613 | 6.984 | 17.659 | 47.498 | 95.460 | 102.489 | 194.382 | 215.489 | 283.490 | 295.698 | 302.499 |
| Mn Oxide | 0.439 | 7.893 | 24.643 | 43.981 | 89.498 | 121.458 | 213.460 | [symbol] | 274.398 | 310.498 | 316.499 |

[symbol]

indicates data missing or illegible when filed

### Example 18

In this example the alginate spheres described in example 15 were coated with poly-L-lysine by incubating them in a solution of 0.05% w/v PLL, 3M sucrose and 750 mM Histidine HCl for 30 minutes prior to the final wash step described in Example 15 of liposome loaded spheres.

The release profile from the preparation in micrograms/mL is listed in the first panel of the table below and the resulting viability of HepG2 cells incubated with the solution is listed in the second panel of the table below.

TABLE 6

| | 0 hrs | 6 hrs | 12 hrs | 1 day | 2 days | 3 days | 4 days | 5 days | 6 days | 7 days | 8 days |
|---|---|---|---|---|---|---|---|---|---|---|---|
| No Contrast | 0.428 | 8.587 | 21.568 | 56.489 | 878.459 | 170.469 | 223.460 | 263.489 | 272.479 | 308.382 | 310.486 |
| Iron Oxide | 0.139 | 6.983 | 18.456 | 48.924 | 112.453 | 183.459 | 201.456 | 240.348 | 263.291 | 320.598 | 321.498 |
| Gd Oxide | 0.417 | 8.954 | 26.132 | 51.232 | 90.456 | 163.598 | 224.498 | 256.433 | 275.432 | 298.987 | 299.849 |
| Mn Oxide | 0.342 | [symbol] | [symbol] | 45.439 | 125.632 | [symbol] | 209.322 | 228.489 | 243.493 | 310.460 | 312.490 |
| No Contrast | 0.922 | 0.630 | 0.359 | 0.233 | 0.025 | 0.042 | 0.065 | 0.080 | 0.017 | 0.035 | 0.019 |
| Iron Oxide | 0.918 | 0.725 | 0.452 | 0.229 | 0.050 | 0.033 | 0.060 | 0.078 | 0.034 | 0.025 | 0.057 |
| Gd Oxide | 0.902 | 0.622 | 0.444 | 0.179 | 0.018 | 0.012 | 0.060 | 0.061 | 0.012 | 0.020 | 0.017 |
| Mn Oxide | 0.926 | 0.532 | 0.317 | 0.256 | 0.026 | 0.035 | 0.060 | 0.038 | 0.017 | 0.027 | 0.017 |

[symbol]

indicates data missing or illegible when filed

### Example 19

In this example the procedure described in example 18 was followed but a secondary layer of alginate was applied. Specifically the spheres were incubated in a 0.15% weight/volume alginate solution containing 300 mM Histidine HCl and 3M sucrose for 10 minutes prior to the final wash step described herein.

The release profile from the preparation in micrograms/mL is listed in the top panel of the table below and the resulting viability of HepG2 cells incubated with the solution is listed in the bottom panel of the table.

TABLE 7

| | 0 hrs | 6 hrs | 12 hrs | 1 day | 2 days | 3 days | 4 days | 5 days | 6 days | 7 days | 8 days |
|---|---|---|---|---|---|---|---|---|---|---|---|
| No Contrast | 0.326 | 7.563 | 20.654 | 51.189 | 87.632 | 185.632 | 201.645 | 213.499 | 248.629 | 298.458 | 299.596 |
| Iron Oxide | 0.162 | 8.126 | 19.845 | 48.316 | 80.439 | 143.346 | 210.342 | 224.476 | 245.596 | 312.590 | [symbol] |
| Gd Oxide | 0.325 | 0.985 | [symbol] | 46.734 | 93.482 | 178.459 | [symbol] | 219.496 | [symbol] | 306.495 | [symbol] |
| Mn Oxide | [symbol] | 8.321 | 26.483 | 50.123 | 131.289 | 165.463 | 231.342 | 245.489 | 265.498 | 296.589 | 298.596 |
| No Contrast | 6.908 | 0.684 | 0.523 | 0.162 | 0.032 | 0.046 | 0.070 | 0.056 | 0.021 | 0.036 | 0.020 |
| Iron Oxide | 0.919 | 0.642 | 0.519 | 0.211 | 0.011 | 0.068 | 0.080 | 0.072 | 0.007 | 0.035 | 0.023 |
| Gd Oxide | 0.947 | 0.735 | 0.630 | 0.244 | 0.029 | 0.012 | 0.050 | 0.083 | 0.015 | 0.010 | 0.614 |
| Mn Oxide | 0.920 | 0.531 | 0.607 | 0.231 | 0.091 | [symbol] | 0.083 | 0.028 | 0.062 | 0.036 | 0.025 |

[symbol]

indicates data missing or illegible when filed

### Example 20

In this example the calcium chloride described in example 15 was substituted with barium chloride at the same concentration.

The release profile from the preparation in micrograms/mL is listed in the top panel of the table below and the resulting viability of HepG2 cells incubated with the solution is listed in the bottom panel of the table.

TABLE 8

| | 0 hrs | 6 hrs | 12 hrs | 1 day | 2 days | 3 days | 4 days | 5 days | 6 days | 7 days | 8 days |
|---|---|---|---|---|---|---|---|---|---|---|---|
| No Contrast | 0.326 | 7.563 | 20.654 | 51.189 | 87.632 | 185.632 | 201.645 | 213.499 | 248.679 | 295.456 | 299.596 |
| Iron Oxide | 0.162 | 8.124 | 18.845 | 48.318 | 80.439 | 143.346 | 210.342 | 224.476 | 245.598 | 312.598 | 314.597 |
| Gd Oxide | 0.325 | 6.985 | 24.564 | 46.734 | 93.462 | 178.458 | 194.532 | 219.496 | 268.598 | 300.495 | 308.587 |
| Mn Oxide | 0.191 | 8.321 | 26.483 | 5.123 | 111.2890 | 165.463 | 231.342 | 245.489 | 265.498 | 296.589 | 298.596 |
| No Contrast | 0.964 | 0.620 | 0.589 | 0.198 | 0.041 | 0.046 | 0.027 | 0.058 | 0.028 | 0.036 | 0.77 |
| Iron Oxide | 0.903 | 0.689 | 0.489 | 0.219 | 0.033 | 0.033 | 0.069 | 0.075 | 0.025 | 0.031 | 0.020 |
| Gd Oxide | 0.942 | 0.564 | 0.407 | 0.286 | 0.023 | 0.049 | 0.096 | 0.098 | 0.016 | 0.037 | 0.053 |
| Mn Oxide | 0.935 | 0.514 | 0.413 | 0.109 | 0.049 | 0.031 | 0.088 | 0.051 | 0.033 | 0.023 | 0.023 |

### Example 21

This example highlights the loading of a liposome polymer matrix utilizing an ammonium gradient. Liposomes were prepared using the extended hydration method. Phosphatidylcholine (Avanti Polar Lipids, Alabaster, Ala.) and cholesterol (Sigma, St. Louis, Mo.) in a 1:1 mole ratio in chloroform were dried in a rotary evaporator. The lipid film was solvated and annealed for 2 hours at 55 C with a solution of 250 mM ammonium sulfate and 3M sucrose. In order to form vesicles of a specific size, the lipid suspension was taken through twenty-one cycles of extrusion (LiposoFast, Avestin, Ottawa, Ontario, Canada) through two stacked polycarbonate filters (100 nm or 400 nm pore size). Unencapsulated citrate was removed using size exclusion chromatography with sephadex G-50 resin (Sigma, St. Louis, Mo.). Dynamic light scattering (DLS) measurements of liposome suspensions was studied with a Malvern Instruments Nanosizer ZS90 (Southborough, Mass.), equipped with a 633 nm He—Ne laser light source. Scattering was detected at 90. All solutions used were filtered with 0.22 [tm filters just prior to vesicle preparation. The liposomes underwent a buffer exchange to remove the external, unentrapped, ethylenediamine citrate. This was accomplished by subjecting the liposomes to tangential flow ultrafiltration. This process removes water and small molecular weight solutes while retaining the liposomes. Using this process, the ammonium sulfate buffer external to the vesicles was replaced with 250 mM Histidine HCl and 3M sucrose.

To this 100 mg/mL liposome solution was added a concentrated solution of poly(ethylene glycol)-diacrylate (PEGDA; Nektar Therapeutics, Huntsville, Ala.) in sterile phosphate-buffered saline (PBS; GIBCO Invitrogen, Carlsbad, Calif.) to make a 15% (w/v) hydrogel. Photoinitiator, Igracure D2959 (Ciba Specialty Chemicals, Tarrytown, N.Y.) was added to the polymer at a concentration of 0.05% (w/v). The mixture is then added drop-wise to a reactant medium made up of an inert bath. An inert bath includes but is not limited to a formulation of 160 grams Span 80 and 3,840 grams USP mineral oil. The inert bath is then stirred at a rate of 200 RPM in a reaction vat. The Vat was then exposed to UV irradiation for 5 minutes to initiate polymerization. Once reacted, the resultant crosslinked PEGDA sphere with the entrapped liposomes were cleaned of mineral oil and Span 8. The cleaning process involved dumping the resultant spheres over a 45-micron screen such that the beads were trapped on the screen while some of the reactants passed through. The spheres were then washed with a solution containing 125 mM lactose and 250 mM ammonium sulfate.

Following polymerization and washing, the liposome polymer matrix underwent a buffer exchange to remove the external, unentrapped, ethylenediamine citrate. This was accomplished by subjecting the microspheres to tangential flow ultrafiltration. This process removes water and small molecular weight solutes while retaining the microspheres. Using this process, the lactose/ethylenediamine citrate buffer external to the vesicles was replaced with 250 mM lactose/50 mM glycine. Additionally to facilitate a method for large scale synthesis the spheres were loaded in a chromatography column and were washed with three exchanges of chilled 250 mM lactose/50 mM glycine solution equal to the packed volume of the spheres.

The spheres that were subjected to ultrafiltration or column washes were stored for a variable amount of time in a minimal amount of 250 mM histidine HCl and 3M lactose to maintain hydration at 20° C. and 0° C. Following storage the loading efficiency of doxorubicin into the spheres was assessed (table 1). For drug loading a procedure that could easily be implement in the pharmacy was employed. Specifically a water bath or dry heat block was allowed to equilibrate at 58° C. (55°-60° C.). For each 1 mL packed volume of microspheres, 50 mg of lyophilized doxorubicin (for storage lactose and parahydroxybenzoate may be added) in a solution of 10 mL was prepared. This solution was then added to the equilibrated heating source for 10 min. Next, the microspheres and 10 mL of doxorubicin were then combined. The mixture was then shaken vigorously and allowed to sit for at least 10 minutes in the heat source and then to minutes at room temperature before use. After this period the spheres can be stored for 24 hours at room temperature.

The release profile from the preparation in micrograms/mL is listed in the top panel of the table below and the resulting viability of HepG2 cells incubated with the solution is listed in the bottom panel of the table.

TABLE 9
0 hours of storage at 5° C. prior to drug loading

| | 0 hrs | 6 hrs | 12 hrs | 1 day | 2 day | 3 day | 4 day | 5 day | 6 day | 7 day | 8 day | 9 day | 10 day | 11 day |
|---|---|---|---|---|---|---|---|---|---|---|---|---|---|---|
| No Contrast | [symbol] | [symbol] | [symbol] | [symbol] | [symbol] | [symbol] | [symbol] | [symbol] | [symbol] | [symbol] | [symbol] | [symbol] | [symbol] | [symbol] |
| Iron Oxide | [symbol] | [symbol] | [symbol] | [symbol] | [symbol] | [symbol] | [symbol] | [symbol] | [symbol] | [symbol] | [symbol] | [symbol] | [symbol] | [symbol] |
| Gd Oxide | [symbol] | [symbol] | [symbol] | [symbol] | [symbol] | [symbol] | [symbol] | [symbol] | [symbol] | [symbol] | [symbol] | [symbol] | [symbol] | [symbol] |
| Mn Oxide | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |

| | 12 day | 13 day | 14 day | 15 day | 16 day | 17 day | 18 day | 19 day | 20 day | 21 day |
|---|---|---|---|---|---|---|---|---|---|---|
| No Contrast | [symbol] | [symbol] | [symbol] | [symbol] | [symbol] | [symbol] | [symbol] | [symbol] | [symbol] | [symbol] |
| Iron Oxide | [symbol] | [symbol] | [symbol] | [symbol] | [symbol] | [symbol] | [symbol] | [symbol] | [symbol] | [symbol] |
| Gd Oxide | [symbol] | [symbol] | [symbol] | [symbol] | [symbol] | [symbol] | [symbol] | [symbol] | [symbol] | [symbol] |
| Mn Oxide | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |

[symbol]

indicates data missing or illegible when filed

TABLE 10
7 days of storage at 5° C. prior to drug loading

| | 0 hrs | 6 hrs | 12 hrs | 1 day | 2 day | 3 day | 4 day | 5 day | 6 day | 7 day | 8 day | 9 day | 10 day | 11 day |
|---|---|---|---|---|---|---|---|---|---|---|---|---|---|---|
| No Contrast | [symbol] | [symbol] | [symbol] | [symbol] | [symbol] | [symbol] | [symbol] | [symbol] | [symbol] | [symbol] | [symbol] | [symbol] | [symbol] | [symbol] |
| Iron Oxide | [symbol] | [symbol] | [symbol] | [symbol] | [symbol] | [symbol] | [symbol] | [symbol] | [symbol] | [symbol] | [symbol] | [symbol] | [symbol] | [symbol] |
| Gd Oxide | [symbol] | [symbol] | [symbol] | [symbol] | [symbol] | [symbol] | [symbol] | [symbol] | [symbol] | [symbol] | [symbol] | [symbol] | [symbol] | [symbol] |
| Mn Oxide | [symbol] | [symbol] | [symbol] | [symbol] | [symbol] | [symbol] | [symbol] | [symbol] | [symbol] | [symbol] | [symbol] | [symbol] | [symbol] | [symbol] |

| | 12 day | 13 day | 14 day | 15 day | 16 day | 17 day | 18 day | 19 day | 20 day | 21 day |
|---|---|---|---|---|---|---|---|---|---|---|
| No Contrast | [symbol] | [symbol] | [symbol] | [symbol] | [symbol] | [symbol] | [symbol] | [symbol] | [symbol] | [symbol] |
| Iron Oxide | [symbol] | [symbol] | [symbol] | [symbol] | [symbol] | [symbol] | [symbol] | [symbol] | [symbol] | [symbol] |
| Gd Oxide | [symbol] | [symbol] | [symbol] | [symbol] | [symbol] | [symbol] | [symbol] | [symbol] | [symbol] | [symbol] |
| Mn Oxide | [symbol] | [symbol] | [symbol] | [symbol] | [symbol] | [symbol] | [symbol] | [symbol] | [symbol] | [symbol] |

| | 0 hrs | 6 hrs | 12 hrs | 1 day | 2 days | 3 days | 4 days | 5 days | 6 days | 7 days | 8 days | 9 days | 10 day | 11 day |
|---|---|---|---|---|---|---|---|---|---|---|---|---|---|---|
| No Contrast | [symbol] | [symbol] | [symbol] | [symbol] | [symbol] | [symbol] | [symbol] | [symbol] | [symbol] | [symbol] | [symbol] | [symbol] | [symbol] | [symbol] |
| Iron Oxide | [symbol] | [symbol] | [symbol] | [symbol] | [symbol] | [symbol] | [symbol] | [symbol] | [symbol] | [symbol] | [symbol] | [symbol] | [symbol] | [symbol] |
| Gd Oxide | [symbol] | [symbol] | [symbol] | [symbol] | [symbol] | [symbol] | [symbol] | [symbol] | [symbol] | [symbol] | [symbol] | [symbol] | [symbol] | [symbol] |
| Mn Oxide | [symbol] | [symbol] | [symbol] | [symbol] | [symbol] | [symbol] | [symbol] | [symbol] | [symbol] | [symbol] | [symbol] | [symbol] | [symbol] | [symbol] |

| | 12 day | 13 day | 14 day | 15 day | 16 day | 17 day | 18 day | 19 day | 20 day | 21 day |
|---|---|---|---|---|---|---|---|---|---|---|
| No Contrast | [symbol] | [symbol] | [symbol] | [symbol] | [symbol] | [symbol] | [symbol] | [symbol] | [symbol] | [symbol] |
| Iron Oxide | [symbol] | [symbol] | [symbol] | [symbol] | [symbol] | [symbol] | [symbol] | [symbol] | [symbol] | [symbol] |
| Gd Oxide | [symbol] | [symbol] | [symbol] | [symbol] | [symbol] | [symbol] | [symbol] | [symbol] | [symbol] | [symbol] |
| Mn Oxide | [symbol] | [symbol] | [symbol] | [symbol] | [symbol] | [symbol] | [symbol] | [symbol] | [symbol] | [symbol] |

[symbol]

indicates data missing or illegible when filed

TABLE 11
14 days of storage at 5° C. prior to drug loading

| | 0 hrs | 6 hrs | 12 hrs | 1 day | 2 day | 3 day | 4 day | 5 day | 6 day | 7 day | 8 day | 9 day | 10 day | 11 day |
|---|---|---|---|---|---|---|---|---|---|---|---|---|---|---|
| No Contrast | [symbol] | [symbol] | [symbol] | [symbol] | [symbol] | [symbol] | [symbol] | [symbol] | [symbol] | [symbol] | [symbol] | [symbol] | [symbol] | [symbol] |
| Iron Oxide | [symbol] | [symbol] | [symbol] | [symbol] | [symbol] | [symbol] | [symbol] | [symbol] | [symbol] | [symbol] | [symbol] | [symbol] | [symbol] | [symbol] |
| Gd Oxide | [symbol] | [symbol] | [symbol] | [symbol] | [symbol] | [symbol] | [symbol] | [symbol] | [symbol] | [symbol] | [symbol] | [symbol] | [symbol] | [symbol] |
| Mn Oxide | [symbol] | [symbol] | [symbol] | [symbol] | [symbol] | [symbol] | [symbol] | [symbol] | [symbol] | [symbol] | [symbol] | [symbol] | [symbol] | [symbol] |
| No Contrast | [symbol] | [symbol] | [symbol] | [symbol] | [symbol] | [symbol] | [symbol] | [symbol] | [symbol] | [symbol] | [symbol] | [symbol] | [symbol] | [symbol] |
| Iron Oxide | [symbol] | [symbol] | [symbol] | [symbol] | [symbol] | [symbol] | [symbol] | [symbol] | [symbol] | [symbol] | [symbol] | [symbol] | [symbol] | [symbol] |
| Gd Oxide | [symbol] | [symbol] | [symbol] | [symbol] | [symbol] | [symbol] | [symbol] | [symbol] | [symbol] | [symbol] | [symbol] | [symbol] | [symbol] | [symbol] |
| Mn Oxide | [symbol] | [symbol] | [symbol] | [symbol] | [symbol] | [symbol] | [symbol] | [symbol] | [symbol] | [symbol] | [symbol] | [symbol] | [symbol] | [symbol] |

| | 12 day | 13 day | 14 day | 15 day | 16 day | 17 day | 18 day | 19 day | 20 day | 21 day |
|---|---|---|---|---|---|---|---|---|---|---|
| No Contrast | [symbol] | [symbol] | [symbol] | [symbol] | [symbol] | [symbol] | [symbol] | [symbol] | [symbol] | [symbol] |
| Iron Oxide | [symbol] | [symbol] | [symbol] | [symbol] | [symbol] | [symbol] | [symbol] | [symbol] | [symbol] | [symbol] |
| Gd Oxide | [symbol] | [symbol] | [symbol] | [symbol] | [symbol] | [symbol] | [symbol] | [symbol] | [symbol] | [symbol] |
| Mn Oxide | [symbol] | [symbol] | [symbol] | [symbol] | [symbol] | [symbol] | [symbol] | [symbol] | [symbol] | [symbol] |
| No Contrast | [symbol] | [symbol] | [symbol] | [symbol] | [symbol] | [symbol] | [symbol] | [symbol] | [symbol] | [symbol] |
| Iron Oxide | [symbol] | [symbol] | [symbol] | [symbol] | [symbol] | [symbol] | [symbol] | [symbol] | [symbol] | [symbol] |
| Gd Oxide | [symbol] | [symbol] | [symbol] | [symbol] | [symbol] | [symbol] | [symbol] | [symbol] | [symbol] | [symbol] |
| Mn Oxide | [symbol] | [symbol] | [symbol] | [symbol] | [symbol] | [symbol] | [symbol] | [symbol] | [symbol] | [symbol] |

[symbol]

indicates data missing or illegible when filed

TABLE 12
1 month of storage at 5° C. prior to drug loading

| | 0 hrs | 6 hrs | 12 hrs | 1 day | 2 day | 3 day | 4 day | 5 day | 6 day | 7 day | 8 day | 9 day | 10 day | 11 day |
|---|---|---|---|---|---|---|---|---|---|---|---|---|---|---|
| No Contrast | [symbol] | [symbol] | [symbol] | [symbol] | [symbol] | [symbol] | [symbol] | [symbol] | [symbol] | [symbol] | [symbol] | [symbol] | [symbol] | [symbol] |
| Iron Oxide | [symbol] | [symbol] | [symbol] | [symbol] | [symbol] | [symbol] | [symbol] | [symbol] | [symbol] | [symbol] | [symbol] | [symbol] | [symbol] | [symbol] |
| Gd Oxide | [symbol] | [symbol] | [symbol] | [symbol] | [symbol] | [symbol] | [symbol] | [symbol] | [symbol] | [symbol] | [symbol] | [symbol] | [symbol] | [symbol] |
| Mn Oxide | [symbol] | [symbol] | [symbol] | [symbol] | [symbol] | [symbol] | [symbol] | [symbol] | [symbol] | [symbol] | [symbol] | [symbol] | [symbol] | [symbol] |
| No Contrast | [symbol] | [symbol] | [symbol] | [symbol] | [symbol] | [symbol] | [symbol] | [symbol] | [symbol] | [symbol] | [symbol] | [symbol] | [symbol] | [symbol] |
| Iron Oxide | [symbol] | [symbol] | [symbol] | [symbol] | [symbol] | [symbol] | [symbol] | [symbol] | [symbol] | [symbol] | [symbol] | [symbol] | [symbol] | [symbol] |
| Gd Oxide | [symbol] | [symbol] | [symbol] | [symbol] | [symbol] | [symbol] | [symbol] | [symbol] | [symbol] | [symbol] | [symbol] | [symbol] | [symbol] | [symbol] |
| Mn Oxide | [symbol] | [symbol] | [symbol] | [symbol] | [symbol] | [symbol] | [symbol] | [symbol] | [symbol] | [symbol] | [symbol] | [symbol] | [symbol] | [symbol] |

| | 12 day | 13 day | 14 day | 15 day | 16 day | 17 day | 18 day | 19 day | 20 day | 21 day |
|---|---|---|---|---|---|---|---|---|---|---|
| No Contrast | [symbol] | [symbol] | [symbol] | [symbol] | [symbol] | [symbol] | [symbol] | [symbol] | [symbol] | [symbol] |
| Iron Oxide | [symbol] | [symbol] | [symbol] | [symbol] | [symbol] | [symbol] | [symbol] | [symbol] | [symbol] | [symbol] |
| Gd Oxide | [symbol] | [symbol] | [symbol] | [symbol] | [symbol] | [symbol] | [symbol] | [symbol] | [symbol] | [symbol] |
| Mn Oxide | [symbol] | [symbol] | [symbol] | [symbol] | [symbol] | [symbol] | [symbol] | [symbol] | [symbol] | [symbol] |
| No Contrast | [symbol] | [symbol] | [symbol] | [symbol] | [symbol] | [symbol] | [symbol] | [symbol] | [symbol] | [symbol] |
| Iron Oxide | [symbol] | [symbol] | [symbol] | [symbol] | [symbol] | [symbol] | [symbol] | [symbol] | [symbol] | [symbol] |
| Gd Oxide | [symbol] | [symbol] | [symbol] | [symbol] | [symbol] | [symbol] | [symbol] | [symbol] | [symbol] | [symbol] |
| Mn Oxide | [symbol] | [symbol] | [symbol] | [symbol] | [symbol] | [symbol] | [symbol] | [symbol] | [symbol] | [symbol] |

[symbol]

indicates data missing or illegible when filed

Next, the method as described herein was performed, wherein EPC:Chol (1:1 molar) liposomes encapsulating 250 mM ammonium sulfate plus 3M sucrose, extruded through 100 nm filters and “washed” with 250 mM histidine HCl plus 3M sucrose were replaced with HSPC:Chol:PEG2000-DSPE (56.4:38:3 molar) liposomes encapsulating 250 mM ammonium sulfate plus 3M sucrose, extruded through 100 nm filters and “washed” with 250 mM histidine HCl plus 3M sucrose. Total lipid concentration: 100 mg/ml.

The release profile from the preparation in micrograms/mL is listed in the top panel of the table below and the resulting viability of HepG2 cells incubated with the solution is listed in the bottom panel of the table.

TABLE 13

| | 0 hrs | 6 hrs | 12 hrs | 1 day | 2 day | 3 day | 4 day | 5 day | 6 day | 7 day | 8 day | 9 day | 10 day | 11 day |
|---|---|---|---|---|---|---|---|---|---|---|---|---|---|---|
| No Contrast | [symbol] | [symbol] | [symbol] | [symbol] | [symbol] | [symbol] | [symbol] | [symbol] | [symbol] | [symbol] | [symbol] | [symbol] | [symbol] | [symbol] |
| Iron Oxide | [symbol] | [symbol] | [symbol] | [symbol] | [symbol] | [symbol] | [symbol] | [symbol] | [symbol] | [symbol] | [symbol] | [symbol] | [symbol] | [symbol] |
| Gd Oxide | [symbol] | [symbol] | [symbol] | [symbol] | [symbol] | [symbol] | [symbol] | [symbol] | [symbol] | [symbol] | [symbol] | [symbol] | [symbol] | [symbol] |
| Mn Oxide | [symbol] | [symbol] | [symbol] | [symbol] | [symbol] | [symbol] | [symbol] | [symbol] | [symbol] | [symbol] | [symbol] | [symbol] | [symbol] | [symbol] |
| No Contrast | [symbol] | [symbol] | [symbol] | [symbol] | [symbol] | [symbol] | [symbol] | [symbol] | [symbol] | [symbol] | [symbol] | [symbol] | [symbol] | [symbol] |
| Iron Oxide | [symbol] | [symbol] | [symbol] | [symbol] | [symbol] | [symbol] | [symbol] | [symbol] | [symbol] | [symbol] | [symbol] | [symbol] | [symbol] | [symbol] |
| Gd Oxide | [symbol] | [symbol] | [symbol] | [symbol] | [symbol] | [symbol] | [symbol] | [symbol] | [symbol] | [symbol] | [symbol] | [symbol] | [symbol] | [symbol] |
| Mn Oxide | [symbol] | [symbol] | [symbol] | [symbol] | [symbol] | [symbol] | [symbol] | [symbol] | [symbol] | [symbol] | [symbol] | [symbol] | [symbol] | [symbol] |

| | 12 day | 13 day | 14 day | 15 day | 16 day | 17 day | 18 day | 19 day | 20 day | 21 day |
|---|---|---|---|---|---|---|---|---|---|---|
| No Contrast | [symbol] | [symbol] | [symbol] | [symbol] | [symbol] | [symbol] | [symbol] | [symbol] | [symbol] | [symbol] |
| Iron Oxide | [symbol] | [symbol] | [symbol] | [symbol] | [symbol] | [symbol] | [symbol] | [symbol] | [symbol] | [symbol] |
| Gd Oxide | [symbol] | [symbol] | [symbol] | [symbol] | [symbol] | [symbol] | [symbol] | [symbol] | [symbol] | [symbol] |
| Mn Oxide | [symbol] | [symbol] | [symbol] | [symbol] | [symbol] | [symbol] | [symbol] | [symbol] | [symbol] | [symbol] |
| No Contrast | [symbol] | [symbol] | [symbol] | [symbol] | [symbol] | [symbol] | [symbol] | [symbol] | [symbol] | [symbol] |
| Iron Oxide | [symbol] | [symbol] | [symbol] | [symbol] | [symbol] | [symbol] | [symbol] | [symbol] | [symbol] | [symbol] |
| Gd Oxide | [symbol] | [symbol] | [symbol] | [symbol] | [symbol] | [symbol] | [symbol] | [symbol] | [symbol] | [symbol] |
| Mn Oxide | [symbol] | [symbol] | [symbol] | [symbol] | [symbol] | [symbol] | [symbol] | [symbol] | [symbol] | [symbol] |

[symbol]

indicates data missing or illegible when filed

### Example 22

The procedure described in the example above was utilized and doxorubicin was substituted for irinotecan to load 50 mg of irinotecan per packed volume of microsphere.

The release profile from the preparation in micrograms/mL is listed in the top panel of the table below and the resulting viability of HepG2 cells incubated with the solution is listed in the bottom panel of the table.

TABLE 14

| | 0 hrs | 6 hrs | 12 hrs | 1 day | 2 day | 3 day | 4 day | 5 day | 6 day | 7 day | 8 day | 9 day | 10 day | 11 day |
|---|---|---|---|---|---|---|---|---|---|---|---|---|---|---|
| No Contrast | [symbol] | [symbol] | [symbol] | [symbol] | [symbol] | [symbol] | [symbol] | [symbol] | [symbol] | [symbol] | [symbol] | [symbol] | [symbol] | [symbol] |
| Iron Oxide | [symbol] | [symbol] | [symbol] | [symbol] | [symbol] | [symbol] | [symbol] | [symbol] | [symbol] | [symbol] | [symbol] | [symbol] | [symbol] | [symbol] |
| Gd Oxide | [symbol] | [symbol] | [symbol] | [symbol] | [symbol] | [symbol] | [symbol] | [symbol] | [symbol] | [symbol] | [symbol] | [symbol] | [symbol] | [symbol] |
| Mn Oxide | [symbol] | [symbol] | [symbol] | [symbol] | [symbol] | [symbol] | [symbol] | [symbol] | [symbol] | [symbol] | [symbol] | [symbol] | [symbol] | [symbol] |
| No Contrast | [symbol] | [symbol] | [symbol] | [symbol] | [symbol] | [symbol] | [symbol] | [symbol] | [symbol] | [symbol] | [symbol] | [symbol] | [symbol] | [symbol] |
| Iron Oxide | [symbol] | [symbol] | [symbol] | [symbol] | [symbol] | [symbol] | [symbol] | [symbol] | [symbol] | [symbol] | [symbol] | [symbol] | [symbol] | [symbol] |
| Gd Oxide | [symbol] | [symbol] | [symbol] | [symbol] | [symbol] | [symbol] | [symbol] | [symbol] | [symbol] | [symbol] | [symbol] | [symbol] | [symbol] | [symbol] |
| Mn Oxide | [symbol] | [symbol] | [symbol] | [symbol] | [symbol] | [symbol] | [symbol] | [symbol] | [symbol] | [symbol] | [symbol] | [symbol] | [symbol] | [symbol] |

| | 12 day | 13 day | 14 day | 15 day | 16 day | 17 day | 18 day | 19 day | 20 day | 21 day |
|---|---|---|---|---|---|---|---|---|---|---|
| No Contrast | [symbol] | [symbol] | [symbol] | [symbol] | [symbol] | [symbol] | [symbol] | [symbol] | [symbol] | [symbol] |
| Iron Oxide | [symbol] | [symbol] | [symbol] | [symbol] | [symbol] | [symbol] | [symbol] | [symbol] | [symbol] | [symbol] |
| Gd Oxide | [symbol] | [symbol] | [symbol] | [symbol] | [symbol] | [symbol] | [symbol] | [symbol] | [symbol] | [symbol] |
| Mn Oxide | [symbol] | [symbol] | [symbol] | [symbol] | [symbol] | [symbol] | [symbol] | [symbol] | [symbol] | [symbol] |
| No Contrast | [symbol] | [symbol] | [symbol] | [symbol] | [symbol] | [symbol] | [symbol] | [symbol] | [symbol] | [symbol] |
| Iron Oxide | [symbol] | [symbol] | [symbol] | [symbol] | [symbol] | [symbol] | [symbol] | [symbol] | [symbol] | [symbol] |
| Gd Oxide | [symbol] | [symbol] | [symbol] | [symbol] | [symbol] | [symbol] | [symbol] | [symbol] | [symbol] | [symbol] |
| Mn Oxide | [symbol] | [symbol] | [symbol] | [symbol] | [symbol] | [symbol] | [symbol] | [symbol] | [symbol] | [symbol] |

[symbol]

indicates data missing or illegible when filed

### Example 23

This example describes the formation of liposomes with PEGDA cores. Liposomes were prepared using the extended hydration method. Phosphatidylcholine (Avanti Polar Lipids, Alabaster, Ala.) and cholesterol (Sigma, St. Louis, Mo.) in a 1:1 mole ratio in chloroform were dried in a rotary evaporator. The lipid film was solvated and annealed for 2 hours at 55 C with a solution of poly(ethylene glycol)-diacrylate (PEGDA; Nektar Therapeutics, Huntsville, Ala.) in sterile phosphate-buffered saline (PBS; GIBCO Invitrogen, Carlsbad, Calif.) to make a 15% (w/v) hydrogel. Photoinitiator, Igracure D2959 (Ciba Specialty Chemicals, Tarrytown, N.Y.) was added to the polymer at a concentration of 0.05% (w/v). In order to form vesicles of a specific size, the lipid suspension was taken through twenty-one cycles of extrusion (LiposoFast, Avestin, Ottawa, Ontario, Canada) through two stacked polycarbonate filters (100 nm or 400 nm pore size).

To the unpolymerized pegda liposome solution was added a concentrated solution of poly(ethylene glycol)-diacrylate (PEGDA; Nektar Therapeutics, Huntsville, Ala.) in sterile phosphate-buffered saline (PBS; GIBCO Invitrogen, Carlsbad, Calif.) to make a 15% (w/v) hydrogel. Photoinitiator, Igracure D2959 (Ciba Specialty Chemicals, Tarrytown, N.Y.) was added to the polymer at a concentration of 0.05% (w/v). The mixture is then added drop-wise to a reactant medium made up of an inert bath. An inert bath includes but is not limited to a formulation of 160 grams Span 80 and 3,840 grams USP mineral oil. The inert bath is then stirred at a rate of 200 RPM in a reaction vat. The Vat was then exposed to UV irradiation for 5 minutes to initiate polymerization. Once reacted, the resultant crosslinked PEGDA sphere with the entrapped liposomes were cleaned of mineral oil and Span 8. The cleaning process involved dumping the resultant spheres over a 45-micron screen such that the beads were trapped on the screen while some of the reactants passed through. The spheres were washed three times as described above with water.

### Example 24

Liposomes were prepared using the extended hydration method. Phosphatidylcholine (Avanti Polar Lipids, Alabaster, Ala.) and cholesterol (Sigma, St. Louis, Mo.) in a 1:1 mole ratio in chloroform were dried in a rotary evaporator. The lipid film was solvated and annealed for 2 hours at 55 C with a solution of PBS. In order to form vesicles of a specific size, the lipid suspension was taken through twenty-one cycles of extrusion (LiposoFast, Avestin, Ottawa, Ontario, Canada) through two stacked polycarbonate filters (100 nm or 400 nm pore size).

To this liposome solution was added a concentrated solution of poly(ethylene glycol)-diacrylate (PEGDA; Nektar Therapeutics, Huntsville, Ala.) in sterile phosphate-buffered saline (PBS; GIBCO Invitrogen, Carlsbad, Calif.) to make a 15% (w/v) hydrogel. Photoinitiator, Igracure D2959 (Ciba Specialty Chemicals, Tarrytown, N.Y.) was added to the polymer at a concentration of 0.05% (w/v). The mixture is then added drop-wise to a reactant medium made up of an inert bath. An inert bath includes but is not limited to a formulation of 160 grams Span 80 and 3,840 grams USP mineral oil. The inert bath is then stirred at a rate of 200 RPM in a reaction vat. The Vat was then exposed to UV irradiation for 5 minutes to initiate polymerization. Once reacted, the resultant crosslinked PEGDA sphere with the entrapped liposomes were cleaned of mineral oil and Span 8. The cleaning process involved dumping the resultant spheres over a 45-micron screen such that the beads were trapped on the screen while some of the reactants passed through. The spheres were washed three times as described above with water.

Next a 200 mg/mL solution of 3BrPa (Sigma Chemical Co., St. Louis, Mo.) buffered to pH 7 was heated to 50° C. and concentrated ethanol was added to the heated mixture to create a final ethanol concentration of 20%. Micropsheres were then added to the heated ethanol/3 bromopyruvate solution and incubated for 10 minutes at 50° C. Using a filter screen, microspheres were immediately removed from the heated solution and placed in chilled PBS.

The release profile from these microspheres was assessed using NMR spectroscopy. Further the content of drug loaded in these spheres was assessed using MR spectroscopy.

Alginate/liposome polymer matrices designed to have high heating potential when utilized with an alternating magnetic field were generated. Alginate was added directly to a Feridex colloidal solution until the alginate concentration was 2%. To this solution the liposomes in example 14 were added and sphere synthesis and drug loading with doxorubicin was followed without exception as described herein.

TABLE 15

| | 0 hrs | 6 hrs | 12 hrs | 1 day | 2 days | 3 days | 4 days | 5 days | 6 days | 7 days | 8 days |
|---|---|---|---|---|---|---|---|---|---|---|---|
| No Contrast | 0.617 | 8.857 | 23.496 | 61.345 | 95.438 | 112.594 | 199.459 | 204.597 | 239.394 | 345.298 | 352.335 |
| Iron Oxide | 0.812 | 10.859 | 16.595 | 42.489 | 80.398 | 134.414 | 235.395 | 210.458 | 224.598 | 320.433 | 323.371 |
| Gd Oxide | 0.553 | 14.584 | 16.456 | 56.485 | 79.321 | 108.594 | 210.410 | 245.983 | 298.486 | 295.453 | 298.453 |
| Mn Oxide | 0.328 | 5.893 | 24.569 | 59.485 | 111.205 | 109.456 | 202.495 | 269.985 | 275.857 | 320.597 | 322.929 |
| No Contrast | 0.947 | 0.650 | 0.468 | 0.385 | 0.066 | 0.049 | 0.038 | 0.026 | 0.085 | 0.035 | 0.057 |
| Iron Oxide | 0.968 | 0.622 | 0.575 | 0.289 | 0.058 | 0.035 | 0.048 | 0.048 | 0.049 | 0.057 | 0.028 |
| Gd Oxide | 0.935 | 0.649 | 0.457 | 0.284 | 0.098 | 0.088 | 0.038 | 0.056 | 0.039 | 0.026 | 0.039 |
| Mn Oxide | 0.969 | 0.687 | 0.587 | 0.185 | 0.098 | 0.038 | 0.016 | 0.039 | 0.055 | 0.031 | 0.057 |

### Example 25

Alginate/liposome polymer matrices designed to have high heating potential when utilized with an alternating magnetic field were generated. A concentrated alginate solution was added directly to a Feridex colloidal solution giving a final concentration of 2% alginate and 60% volume/volume feridex. To this solution the liposomes in example 1 were added and sphere synthesis and drug loading with doxorubicin was followed without exception as described previously.

### Example 26

PEGDA liposome polymer matrices described herein (e.g. example 14) were prepared. The polymer matrices were then lyophilized under the following conditions. Shelf loading temperature 0.degree. C. Product ramp time to freezing temperature 5.5 hr Shelf freezing temperature −50° C. Primary drying temperature −5° C. Primary drying pressure 40 mm Hg Primary drying time 51 hr Secondary drying temperature 25° C. Secondary drying pressure 75 mm Hg Secondary drying time 67 hr.

The liposome polymer matrices were subsequently reconstituted by directly placing heated spheres in sodium carbonate solution as described in example 1 and the loading process as described in example 14 was followed without exception. The uptake of doxorubicin was compared to the non-lyophilized polymer liposome matrix.

The release profile from the preparation in micrograms/mL is listed in the top panel of the table below and the resulting viability of HepG2 cells incubated with the solution is listed in the bottom panel of the table.

TABLE 16

| | 0 hrs | 6 hrs | 12 hrs | 1 day | 2 days | 3 days | 4 days | 5 days | 6 days | 7 days | 8 days |
|---|---|---|---|---|---|---|---|---|---|---|---|
| No Contrast | 0.162 | 9.145 | 24.583 | 49.391 | 123.625 | 123.590 | 198.350 | 230.459 | 284.578 | 302.459 | 304.579 |
| Iron Oxide | 0.322 | 6.832 | 27.931 | 54.678 | 103.629 | 174.569 | 195.873 | 229.893 | 260.487 | 299.480 | 301.460 |
| Gd Oxide | 0.158 | [symbol] | 22.456 | 52.165 | 132.982 | [symbol] | 220.312 | 245.458 | 222.498 | 321.568 | 322.486 |
| Mn Oxide | 0.253 | 6.478 | [symbol] | 48.785 | 120.857 | 155.349 | 210.587 | 238.457 | 254.938 | 301.460 | 306.569 |
| No Contrast | 0.907 | 0.631 | 0.417 | 0.309 | 0.031 | 0.050 | 0.066 | 0.011 | 0.021 | 0.039 | 0.019 |
| Iron Oxide | 0.932 | 0.679 | 0.543 | 0.231 | 0.031 | 0.021 | 0.073 | 0.079 | 0.021 | 0.014 | 0.021 |
| Gd Oxide | 0.941 | [symbol] | 0.435 | 0.126 | 0.020 | 0.046 | 0.087 | 0.043 | 0.017 | 0.036 | 0.025 |
| Mn Oxide | 0.892 | 0.639 | 0.570 | 0.324 | 0.032 | 0.058 | 0.023 | 0.011 | 0.022 | 0.043 | 0.067 |

[symbol]

indicates data missing or illegible when filed

### Example 27

PEGDA liposome polymer matrices as described in example 14 were prepared. The polymer matrices were then lyophilized under the following conditions. Shelf loading temperature 0.degree. C. Product ramp time to freezing temperature 4.9 hr Shelf freezing temperature −50° C. Primary drying temperature −25° C. Primary drying pressure 75 mmHg Primary drying time 80.1 hr Secondary drying temperature 25° C. Secondary drying pressure 75 mmHg. Secondary drying time 25 hr.

The liposome polymer matrices were subsequently reconstituted by directly placing heated spheres in sodium carbonate solution as described in example 1 and the loading process as described in example 1 was followed without exception. The uptake of doxorubicin was compared to the non-lyophilized polymer liposome matrix. Under these conditions, the liposome polymer matrices showed a 4% decrease in amount of doxoroubicn loaded into liposomes.

The release profile from the preparation in micrograms/mL is listed in the first table below and the resulting viability of HepG2 cells incubated with the solution is listed in the second table.

TABLE 17

| | 0 hrs | 6 hrs | 12 hrs | 1 day | 2 days | 3 days | 4 days | 5 days | 6 days | 7 days | 8 days |
|---|---|---|---|---|---|---|---|---|---|---|---|
| No Contrast | 0.149 | 8.345 | 20.452 | 43.879 | 120.557 | 172.460 | 180.453 | 210.489 | 265.498 | 298.516 | 301.460 |
| Iron Oxide | 0.253 | 11.234 | 20.385 | 51.483 | 101.589 | 178.655 | 190.438 | 234.598 | 283.480 | 321.568 | 326.495 |
| Gd Oxide | 0.288 | 9.321 | 19.245 | 54.093 | 80.439 | 154.983 | 203.478 | 243.389 | 293.498 | 304.563 | 306.520 |
| Mn Oxide | 0.438 | 12.485 | 22.459 | 36.695 | 120.695 | 124.589 | 201.548 | [symbol] | 268.589 | 298.439 | 314.522 |
| No Contrast | 0.943 | 0.623 | 0.468 | 0.297 | 0.042 | 0.023 | 0.082 | 0.010 | 0.028 | 0.018 | 0.025 |
| Iron Oxide | 0.923 | 0.735 | 0.369 | 0.344 | 0.021 | 0.046 | 0.087 | 0.049 | [symbol] | 0.036 | 0.025 |
| Gd Oxide | 0.937 | 0.358 | 0.436 | 0.375 | 0.078 | 0.058 | 0.035 | 0.024 | 0.048 | 0.049 | 0.034 |
| Mn Oxide | 0.955 | 0.590 | 0.398 | 0.298 | 0.057 | 0.024 | 0.076 | 0.056 | 0.038 | 0.087 | 0.034 |

[symbol]

indicates data missing or illegible when filed

### Example 28

PEGDA liposome polymer matrices as described in example 8 were prepared. The polymer matrices were then lyophilized under the following conditions. Shelf loading temperature 0.degree. C. Product ramp time to freezing temperature 4.9 hr Shelf freezing temperature −50° C. Primary drying temperature −25° C. Primary drying pressure 75 mmHg Primary drying time 80.1 hr Secondary drying temperature 25° C. Secondary drying pressure 75 mmHg. Secondary drying time 25 hr.

For each 1 mL packed volume of microspheres, 50 mg of lyophilized doxorubicin (for storage lactose and parahydroxybenzoate may be added) in a solution of 10 mL was prepared. This solution was then added to the equilibrated heating source for 10 min. Next, the microspheres and 10 mL of doxorubicin were then combined. The mixture was then shaken vigorously and allowed to sit for at least 10 minutes in the heat source and then to minutes at room temperature before use. After this period the spheres can be stored for 24 hours at room temperature.

The release profile from the preparation in micrograms/mL is listed in the top panel of the table below and the resulting viability of HepG2 cells incubated with the solution is listed in the bottom panel of the table.

After storage of lyophilized spheres at room temperature for 0 hours prior to drug loading. Table 18

TABLE 18

| | 0 hrs | 6 hrs | 12 hrs | 1 day | 2 day | 3 day | 4 day | 5 day | 6 day | 7 day | 8 day | 9 day | 10 day | 11 day |
|---|---|---|---|---|---|---|---|---|---|---|---|---|---|---|
| No Contrast | [symbol] | [symbol] | [symbol] | [symbol] | [symbol] | [symbol] | [symbol] | [symbol] | [symbol] | [symbol] | [symbol] | [symbol] | [symbol] | [symbol] |
| Iron Oxide | [symbol] | [symbol] | [symbol] | [symbol] | [symbol] | [symbol] | [symbol] | [symbol] | [symbol] | [symbol] | [symbol] | [symbol] | [symbol] | [symbol] |
| Gd Oxide | [symbol] | [symbol] | [symbol] | [symbol] | [symbol] | [symbol] | [symbol] | [symbol] | [symbol] | [symbol] | [symbol] | [symbol] | [symbol] | [symbol] |
| Mn Oxide | [symbol] | [symbol] | [symbol] | [symbol] | [symbol] | [symbol] | [symbol] | [symbol] | [symbol] | [symbol] | [symbol] | [symbol] | [symbol] | [symbol] |
| No Contrast | [symbol] | [symbol] | [symbol] | [symbol] | [symbol] | [symbol] | [symbol] | [symbol] | [symbol] | [symbol] | [symbol] | [symbol] | [symbol] | [symbol] |
| Iron Oxide | [symbol] | [symbol] | [symbol] | [symbol] | [symbol] | [symbol] | [symbol] | [symbol] | [symbol] | [symbol] | [symbol] | [symbol] | [symbol] | [symbol] |
| Gd Oxide | [symbol] | [symbol] | [symbol] | [symbol] | [symbol] | [symbol] | [symbol] | [symbol] | [symbol] | [symbol] | [symbol] | [symbol] | [symbol] | [symbol] |
| Mn Oxide | [symbol] | [symbol] | [symbol] | [symbol] | [symbol] | [symbol] | [symbol] | [symbol] | [symbol] | [symbol] | [symbol] | [symbol] | [symbol] | [symbol] |

| | 12 day | 13 day | 14 day | 15 day | 16 day | 17 day | 18 day | 19 day | 20 day | 21 day |
|---|---|---|---|---|---|---|---|---|---|---|
| No Contrast | [symbol] | [symbol] | [symbol] | [symbol] | [symbol] | [symbol] | [symbol] | [symbol] | [symbol] | [symbol] |
| Iron Oxide | [symbol] | [symbol] | [symbol] | [symbol] | [symbol] | [symbol] | [symbol] | [symbol] | [symbol] | [symbol] |
| Gd Oxide | [symbol] | [symbol] | [symbol] | [symbol] | [symbol] | [symbol] | [symbol] | [symbol] | [symbol] | [symbol] |
| Mn Oxide | [symbol] | [symbol] | [symbol] | [symbol] | [symbol] | [symbol] | [symbol] | [symbol] | [symbol] | [symbol] |
| No Contrast | [symbol] | [symbol] | [symbol] | [symbol] | [symbol] | [symbol] | [symbol] | [symbol] | [symbol] | [symbol] |
| Iron Oxide | [symbol] | [symbol] | [symbol] | [symbol] | [symbol] | [symbol] | [symbol] | [symbol] | [symbol] | [symbol] |
| Gd Oxide | [symbol] | [symbol] | [symbol] | [symbol] | [symbol] | [symbol] | [symbol] | [symbol] | [symbol] | [symbol] |
| Mn Oxide | [symbol] | [symbol] | [symbol] | [symbol] | [symbol] | [symbol] | [symbol] | [symbol] | [symbol] | [symbol] |

[symbol]

indicates data missing or illegible when filed

After storage of lyophilized spheres at room temperature for 1 week prior to drug loading.

TABLE 19

| | 0 hrs | 6 hrs | 12 hrs | 1 day | 2 day | 3 day | 4 day | 5 day | 6 day | 7 day | 8 day | 9 day | 10 day | 11 day |
|---|---|---|---|---|---|---|---|---|---|---|---|---|---|---|
| No Contrast | [symbol] | [symbol] | [symbol] | [symbol] | [symbol] | [symbol] | [symbol] | [symbol] | [symbol] | [symbol] | [symbol] | [symbol] | [symbol] | [symbol] |
| Iron Oxide | [symbol] | [symbol] | [symbol] | [symbol] | [symbol] | [symbol] | [symbol] | [symbol] | [symbol] | [symbol] | [symbol] | [symbol] | [symbol] | [symbol] |
| Gd Oxide | [symbol] | [symbol] | [symbol] | [symbol] | [symbol] | [symbol] | [symbol] | [symbol] | [symbol] | [symbol] | [symbol] | [symbol] | [symbol] | [symbol] |
| Mn Oxide | [symbol] | [symbol] | [symbol] | [symbol] | [symbol] | [symbol] | [symbol] | [symbol] | [symbol] | [symbol] | [symbol] | [symbol] | [symbol] | [symbol] |
| No Contrast | [symbol] | [symbol] | [symbol] | [symbol] | [symbol] | [symbol] | [symbol] | [symbol] | [symbol] | [symbol] | [symbol] | [symbol] | [symbol] | [symbol] |
| Iron Oxide | [symbol] | [symbol] | [symbol] | [symbol] | [symbol] | [symbol] | [symbol] | [symbol] | [symbol] | [symbol] | [symbol] | [symbol] | [symbol] | [symbol] |
| Gd Oxide | [symbol] | [symbol] | [symbol] | [symbol] | [symbol] | [symbol] | [symbol] | [symbol] | [symbol] | [symbol] | [symbol] | [symbol] | [symbol] | [symbol] |
| Mn Oxide | [symbol] | [symbol] | [symbol] | [symbol] | [symbol] | [symbol] | [symbol] | [symbol] | [symbol] | [symbol] | [symbol] | [symbol] | [symbol] | [symbol] |

| | 12 day | 13 day | 14 day | 15 day | 16 day | 17 day | 18 day | 19 day | 20 day | 21 day |
|---|---|---|---|---|---|---|---|---|---|---|
| No Contrast | [symbol] | [symbol] | [symbol] | [symbol] | [symbol] | [symbol] | [symbol] | [symbol] | [symbol] | [symbol] |
| Iron Oxide | [symbol] | [symbol] | [symbol] | [symbol] | [symbol] | [symbol] | [symbol] | [symbol] | [symbol] | [symbol] |
| Gd Oxide | [symbol] | [symbol] | [symbol] | [symbol] | [symbol] | [symbol] | [symbol] | [symbol] | [symbol] | [symbol] |
| Mn Oxide | [symbol] | [symbol] | [symbol] | [symbol] | [symbol] | [symbol] | [symbol] | [symbol] | [symbol] | [symbol] |
| No Contrast | [symbol] | [symbol] | [symbol] | [symbol] | [symbol] | [symbol] | [symbol] | [symbol] | [symbol] | [symbol] |
| Iron Oxide | [symbol] | [symbol] | [symbol] | [symbol] | [symbol] | [symbol] | [symbol] | [symbol] | [symbol] | [symbol] |
| Gd Oxide | [symbol] | [symbol] | [symbol] | [symbol] | [symbol] | [symbol] | [symbol] | [symbol] | [symbol] | [symbol] |
| Mn Oxide | [symbol] | [symbol] | [symbol] | [symbol] | [symbol] | [symbol] | [symbol] | [symbol] | [symbol] | [symbol] |

[symbol]

indicates data missing or illegible when filed

After storage of lyophilized spheres at room temperature for 1 month prior to drug loading.

TABLE 20

| | 0 hrs | 6 hrs | 12 hrs | 1 day | 2 day | 3 day | 4 day | 5 day | 6 day | 7 day | 8 day | 9 day | 10 day | 11 day |
|---|---|---|---|---|---|---|---|---|---|---|---|---|---|---|
| No Contrast | [symbol] | [symbol] | [symbol] | [symbol] | [symbol] | [symbol] | [symbol] | [symbol] | [symbol] | [symbol] | [symbol] | [symbol] | [symbol] | [symbol] |
| Iron Oxide | [symbol] | [symbol] | [symbol] | [symbol] | [symbol] | [symbol] | [symbol] | [symbol] | [symbol] | [symbol] | [symbol] | [symbol] | [symbol] | [symbol] |
| Gd Oxide | [symbol] | [symbol] | [symbol] | [symbol] | [symbol] | [symbol] | [symbol] | [symbol] | [symbol] | [symbol] | [symbol] | [symbol] | [symbol] | [symbol] |
| Mn Oxide | [symbol] | [symbol] | [symbol] | [symbol] | [symbol] | [symbol] | [symbol] | [symbol] | [symbol] | [symbol] | [symbol] | [symbol] | [symbol] | [symbol] |
| No Contrast | [symbol] | [symbol] | [symbol] | [symbol] | [symbol] | [symbol] | [symbol] | [symbol] | [symbol] | [symbol] | [symbol] | [symbol] | [symbol] | [symbol] |
| Iron Oxide | [symbol] | [symbol] | [symbol] | [symbol] | [symbol] | [symbol] | [symbol] | [symbol] | [symbol] | [symbol] | [symbol] | [symbol] | [symbol] | [symbol] |
| Gd Oxide | [symbol] | [symbol] | [symbol] | [symbol] | [symbol] | [symbol] | [symbol] | [symbol] | [symbol] | [symbol] | [symbol] | [symbol] | [symbol] | [symbol] |
| Mn Oxide | [symbol] | [symbol] | [symbol] | [symbol] | [symbol] | [symbol] | [symbol] | [symbol] | [symbol] | [symbol] | [symbol] | [symbol] | [symbol] | [symbol] |

| | 12 day | 13 day | 14 day | 15 day | 16 day | 17 day | 18 day | 19 day | 20 day | 21 day |
|---|---|---|---|---|---|---|---|---|---|---|
| No Contrast | [symbol] | [symbol] | [symbol] | [symbol] | [symbol] | [symbol] | [symbol] | [symbol] | [symbol] | [symbol] |
| Iron Oxide | [symbol] | [symbol] | [symbol] | [symbol] | [symbol] | [symbol] | [symbol] | [symbol] | [symbol] | [symbol] |
| Gd Oxide | [symbol] | [symbol] | [symbol] | [symbol] | [symbol] | [symbol] | [symbol] | [symbol] | [symbol] | [symbol] |
| Mn Oxide | [symbol] | [symbol] | [symbol] | [symbol] | [symbol] | [symbol] | [symbol] | [symbol] | [symbol] | [symbol] |
| No Contrast | [symbol] | [symbol] | [symbol] | [symbol] | [symbol] | [symbol] | [symbol] | [symbol] | [symbol] | [symbol] |
| Iron Oxide | [symbol] | [symbol] | [symbol] | [symbol] | [symbol] | [symbol] | [symbol] | [symbol] | [symbol] | [symbol] |
| Gd Oxide | [symbol] | [symbol] | [symbol] | [symbol] | [symbol] | [symbol] | [symbol] | [symbol] | [symbol] | [symbol] |
| Mn Oxide | [symbol] | [symbol] | [symbol] | [symbol] | [symbol] | [symbol] | [symbol] | [symbol] | [symbol] | [symbol] |

[symbol]

indicates data missing or illegible when filed

After storage of lyophilized spheres at room temperature for 2 months prior to drug loading

TABLE 21

| | 0 hrs | 6 hrs | 12 hrs | 1 day | 2 day | 3 day | 4 day | 5 day | 6 day | 7 day | 8 day | 9 day | 10 day | 11 day |
|---|---|---|---|---|---|---|---|---|---|---|---|---|---|---|
| No Contrast | [symbol] | [symbol] | [symbol] | [symbol] | [symbol] | [symbol] | [symbol] | [symbol] | [symbol] | [symbol] | [symbol] | [symbol] | [symbol] | [symbol] |
| Iron Oxide | [symbol] | [symbol] | [symbol] | [symbol] | [symbol] | [symbol] | [symbol] | [symbol] | [symbol] | [symbol] | [symbol] | [symbol] | [symbol] | [symbol] |
| Gd Oxide | [symbol] | [symbol] | [symbol] | [symbol] | [symbol] | [symbol] | [symbol] | [symbol] | [symbol] | [symbol] | [symbol] | [symbol] | [symbol] | [symbol] |
| Mn Oxide | [symbol] | [symbol] | [symbol] | [symbol] | [symbol] | [symbol] | [symbol] | [symbol] | [symbol] | [symbol] | [symbol] | [symbol] | [symbol] | [symbol] |
| No Contrast | [symbol] | [symbol] | [symbol] | [symbol] | [symbol] | [symbol] | [symbol] | [symbol] | [symbol] | [symbol] | [symbol] | [symbol] | [symbol] | [symbol] |
| Iron Oxide | [symbol] | [symbol] | [symbol] | [symbol] | [symbol] | [symbol] | [symbol] | [symbol] | [symbol] | [symbol] | [symbol] | [symbol] | [symbol] | [symbol] |
| Gd Oxide | [symbol] | [symbol] | [symbol] | [symbol] | [symbol] | [symbol] | [symbol] | [symbol] | [symbol] | [symbol] | [symbol] | [symbol] | [symbol] | [symbol] |
| Mn Oxide | [symbol] | [symbol] | [symbol] | [symbol] | [symbol] | [symbol] | [symbol] | [symbol] | [symbol] | [symbol] | [symbol] | [symbol] | [symbol] | [symbol] |

| | 12 day | 13 day | 14 day | 15 day | 16 day | 17 day | 18 day | 19 day | 20 day | 21 day |
|---|---|---|---|---|---|---|---|---|---|---|
| No Contrast | [symbol] | [symbol] | [symbol] | [symbol] | [symbol] | [symbol] | [symbol] | [symbol] | [symbol] | [symbol] |
| Iron Oxide | [symbol] | [symbol] | [symbol] | [symbol] | [symbol] | [symbol] | [symbol] | [symbol] | [symbol] | [symbol] |
| Gd Oxide | [symbol] | [symbol] | [symbol] | [symbol] | [symbol] | [symbol] | [symbol] | [symbol] | [symbol] | [symbol] |
| Mn Oxide | [symbol] | [symbol] | [symbol] | [symbol] | [symbol] | [symbol] | [symbol] | [symbol] | [symbol] | [symbol] |
| No Contrast | [symbol] | [symbol] | [symbol] | [symbol] | [symbol] | [symbol] | [symbol] | [symbol] | [symbol] | [symbol] |
| Iron Oxide | [symbol] | [symbol] | [symbol] | [symbol] | [symbol] | [symbol] | [symbol] | [symbol] | [symbol] | [symbol] |
| Gd Oxide | [symbol] | [symbol] | [symbol] | [symbol] | [symbol] | [symbol] | [symbol] | [symbol] | [symbol] | [symbol] |
| Mn Oxide | [symbol] | [symbol] | [symbol] | [symbol] | [symbol] | [symbol] | [symbol] | [symbol] | [symbol] | [symbol] |

[symbol]

indicates data missing or illegible when filed

### Example 29

50 nm streptavidin coated gold nanoparticles were added at a 20% vol/vol concentration to a solution of poly(ethylene glycol)-diacrylate (PEGDA; Nektar Therapeutics, Huntsville, Ala.) in sterile phosphate-buffered saline (PBS; GIBCO Invitrogen, Carlsbad, Calif.) to make a 15% (w/v) hydrogel. Photoinitiator, Igracure D2959 (Ciba Specialty Chemicals, Tarrytown, N.Y.) was added to the polymer at a concentration of 0.05% (w/v). The mixture is then added drop-wise to a reactant medium made up of an inert bath. An inert bath includes but is not limited to a formulation of 160 grams Span 80 and 3,840 grams USP mineral oil. The inert bath is then stirred at a rate of 200 RPM in a reaction vat. The Vat was then exposed to UV irradiation for 5 minutes to initiate polymerization. Once reacted, the resultant crosslinked PEGDA sphere with the entrapped liposomes were cleaned of mineral oil and Span 8. The cleaning process involved dumping the resultant spheres over a 45-micron screen such that the beads were trapped on the screen while some of the reactants passed through. The spheres were washed three times as described above with water.

Microspheres were then incubated in a 200 micromolar biotin-fluoroscein solution for 10 minutes on a rocking platform at room temperature. Micropsheres were removed from the biotin-rhodamine containing solution and place in a normal saline solution and examined for fluorescence. Microspheres demonstrated high labeling efficiency with undetectable leaching of fluorescein from the spheres.

### Example 30

A macroporous PTFE membrane was sewn on a Palmaz balloon expandable stent from Cordis with a Ethilon nylon suture. The stent was loaded on a metal rod and placed in tubing slightly larger than the stent. The remaining open area between the stent wall and the tubing was filled with a solution containing 20% streptavidin coated gold nanoparticles (Nanocs) and poly(ethylene glycol)-diacrylate (PEGDA; Nektar Therapeutics, Huntsville, Ala.) in sterile phosphate-buffered saline (PBS; GIBCO Invitrogen, Carlsbad, Calif.) to make a 15% (w/v) hydrogel. Photoinitiator, Igracure D2959 (Ciba Specialty Chemicals, Tarrytown, N.Y.) was added to the polymer at a concentration of 0.05% (w/v). The solution was then polymerized with UV light for 1 minute. The inner metal rod and the outer tubing were then removed and the stent was incubated in a solution of fluorescein biotin. The microscopic fluorescence of the membrane before and after incubation in fluorescein biotin is shown in the Figure.

### Example 31

Lipid foams were prepared by dissolving lipids (DSPC:DSPG:CHOL (7:2:1 mol ratio)) mixed at a concentration of 100 mg lipid/ml final concentration into a chloroform:methanol:H 20 mixture (95:4:1 vol/vol). The solvent was then removed by vacuum evaporation and the resulting lipid foams were hydrated with a solution consisting of 100 mM Cu(gluconate) 2, 220 mM triethanolamine (TEA), pH 7.4. The resulting MLVs were extruded 10 times at 70° C. to generate large unilamellar vesicles.

To this liposome solution was added a concentrated solution of poly(ethylene glycol)-diacrylate (PEGDA; Nektar Therapeutics, Huntsville, Ala.) in sterile phosphate-buffered saline (PBS; GIBCO Invitrogen, Carlsbad, Calif.) to make a 15% (w/v) hydrogel. Photoinitiator, Igracure D2959 (Ciba Specialty Chemicals, Tarrytown, N.Y.) was added to the polymer at a concentration of 0.05% (w/v). The mixture is then added drop-wise to a reactant medium made up of an inert bath. An inert bath includes but is not limited to a formulation of 160 grams Span 80 and 3,840 grams USP mineral oil. The inert bath is then stirred at a rate of 200 RPM in a reaction vat. The Vat was then exposed to UV irradiation for 5 minutes to initiate polymerization. Once reacted, the resultant crosslinked PEGDA sphere with the entrapped liposomes were cleaned of mineral oil and Span 8. The cleaning process involved dumping the resultant spheres over a 45-micron screen such that the beads were trapped on the screen while some of the reactants passed through. The spheres were then washed with a solution containing 100 mM Cu(gluconate) 2, 220 mM triethanolamine (TEA), and 125 mM lactose.

Following polymerization and washing, the liposome polymer matrix underwent a buffer exchange to remove the external, unentrapped, Cu(gluconate) and TEA. This was accomplished by subjecting the microspheres to tangential flow ultrafiltration. This process removes water and small molecular weight solutes while retaining the microspheres. Using this process, the buffer external to the vesicles was replaced with 250 mM lactose/50 mM glycine.

For drug loading a procedure that could easily be implement in the pharmacy was employed. Specifically a water bath or dry heat block was allowed to equilibrate at 58° C. (55°-60° C.). For each 1 mL packed volume of microspheres, 50 mg of irinotecan in a solution of 10 mL was prepared. This solution was then added to the equilibrated heating source for 10 min. Next, the microspheres and 10 mL of irinotecan were then combined. The mixture was then shaken vigorously and allowed to sit for at least 10 minutes in the heat source and then to minutes at room temperature before use.

The release profile from the preparation in micrograms/mL is listed in the top panel of the table below and the resulting viability of HepG2 cells incubated with the solution is listed in the bottom panel of the table.

TABLE 22

| | 0 hrs | 6 hrs | 12 hrs | 1 day | 2 days | 3 days | 4 days | 5 days | 6 days | 7 days | 8 days |
|---|---|---|---|---|---|---|---|---|---|---|---|
| No Contrast | 0.617 | 8.857 | 23.496 | 61.345 | 95.438 | 112.594 | 199.459 | 204.592 | [symbol] | 345.298 | 352.335 |
| Iron Oxide | 0.812 | 10.859 | 16.596 | 42.489 | 80.598 | 134.414 | 235.595 | 210.458 | 224.598 | 320.433 | 322.371 |
| Gd Oxide | 0.553 | 14.584 | 18.456 | 58.485 | 79.321 | 108.594 | 230.416 | [symbol] | 258.486 | 295.453 | 298.453 |
| Mn Oxide | 0.328 | [symbol] | 24.569 | 50.486 | 111.205 | 109.456 | 202.495 | 265.985 | 275.857 | 320.597 | 322.929 |
| No Contrast | 0.947 | 0.650 | 0.488 | 0.385 | 0.068 | 0.049 | 0.038 | 0.028 | 0.085 | 0.035 | 0.057 |
| Iron Oxide | 0.958 | 0.622 | 0.575 | 0.289 | 0.038 | 0.035 | 0.048 | 0.048 | 0.049 | 0.057 | 0.028 |
| Gd Oxide | 0.935 | 0.649 | 0.457 | 0.284 | 0.048 | 0.089 | 0.038 | 0.056 | 0.039 | 0.026 | 0.039 |
| Mn Oxide | 0.959 | 0.687 | 0.587 | 0.185 | 0.098 | 0.038 | 0.016 | 0.039 | 0.055 | 0.031 | 0.057 |

[symbol]

indicates data missing or illegible when filed

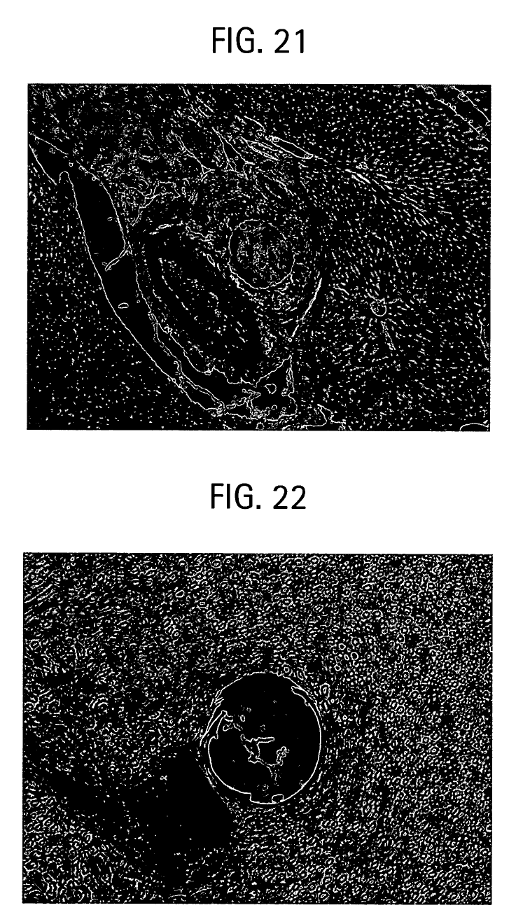

FIG. 21 shows H&E stained histological section of embolized bland manganese oxide loaded chemosphere. The chemospere demonstrates high biocompatibility with little sign of inflammation.

FIG. 22 shows H&E stained histological section of embolized bland manganese oxide loaded chemosphere. The chemosphere demonstrates high biocompatibility with little sign of inflammation.

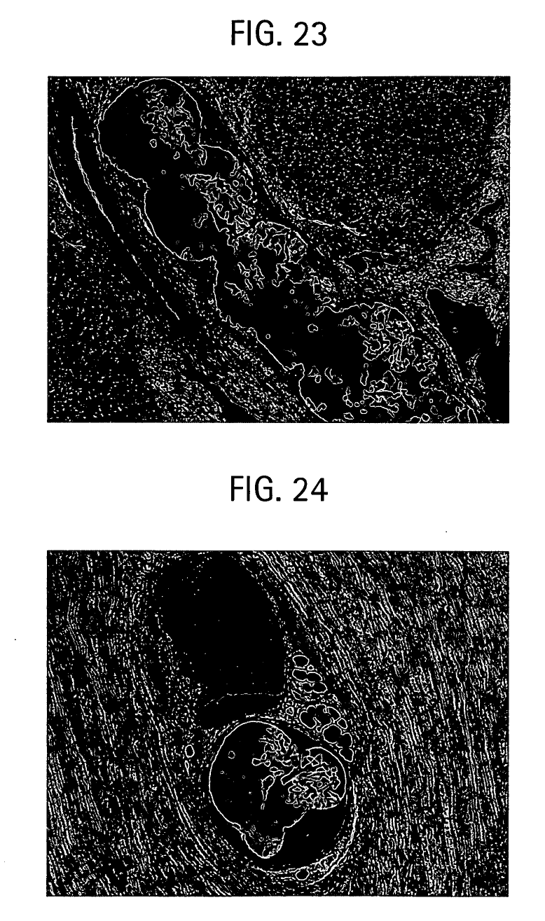

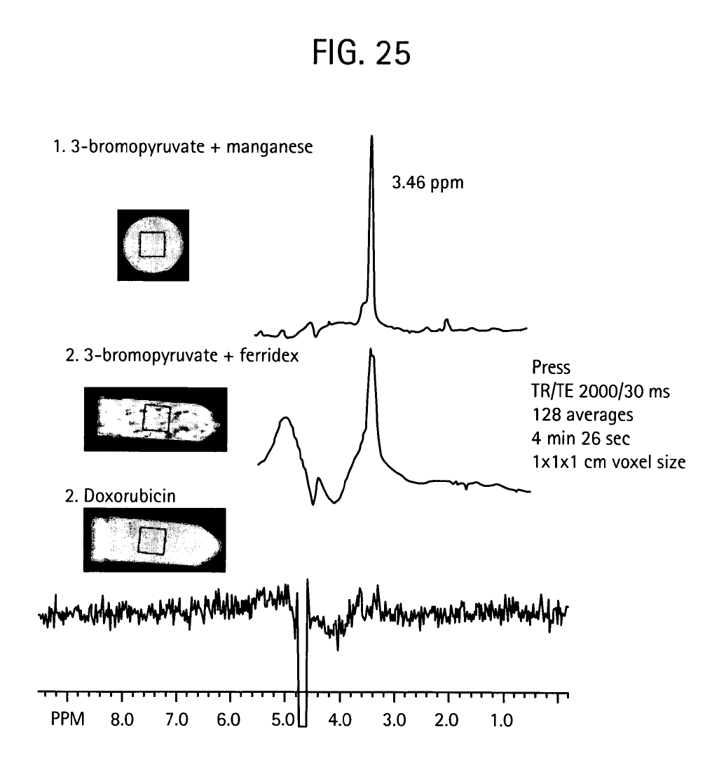

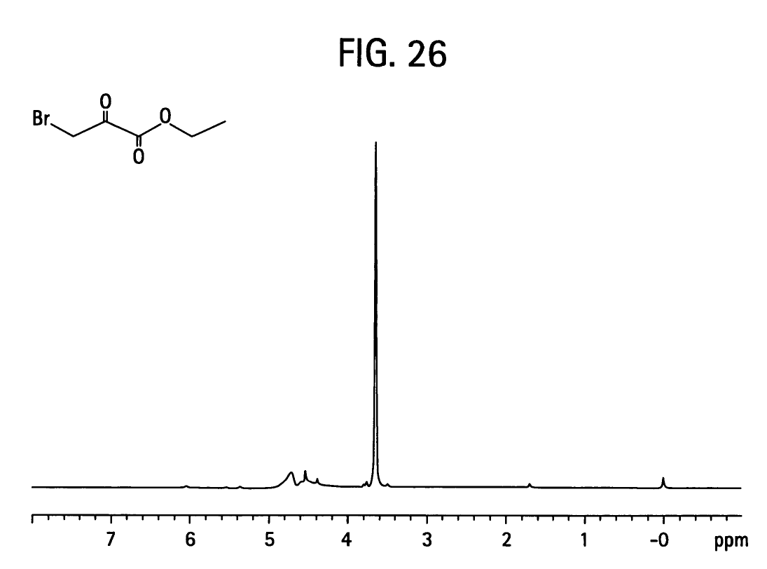

FIG. 23 shows H&E stained histological section of iron oxide loaded chemospheres in vessel. The chemospheres demonstrate compressibility and serial embolization of adjacent spheres

FIG. 24 shows H&E stained histological section of iron oxide loaded chemosphere. The chemosphere demonstrates mild inflammation surrounding the site of embolization.

### Example 32

### Microsphere Embolization

Tubal Embolization

The optimal setup for performing fluoroscopy guided transcervical tubal embolization includes an all-purpose fluoroscopy room with a rotating c-arm. The use of lithotomy stirrups is highly recommended. A kit for sterilely preparing the perineum and vagina and draping the legs and abdomen is necessary. Either a metal speculum that can be resterilized after use or a disposable plastic speculum can be used to visualize and access the cervix. A transcervical catheter is needed; we use a 12 French catheter with a 5 French inner diameter and a side arm (Cook, Bloomington Ind.) for injecting contrast and saline. A sterile cervical tenaculum for traction and sterile set of disposable cervical dilators (Cook) should be available for every case but may not necessarily be needed. Long-handle sponge forceps are useful to keep the vaginal vault dry during and after the procedure. Iodinated contrast (Visipaque 320; Amersham Health, Princeton, N.J.) is used to perform the initial hysterosalpinogram (HSG) with selective salpingograms, if necessary. A 40-cm angled-tip 4 French catheter (Cook) is preferred for the selective salpingograms.

This procedure can be performed under intravenous conscious sedation (IVCS) (ie, midazolam and fentanyl) or with local anesthetic utilizing paracervical block anesthesia. If a paracervical block without IVCS is used for pain control during placement, several anatomic factors should be considered. The uterovaginal plexus lies predominantly lateral and posterior to the junction of the uterus and cervix. The cardinal ligaments transmit uterine nerves at the 3 and 9 o'clock positions and similarly, the uterosacral ligaments transmit nerves at the 5 and 7 o'clock positions. While injections of 1% lidocaine at the 3, 5, 7, and 9 o'clock positions at the cervicovaginal junction place the local anesthetic adjacent to the appropriate nerves, the injections at the 3 and 9 o'clock positions along the cervix risk entering the uterine arteries or veins that are present in the neurovascular bundles. Lidocaine injections with 3 to 5 mL at the 4 and 8 o'clock or 5 and 7 o'clock positions are recommended to maximize anesthesia and minimize risk to adjacent vessels.

The procedure begins with the patient in the lithotomy stirrups on the angiography table. The vulvar and perineal areas are sterilely prepared with an iodine-based solution followed by the placement of sterile drapes over the legs and the abdomen. A sterile speculum is placed in the vaginal vault and the cervix identified. Both the vaginal vault and cervix are prepared with the iodine solution. A 12F-balloon cannula is used to access the uterine cavity transcervically. The internal balloon is inflated to seal against contrast leakage. A cervical tenaculum can be used if needed for traction on the uterus. An HSG is performed with an injection of contrast through the cannula sidearm. If neither or only one of the fallopian tubes is identified on the HSG, a 4 F angled-tip catheter is used for selective salpingogram(s). Through the 4F catheter chemospheres ranging from 50 to 200 micrometers are injected. In some instances it may be optimal to first embolize with chemospheres that contain a sclerosing agent such as sodium morhuate that is incorporated into liposomes. In other cases bland embolization may be preferable. To ensure proper embolization a repeated salpingogram is performed and the presence of retrograde contrast away from the embolization site is ensured. In some instances embolization may be followed by coil deployment to ensure spheres remain embolized. One example of a suitable coil to follow embolization is the Ensure device.

Following the embolization procedure a repeat procedure should be performed on the contralateral fallopian tube. The internal balloon can then be deflated and the cervical cannula is then removed from the patient. The long-handle sponge forceps are used if needed to clean the vaginal vault of contrast and/or blood. The patient is taken to a holding or observation area for recovery and monitoring by a nurse postprocedure.

The patient and her significant other are instructed to use another form of contraception until correct coil position and tubal occlusion can be documented by HSG at the 3 month follow-up appointment. Alternate forms of birth control do not include a condom alone (it must be used in combination with another method) or an intrauterine method (ie, intrauterine device or intrauterine system, these may cause problems with the coils).

In addition to embolization with chemospheres in some instances embolization with chemogel may be preferable. In this instance a 4F fiberoptic catheter as described for aneurysm embolization should be employed. Through the lumen liquid PEGDA with photoinitiator is injected. In certain instances an inflatable balloon cuff around the fiberoptic catheter is required to prevent flow of unpolymerized PEGDA towards the uterus. In this instance a hydrogel plug as opposed to multiple microspheres is formed in the fallopian tube.

In other instances dissolvable polymers such as alginate that can be dissolved by alginate lyase/edta may be preferable to deliver in either microsphere or gel form to enable selective dissolution of embolization and the possibility of pregnancy.

Exemplary techniques are described, for example, by McSwain et al (Techniques in Vascular and Interventional Radiology Volume 9, Issue 1, March 2006, Pages 24-29), incorporated by reference in its entirety herein.

Transcatheter Arterial Embolization

Embolization of the prostate can be performed according to methods described in the art, preferably using chemospheres approximately 300 to 500 micrometers in size. Sun et al. (Radiology: Volume 246: Number 3, March 2008), incorporated by reference in its entirety herein, describe methods of transcatheter arterial embolization (TAE) of the prostate.

### Example 33

Doxorubicin or calcein containing liposome were first suspended in 2% w/v ultrapurified sodium PROTANAL HF alginate (FMC Biopolymers) and 20% vol/vol MnO or GdO nanoparticle solution (Reade, R.I.) in normal saline (5% MnO or GdO). This solution was passed through an electrostatic droplet generator at a flow rate of about 200 μl/min. Droplets were collected in a Petri dish containing 100 mM CaCl2 and then washed three times in saline.

Vx-2 Tumor Model

The institution's Animal Care Committee approved the study, and experiments were done in accordance with institutional guidelines. For the purposes of this study, which was to study a new drug delivery system in liver tumors, the rabbit Vx-2 tumor model, a known model of hypervascular tumor (Geschwind, et al. JVIR 2000), was selected for implantation in the liver because of the similarities of its blood supply to that of human liver tumors. The tumors were allowed to grow for another 10 to 14 days, at which time they reached an ellipsoidal shape with dimensions ranging between 2.5 and 3.5 cm

Intra-Arterial Administration of EmboCaps

Preanesthetics were administered, and anesthesia was carried out as described above. Transcatheter hepatic artery administration of the EmboCaps was done under fluoroscopy. The animals were brought to the angiography suite and intubated using a size 3.0-mm endrotracheal tube (Mallinkrodt Medical, St. Louis, Mo.) but not ventilated. Surgical cut down was done to gain access into the common femoral artery, after which a 4-French sheath (Cook, Inc., Bloomington, Ind.) was placed. A specially manufactured 2-French catheter with a tip in the shape of a hockey stick (JB1 catheter; Cook) was manipulated into the celiac axis, after which a celiac arteriogram was done to delineate the blood supply to the liver and confirm the location of the tumor. The tumor could readily be visualized as a region of hypervascular blush located on the left side of the liver near the gastric fundus. The left hepatic artery, which nearly exclusively provided flow to the tumor, was then selectively catheterized off the common hepatic artery with the aid of a steerable guide wire (0.010-0.014 in. Transsend wire; Boston Scientific Oncology, Natick, Mass.). After having ensured adequate positioning of the catheter, the EmboCaps were carefully infused directly into the artery. After completion of the infusion, the catheter was removed, and the common femoral artery was ligated using resorbable suture material to obtain hemostasis. The animals were returned to cages and followed-up daily until their respective times of sacrifice.

3T MR Imaging

For assessment of EmboCap contrast, EmboCaps were suspended in 2% agarose. Phantom imaging was performed on a Siemens Trio 3T scanner with a clinical knee coil. For T1 weighted FFE sequence which provides hyperintense contrast, the imaging pararamaters were: repetition time (TR)=25 ms; echo time (TE)=3.2 ms; flip angle (FA)=15′; field of view (FOV)=22 cm; matrix=512×512; slice thickness=1 mm. Identical scanner, coil and imaging parameters were used for in vivo imaging of the rabbit VX-2 tumor post embolization.

MR Imaging of EmboCaps in Rabbit

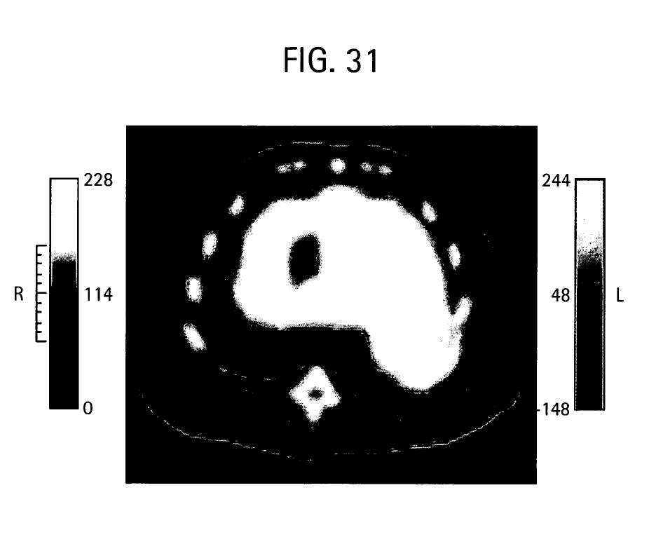

Doxorubicin and MnO or GdO containing EmboCaps transarterially infused into the kidney and hepatic VX-2 tumor were readily visualized. Signal foci corresponding to EmboCaps tended to aggregate more peripherally in the renal cortex (FIG. 31). For VX-2 tumor embolization, non-target embolization was minimized by a slow rate of particle injection. Lack of significant non-target embolization with this technique was suggested by the fact that positive contrast was well localized at the site of the tumor (FIG. 31). FIG. 31 shows Rabbit Vx-2 PET/CT scan 24 hrs after embolization with 3 BromoPyruvate loaded iron oxide PEGDA chemospheres.

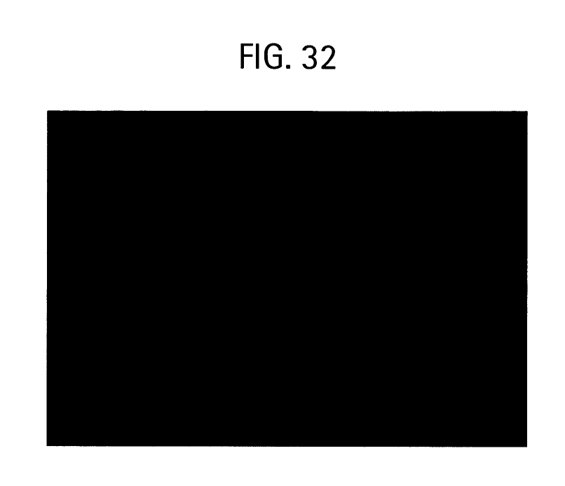

FIG. 32 shows PEGDA microspheres containing streptavidin coated gold nanoparticles incubated in biotin-4-fluorescein indicates proof of principle labeling of spheres with biotin conjugated therapeutic agent (ie yttrium-90 biotin).

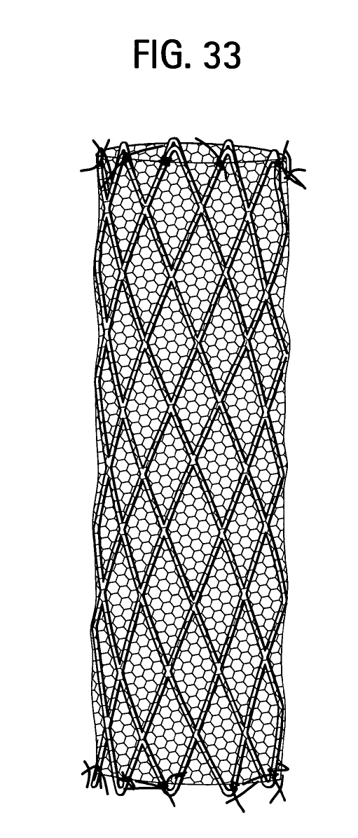

FIG. 33 shows a stent coated with a macroporous PTFE membrane. By polymerizing hydrogel containing streptavidin coated nanoparticles on the membrane or alternatively directly on struts such a stent is loaded with therapeutic agent conjugated to biotin.

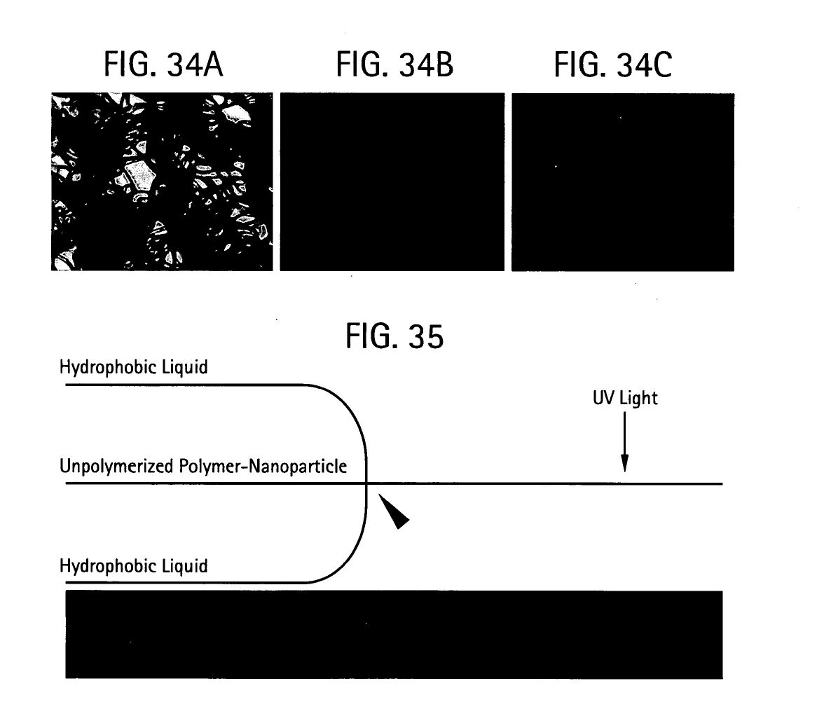

FIG. 34 (A-C) shows (A) macroscopic image of pegda containing streptavidin coated gold nanoparticles polymerized on porous stent-graft pictured in FIG. 33. (B)—Auto-fluorescence of membrane prior to incubation with biotin-4-fluorescein. (C) Fluorescence of stent membrane after incubation with biotin-4-fluorescein shows high labeling efficiency. Proof of principle that such a strategy could be employed to make a loadable stent or other device decorated with streptavidin.

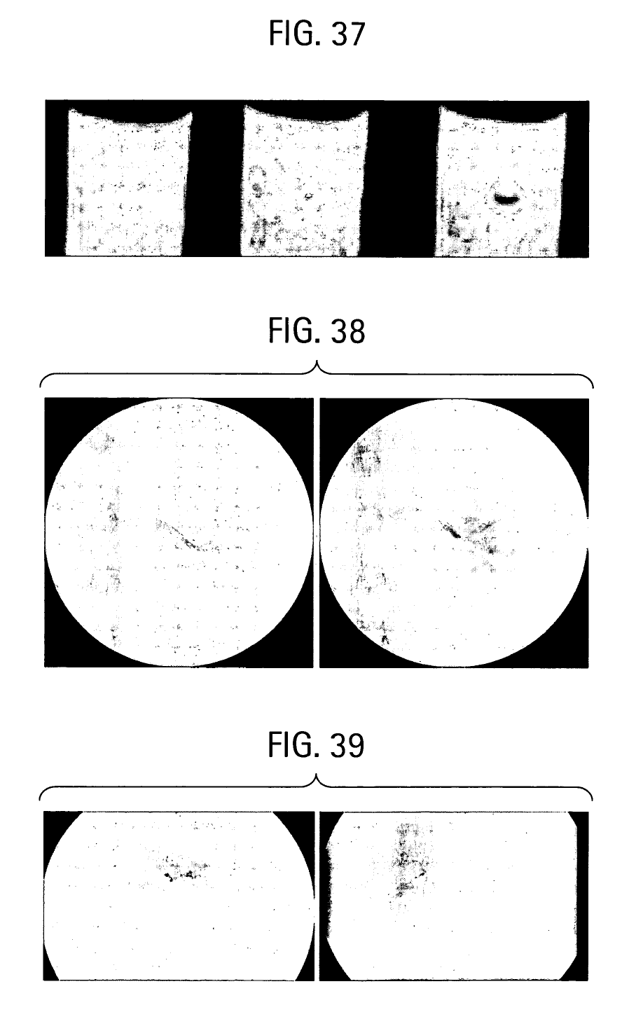

FIG. 37 shows gadolinium oxide loaded pegda chemospheres loaded in gelatin loaded 50 mL tube. Concentration of gadolinium oxide per sphere must be optimized to ensure solely hyperintense signal on standard T1 weighted scan. Note in the second and third gelatin phantoms from the left a hypointense center surrounded by a hyperintense rim whereas in image at far right only a hyperintense image is seen.

### Example 34

Unpolymerized hydrogel solutions solutions were pumped using a 100 microliter Hamilton Gastight syringes (1700 series, TLL) or 50 microliters SGE gastight syringe. Mineral Oil was pumped using 1 mL Hamilton Gastight syringes (1700 series, TLL). The syringes were attached to the microfluidic device by means of HamiltonTeflon needles (30 gauge, 1 hum). Syringe pumps from Harvard Apparatus (Nanomite syringe pump) and Razel (precision syringe pump) were used to infuse the aqueous hydrogel solution and the oil. Oils and aqueous unpolymerized hydrogel solutions were pumped through devices at volumetric flow rates ranging from 0.1 μL/min to about 10.0 μL/min.

Oils used included mineral oil (sigma-aldrich), perfluorocarbon (PFC), e.g., perfluoro-15-crown-5-ether (PFCE), perfluorooctylbromide (PFOB) perfluorodecaline (mixture of cis and trans, 95%, Acros Organics), perfluoroperhydrophenathrene (tech, Alfa-Aesar) or 1H, 1H, 2H, 2H-perfluorooctanol (98%, Alfa-Aesar). perfluorocarbons (PFCs). Representative perfluorocarbons include bis(F-alkyl)ethanes such as F-44E, i-F-i36E, and F-66E; cyclic fluorocarbons, such as F-decalin, perfluorodecalin or “FDC), F-adamantane (“FA”), F-methyladamantane (“FMA”), F-1,3-dimethyladamantane (“FDMA”), F-di- or F-trimethylbicyclo[3,3,1]nonane (“nonane”); perfluorinated amines, such as F-tripropylamine (“FTPA”) and F-tri-butylamine (“FTBA”), F-4-methyloctahydroquinolizine (“FMOQ”), F-n-methyl-decahydroisoquinoline (“FMIQ”), F-n-methyldecahydroquinoline (“FHQ”), F-n-cyclohexylpurrolidine (“FCHP”) and F-2-butyltetrahydrofuran (“FC-75” or “RM101”). Brominated perfluorocarbons include 1-bromo-heptadecafluoro-octane (sometimes designated perfluorooctylbromide or “PFOB”), 1-bromopenta-decafluoroheptane, and I-bromotridecafluorohexane (sometimes known as perfluorohexylbromide or “PFHB”). PFOB is a preferred labeling agent for use in the methods of the invention. Other brominated fluorocarbons are disclosed in U.S. Pat. No. 3,975,512. Other suitable perfluorocarbons are mentioned in EP 908 178 A1. The experiments were typically performed using 10:1 mixtures of perfluorodecaline and 1H, 1H, 2H, 2H-perfluorooctanol.

Commercially available fluorinated such as fluoroinert liquids (3M, St. Paul Minn) can also be used. Further in certain instances, especially to reduce toxicity in the case of cell encapsulation it may be optimal to attach a hydrophilic head group such as PEG or DMP to the fluorinate carrier (ie PEG-PFPE, DMP-PFPE).

Surfactants were also used to reduce shear force include the use of Tween 20, Span 80 and Zonyl (Dupont, Wilmington). Fluorinated surfactants, such as those with a hydrophilic head group, are preferred when the carrier-fluid is a fluorinated fluid and the plug-fluid is an aqueous solution. This use of proper surfactant allows for formation of microspheres that do not stick to the walls of the microfluidic channel.

Microspheres are formed in the oil carrier fluid stream by modifying the relative pressure exerted at say a T junction as depicted in FIG. 35. The shear force exerted on the aqueous unpolymerized hydrogel solution causes unpolymerized microspheres to be carried by the inert oil carrier fluid stream. Once in the inert stream the carrier fluid with unpolymerized microspheres is exposed to conditions that will induce polymerization in the hydrogels. In the case of PEGDA microspheres, poly(ethylene glycol)-diacrylate (PEGDA; Nektar Therapeutics, Huntsville, Ala.) in sterile phosphate-buffered saline (PBS; GIBCO Invitrogen, Carlsbad, Calif.) to make a 15% (w/v) hydrogel. Photoinitiator, Igracure D2959 (Ciba Specialty Chemicals, Tarrytown, N.Y.) is added to the polymer at a concentration of 0.05% (w/v) comprises the aqueous phase.

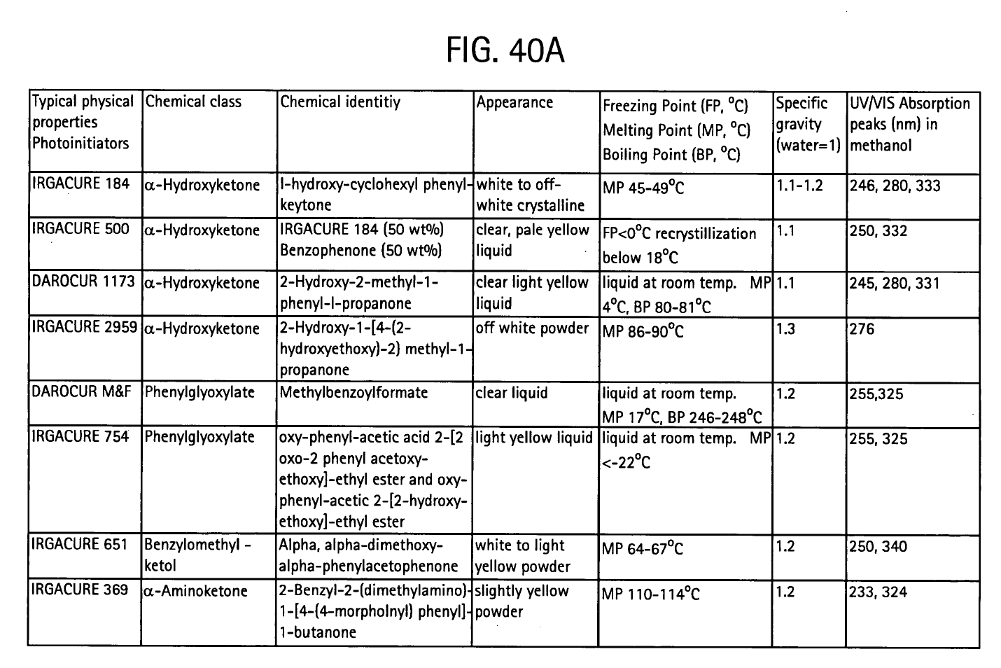

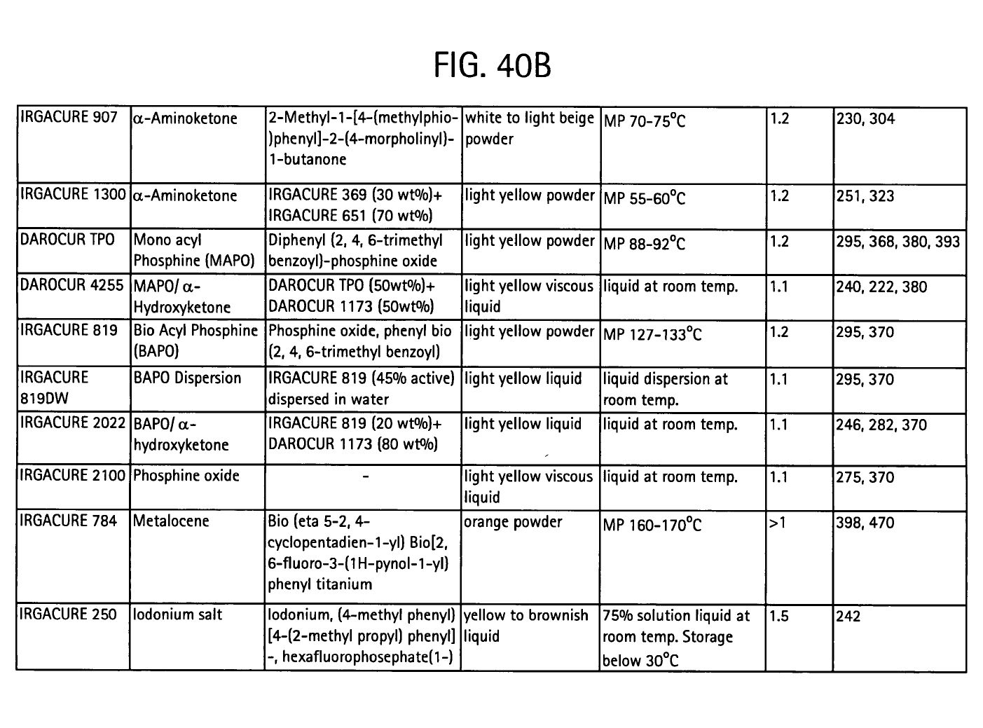

To induce polymerization the inert carrier fluid with unpolymerized microspheres is channel into a quartz or PDMS polymerization chamber or tubing such as PTFE tubing that enable passage of UV light should be utilized. Microspheres can be polymerized with the oil phase stationary or in certain instances it is preferable to induced polymerization in a spiral polymerization chamber that provides for constant movement of the inert fluid containing spheres within the path of UV light exposure. For optimal polymerization, 1 to 10 minutes of UV light exposure should be employed to induce polymerization of the microspheres. The choice of UV light is dependent on the choice of photoinitator. Examples of suitable photoinitaors all available from Ciba include Darocur 1173, Darocur BP, Darocur MBF, Igracure 500, Irgacure 651, Irgacure 369, Irgacure 907, Irgacure 1300, Irgacure 784, Irgacure 819, Darocur 4265, Irgacure 250. For each of the optimal wavelength for excitation see the Table in FIG. 40 as published by Ciba.

### Example 35

Any material that is isodense with the aqueous phase can readily be incorporated with such a microfluidic setup by simply mixing this material with the aqueous hydrogel phase prior to polymerization. The isodense nature of such materials is required to prevent rapid settling of the agent to the lower edge of the unpolymerized aqueous microsphere. We specifically incorporated in the microsphere compartment HepG2 cells, MIN 6 cells, Ins 1 cells and CHO cells. All when mixed with poly(ethylene glycol)-diacrylate (PEGDA; Nektar Therapeutics, Huntsville, Ala.) in sterile phosphate-buffered saline (PBS; GIBCO Invitrogen, Carlsbad, Calif.) to make a 15% (w/v) hydrogel with Photoinitiator, Igracure D2959 (Ciba Specialty Chemicals, Tarrytown, N.Y.) added to the polymer at a concentration of 0.05% (w/v) could be readily entrapped in the PEGDA microspheres. A range of seeding densities including 10 cells per mL, 50 cells per mL, 500 cells per mL, 1,000 cells per mL, 2,000 cells per mL, 4,000 cells per mL, 8000 cells per mL, 15,000 cells per mL, 30,000 cells per mL, 50,000 cells per mel, 100,000 cells per mL or 200,000 cells per mL of aqueous unpolymerized PEGDA was explored in order to entrap from 1-20 cells per 50 micrometer chemosphere and 1-3000 cells per 200 micrometer chemosphere.

### Example 36

As will be obvious to one skilled in the art for generation of 200 micrometers microspheres, 100 micrometer microspheres, 70 micrometer microspheres and 50 micrometer microspheres different microfluidic devices are needed. For example to generate microspheres with size of approximately 70±10 μm an etch depth of 50 micrometers is required in the microfluidic chip. To make microspheres of approximately 50 micrometers an etch depth of 36 micrometers and channel depth of 41 micrometers is required for the channel that releases PEGDA (see FIG. 39). By altering the etch depth and the size of channels in the microfluidic device the size of spheres can be altered. To make large spheres a macrofluidic device is required with etch depth of approximately 150 micrometers. The size of the resulting microspheres is also dependent on the flow rate ratio, viscosity of fluids and the quantity of surfactant in the fluids. In general the use of a microfluidic droplet generator enables formation of spheres with far less range in size as compared to traditional vat style synthesis in which aqueous polymer is dripped into a agitated bath of organic carrier fluid. The uniformity of size found with microfluidic devices is largely due to the equal pressure exerted on microspheres in a microfluidic channel as compared into a large volume vat (ie forces at outer perimeter of vat is quite different than force at center of vat. For this reason, microfluidic droplet generation enables formation of uniform sized spheres and abrogates the need for a sieve size exclusion step to properly “size” the particles. Further such technology enables a much tighter size distribution than is currently available commercially.

### Example 37

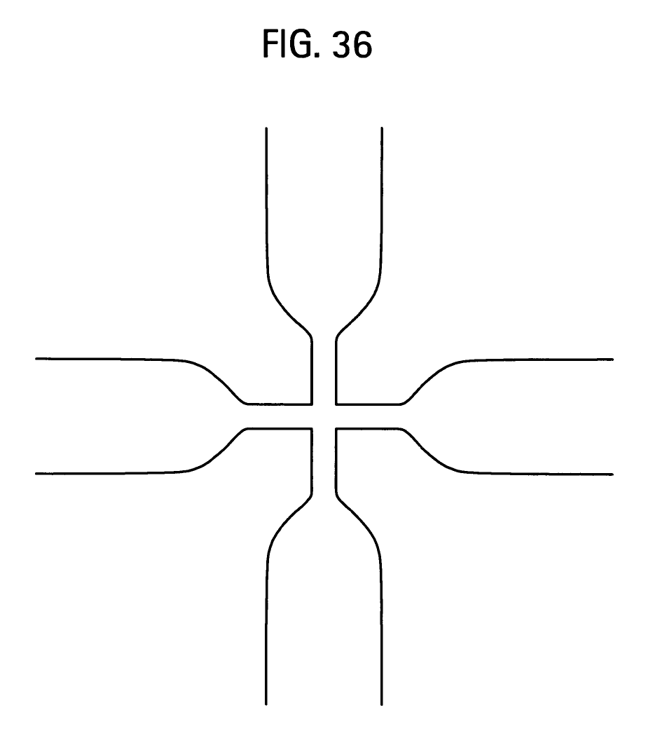

In addition to cells liposomes and colloidal contrast agents are isodense with the aqueous polymer phase and can be readily incorporated into microfluidic generated microspheres. In this example we incorporated liposomes synthesized with Phosphatidylcholine (Avanti Polar Lipids, Alabaster, Ala.) and cholesterol (Sigma, St. Louis, Mo.) in a 1:1 mole ratio in chloroform were dried in a rotary evaporator. The lipid film was solvated and annealed for 2 hours at 55 C with a solution of fluorsceiine (Sigma, St. Louis, Mo.). In order to form vesicles of a specific size, the lipid suspension was taken through twenty-one cycles of extrusion (LiposoFast, Avestin, Ottawa, Ontario, Canada) through two stacked polycarbonate filters (100 nm or 400 nm pore size). Unencapsulated drug was removed using size exclusion chromatography with sephadex G-50 resin (Sigma, St. Louis, Mo.). All buffer solutions used were filtered with 0.22 μm filters just prior to vesicle preparation. Liposomes were then mixed in poly(ethylene glycol)-diacrylate (PEGDA; Nektar Therapeutics, Huntsville, Ala.) in sterile phosphate-buffered saline (PBS; GIBCO Invitrogen, Carlsbad, Calif.) to make a 15% (w/v) hydrogel with Photoinitiator, Igracure D2959 (Ciba Specialty Chemicals, Tarrytown, N.Y.) added to the polymer at a concentration of 0.05% (w/v). This solution was loaded into a glass microfluidic device with dimensions as shown in FIG. 36. Spheres approximately 50 micrometer were formed in the droplet generator and were forced into PTFE tubing with inner diameter of 70 micrometers and was exposed to UV light for 5 minutes to induce polymerization.

### Example 38

For NMR samples of 3 Bromopyruvate either eluted from spheres or found in rabbit. All spectra were collected on a 500 MHz Varian spectrometer in a triple-axis probe equipped with z-gradients. To each sample, DSS and 5% D2O was added to each sample for chemical shift referencing and locking respectively. Temperature was held at 37 C. A simple spin-echo with presaturation for watersupression was used. Spectra were processed with zero-filling and 1.5 Hz linebroadening.

### Example 39

### MR Spectroscopy of 3 Bromopyruvate Loaded Chemospheres

MR spectroscopy of 3 Bromo Pyruvate loaded chemospheres was carried out. 8-channel Siemens Knee Coil were used in the following experiments. Samples were provided in 50 cc centrifuge tubes as follows.

### Sample #1.

Manganese oxide+200 mg/ml polymer+liposome+200 mg 3-bromopyruvate+gelatin, 40 mL total volume, MW 166.95
->3-BP concentration=(200/166.95)/0.04=29.95 mM
Scan Parameters: 13×25×13 mm=4.225 cm3, TR/TE 2000/30 ms, 128 averages scan time 4 min 26 sec. Water suppression 75 Hz BW. Remove oversampling checked.
TE 140 13×25×13 mmTE 30 10×10×10 mm (=1 cc) No water suppression (8 averages)

### Sample #2.

Same as #1, except feridex instead of MnO. 20% by volume 11.2 mg Fe+5.6−9.1 mg dextran/mL TE=30 ms, 13×25×13 mm 128 averages TE=30 no water suppression (8 averages) TE=30 ms, 10×10×10 mm 128 averages TE=30 no water suppression (8 averages)

### Sample #3.

50 mM doxorubicin (adriamycin) TE=30 ms, 13×25×13 mm 128 averages, TE=30 no water suppression (8 averages)

### Example 40

### Injection of Alginate or PEGDA Microspheres

One objective of inserting or implanting a filler agent into a tissue, for example into the skin, is either to fill an area in which there is currently a deficit of material that should normally be present, or to produce a desired structural change. There are various known filler materials, such as collagen, elastin, fibrillin, fibulin, decorin, biglycan, hyaluronic acid, calcium hydroxyapatite, silicone, cells, and poly L-lactic acid, which have been used to augment the skin and treat various cosmetic conditions. When injected into the skin, however, these materials are often not removable or correctable at will in case the implantation procedure is not carried out as intended. This can result in unwanted bumps or other types of protrusions, which are difficult, if not impossible, to remedy without surgical intervention.

Here, alginate or PEGDA microspheres, preferably of a size 50 micrometer and smaller, are injected as a dermal filler. Preferably, alginate or PEGDA is injected in the form of beads.

Approximately 300-500 mg of the beads that are preferable about 50 micrometers in diameter are suspended in an appropriate solution (for example, 1 ml of distilled water). The beads are also be suspended in a delivery vehicle, for example 50% (v/v) concentration of chondroitin sulfate (CS) proteoglycan:elastin (between 1:3 and 3:1) that carries approximately 500 mg of the beads and is delivered to a tissue site. Upon injection into a tissue site, these microparticles occupy space within the tissue and change the shape, texture, or morphology of the tissue.

In certain cases the tissue filler materials can be colorless, transparent, or can include a color to mimic the surrounding tissue. In certain situations, it may be desirable to include a chromophore or pigment within the microparticles that either mimics the color of the tissue to be implanted or a color that has a desired cosmetic appearance, such as red, blue, or black. Cosmetic pigments of many different varieties and colors are known and can be used herein.

### Methods

The invention was carried out using, but not limited to, various methods described below.

Liposome Release Profile

The release profile of liposome polymer matrices of various formulations and storage conditions was assessed by measuring the fluorescene and tumor kill of the solution eluted. HepG2 cells were cultured in EMEM substituted with 2 mM L [symbol] Glutamine, 1 mM Sodium pyruvate, 0.1 mM non-essential amino acids, 1.5 g/L sodium bicarbonate and 10% FBS in a humidified CO2 incubator at 37° C. and a 5% CO2 atmosphere. Cells were cultured in tissue culture plates and culture media was replaced every 3 days. Hydrated loaded beads (100 mg) were weighed into a 15 mL conical tube and 10 mL saline was added. The sample was placed in an incubator shaker at 37 degrees C. and 100-200 rpm. At selected time intervals, the sample was removed from the incubator, centrifuged (eppendorf, 1000 rpm, 2 min) and 100 microliters of supernatant was removed and replaced with 100 microliters of fresh saline.

Assessing Tumor Kill

Tumor kill of the eluted solution was tested on the Hep-G2 cell line. Following drug exposure, MTT reagent (1 mg/mL phosphate buffered salt solution) is added to each well at a volume of 50 [IL per well and incubated for 4 hours. The well contents are then aspirated and 150 [IL of dimethylsulfoxide (DMSO) is added to each well to disrupt the cells and to solubilize the formazan precipitate within the cells. The 96-well plates are shaken on a plate shaker for a minimum of 2 minutes, and read on a microplate spectrophotometer set at a wavelength of 570 nm. The optical density (OD) readings are recorded and the OD values of the blank wells containing media alone are subtracted from all the wells containing cells. The cell survival following exposure to agents is based as a percentage of the control wells cells not exposed to drug. All wells are performed in triplicate and mean values are calculated.

For irinotecan fluorescence measurements, the excitation wavelength was set at 400 nm and the emission scans were obtained from 425 nm to 650 nm. The slits were set at 2.5 nm. Measurements were made at ambient temperature using a quartz cell with a 1 cm path length. For doxorubicin fluorescence a krypton-argon laser line (488 nm) was employed for excitation.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

### REFERENCES

1. Williams C G, Kim T K, Taboas A, Malik A, Manson P, Elisseeff J. In vitro chondrogenesis of bone marrow-derived mesenchymal stem cells in a photopolymerizing hydrogel. Tissue Eng. 2003 August; 9(4):679-88.
2. Elisseeff J, Anseth K, Sims D, McIntosh W, Randolph M, Langer R. Transdermal photopolymerization for minimally invasive implantation. Proc Natl Acad Sci USA. 1999 Mar. 16; 96(6):3104-7.

1. A polymer matrix comprising nanoparticles, wherein the nanoparticles are loaded after polymerization with one or more bioactive agents.

2. The polymer matrix of claim 1, wherein the one or more bioactive agents is selected from the group consisting of: a diagnostic agent, an imaging agent, a contrast agent, a radioactive isotope, and a therapeutic agent.

3. The polymer matrix of claim 1, in which the nanoparticles irreversibly interact with the bioactive agent.

4. The polymer matrix of claim 1, in which the nanoparticles reversibly interact with the bioactive agent.

5. The polymer matrix of claim 1 in which nanoparticles are undecorated metallic nanoparticles.

6. The polymer matrix of claim 1 in which nanoparticles are decorated metallic nanoparticles.

7. The polymer matrix of claim 6 wherein the decorated metallic nanoparticles are decorated with one or more elements selected from the group consisting of: dextran, PEG, streptavidin, biotin, antibodies, antibody fragments, ligands and aptamers.

8. The polymer matrix of claim 1, wherein the nanoparticles are coated with streptavidin.

9. The polymer matrix of claim 1, wherein the bioactive agent is conjugated to biotin.

10-12. (canceled)

13. A method of producing a therapeutic polymer matrix comprising nanoparticles loaded with one or more bioactive agents comprising:
polymerizing a matrix comprising polymers and nanoparticles; and
incubating the nanoparticles and the polymer matrix after polymerization with one or more bioactive agents for a time sufficient to load the nanoparticle and polymer matrix with the agent,
thereby producing a polymer matrix comprising nanoparticles loaded with one or more bioactive agents.

14-15. (canceled)

16. A method of producing a therapeutic polymer matrix in a subject tissue comprising:
administering a nanoparticle and polymer matrix to a subject; and
loading the nanoparticles with a bioactive agent,
thereby producing a therapeutic polymer matrix in a subject tissue.

17-19. (canceled)

20. A method of embolizing polymer microspheres containing streptavidin coated nanoparticles in a patient tumor comprising systemically infusing yttrium 90-y biotin into the patient so that yttrium 90-y biotin accumulates at the site of embolization and interacts with the streptavidin coated nanoparticles.

21-22. (canceled)

23. A method of producing a polymer matrix comprising nanoparticles wherein the nanoparticles are liposomes with an aqueous core comprising:
producing a liposome suspension; and
forcing the liposome suspension through a calibrated porous membrane before introduction of the liposome into the polymer,
thereby producing a polymer matrix comprising nanoparticles wherein the nanoparticles are liposomes with an aqueous core.

24-27. (canceled)

28. A method of treating a subject comprising administering an alginate liposome polymer matrix to a treatment area of the subject, wherein the polymer matrix comprises a potentially radioactive isotope;
exposing the treatment area of the subject to neutrons, protons, electrons, or high energy photons to activate the potentially radioactive isotope; and
providing AMF thermotherapy,
thereby treating the subject.

29. The method of claim 28, wherein the alginate liposome polymer matrix further comprises one or more of a radioisotope, potential radioactive isotope or a chemotherapeutic agent.

30-33. (canceled)

34. A kit comprising the polymer matrix comprising nanoparticles wherein the nanoparticles are loaded after polymerization with one or more bioactive agents, and instructions for use, or
a kit comprising the polymer matrix comprising nanoparticles wherein the nanoparticles are loaded after polymerization with one or more bioactive agents, and instructions for use in embolization, or
a kit comprising the polymer matrix comprising nanoparticles wherein the nanoparticles are loaded after polymerization with one or more bioactive agents, and instructions for therapeutic use, or
a composition comprising a hydrogel and a bioactive agent that forms one or more microspheres.

35-46. (canceled)

47. A method of forming one or more microspheres comprising bioactive agent, the method comprising:
adding bioactive agent to a hydrogel solution;
adding the agent and hydrogel solution to a mineral oil bath; and
initiating polymerization;
thereby forming one or more microspheres comprising bioactive agent.

48-52. (canceled)

53. A method for treating a subject having a vascular or non-vascular condition, the method comprising the step of administering to the subject a composition comprising a hydrogel and a bioactive agent that forms one or more microspheres, or
a method for treating a subject having a vascular or non-vascular hemorrhage, the method comprising the step of administering to the subject a composition comprising a hydrogel and a bioactive agent that forms one or more microspheres, or
a method for treating a subject having a neoplastic growth, the method comprising the step of administering to the subject a composition comprising a hydrogel and a bioactive agent that forms one or more microspheres, thereby treating the subject, or
a method for the selective delivery of a therapeutic agent to a targeted non-occluded vessel by administering to the subject a hydrogel and a bioactive agent that forms one or more microspheres based biomaterial, or
a method for the controlled release of an agent in a subject, the method comprising the steps of: administering to the subject a hydrogel and a bioactive agent that forms one or more microspheres, or
a method for the controlled release of a label in a subject, the method comprising the steps of administering to the subject a hydrogel and a bioactive agent that forms one or more microspheres, or
a method for the controlled release of a contrast agent in a subject, the method comprising the steps of administering to the subject a hydrogel and a bioactive agent that forms one or more microspheres, or
a method comprising administering a cell or therapeutic compound in a hydrogel that forms one or more microspheres to a target area in a patient, or
a method for the selective control of bulking or remodeling in a subject, the method comprising the steps of:
(i) administering to the subject a hydrogel based biomaterial to a targeted area; and
(ii) polymerizing the hydrogel, thereby controlling bulking or remodeling in a subject.

54-105. (canceled)
